(19) United States
(12) Patent Application Publication
(10) Pub. No.: US 2004/0024181 A1
(43) Pub. Date: Feb. 5, 2004

(54) Novel human proteins, polynucleotides encoding them and methods of using the same

(76) Inventors: Esha A. Gangolli, Madison, CT (US); Kimberly A. Spytek, New Haven, CT (US); Jennifer Gilbert, Madison, CT (US); Stacie Casman, North Haven, CT (US); Angela Blalock, Branford, CT (US); Li Li, Branford, CT (US); Corine Vernet, Branford, CT (US); Suresh Shenoy, Branford, CT (US); Vishnu S. Mishra, Gainesville, FL (US); Katarzyna Furtak, Ansonia, CT (US); Valerie L. Gerlach, Branford, CT (US); Shlomit Edinger, New Haven, CT (US); Uriel Malyanker, Branford, CT (US); David Stone, Guilford, CT (US); Isabelle Millet, Milford, CT (US); Glennda Smithson, Guilford, CT (US); Erik Gunther, Branford, CT (US); Karen Ellerman, Branford, CT (US); Muralidhara Padigaru, Branford, CT (US); Raymond J. Taupier JR., East Haven, CT (US); David W. Anderson, Branford, CT (US)

Correspondence Address: MINTZ, LEVIN, COHN, FERRIS,, GLOVSKY AND POPEO, P.C., One Financial Center, Boston, MA, 02111, US

(21) Appl. No.: 10/055,569

(22) Filed: Oct. 26, 2001

Related U.S. Application Data
(60) Provisional application No. 60/243,642, filed on Oct. 26, 2000.
(60) Provisional application No. 60/243,320, filed on Oct. 26, 2000.
(60) Provisional application No. 60/243,592, filed on Oct. 26, 2000.
(60) Provisional application No. 60/243,681, filed on Oct. 27, 2000.
(60) Provisional application No. 60/243,863, filed on Oct. 27, 2000.
(60) Provisional application No. 60/244,443, filed on Oct. 31, 2000.
(60) Provisional application No. 60/245,029, filed on Nov. 1, 2000.
(60) Provisional application No. 60/244,995, filed on Nov. 1, 2000.
(60) Provisional application No. 60/245,293, filed on Nov. 2, 2000.
(60) Provisional application No. 60/245,315, filed on Nov. 2, 2000.
(60) Provisional application No. 60/245,316, filed on Nov. 2, 2000.
(60) Provisional application No. 60/262,994, filed on Jan. 19, 2001.
(60) Provisional application No. 60/269,056, filed on Feb. 15, 2001.
(60) Provisional application No. 60/272,923, filed on Mar. 2, 2001.
(60) Provisional application No. 60/276,565, filed on Mar. 15, 2001.
(60) Provisional application No. 60/318,119, filed on Sep. 7, 2001.

(51) Int. Cl. C07K 14/705; C12P 21/02; C12N 5/06; C07H 21/04

(52) U.S. Cl. 530/350; 536/23.5; 435/69.1; 435/320.1; 435/325

(57) ABSTRACT

Disclosed herein are nucleic acid sequences that encode novel polypeptides. Also disclosed are polypeptides encoded by these nucleic acid sequences, and antibodies, which immunospecifically-bind to the polypeptide, as well as derivatives, variants, mutants, or fragments of the aforementioned polypeptide, polynucleotide, or antibody. The invention further discloses therapeutic, diagnostic and research methods for diagnosis, treatment, and prevention of disorders involving any one of these novel human nucleic acids and proteins.

### RELATED APPLICATIONS

This application claims priority to U.S. S. No. 60/243,642, filed Oct. 26, 2000; U.S. S. No. 60/243,320, filed Oct. 26, 2000; U.S. S. No. 60/243,592, filed Oct. 26, 2000; U.S. S. No. 60/243,681, filed Oct. 27, 2000; U.S. S. No. 60/243,863, filed Oct. 27, 2000; U.S. S. No. 60/244,443, filed Oct. 31, 2000; U.S. S. No. 60/245,029, filed Nov. 1, 2000; U.S. S. No. 60/244,995, filed Nov. 1, 2000; U.S. S. No. 60/245,293, filed Nov. 2, 2000; U.S. S. No. 60/245,315, filed Nov. 2, 2000; U.S. S. No. 60/245,316, filed Nov. 2, 2000; U.S. S. No. 60/262,994, filed Jan. 19, 2001; U.S. S. No. 60/269,056, filed Feb. 15, 2001, U.S. S. No. 60/272,923, filed Mar. 2, 2001, U.S. S. No. 60/276,565, filed Mar. 15, 2001, and U.S. S. No. 60/318,119, filed Sep. 17, 2001 each of which is incorporated by reference in its entirety.

### FIELD OF THE INVENTION

The invention relates to polynucleotides and the polypeptides encoded by such polynucleotides, as well as vectors, host cells, antibodies and recombinant methods for producing the polypeptides and polynucleotides, as well as methods for using the same.

### BACKGROUND OF THE INVENTION

The present invention is based in part on nucleic acids encoding proteins that are new members of the following protein families: Calpain-like, Epsin-like, Low Density Lipoprotein B-like, purinoceptor-like, CG8841-like, Synaptotagmin-like, Serine Protease TLSP-like, Glypican-2 Precursor-like, Mitogen-activated protein kinase kinase-like, Zinc finger protein 276 C2H2 type protein and Thymosin beta10-like. More particularly, the invention relates to nucleic acids encoding novel polypeptides, as well as vectors, host cells, antibodies, and recombinant methods for producing these nucleic acids and polypeptides.

Calpains are intracellular cysteine proteases that are regulated by calcium. They are known to be involved in a number of cellular processes, such as apoptosis, protein processing, cell differentiation, metabolism etc. As such, their role in pathophysiologies extends to—but is not restricted to—tissue remodeling and regeneration (in response to a variety of injury models in the eye, brain, spinal cord, kidney etc.), fertility, tumorigenesis and myopathies. One of the genes identified in susceptibility to type II diabetes is a calpain (calpain-10) (Horikaw a et al., Nat Genet 2602):163-75, 2000). Polymorphisms within this gene are correlated with insulin resistance. Therapies targeting calpain are relevant to disease areas such as cataract, spinal cord injury, Alzheimer's disease, muscular dystrophy, acoustic trauma, diabetes, cancer, learning and memory defects and infertility. Knockout and transgenic models of various calpains also point to a potential role for this family of proteases in a number of cellular and disease processes.

Epsins are a family of proteins that bind to ENTH domain proteins such as Eps15. They are involved in clathrin-mediated endocytosis as well as intracellular protein sorting. Some members of this family undergo phosphorylation during mitosis. In addition, epsins are involved in endocytosis at synapses to compensate for secretion of neuro-transmitter containing vesicles. The interaction of epsin 1 with a transcription factor (promyelocytic leukemia zinc finger protein) has recently been demonstrated, making it likely that the endocytotic machinery can cross-talk with nuclear function. Perturbation of epsin function can lead to defects in the endocytosis of membrane receptors as well as secreted proteins like transferrin, with consequent side-effects. Defects in epsin may potentially lead to aberrant cell-cell signalling, developmental defects, aberrant neurotransmitter signalling etc.

Low density lipoprotein (LDL) particles are the major cholesterol carriers in circulation and their physiological function is to carry cholesterol to the cells. In the process of atherogenesis these particles are modified and they accumulate in the arterial wall. Elevated serum cholesterol bound to low density lipoprotein (LDL) is a characteristic of familial hypercholesterolemia. Individuals with coronary artery disease have a significantly higher mean lipoprotein concentration than those without coronary heart disease, suggesting that lipoprotein measurements may help predict the risk of coronary heart disease in individuals with familial hypercholesterolemia.

Many cells express plasma membrane receptors for extracellular molecules, termed purinoceptors, which appear to be coupled to a plasma membrane pore. Purinoceptors are primitive, widespread and serve many different systems. There are several subclasses of purinoceptors; receptors for adenosine (P 1-purinoceptors) and receptors for ATP (P2-purinoceptors). As for other major transmitters such as acetylcholine, GABA, glutamate and 5-HT, receptors of two major families are activated by ATP, one (the P2X-purinoceptor family) mediates fast responses via ligand-gated ion channels, while the other (the P2Y-purinoceptor family) mediates slower responses via G-proteins.

Synaptotagmins (Syts) are brain-specific Ca2+/phospholipid-binding proteins (Li et.al., Nature 375(6532):594-9, 1995). In hippocampal synapses, Syt I is essential for fast Ca(2+)-dependent synaptic vesicle exocytosis but not for Ca(2+)-independent exocytosis. In vertebrates and invertebrates, Syt may therefore participate in Ca(2+)-dependent synaptic membrane fusion, either by serving as the Ca2+ sensor in the last step of fast Ca(2+)-triggered neurotransmitter release, or by collaborating with an additional Ca2+ sensor. While Syt I binds Ca2+(refs 10, 11), its phospholipid binding is triggered at lower calcium concentrations (EC50=3-6 microM) than those required for exocytosis. Furthermore, Syts bind clathrin-AP2 with high affinity, indicating that they may play a general role in endocytosis rather than being confined to a specialized function in regulated exocytosis. Here we resolve this apparent contradiction by describing four Syts, three of which (Syt VI, VII and VIII) are widely expressed in non-neural tissues. All Syts tested share a common domain structure, with a cytoplasmic region composed of two C2 domains that interacts with clathrin-AP2 (Kd=0.1-1.0 nM) and with neural and non-neural syntaxins. The first C2 domains of Syt I, III, III, V and VII, but not of IV, VI or VIII, bind phospholipids with a similar Ca(2+)-concentration dependence (EC50=3-6 microM). The same C2 domains also bind syntaxin as a function of Ca2+ but the Ca(2+)-concentration dependence of Syt I, II and V (>200 microM) differs from that of Syt III and VII (<10 microM).

Proteolytic enzymes that exploit serine in their catalytic activity are ubiquitous, being found in viruses, bacteria and eukaryotes. They include a wide range of peptidase activity, including exopeptidase, endopeptidase, oligopeptidase and omega-peptidase activity. Over 20 families (denoted S1-S27) of serine protease have been identified, these being grouped into 6 clans (SA, SB, SC, SE, SF and SG) on the basis of structural similarity and other functional evidence. Structures are known for four of the clans (SA, SB, SC and SE): these appear to be totally unrelated, suggesting at least four evolutionary origins of serine peptidases and possibly many more. Notwithstanding their different evolutionary origins, there are similarities in the reaction mechanisms of several peptidases. Chymotrypsin, subtilisin and carboxypeptidase C clans have a catalytic triad of serine, aspartate and histidine in common: senne acts as a nucleophile, aspartate as an electrophile, and histidine as a base. The geometric orientations of the catalytic residues are similar between families, despite different protein folds. The linear arrangements of the catalytic residues commonly reflect clan relationships. For example the catalytic triad in the chymotrypsin clan (SA) is ordered HDS, but is ordered DHS in the subtilisin clan (SB) and SDH in the carboxypeptidase clan (SC).

Glypicans are a family of heparan sulfate proteoglycans that are anchored to the plasma membrane via a glycosylphosphatidylinositol modification. The six glypican genes identified so far show distinct developmental and tissue expression patterns in mice. Glypicans could potentially also be secreted away from the membrane by proteolysis and the soluble protein could potentially act as a dominant-negative inhibitor of the intact protein. This family of proteins has been implicated in neuronal development, guidance and regeneration. It may thus have a role in synaptic plasticity. One of the glypican genes in Drosophila is involved in the wingless and decapentaplegic signaling pathways. Deficiencies in glypican-3 in mice lead to a congenital overgrowth syndrome. In humans, deletions and translocations involving the glypican-3 gene have been associated with an X-linked recessive gigantism syndrome. In addition, the expression of this protein is silenced in an in vitro model of malignant mesothelioma. The novel protein, therefore, may play a role in tissue morphogenesis and patterning, cell division and cell signaling.

Mitogen-activated protein kinase kinase (MAPKK) is a dual-specificity protein kinase which phosphorylates and activates mitogen-activated protein kinase (MAPK). cDNAs encoding two isoforms of MAPKK, MAPKK1 and MAPKK2 (also known as MEK1 and MEK2), have been cloned in mammalian cells (Moriguchi et al., Eur J Biochem 234(1):32-8, 1995). Mitogen-activated protein kinase kinase 1 (MAPKK1) and MAPKK2 function downstream of the proto-oncogene product Raf in signaling pathways that affect cell proliferation and differentiation. The isoforms have been shown to be differentially regulated in two significant ways: MAPKK1, but not MAPKK2, was phosphorylated and inactivated by the cyclin-dependent kinase p34cdc2; and p21 Ras formed a ternary complex with Raf/MAPKKT but not with Raf/MAPKK2 (Mansour et al., Cell Growth Differ 7(2):243-50, 1996). In a study of mouse tissues, MAPKK1 was shown to be highly enriched in the brain while MAPKK2 is present realtively evenly. Both isoforms were shown to reside in the cytoplasm and both are activated in response to nerve growth factor (NGF) and epidermal growth factor (EGF) (Moriguchi et al., Eur J Biochem 234(1):32-8, 1995).

A startling number of cDNA clones encode proteins that contain one or more sequences that match the zinc finger consensus domain, revealing that zinc finger proteins represent perhaps the largest class of DNA binding proteins in eukaryotes and that zinc finger protein-controlled gene expression may be a fundamental aspect of development as well as other processes. Structurally distinct clusters of zinc finger modules define an extremely large superfamily of nucleic acid binding proteins with several hundred, perhaps thousands of different members in vertebrates. C₂H2 type zinc finger proteins (ZFPs) are one of the most complex members of zinc finger modules (Pieler et al., Mol Biol Rep 20(1):1-8, 1994 and Berg et al., Annu Rev Biophys Biophys Chem 19:405-21, 1990).

The beta-thymosins comprise a family of structurally related, highly conserved acidic polypeptides, originally isolated from calf thymus. A number of peptides belong to this family. They include, thymosin beta-4 is a small polypeptide that was first isolated as a thymic hormone and induced terminal deoxynucleotidyltransferase, thymosin beta-9 (and beta-8) in bovine and pig, thymosin beta-10 in man and rat, thymosin beta-11 and beta-12 in trout and human Nb thymosin beta. They found in high quantity in thymus and spleen but are also widely distributed in many tissues. They have been shown to bind to actin monomers and thus to inhibit actin polymerization

Thymosin beta10 is a small conserved acidic protein involved in the inhibition of actin polymerization. Studies have demonstrated that thymosin beta 10 expression is regulated by extracellular signals that stimulate growth of thyroid cells both in vitro and in vivo, and suggest a role for this protein in thyroid diseases characterized by proliferation of follicular cells (10366416). Other studies have demonstrated that thymosin beta-10 is overexpressed in rat thyroid transformed cell lines and in human thyroid carcinoma tissues and cell lines. This evidence suggests that thymosin beta-10 detection may be considered a potential tool for the diagnosis of several human neoplasias (10487837).

### SUMMARY OF THE INVENTION

The invention is based in part upon the discovery of nucleic acid sequences encoding novel polypeptides. The novel nucleic acids and polypeptides are referred to herein as NOVX, or NOV1, NOV2, NOV3, NOV4, NOV5, NOV6, NOV7, NOV8, NOV9, NOV10 and NOV11 nucleic acids and polypeptides. These nucleic acids and polypeptides, as well as derivatives, homnologs, analogs and fragments thereof, will hereinafter be collectively designated as “NOVX” nulcleic acid or polypeptide sequences.

In one aspect, the invention provides an isolated NOVX nucleic acid molecule encoding a NOVX polypeptide that includes a nucleic acid sequence that has identity to the nucleic acids disclosed in SEQ ID NOS:1, 3, 5, 7, 9, 11, 13, 15, 17, 19, 21, 23, 25, 27, 29, 31 and 33. In some embodiments, the NOVX nucleic acid molecule will hybridize under stringent conditions to a nucleic acid sequence complementary to a nucleic acid molecule that includes a protein-coding sequence of a NOVX nucleic acid sequence. The invention also includes an isolated nucleic acid that encodes a NOVX polypeptide, or a fragment, homolog, analog or derivative thereof. For example, the nucleic acid can encode a polypeptide at least 80% identical to a polypeptide comprising the amino acid sequences of SEQ ID NOS:2, 4, 6, 8, 10, 12, 14, 16, 18, 20, 22, 24, 26, 28, 30, 32 and 34. The nucleic acid can be, for example, a genomic DNA fragment or a cDNA molecule that includes the nucleic acid sequence of any of SEQ ID NOS:1, 3, 5, 7, 9, 11, 13, 15, 17, 19, 21, 23, 25, 27, 29, 31 and 33.

Also included in the invention is an oligonucleotide, e.g., an oligonucleotide which includes at least 6 contiguous nucleotides of a NOVX nucleic acid (e.g., SEQ ID NOS:1, 3, 5, 7, 9, 11, 13, 15, 17, 19, 21, 23, 25, 27, 29, 31 and 33) or a complement of said oligonucleotide. Also included in the invention are substantially purified NOVX polypeptides (SEQ ID NOS:2, 4, 6, 8, 10, 12, 14, 16, 18, 20, 22, 24, 26, 28, 30, 32 and 34). In certain embodiments, the NOVX polypeptides include an amino acid sequence that is substantially identical to the amino acid sequence of a human NOVX polypeptide.

The invention also features antibodies that immunoselectively bind to NOVX polypeptides, or fragments, homologs, analogs or derivatives thereof.

In another aspect, the invention includes pharmaceutical compositions that include therapeutically- or prophylactically-effective amounts of a therapeutic and a pharmaceutically-acceptable carrier. The therapeutic can be, e.g., a NOVX nucleic acid, a NOVX polypeptide, or an antibody specific for a NOVX polypeptide. In a further aspect, the invention includes, in one or more containers, a therapeutically- or prophylactically-effective amount of this pharmaceutical composition.

In a further aspect, the invention includes a method of producing a polypeptide by culturing a cell that includes a NOVX nucleic acid, under conditions allowing for expression of the NOVX polypeptide encoded by the DNA. If desired, the NOVX polypeptide can then be recovered.

In another aspect, the invention includes a method of detecting the presence of a NOVX polypeptide in a sample. In the method, a sample is contacted with a compound that selectively binds to the polypeptide under conditions allowing for formation of a complex between the polypeptide and the compound. The complex is detected, if present, thereby identifying the NOVX polypeptide within the sample.

The invention also includes methods to identify specific cell or tissue types based on their expression of a NOVX.

Also included in the invention is a method of detecting the presence of a NOVX nucleic acid molecule in a sample by contacting the sample with a NOVX nucleic acid probe or primer, and detecting whether the nucleic acid probe or primer bound to a NOVX nucleic acid molecule in the sample.

In a further aspect, the invention provides a method for modulating the activity of a NOVX polypeptide by contacting a cell sample that includes the NOVX polypeptide with a compound that binds to the NOVX polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

Also within the scope of the invention is the use of a therapeutic in the manufacture of a medicament for treating or preventing disorders or syndromes including, e.g., Von Hippel-Lindau (VHL) syndrome, cirrhosis, transplantation disorders, pancreatitis, obesity, diabetes, autoimmune disease, renal artery stenosis, interstitial nephritis, glomerulonephritis, polycystic kidney disease, systemic lupus erythematosus, renal tubular acidosis, IgA nephropathy, hypercalcemia, Lesch-Nyhan syndrome, developmental defects, cataract, spinal cord injury, Alzheimer's disease, muscular dystrophy, acoustic trauma, cancer, learning and memory defects, infertility, cardiomyopathies, atherosclerosis, hypertension, congenital heart defects, aortic stenosis, atrial septal defect, atrioventricular canal defect, ductus arteriosus, pulmonary stenosis, subaortic stenosis, ventricular septal defect, valve diseases, tuberous sclerosis, scleroderma, endometriosis, hemophilia, hypercoagulation, idiopathic thrombocytopenic purpura, immunodeficiencies, graft versus host disease, dementia, stroke, Parkinson's disease, Huntington's disease, cerebral palsy, epilepsy, multiple sclerosis, ataxia-telangiectasia, leukodystrophies, behavioral disorders, addiction, anxiety, pain, neurodegeneration, Familial hypercholesterolemia, hyperlipoproteinemia II phenotype, tendinous xanthomas, corneal arcus, coronary artery disease, planar xanthomas, webbed digits, hypercholesterolemia, fertility, xanthomatosis, Hepatitis C infection, regulation, synthesis, transport, recycling, or turnover of LDL receptors, Cerebral arteriopathy with subcortical infarcts and leukoencephalopathy, Epiphyseal dysplasia, multiple 1, Ichthyosis, nonlamellar and nonerythrodermic, congenital, Leukemia, T-cell acute lymphoblastoid, Pseudoachondroplasia, SCID, autosomal recessive, T-negative/B-positive type, C3 deficiency, Diabetes mellitus, insulin-resistant, with acanthosis nigricans, Glutaricaciduria, type I, Hypothyroidism, congenital, Leprechaunism, Liposarcoma, Mucolipidosis IV, Persistent Mullerian duct syndrome, type I, Rabson-Mendenhall syndrome, Thyroid carcinoma, nonmedullary, with cell oxyphilia, Erythrocytosis, familial, Malaria, cerebral, susceptibility to, Bleeding disorder due to defective thromboxane A2 receptor, Cerebellar ataxia, Cayman type, Convulsions, familial febrile, 2, Cyclic hematopoiesis, Fucosyltransferase-6 deficiency, GAMT deficiency, Cirrhosis, Psoriasis, Actinic keratosis, Tuberous sclerosis, Acne, Hair growth, allopecia, pigmentation disorders, endocrine disorders, trauma, immunological disease, respiratory disease, gastro-intestinal diseases, reproductive health, neurological diseases, bone marrow transplantation, metabolic and endocrine diseases, allergy and inflammation, nephrological disorders, hematopoietic disorders, urinary system disorders, Atopy; Osteoporosis-pseudoglioma syndrome; Smith-Lemli-Opitz syndrome, type I; Smith-Lemli-Opitz syndrome, type II; Xeroderma pigmentosum, group E, subtype 2; Asthma, atopic, susceptibility to; Diabetes mellitus, insulin-dependent, 4; Susceptibility to IDDM; Angioedema, hereditary; Paraganglioma, familial nonchromaffin, 2; neuroprotection; Lambert-Eaton myasthenic syndrome, digestive system disorders, all or some of the protease/protease inhibitor deficiency disorders, diabetes mellitus non-insulin dependent, Acyl-CoA dehydrogenase, deficiency of long chain, Brachydactyly, type A1, Carbamoylphosphate synthetase I deficiency, Cardiomyopathy dilated II, Cataract Coppock-like, Cataract crystalline aculeiform, Cataract polymorphic congenital, Cataract variable zonular pulverulent, Cataracts punctate progressive juvenile-onse, Choreoathetosis familial paroxysmal, Craniofacial-deafness-hand syndrome, Ichthyosis lamellar, type 2, Myopathy, desmin-related cardioskeletal, Resistance/susceptibility to TB, Rhabdomyosarcoma alveolar, Waardenburg syndrome type I and type III, Alport syndrome autosomal recessive, Bjornstad syndrome, Hematuria, familial benign, Hyperoxaluria primary, type 1, Syndactyly type 1, Hyperproglucagonemia, Bethlem myopathy, Brachydactyly type E, Brachydactyly-mental retardation syndrome, Finnish lethal neonatal metabolic syndrome, susceptibility to 2, Simpson-Golabi-Behmel syndrome, type I and type 2, Beckwith-Wiedemann syndrome, pathogen infections, heart disease, prostate cancer, angiogenesis and wound healing, modulation of apoptosis, neuropsychiatric disorders, age-related disorders, pathological disorders involving spleen, thymus, lung, and peritoneal macrophages and/or other pathologies and disorders of the like.

The therapeutic can be, e.g., a NOVX nucleic acid, a NOVX polypeptide, or a NOVX-specific antibody, or biologically-active derivatives or fragments thereof.

For example, the compositions of the present invention will have efficacy for treatment of patients suffering from the diseases and disorders disclosed above and/or other pathologies and disorders of the like. The polypeptides can be used as immunogens to produce antibodies specific for the invention, and as vaccines. They can also be used to screen for potential agonist and antagonist compounds. For example, a cDNA encoding NOVX may be useful in gene therapy, and NOVX may be useful when administered to a subject in need thereof. By way of non-limiting example, the compositions of the present invention will have efficacy for treatment of patients suffering from the diseases and disorders disclosed above and/or other pathologies and disorders of the like.

The invention further includes a method for screening for a modulator of disorders or syndromes including, e.g., the diseases and disorders disclosed above and/or other pathologies and disorders of the like. The method includes contacting a test compound with a NOVX polypeptide and determining if the test compound binds to said NOVX polypeptide. Binding of the test compound to the NOVX polypeptide indicates the test compound is a modulator of activity, or of latency or predisposition to the aforementioned disorders or syndromes.

Also within the scope of the invention is a method for screening for a modulator of activity, or of latency or predisposition to disorders or syndromes including, e.g., the diseases and disorders disclosed above and/or other pathologies and disorders of the like by administering a test compound to a test animal at increased risk for the aforementioned disorders or syndromes. The test animal expresses a recombinant polypeptide encoded by a NOVX nucleic acid. Expression or activity of NOVX polypeptide is then measured in the test animal, as is expression or activity of the protein in a control animal which recombinantly-expresses NOVX polypeptide and is not at increased risk for the disorder or syndrome. Next, the expression of NOVX polypeptide in both the test animal and the control animal is compared. A change in the activity of NOVX polypeptide in the test animal relative to the control animal indicates the test compound is a modulator of latency of the disorder or syndrome.

In yet another aspect, the invention includes a method for determining the presence of or predisposition to a disease associated with altered levels of a NOVX polypeptide, a NOVX nucleic acid, or both, in a subject (e.g., a human subject). The method includes measuring the amount of the NOVX polypeptide in a test sample from the subject and comparing the amount of the polypeptide in the test sample to the amount of the NOVX polypeptide present in a control sample. An alteration in the level of the NOVX polypeptide in the test sample as compared to the control sample indicates the presence of or predisposition to a disease in the subject. Preferably, the predisposition includes, e.g., the diseases and disorders disclosed above and/or other pathologies and disorders of the like. Also, the expression levels of the new polypeptides of the invention can be used in a method to screen for various cancers as well as to determine the stage of cancers.

In a further aspect, the invention includes a method of treating or preventing a pathological condition associated with a disorder in a mammal by administering to the subject a NOVX polypeptide, a NOVX nucleic acid, or a NOVX-specific antibody to a subject (e.g., a human subject), in an amount sufficient to alleviate or prevent the pathological condition. In preferred embodiments, the disorder, includes, e.g., the diseases and disorders disclosed above and/or other pathologies and disorders of the like.

In yet another aspect, the invention can be used in a method to identity the cellular receptors and downstream effectors of the invention by any one of a number of techniques commonly employed in the art. These include but are not limited to the two-hybrid system, affinity purification, co-precipitation with antibodies or other specific-interacting molecules.

NOVX nucleic acids and polypeptides are further useful in the generation of antibodies that bind immuno-specifically to the novel NOVX substances for use in therapeutic or diagnostic methods. These NOVX antibodies may be generated according to methods known in the art, using prediction from hydrophobicity charts, as described in the “Anti-NOVX Antibodies” section below. The disclosed NOVX proteins have multiple hydrophilic regions, each of which can be used as an immunogen. These NOVX proteins can be used in assay systems for functional analysis of various human disorders, which will help in understanding of pathology of the disease and development of new drug targets for various disorders.

The NOVX nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The present invention provides novel nucleotides and polypeptides encoded thereby. Included in the invention are the novel nucleic acid sequences and their encoded polypeptides. The sequences are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table A provides a summary of the NOVX nucleic acids and their encoded polypeptides.

TABLE A
Sequences and Corresponding SEQ ID Numbers

| | | SEQ ID | | |
|---|---|---|---|---|
| | | NO | | |
| NOVX | | (nucleic | SEQ ID NO | |
| Assignment | Internal Identiflcation | acid) | (polypeptide) | Homology |
| 1 | 3352274 | 1 | 2 | Calpain-like |
| 2 | 21421174 | 3 | 4 | Epsin-like |
| 3 | AC025263_da1 | 5 | 6 | Low Density Lipoprotein B-like |
| 4 | AC026756_da1 | 7 | 8 | Purinoceptor |
| 5a | sggc_draft_dj895c5_ | 9 | 10 | CG8841-like |
| | 20000811_da1 | | | |
| 5b | CG54443-02 | 11 | 12 | CG8841-like |
| 6a | SC134912642_da1 | 13 | 14 | Synaptotagmin-like |
| 6b | CG56106-01 | 15 | 16 | Synaptotagmin-like |
| 7 | wugc_draft_h_nh0781m | 17 | 18 | Serine Protease TLSP-like |
| | 21_20000809_da1 | | | |
| 8a | 134913441_EXT | 19 | 20 | Glypican-2 Precursor-like |
| 8b | CG50970-02 | 21 | 22 | Glypican-2 Precursor-like |
| 8c | CG50970-03 | 23 | 24 | Glypican-2 Precursor-like |
| 8d | CG50970-04 | 25 | 26 | Glypican-2 Precursor-like |
| 9 | AC011005_da2/1399435 | 27 | 28 | Mitogen-activated protein kinase |
| | 78 | | | kinase 2-like |
| 10 | sggc_draft_c333e1_ | 29 | 30 | Zinc Finger Protein 276 C2H2-type |
| | 20000804_da2 | | | |
| 11a | GMAC079400_A | 31 | 32 | Thymosin beta 10-like |
| 11b | CG109754-01 | 33 | 34 | Thymosin beta 10-like |

NOVX nucleic acids and their encoded polypeptides are useful in a variety of applications and contexts. The various NOVX nucleic acids and polypeptides according to the invention are useful as novel members of the protein families according to the presence of domain and sequence relatedness to previously described proteins. Additionally, NOVX nucleic acids and polypeptides can also be used to identify proteins that are members of the family to which the NOVX polypeptides belong.

NOV1 is homologous to a Calpain-like family of proteins. Thus, the NOV1 nucleic acids, polypeptides, antibodies and related compounds according to the invention will be useful in therapeutic and diagnostic applications implicated in, for example; Von Hippel-Lindua (VHL) syndrome, obesity, diabetes, autoimmune disease, systemic lupus erythematosus, Lesch-Nyhan syndrome, developmental defects, Alzheimer's disease, muscular dystrophy, acoustic trauma, cancer, learning and memory defects, infertility and/or other pathologies/disorders.

NOV2 is homologous to a Espin-like family of proteins. Thus NOV2 nucleic acids, polypeptides, antibodies and related compounds according to the invention will be useful in therapeutic and diagnostic applications implicated in, for example; cardiomyopathies, atherosclerosis, hypertension, congenital heart defects, obesity, infertility, cancer, autoimmune diseases, allergies, developmental defects, dementia, Von Hippel-Lindau (VHL) syndrome, Alzheimer's disease, stroke, Parkinson's disease, Huntington's disease, cerebral palsy, epilepsy, Lesch-Nyhan syndrome, multiple sclerosis, leukodystrophies, neurodegeneration and/or other pathologies/disorders.

NOV3 is homologous to a family of Low Density Lipoprotein B-like proteins. Thus, the NOV3 nucleic acids and polypeptides, antibodies and related compounds according to the invention will be useful in therapeutic and diagnostic applications implicated in, for example: Familial hypercholesterolemia, coronary artery disease, diabeties, atherosclerosis, Hepatitis C infection, Thyroid caizinoma, Von Hippel-Lindau (VHL) syndrome, Cirrhosis, Transplantation, Psoriasis, Actinic keratosis, Tuberous sclerosis, Acne, Hair growth, allopecia, pigmentation disorders, endocrine disorders and/or other pathologies/disorders.

NOV4 is homologous to the Purinoceptor-like family of proteins. Thus, NOV4 nucleic acids, polypeptides, antibodies and related compounds according to the invention will be useful in therapeutic and diagnostic applications implicated in various disease, pathologies and disorders.

NOV5 is homologous to the CG8841-like protein family. Thus NOV5 nucleic acids, polypeptides, antibodies and related compounds according to the invention will be useful in therapeutic and diagnostic applications implicated in, for example: cancer, trauma, immunological disease, respiratory disease, gastro-intestinal diseases, reproductive health, neurological and neurodegenerative diseases, bone marrow transplantation, metabolic and endocrine diseases, allergy and inflammation, nephrological disorders, hematopoietic disorders, urinary system disorders and/or other pathologies/disorders.

NOV6 is homologous to the Synaptotagmin-like family of proteins. Thus NOV6 nucleic acids, polypeptides, antibodies and related compounds according to the invention will be useful in therapeutic and diagnostic applications implicated in, for example: Atopy; Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection; metabolic disorders, Lambert-Eaton myasthenic syndrome and/or other pathologies/disorders.

NOV7 is homologous to members of the Serine Protease TLSP-like family of proteins. Thus, the NOV7 nucleic acids, polypeptides, antibodies and related compounds according to the invention will be useful in therapeutic and diagnostic applications implicated in, for example; cancer, neurological disorders, digestive system disorders, all or some of the protease/protease inhibitor deficiency disorders and/or other pathologies/disorders.

NOV8 is homologous to the Glypican-2 Precursor-like family of proteins. Thus, NOV8 nucleic acids and polypeptides, antibodies and related compounds according to the invention will be useful in therapeutic and diagnostic applications implicated in, for example; diabetes, diabetes mellitus non-insulin dependent, autoimmune disease, systemic lupus erythematosus, Von Hippel-Lindau (VHL) syndrome, Alzheimer's disease, stroke, Parkinson's disease, Huntington's disease, cerebral palsy, epilepsy, multiple sclerosis, ataxia-telangiectasia, leukodystrophies, neurodegeneration, cancer, Cardiomyopathy, various cataract disorders Waardenburg syndrome type I and type III, Bjornstad syndrome, Simpson-Golabi-Behmel syndrome, type 1 and type 2, Beckwith-Wiedemann syndrome and/or other pathologies/disorders.

NOV9 is homologous to members of the Mitogen Activated Protein Kinase Kinase 2-like family of proteins. Thus, the NOV9 nucleic acids, polypeptides, antibodies and related compounds according to the invention will be useful in therapeutic and diagnostic applications implicated in, for example; atherosclerosis, metabolic diseases, pathogen infections, neurological diseases and/or other pathologies/disorders.

NOV10 is homologous to members of the Zinc Finger Protein 276 C₂H2 type family of proteins. Thus, the NOV10 nucleic acids, polypeptides, antibodies and related compounds according to the invention will be useful in therapeutic and diagnostic applications implicated in, for example; cancer, trauma, immunological disease, respiratory disease, heart disease, gastro-intestinal diseases, reproductive health, neurological and neurodegenerative diseases, bone marrow transplantation, metabolic and endocrine diseases, allergy and inflammation, nephrological disorders, hematopoietic disorders, urinary system disorders and/or other pathologies/disorders.

NOV11 is homologous to members of the Thymosin beta 10-like family of proteins. Thus, the NOV11 nucleic acids, polypeptides, antibodies and related compounds according to the invention will be useful in therapeutic and diagnostic applications implicated in, for example; prostate cancer, immunological and autoimmune disorders (ie hyperthyroidism), angiogenesis and wound healing, modulation of apoptosis, neurodegenerative and neuropsychiatric disorders, age-related disorders, pathological disorders involving spleen, thymus, lung, and peritoneal macrophages and/or other pathologies/disorders. The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit, e.g., neurogenesis, cell differentiation, cell proliferation, hematopoiesis, wound healing and angiogenesis. Additional utilities for the NOVX nucleic acids and polypeptides according to the invention are disclosed herein.

NOV1

A disclosed NOV1 nucleic acid of 1947 nucleotides (also referred to as 3352274) 15 encoding a novel Calpain-like protein is shown in Table 1A. An open reading frame was identified beginning with an ATG initiation codon at nucleotides 1-3 and ending with a TAG codon at nucleotides 1945-1947. The start and stop codons are in bold letters in Table 1A.

TABLE 1A
NOV1 Nucleotide Sequence.

(SEQ ID NO:1)

ATGGCATCCAGCAGTGGGAGGGTCACCATCCAGCTCGTGGATGAGGAGGCTGGGGTCGGAGCCGGGCGCCTG
CAGCTTTTTCGGGGCCAGAGCTATGAGGCAATTCGGGCAGCCTGCCTGGATTCGGGGATCCTGTTCCGCGAC
CCTTACTTCCCTGCTGGCCCTGATGCCCTTGGCTATGACCAGCTGGGGCCGGACTCGGAGAAGGCCAAAGGC
GTGAAATGGATGAGGCCACAGGAGTTCTGTGCTGAGCCGAAGTTCATCTGTGAAGACATGAGCCGCACAGAC
GTGTGTCAGGGGAGCCTGGGTAACTGCTGGTTCCTTGCAGCTGCCGCCTCCCTTACTCTGTATCCCCGGCTC
CTGCGCCGGGTGGTCCCTCCTGGACAGGATTTCCAGCATGGCTACGCAGGCGTCTTCCACTTCCAGCTCTGG
CAGTTTGGCCGCTGGATGGACGTCGTGGTGGATGACAGGCTGCCCGTGCGTGAGGGGAAGCTGATGTTCGTG
CGCTCGGAACAGCGGAATGAGTTCTGGGCCCCACTCCTGGAGAAGGCCTACGCCAAGCTCCACGGCTCCTAT
GAGGTGATGCGGGGCGGCCACATGAATGAGGCTTTTGTGGATTTCACAGGCGGCGTGGGCGAGGTGCTCTAT
CTGAGACAAAACAGCATGGGGCTGTTCTCTGCCCTGCGCCATGCCCTGGCCAAGGAGTCCCTCGTGGGCGCC
ACTGCCCTGAGTGATCGGGGTGAGTACCGCACAGAAGAGGGCCTGGTAAAGGGACACGCGTATTCCATCACG
GGCACACACAAGGTAAGTCTGGGCTTCACCAAGGTGCGGCTGCTGCGGCTGCGGAACCCATGGGGCTGCGTG
GAGTGGACGGGGGCCTGGAGCGACAGCTGCCCACGCTGGGACACACTCCCCACCGAGTGCCGCGATGCCCTG
CTGGTGAAAAAGGAGGATGGCGAGTTCTGGATGGAGCTGCGGGACTTCCTCCTCCATTTCGACACCGTGCAG
ATCTGCTCGCTGAGCCCGGAGGTGCTGGGCCCCAGCCCGGAGGGGGGCGGCTGGCACGTCCACACCTTCCAA
GGCCGCTGGGTGCGTGGCTTCAACTCCGGCGGGAGCCAGCCTAATGCTGAAACCTTCTGGACCAATCCTCAG
TTCCGTTTAACGCTGCTGGAGCCTGATGAGGAGGATGACGAGGATGAGGAAGGGCCCTGGGGGGGCTGGGGG
GCTGCAGGGGCACGGGGCCCAGCGCGGGGGGGCCGCACGCCCAAGTGCACGGTCCTTCTGTCCCTCATCCAG
CGCAACCGGCGGCGCCTGAGAGCCAAGGGCCTCACTTACCTCACCGTTGGCTTCCACGTGTTCCAGGTGGAG
ATCGACGACGTGATCAGCGCAGACCTGCAGTCTCTCCAGGGCCCCTACCTGCCCCTGGAGCTGGGGTTGGAG
CAGCTGTTTCAGGAGCTGGCTGGAGAGGAGGAAGAACTCAATGCCTCTCAGCTCCAGGCCTTACTAAGCATT
GCCCTGGAGCCTGCCAGGGCCCATACCTCCACCCCCAGAGAGATCGGGCTCAGGACCTGTGAGCAGCTGCTG
CAGTGTTTCGGGGGGCAAAGCCTGGCCTTACACCACTTCCAGCAGCTCTGGGGCTACCTCCTGGAGTGGCAG
GCCATATTTAACAAGTTCGATGAGGACACCTCTGGAACCATGAACTCCTACGAGCTGAGGCTGGCACTGAAT
GCAGCAGGTTTCCACCTGAACAACCAGCTGACCCAGACCCTCACCAGCCGCTACCGGGATAGCCGTCTGCGT
GTGGACTTCGAGCGGTTCGTGTCCTGTGTGGCCCACCTCACCTGCATCTTCCACTGCAGCCAGCACCTGGAT
GGGGGTGAGGGGGTCATCTGCCTGACCCACAGACAGGTGAGCCAGGTGTGGATGGAGGTGGCCACCTTCTCC
TAG

The NOV1 nucleic acid sequence maps to chromosome 19 and has 430 of 631 bases (68%) identical to a Gallus gallus calcium protease mRNA (gb:GENBANK-ID:GGCPROT|acc:X01415) (E=1.4e⁻⁹⁰). Similiarity information was assessed using public nucleotide databases including all GenBank databases and the GeneSeq patent database. Chromosome information was assigned using OMIM and the electronic northern tool from Curatools to derive the the chromosomal mapping of the SeqCalling assemblies, Genomic clones, and/or EST sequences that were included in the invention.

In all BLAST alignments herein, the “E-value” or “Expect” value is a numeric indication of the probability that the aligned sequences could have achieved their similarity to the BLAST query sequence by chance alone, within the database that was searched. For example, the probability that the subject (“Sbjct”) retrieved from the NOV1 BLAST analysis, e.g., Gallus gallus calcium protease mRNA, matched the Query NOV1 sequence purely by chance is 1.4e⁻⁹⁰. The Expect value (E) is a parameter that describes the number of hits one can “expect” to see just by chance when searching a database of a particular size. It decreases exponentially with the Score (S) that is assigned to a match between two sequences. Essentially, the E value describes the random background noise that exists for matches between sequences.

The Expect value is used as a convenient way to create a significance threshold for reporting results. The default value used for blasting is typically set to 0.0001. In BLAST 2.0, the Expect value is also used instead of the P value (probability) to report the significance of matches. For example, an E value of one assigned to a hit can be interpreted as meaning that in a database of the current size one might expect to see one match with a similar score simply by chance. An E value of zero means that one would not expect to see any matches with a similar score simply by chance. See, e.g., http://www.ncbi.nlm.nih.gov/Education/BLASTinfo/. Occasionally, a string of X's or N's will result from a BLAST search. This is a result of automatic filtering of the query for low-complexity sequence that is performed to prevent artifactual hits. The filter substitutes any low-complexity sequence that it finds with the letter “N” in nucleotide sequence (e.g., “NNNNNNNN”) or the letter “X” in protein sequences (e.g., “XXX”). Low-complexity regions can result in high scores that reflect compositional bias rather than significant position-by-position alignment. Wootton and Federhen, Methods Enzymol 266:554-571, 1996.

The disclosed NOV1 polypeptide (SEQ ID NO:2) encoded by SEQ ID NO:1 has 648 amino acid residues and is presented in Table 1B using the one-letter amino acid code. Signal P, Psort and/or Hydropathy results predict that NOV1 does not contain a signal peptide and is likely to be localized in the cytoplasm with a certainty of 0.7480.

TABLE 1B
Encoded NOV1 protein sequence.

(SEQ ID NO:2)

MASSSGRVTIQLVDEEAGVGAGRLQLFRGQSYEAIRAACLDSGILFRDPYFPAGPDALGYDQLGPDSEKAKG
VKWMRPQEFCAEPKFICEDMSRTDVCQGSLGNCWFLAAAASLTLYPRLLRRVVPPGQDFQHGYAGVFHFQLW
QFGRWMDVVVDDRLPVREGKLMFVRSEQRNEFWAPLLEKAYAKLHGSYEVMRGGHMNEAFVDFTGGVGEVLY
LRQNSMGLFSALRHALAKESLVGATALSDRGEYRTEEGLVKGHAYSITGTHKVSLGFTKVRLLRLRNPWGCV
EWTGAWSDSCPRWDTLPTECRDALLVKKEDGEFWMELRDFLLHFDTVQICSLSPEVLGPSPEGGGWHVHTFQ
GRWVRGFNSGGSQPNAETFWTNPQFRLTLLEPDEEDDEDEEGPWGGWGAAGARGPARGGRTPKCTVLLSLIQ
RNRRRLRAKGLTYLTVGFHVFQVEIDDVISADLQSLQGPYLPLELGLEQLFQELAGEEEELNASQLQALLSI
ALEPARAHTSTPREIGLRTCEQLLQCFGGQSLALHHFQQLWGYLLEWQAIFNKFDEDTSGTMNSYELRLALN
AAGFHLNNQLTQTLTSRYRDSRLRVDFERFVSCVAHLTCIFHCSQHLDGGEGVICLTHRQVSQVWMEVATFS

The NOV1 amino acid sequence has 405 of 456 amino acid residues (88%) identical to, and 429 of 456 amino acid residues (94%) similar to, a Mus musculus 720 amino acid residue protein (ptnr:TREMBLNEW-ACC:CAC10066) (E=4.1e⁻³¹¹).

NOV1 is expressed in at least the following tissues: Placenta, whole organism, kidney, liver, pancreas, small intestine. This information was derived by determining the tissue sources of the sequences that were included in the invention.

The disclosed NOV1 polypeptide has homology to the amino acid sequences shown in the BLASTP data listed in Table 1C.

TABLE 1C
BLAST results for NOV1

| Gene Index/ | | Length | Identity | Positives | |
|---|---|---|---|---|---|
| Identifier | Protein/Organism | (aa) | (%) | (%) | Expect |
| gi\|10303329\|emb\|CAC | calpain 12 [Mus | 720 | 404/456 | 429/456 | 0.0 |
| 10066.1\| (AJ289241) | musculus] | | (88%) | (93%) | |
| gi\|10303331\|emb\|CAC | calpain 12 [Mus | 462 | 404/456 | 429/456 | 0.0 |
| 10068.1\| (AJ289241) | musculus] | | (88%) | (93%) | |
| gi\|10303330\|emb\|CAC | calpain 12 [Mus | 502 | 404/456 | 429/456 | 0.0 |
| 10067.1\| (AJ289241) | musculus] | | (88%) | (93%) | |
| gi\|11230800\|ref\|NP_ | calpain 12 [Mus | 449 | 300/342 | 320/342 | 1e−166 |
| 068694.1\| | musculus] | | (87%) | (92%) | |
| gi\|5901916\|ref\|NP_008989.1\| | calpain 11 [Homo | 702 | 274/706 | 380/706 | 1e−125 |
| | sapiens] | | (38%) | (53%) | |

The homology between these and other sequences is shown graphically in the ClustalW analysis shown in Table 1D. In the ClustalW alignment of the NOV1 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

The presence of identifiable domains in NOV1, as well as all other NOVX proteins, was determined by searches using software algorithms such as PROSITE, DOMAIN, Blocks, Pfam, ProDomain, and Prints, and then determining the Interpro number by crossing the domain match (or numbers) using the Interpro website (http:www.ebi.ac.ukl/interpro). DOMAIN results for NOV1, as disclosed in Tables 1E and 1F, were collected from the Conserved Domain Database (CDD) with Reverse Position Specific BLAST analyses. This BLAST analysis software samples domains found in the Smart and Pfam collections. For Tables1E, 1F and all successive DOMAIN sequence alignments, fully conserved single residues are indicated by black shading or by the sign (|) and “strong” semi-conserved residues are indicated by grey shading or by the sign (+). The “strong” group of conserved amino acid residues may be any one of the following groups of amino acids: STA, NEQK, NHQK, NDEQ, QHRK, MILV, MILF, HY, FYW.

Tables 1E and 1F lists the domain description from DOMAIN analysis results against NOV1. This indicates that the NOV1 sequence has properties similar to those of other proteins known to contain these domains.

TABLE 1E
Domain Analysis of NOV1

gnl|Pfam|pfam00648, Peptidase_C2, Calpain family cysceine protease.
(SEQ ID NO:89)
Length = 298 residues, 100.0% aligned
Score = 343 bits (881), Expect = 1e−95

| | | | |
|---|---|---|---|
| NOV1 | 45 | LFRDPYFPAGPDALGYDQLGPDSEKAKGVKWMRPQEFCAEPKFICEDMSRTDVCQGSLGN | 104 |
| | | \|\| \|\| \|\|\| \| +\|\|\| \|\|\| +\|++\| \|\| \| \|+\|\| +\|\|\|+\|\|\|+\|\|+ | |
| 00648 | 1 | LFVDPSFPAAPKSLGYKPLGP-----RGIEWKRPHEINENPQFIVGGATRTDICQGALGD | 55 |
| NOV1 | 105 | CWFLAAAASLTLYPRLLRRVVPPGQDFQHGYAGVFHFQLWQFGRWMDVVVDDRLPVREGK | 164 |
| | | \|\| \|\|\| \|\|\|\|\| \|\| \|\|\|\| \| \|\| \|\|\|+\|\|\|+ \|\|\|\| \|+\|\|\|\|\|\| \|\| ++\|\| | |
| 00648 | 56 | CWLLAALASLTLNEPLLLRVVPHDQSFQENYAGIFHFRFWQFGEWVDVVVDDLLPTKDGK | 115 |
| NOV1 | 165 | LMFVRSEQRNEFWAPLLEKAYAKLHGSYEVMRGGHMNEAFVDFTGGVGEVLYLRQNS--- | 221 |
| | | \|+\|\| \| +\|\|\|\|\|+ \|\|\|\|\|\|\|\|\|+\| \|\| + \|\| \|\| \| \|\|\|\| \| \|+ | |
| 00648 | 116 | LLFVHSAERNEFWSALLEKAYAKLNGCYEALSGGSTTEALEDLTGGVCESYELKLAPSSM | 175 |
| NOV1 | 222 | MGLFSALRHALAKESLVGA---TALSDRGEYRTEEGLVKGHAYSITGTHKVSLGFTKVRL | 278 |
| | | + \| + ++ \| + \|\|+\| \| \| +\|\|\|\|\|\|\|\|\|+\|\| +\|+ \|+\| | |
| 00648 | 176 | LNLGNIIKKMLERGSLLGCSIDITSPVDMEARMAKGLVKGHAYSVTGVKEVNYRGEGVKL | 235 |
| NOV1 | 279 | LRLRNPWGCVEWTGAWSDSCPRWDTLPTECRDALLVKKEDGEFWMELRDFLLHFDTVQIC | 338 |
| | | +\|\|\|\|\|\|\| \|\|\|\|\| \|\|\|\| \| \|+ + + + \| +\| \|\|\|\|\|\|\| \|\|\| \|\| ++\|\| | |
| 00648 | 236 | IRLRNPWGQVEWTGDWSDSSPDWNIVDPDEKARLQLKFEDGEFWMSFEDFLRHFSRLEIC | 295 |
| NOV1 | 339 | SLS | 341 |
| | | +\|+ | |
| 00648 | 296 | NLT | 298 |

TABLE 1F
Domain Analysis of NOV1

gnl|Smart|smart00230, CysPc, Calpain-like thiol protease family.;
Calpain-like thiol protease family (peptidase family C2). Calcium
activated neutral protease (large subunit). (SEQ ID NO:90)
Length = 323 residues, 99.1% aligned
Score = 342 bits (877), Expect = 4e−95

| | | | |
|---|---|---|---|
| NOV1 | 27 | FRGQSYEAIRAACLDSGILFRDPYFPAGPDALGYDQLGPDSEKAKGVKWMRPQEFCAEPK | 86 |
| | | \| \| \|\| +\| \|\|+ \| \|\| \|\| \|\|\| \| +\| + \|\| \| \| \|\| \| +\| | |
| 00230 | 1 | FENQDYEELRQECLEEGGLFVDPLFPAKPSSLFFSQLQRK-----FVVWKRPHEIFEDPP | 55 |
| NOV1 | 87 | FICEDMSRTDVCQGSLGNCWFLAAAASLTLYPRLLRRVVPPGQDFQHGYAGVFHFQLWQF | 146 |
| | | \| \|\|\|\|+\|\|\| \|\|+\|\| \|\|\| \|+\|\|\| \|\| \|\|+\| \|+\| \|\|\|++\|\|+ \|++ | |
| 00230 | 56 | LIVGGASRTDICQGVLGDCWLLAALAALTLREELLARVIPKDQEFSENYAGIYHFRFWRY | 115 |
| NOV1 | 147 | GRWMDVVVDDRLPVREGKLMFVRSEQRNEFWAPLLEKAYAKLHGSYEVMRGGHMNEAFVD | 206 |
| | | \|+\|+\|\|\|+\|\|\|\|\| \| \|+\|+ \| \|\|\|\|\|+ \|\|\|\|\|\|\|\|\| \| \|\| ++\|\| \|\| \| | |
| 00230 | 116 | GKWVDVVIDDRLPTYNGDLLFMHSNSRNEFWSALLEKAYAKLRGCYEALKGGSTTEALED | 175 |
| NOV1 | 207 | FTGGVGEVLYLRQNSMG---LFSALRHALAKESLVGATALSDRG---EYRTEEGLVKGHA | 260 |
| | | \|\|\|\| \| + \|++ \| \|\| \|+ \| + \|\|+\| + + \| + \|\|\|\|\|\|\| | |
| 00230 | 176 | LTGGVAESIELKKISKDPDELFKDLKKAFERGSLMGCSIGAGTAVEEEEQKRNGLVKGHA | 235 |
| NOV1 | 261 | YSITGTHKVSLGFTKVRLLRLRNPWGCVEWTGAWSDSCPRWDTLPTECRDAL-LVKKEDG | 319 |
| | | \|\|+\| +\| + +\|\|\|\|\|\|\|\|\| \|\| \| \|\|\| \| \| ++ \| + \| \| +\|\| | |
| 00230 | 236 | YSVTDVREVDGR-RRQKLLRLRNPWGESEWNGPWSDDSPEWRSVSAEEKKNLGLTMDDDG | 294 |
| NOV1 | 320 | EFWMELRDFLLHFDTVQICSLSPEVL | 345 |
| | | \|\|\|\| \|\|\| \|\| \|+\|\|+\| \|+ | |
| 00230 | 295 | EFWMSFEDFLRHFTKVEICNLRPDWF | 320 |

Cysteine protease activity is dependent on an active dyad of cysteine and histidine, the order and spacing of these residues varying in the 20 or so known families. Families C1, C2 and C10 are loosely termed papain-like, and nearly half of all cysteine proteases are found exclusively in viruses. Calpain is an intracellular protease involved in many important cellular functions that are regulated by calcium. The protein is a complex of 2 polypeptide chains (light and heavy), with three known forms in mammals: a highly calcium-sensitive (i.e., micro-molar range) form known as mu-calpain, mu-CANP or calpain I; a form sensitive to calcium in the milli-molar range, known as m-calpain, m-CANP or calpain II; and a third form, known as p94, which is found in skeletal muscle only. All three forms have identical light but different heavy chains. The heavy chain comprises four domains: domain 2 contains the catalytic region; domain 4 binds calcium and regulates activity. Domain 2 shows low levels of sequence similarity to papain; although the catalytic His has not been located by biochemical means, it is likely that calpain and papain are related. Domain 4 has four EF hand calcium-binding regions and is simmilar to sorcin and the Ca2+-binding region of calpain light chain. Calpain shows preferential cleavage for Tyr-with leucine or valine as the P2 residue.

Calpain is unique among the cysteine protease family of enzymes in that it combines thiol protease activity with calmodulin-like activity. The enzyme is implicated in a number of pathophysiological conditions (Donkor, Curr Med Chem 7(12):1171-1188, 2000). Proteases of the caspase and calpain families have been implicated in neurodegenerative processes, as their activation can be triggered by calcium influx and oxidative stress (Chan and Mattson, J Neurosci Res 58(1):167-90, 1999). Mitochondrial calpain plays an essential role in apoptotic commitment by cleaving Bax at its N-terminus and generating the Bax/p 18 fragment, which in turn mediates cytochrome c release and initiates apoptotic execution (Gao and Dou, J Cell Biochem 80(1):53-72, 2001). Deficiency of the nCL-4 calpain protease has been implicated in neoplastic transformation (Liu et al., J Biol Chem 275(40):31093-8, 2000). Calpain proteases have been implicated in axon and myelin destruction following injury since they degrade structural proteins in the axon-myelin unit and may be responsible for destruction of myelinated axons adjacent to the lesion site following traumatic injury of the spinal cord (Shields et al., J Neurosci Res 61(2):146-50, 2000). Sperm calpain has been shown to be a novel component of the biochemical processes that regulate the fertilizing capacity of human spermatozoa (Rojas and Moretti-Rojas, Int J Androl 23(3):163-8, 2000). Findings have indicated that modulation of calpain activity contributes to muscular dystrophies by disrupting normal regulatory mechanisms influenced by calpains (Tidball and Spencer, Int J Biochem Cell Biol 32(1):l-5, 2000).

The above defined information for NOV1 suggests that this calpain-like protein may function as a member of the calpain family. Therefore, the NOV1 nucleic acids and proteins of the invention are useful in potential therapeutic applications implicated in various diseases and disorders described below and/or other pathologies. For example, the NOV1 compositions of the present invention will have efficacy for treatment of patients suffering from Von Hippel-Lindau (VHL) syndrome, cirrhosis, transplantation disorders, pancreatitis, obesity, diabetes, autoimmune disease, renal artery stenosis, interstitial nephritis, glomerulonephritis, polycystic kidney disease, systemic lupus erythematosus, renal tubular acidosis, IgA nephropathy, hypercalcemia, Lesch-Nyhan syndrome, developmental defects, cataract, spinal cord injury, Alzheimer's disease, muscular dystrophy, acoustic trauma, cancer, learning and memory defects and infertility. The NOV1 nucleic acid encoding calpain-like protein, and the calpain-like protein of the invention, or fragments thereof, may further be useful in diagnostic applications, wherein the presence or amount of the nucleic acid or the protein are to be assessed.

NOV2

A disclosed NOV2 nucleic acid of 1796 nucleotides (also referred to as 21421174) encoding a novel Epsin-like protein is shown in Table 2A. An open reading frame was identified beginning with an ATG initiation codon at nucleotides 40-42 and ending with a TAA codon at nucleotides 1771-1773. Putative untranslated regions upstream from the intiation codon and downstream from the termination codon are underlined in Table 2A. The start and stop codons are in bold letters.

TABLE 2A
NOV2 nucleotide sequence.

(SEQ ID NO:3)

ATCGGGGCCCTGTGCCCCTTGCTGCTGCAGCCGGGCACCATGTCGACCTCGTCCTTGAGGCGCCAGATGAAG
AACATCGTCCACAACTACTCAGAGGCGGAGATCAAGGTTCGAGAGGCCACGAGCAATGACCCCTGGGGCCCA
TCCAGCTCCCTCATGTCAGAGATTGCCGACCTCACCTACAACGTTGTCGCCTTCTCGGAGATCATGAGCATG
ATCTGGAAGCGGCTCAATGACCATGGCAAGAACTGGCGTCACGTTTACAAGGCCATGACGCTGATGGAGTAC
CTCATCAAGACCGGCTCGGAGCGCGTGTCGCAGCAGTGCAAGGAGAACATGTACGCCGTGCAGACGCTGAAG
GACTTCCAGTACGTGGACCGCGACGGCAAGGACCAGGGCGTGAACGTGCGTGAGAAAGCTAAGCAGCTGGTG
GCCCTGCTGCGCGACGAGGACCGGCTGCGGGAAGAGCGGGCGCACGCGCTCAAGACCAAGGAAAAGCTGGCA
CAGACCGCCACGGCCTCATCAGCAGCTGTGGGCTCAGGCCCCCCTCCCGAGGCGGAGCAGGCGTGGCCGCAG
AGCAGCGGGGAGGAGGAGCTGCAGCTCCAGCTGGCCCTGGCCATGAGCAAGGAGGAGGCCGACCAGCCCCCG
TCCTGCGGCCCCGAGGACGACGCCCAGCTCCAGCTGGCCCTTAGTTTGAGCCGAGAAGAGCATGATAAGGAG
GAGCGGATCCGTCGCGGGGATGACCTGCGGCTGCAGATGGCAATCGAGGAGAGCAAGAGGGAGACTGGGGGC
AAGGAGGAGTCGTCCCTCATGGACCTTGCTGACGTCTTCACGGCCCCAGCTCCTGCCCCGACCACAGACCCC
TGGGGGGGCCCAGCACCCATGGCTGCTGCCGTCCCCACGGCTGCCCCCACCTCGGACCCCTGGGGCGGCCCC
CCTGTCCCTCCAGCTGCTGATCCCTGGGGAGGTCCAGCCCCCACGCCGGCCTCTGGGGACCCCTGGAGGCCT
GCTGCCCCTGCAGGACCCTCAGTTGACCCTTGGGGTGGGACCCCAGCCCCTGCAGCTGGGGAGGGGCCCACG
CCTGATCCATGGGGAAGTTCCGATGGTGGTGGGGTCCCGGTCAGTGGGCCCTCAGCCTCCGATCCCTGGACA
CCGGCCCCGGCCTTCTCAGATCCCTGGGGAGGGTCACCTGCCAAGCCCAGCACCAATGGCACAGCAGCCGGG
GGATTCGACACGGAGCCCGACGAGTTCTCTGACTTTGACCGACTCCGCACGGCACTGCCGCCCCTCTCCCGG
ATCCTTCCAGGAGAGCTGGAGCTGCTGGCAGGAGAGGTGCCGGCCCGAAGCCCTGGGGCGTTTGACATGAGT
GGGGTCAGGGGATCTCTGGCTGAGGCTGTGGGCAGCCCCCCACCTGCAGCCACACCAACTCCCACGCCCCCC
ACCCGGAAGACGCCGGAGTCATTCCTGGGGCCCAATGCAGCCCTCGTCGACCTGGACTCGCTGGTGAGCCGG
CCGGGCCCCACGCCGCCTGGAGCCAAGGCCTCCAACCCCTTCCTGCCAGCAGGAGGCCCAGCCACTGGCCCT
TCCGTCACCAACCCCTTCCAGCCCGCGCCTCCCGCGACGCTCACCCTGAACCAGCTCCGTCTCAGTCCTGTG
CCTCCCGTCCCTGGAGCGCCACCCACGTACATCTCTCCCCTTGGCGGGGGCCCTGGCCTGCCCCCCATGATG
CCCCCGGGCCCCCCGGCCCCCAACACTAATCCCTTCCTCCTATAATCCAGGGCGGAAGGGGGCCTGGC

The disclosed NOV2 nucleic acid sequence, localized to chromsome 19, has 1338 of 1563 bases (85%) identical to a Homo sapiens EH domain-binding mitotic phosphoprotein (EPSIN) mRNA (gb:GENBANK-ID:AF073727|acc:AF073727) (E=1.4⁻²³⁷).

A NOV2 polypeptide (SEQ ID NO:4) encoded by SEQ ID NO:3 has 577 amino acid residues and is presented using the one-letter code in Table 2B. Signal P, Psort and/or Hydropathy results predict that NOV2 does not contain a signal peptide and is likely to be localized to the mitochondrial matrix space with a certainty of 0.4600 and to the cytoplasm with a certainty of 0.4500.

TABLE 2B
Encoded NOV2 protein sequence.

(SEQ ID NO:4)

MSTSSLRRQMKNIVHNYSEAEIKVREATSNDPWGPSSSLMSEIADLTYNVVAFSEIMSMIWKRLNDHGKNWR
HVYKAMTLMEYLIKTGSERVSQQCKENMYAVQTLKDFQYVDRDGKDQGVNVREKAKQLVALLRDEDRLREER
AHALKTKEKLAQTATASSAAVGSGPPPEAEQAWPQSSGEEELQLQLALAMSKEEADQPPSCGPEDDAQLQLA
LSLSREEHDKEERIRRGDDLRLQMAIEESKRETGGKEESSLMDLADVFTAPAPAPTTDPWGGPAPMAAAVPT
AAPTSDPWGGPPVPPAADPWGGPAPTPASGDPWRPAAPAGPSVDPWGGTPAPAAGEGPTPDPWGSSDGGGVP
VSGPSASDPWTPAPAFSDPWGGSPAKPSTNGTAAGGFDTEPDEFSDFDRLRTALPPLSRILPGELELLAGEV
PARSPGAFDMSGVRGSLAEAVGSPPPAATPTPTPPTRKTPESFLGPNAALVDLDSLVSRPGPTPPGAKASNP
FLPAGGPATGPSVTNPFQPAPPATLTLNQLRLSPVPPVPGAPPTYISPLGGGPGLPPMMPPGPPAPNTNPFLL

The NOV2 amino acid sequence has 569 of 577 amino acid residues (98%) identical to, and 569 of 577 amino acid residues (98%) similar to, a Homo sapiens 576 amino acid residue protein (ptnr:TREMBLNEW-ACC:BAB14041) (cDNA FLJ12392 FIS, clone MAMMA1002699, highly similar to Rattits norvegicus eh domain binding protein epsin mRNA (GENBANK-ID:CAB61412) (E=3.0e⁻³¹³).

The disclosed NOV2 is expressed in at least the following tissues: Retinoblastoma, leiomyomas, mammary gland, bone trabecular cells, ovary, bone marrow, spleen, placenta, heart. This information was derived by determining the tissue sources of the sequences that were included in the invention. In addition, the sequence is predicted to be expressed in brain tissue because of the expression pattern of a closely related Homo sapiens EH domain-binding mitotic phosphoprotein (Epsin) mRNA (GENBANK-ID: gb:GENBANK-ID:AF073727|acc:AF073727).

NOV2 also has homology to the amino acid sequences shown in the BLASTP data listed in Table 2C.

TABLE 2C
BLAST results for NOV2

| Gene Index/ | | Length | Identity | Positives | |
|---|---|---|---|---|---|
| Identifier | Protein/Organism | (aa) | (%) | (%) | Expect |
| gi\|14758059\|ref\|XP_ | EH domain- | 576 | 569/578 | 569/578 | 0.0 |
| 034403.1\| | binding | | (98%) | (98%) | |
| | mitotic | | | | |
| | phosphoprotein | | | | |
| | [Homo | | | | |
| | sapiens] | | | | |
| gi\|3249559\|gb\|AAC33823.1\| | EH domain | 575 | 541/577 | 548/577 | 0.0 |
| (AF018261) | binding | | (93%) | (94%) | |
| | protein Epsin | | | | |
| | [Rattus norvegicus] | | | | |
| gi\|7019369\|ref\|NP_037465.1\| | EH domain- | 551 | 544/578 | 544/578 | 0.0 |
| | binding | | (94%) | (94%) | |
| | mitotic | | | | |
| | phosphoprotein | | | | |
| | [Homo sapiens] | | | | |
| gi\|2072301\|gb\|AAC60123.1\| | mitotic | 609 | 356/613 | 402/613 | 1e−126 |
| (U95102) | phosphoprotein | | (58%) | (65%) | |
| | 90 [Xenopus | | | | |
| | laevis] | | | | |
| gi\|3894395\|gb\|AAC78608.1\| | epsin 2a | 584 | 292/611 | 348/611 | 1e−102 |
| (AF062084) | [Homo sapiens] | | (47%) | (56%) | |

The homology of these sequences is shown graphically in the ClustalW analysis shown in Table 2D.

[image]

[image]

Tables 2E and 2F list the domain description from DOMAIN analysis results against NOV2. This indicates that the NOV2 sequence has properties similar to those of other proteins known to contain these domains.

TABLE 2E
Domain Analysis of NOV2

gnl|Pfam|pfam01417, ENTH, ENTH domain. The ENTH (Epsin N-terminal
homology) domain is found in proteins involved in endocytosis and
cytoskeletal machinery. The function of the ENTH domain is unknown.
(SEQ ID NO:91)
Length = 123 residues, 92.7% aligned
Score = 173 bits (439) , Expect = 2e−44

| | | | |
|---|---|---|---|
| NOV2 | 17 | YSEAEIKVREATSNDPWGPSSSLMSEIADLTYNVVAFSEIMSMIWKRLNDHGKNWRHVYK | 76 |
| | | \|\|\| \| \|\|+\|\|+\|\|\|\|\|\| + \|\| \|\|+ +\| \|\|\| \|+ \|\|\| + \|\|\|\|\| \|\|\| | |
| 01417 | 1 | YSELEKAVRKATNNDPWGPKGKHLDEILQGTYDEKSFPEIMDMLDKRLLE-GKNWRVVYK | 59 |
| NOV2 | 77 | AMTLMEYLIKTGSERVSQQCKENMYAVQTLKDFQYVDRDGKDQGVNVREKAKQLV | 131 |
| | | \|+ \|+ \|\|++ \|\|\|\|\| \|+ + \| \| ++ \|+\|\|+ \|\| \|\|\|\|\| \|+\| \|\| \|+ | |
| 01417 | 60 | ALILLHYLLRNGSERVVQEARRNNYRIRELEDFRKVDSSGKDQGANIRTYAKYLL | 114 |

TABLE 2F
Domain Analysis of NOV2

gnl|Smart|smart00273, ENTH, Epsin N-terminal homology (ENTH) domain
(SEQ ID NO:92)
Length = 127 residues, 89.8% aligned
Score = 149 bits (377), Expect = 3e−37

| | | | |
|---|---|---|---|
| NOV2 | 18 | SEAEIKVREATSNDPWGPSSSLMSEIADLTYNV-VAFSEIMSMIWKRLNDHGKNWRHVYK | 76 |
| | | \|+ \|+\|\|\|+\|\|+\|\| \|\|\| + \|\| \|+\| + +\|\|\|+++\|+\|\|\|\| \|\|\|\| \|\|\| | |
| 01273 | 1 | SDLEVKVRKATNNDEWGPKGKHLREIIQGTHNEKSSVAEIMAVLWRRLNDT-KNWRVVYK | 59 |
| NOV2 | 77 | AMTLMEYLIKTGSERVSQQCKENMYAVQTLKDFQYVDRDGKDQGVNVREKAKQLV | 131 |
| | | \|+ \|+ \|\|++ \|\| \| + \| + \|\| \| + +\| \|\|\|\|\| \|+\| \|\| \|+ | |
| 01273 | 60 | ALILLHYLLRNGSPNVVLEALRNRNRILTLSDFRDIDSRGKDQGANIRTYAKYLL | 114 |

TABLE 2G
BLASTX results for NOV2

| | | Smallest | | |
|---|---|---|---|---|
| Sequences producing | Reading | Sum | | |
| High-scoring | High | Prob | | |
| Segment Pairs: | Frame | Score | P(N) | N |
| patp.AAB24234 Vesicle | +1 | 3006 | 1.7e−312 | 1 |
| associated prot 13 - Homo sap, | | | | |
| 576 aa . . . | | | | |

Epsin (Eps15 interactor) is a cytosolic protein involved in clathrin-mediated endocytosis via its direct interactions with clathrin, the clathrin adaptor AP-2, and Eps15. The NH(2)-terminal portion of epsin contains a phylogenetically conserved module of unknown function, known as the ENTH domain (epsin NH(2)-terminal homology domain). Findings suggest that epsin 1 may function in a signaling pathway connecting the endocytic machinery to the regulation of nuclear function (Hyman et al., J Cell Biol 149(3):537-46, 2000).

During endocytosis, clathrin and the clathrin adaptor protein AP-2, assisted by a variety of accessory factors, help to generate an invaginated bud at the cell membrane. One of these factors is Eps15, a clathrin-coat-associated protein that binds the alpha-adaptin subunit of AP-2. It has been proposed that epsin may participate, together with Eps15, in the molecular rearrangement of the clathrin coats that are required for coated-pit invagination and vesicle fission (Chen et al., Nature 394(6695):793-7, 1998).

It has been shown that both rat epsin and Eps15 are mitotic phosphoproteins and that their mitotic phosphorylation inhibits binding to the appendage domain of alpha-adaptin. Both epsin and Eps15, like other cytosolic components of the synaptic vesicle endocytic machinery, undergo constitutive phosphorylation and depolarization-dependent dephosphorylation in nerve terminals. Furthermore, their binding to AP-2 in brain extracts is enhanced by dephosphorylation. Epsin together with Eps 15 is proposed to assist the clathrin coat in its dynamic rearrangements during the invagination/fission reactions. Their mitotic phosphorylation may be one of the mechanisms by which the invagination of clathrin-coated pits is blocked in mitosis and their stimulation-dependent dephosphorylation at synapses may contribute to the compensatory burst of endocytosis after a secretory stimulus (Chen et al., J Biol Chem Feb. 5, 1999;274(6):3257-60).

The above defined information for NOV2 suggests that the NOV2 protein may function as a member of a family of novel Espin-like proteins. Therefore, the NOV2 nucleic acids and proteins of the invention are useful in potential therapeutic applications implicated in various diseases and disorders described below and/or other pathologies. For example, the NOV2 compositions of the present invention will have efficacy for treatment of patients suffering from cardiomyopathies, atherosclerosis, hypertension, congenital heart defects, aortic stenosis, atrial septal defect, atrioventricular canal defect, ductus arteriosus, pulmonary stenosis, subaortic stenosis, ventricular septal defect, valve diseases, tuberous sclerosis, scleroderma, obesity, transplantation disorders, endometriosis, infertility, cancer, hemophilia, hypercoagulation, idiopathic thrombocytopenic purpura, autoimmune diseases, allergies, immunodeficiencies, graft versus host disease, developmental defects, dementia, Von Hippel-Lindau (VHL) syndrome, Alzheimer's disease, stroke, hypercalcemia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration. The NOV2 nucleic acid encoding Espin-like proteins, and the Espin-like proteins of the invention, or fragments thereof, may further be useful in diagnostic applications, wherein the presence or amount of the nucleic acid or the protein are to be assessed.

NOV3

A disclosed NOV3 nucleic acid of 2973 nucleotides (also referred to as AC025263_da1) encoding a novel Low Density Lipoprotein B(LDLB)-like protein is shown in Table 3A. An open reading frame was identified beginning with a ATG initiation codon at nucleotides 1-3 and ending with a TAA codon at nucleotides 2971-2973. The start and stop codons are in bold letters in Table 3A.

TABLE 3A
NOV3 Nucleotide Sequence

(SEQ ID NO:5)

ATGGCCACCGCGGCAACCTCACCCGCGCTGAAGCGGCTGGATCTGCGCGACCCTGCGGCTCTTTTCGAGACG
CATGGAGCGGAGGAGATCCGCGGGCTGGAGCGCCAGGTTCGGGCCGAGATCGAGCACAAGAAGGAGGAGCTG
CGGCAGATGGTGGGCGAACGGTACCGCGACCTGATCGAGGCGGCCGACACCATCGGCCAGATGCGCCGCTGC
GCCGTGGGGCTAGTGGACGCCGTGAAGGCCACCGACCAGTACTGCGCCCGCCTCCGCCAGGCCGGCTCGGCC
GCGCCCCGGCCACCGCGGGCCCAGCAGGTCAGTCCCCGTGCCCCCACCCTGCGACCCGCAGGCGGGTCCCGG
AGCCCCTGGCCTTGCAGGTCAACCCCGCCCCCCTCTGTCAGTCCCAGACCCCGCGAGTCCTCACCTTCCTTA
GCCAGGAGCCTATCCGCCCCTCACCCTTTGGGCCTCTACCTGCTCTGCTGCCACCTCCACAGCCTGCTCCAG
CTGGATTCTTCTAGTTCCCGATACAGTCCCGTCCTCTCCCGGTTTCCTATACTCATCCGGCAGGTGGCAGCC
GCCAGCCACTTCCGGTCAACTATTCTGCATGAAAGCAAGATGTTGCTCAAATGCCAAGGTGTGTCTGACCAA
GCTGTGGCCGAGGCCCTGTGCTCTATAATGCTCTTAGAAGAGAGTTCTCCTCGCCAAGCCCTCACAGACTTC
CTGCTGGCCAGAAAGGCAACTATTCAGAAACTTCTCAACCAGCCACACCATGGTGCTGGTATCAAGGCTCAG
ATTTGCTCATTAGTGGAGTTGCTGGCCACCACTCTGAAGCAAGCTCATGCCCTTTTCTACACTTTGCCAGAA
GGACTGCTGCCAGATCCAGCCCTGCCATGTGGCTTGCTCTTCTCTACTCTGGAGACCATCACAGGCCAGCAT
CTGCCGAAGGGCACTGGTGTCCTGCAGGAAGAGATGAAACTCTGCAGCTGGTTTAAACACCTGCCAGCATCC
ATCGTCGAGTTCCAGCCAACACTCCGAACCCTTGCACATCCCATCAGTCAGGAATACCTGAAAGACACGCTG
CAGAAATGGATCCACATGTGTAATGAAGACATTAAAAATGGGATCACCAACCTGCTCATGTACGTGAAGAGC
ATGAAGGGTCTCGCGGGAATCCGGGACGCCATGTGGGAGTTACTTACCAATGAGTCCACCAATCACAGCTGG
GATGTGCTATGTCGGCGGCTTCTGGAGAAGCCGCTCTTGTTCTGGGAAGATATGATGCAGCAACTGTTCCTT
GACCGATTACAGACTCTGACAAAAGAAGGCTTTGACTCCATCTCCAGTAGCTCCAAGGAGCTCTTGGTTTCA
GCTTTGCAGGAACTTGAAAGCAGCACCAGCAACTCCCCTTCAAATAAGCACATCCACTTTGAGTACAACATG
TCGCTCTTCCTCTGGTCTGAGAGTCCTAATGACCTGCCTTCCGATGCGGCCTGGGTCAGCGTGGCAAACCGG
GGTCAGTTAGGGGTCGCTGGCCTCTCTATGAAAGCACAAGCCATCAGCCCTTGTGTACAGAACTTCTGTTCT
GCCCTGGATTCTAAGCTGAAGGTTAAACTAGATGACCTCCTGGCTTACCTCCCCTCTGATGACTCATCACTG
CCCAAGGACGTTTCTCCCACACAGGCCAAGAGTTCTGCCTTTGACAGATACGCAGATGCGGGGACCGTGCAG
GAGATGCTGCGGACTCAGTCCGTGGCATGCATCAAGCACATCGTGGACTGCATCCGGGCAGAGCTACAGAGC
ATTGAAGAAGGTGTGCAAGGGCAACAGGATGCCCTCAACAGTGCCAAGCTGCACTCAGTTCTTTTCATGGCC
AGACTCTGCCTGTCCCTGGGAGAGCTGTGCCCCCATCTGAAGCAGTGCATCCTGGGAAAATCAGAGAGCTCA
GAGAAACCAGCAAGGGAGTTTAGGGCTCTGAGAAAACAGGGAAAGGTGAAAACTCAGGAAATCATTCCTACA
CAGGCCAAGTGGCAAGAGGTTAAAGAAGTACTCCTCCAGCAGAGCGTGATGGGCTACCAGGTCTGGAGCAGT
GCAGTTGTGAAAGTTTTGATTCATGGATTCACCCAGTCATTACTTCTAGATGATGCTGGCTCAGTTCTGGCC
ACAGCCACCAGCTGGGATGAGCTAGAAATTCAGGAGGAGGCAGAGTCTGGCAGCAGTGTCACATCCAAGATC
CGACTCCCTGCACAGCCGTCCTGGTATGTACAGTCCTTCCTGTTTAGTTTATGCCAGGAAATTAATCGGGTT
GGAGGCCATGCCTTGCCAAAGGTGACATTACAGGAGATGCTGAAAAGCTGTATGGTTCAAGTAGTAGCTGCC
TATGAGAAACTCTCCGAAGAAAAACAGATTAAGAAAGAAGGTGCATTTCCAGTCACCCAGAACCGGGCGCTG
CAGCTGCTTTATGATCTGCGTTACCTCAACATTGTTCTGACAGCCAAGGGTGACGAGGTGAAGAGTGGCCGG
AGCAAGCCAGACTCCAGGATTGAGAAAGTGACTGACCACCTGGAAGCCCTCATTGATCCATTTGACCTGGAC
GTTTTCACGCCACACCTCAACAGCAACCTTCATCGCCTGGTGCAGCGAACTTCTGTGCTGTTTGGATTGGTG
ACTGGTACAGAGAATCAGCTCGCCCCCCGGAGCAGTACGTTCAACTCCCAAGAACCCCATAACATCCTGCCA
CTGGCATCCAGTCAGATCAGGAGGTTTGGACTTCTCCCACTGAGCATGACAAGCACTCGAAAGGCTAAATCA
ACCAGAAACATCGAAACAAAAGCTCAGGTTGGTCCCCCGGCACGCTCCACAGCTGGTGACCCGACAGTTCCT
GGCTCCTTGTTCAGACAGCTTGTCAGTGAAGAAGACAACACGTCTGCACCTTCATTATTCAAACTTGGCTGG
CTCTCTAGTATGACTAAGTAA

The disclosed NOV3 nucleic acid sequence maps to chromosome 19p13.1-13.3 and has 2360 of 2957 bases (79%) identical to a Mus musculus ldlBp (LDLB) mRNA (gb:GENBANK-ID:AF109377|acc:AF109377) (E=0.0).

A disclosed NOV3 protein (SEQ ID NO:6) encoded by SEQ ID NO:5 has 990 amino acid residues, and is presented using the one-letter code in Table 3B. Signal P, Psort and/or Hydropathy results predict that NOV3 does not contain a signal peptide, and is likely to be localized to the nucleus with a certainty of 0.7600 and to the mitochondrial matrix space with a certainty of 0.4824.

TABLE 3B
Encoded NOV3 protein sequence.

(SEQ ID NO:6)

MATAATSPALKRLDLRDPAALFETHGAEEIRGLERQVRAEIEHKKEELRQMVGERYRDLIEAADTIGQMRRC
AVGLVDAVKATDQYCARLRQAGSAAPRPPRAQQVSPRAPTLRPAGGSRSPWPCRSTPPPSVSPRPRESSPSL
ARSLSAPHPLGLYLLCCHLHSLLQLDSSSSRYSPVLSRFPILIRQVAAASHFRSTILHESKMLLKCQGVSDQ
AVAEALCSIMLLEESSPRQALTDFLLARKATIQKLLNQPHHGAGIKAQICSLVELLATTLKQAHALFYTLPE
GLLPDPALPCGLLFSTLETITGQHLPKGTGVLQEEMKLCSWFKHLPASIVEFQPTLRTLAHPISQEYLKDTL
QKWIHMCNEDIKNGITNLLMYVKSMKGLAGIRDAMWELLTNESTNHSWDVLCRRLLEKPLLFWEDMMQQLFL
DRLQTLTKEGFDSISSSSKELLVSALQELESSTSNSPSNKHIHFEYNMSLFLWSESPNDLPSDAAWVSVANR
GQLGVAGLSMKAQAISPCVQNFCSALDSKLKVKLDDLLAYLPSDDSSLPKDVSPTQAKSSAFDRYADAGTVQ
EMLRTQSVACIKHIVDCIRAELQSIEEGVQGQQDALNSAKLHSVLFMARLCLSLGELCPHLKQCILGKSESS
EKPAREFRALRKQGKVKTQEIIPTQAKWQEVKEVLLQQSVMGYQVWSSAVVKVLIHGFTQSLLLDDAGSVLA
TATSWDELEIQEEAESGSSVTSKIRLPAQPSWYVQSFLFSLCQEINRVGGHALPKVTLQEMLKSCMVQVVAA
YEKLSEEKQIKKEGAFPVTQNRALQLLYDLRYLNIVLTAKGDEVKSGRSKPDSRIEKVTDHLEALIDPFDLD
VFTPHLNSNLHRLVQRTSVLFGLVTGTENQLAPRSSTFNSQEPHNILPLASSQIRRFGLLPLSMTSTRKAKS
TRNIETKAQVGPPARSTAGDPTVPGSLFRQLVSEEDNTSAPSLFKLGWLSSMTK

The NOV3 amino acid sequence has 807 of 990 amino acid residues (81%) identical to, and 877 of 990 amino acid residues (88%) similar to, a Mus musculus 980 amino acid residue protein (ptnr:SPTREMBL-ACC:Q9Z160) (E=0.0). The global sequence homology is 62.396% amino acid homology and 54.576% amino acid identity. NOV3 is expressed in at least the following tissues based on literature sources: ovaries, liver, epidermis, fibroblast, blood leukocytes.

NOV3 also has homology to the amino acid sequences shown in the BLASTP data listed in Table 3C.

TABLE 3C
BLAST results for NOV3

| Gene Index/ | | Length | Identity | Positives | |
|---|---|---|---|---|---|
| Identifier | Protein/Organism | (aa) | (%) | (%) | Expect |
| gi\|14776518\|ref\|XP_ | hypothetical | 912 | 844/902 | 853/902 | 0.0 |
| 040307.1\| | protein | | (93%) | (93%) | |
| | DKFZp762L1710 | | | | |
| | [Homo | | | | |
| | sapiens] | | | | |
| gi\|15011849\|ref\|NP_ | low density | 980 | 799/994 | 871/994 | 0.0 |
| 038609.2\| | lipoprotein B | | (80%) | (87%) | |
| | [Mus | | | | |
| | musculus] | | | | |
| gi\|14776514\|ref\|XP_ | hypothetical | 666 | 660/667 | 661/667 | 0.0 |
| 040308.1\| | protein | | (98%) | (98%) | |
| | DKFZp762L1710 | | | | |
| | [Homo | | | | |
| | sapiens] | | | | |
| gi\|7243143\|dbj\|BAA92619.1\| | KIAA1381 | 961 | 892/951 | 901/951 | 0.0 |
| (AB037802) | protein [Homo | | (93%) | (93%) | |
| | sapiens] | | | | |
| gi\|11360291\|pir\| \|T50629 | hypothetical | 438 | 436/439 | 436/439 | 0.0 |
| | protein | | (99%) | (99%) | |
| | DKFZp762L1710.1 | | | | |
| | [Homo | | | | |
| | sapiens] | | | | |

The homology of these sequences is shown graphically in the ClustalW analysis shown in Table 3D.

[image]

[image]

[image]

The amino acid sequence of NOV3 has high homology to other proteins as shown in Table 3E.

TABLE 3
BLASTX results for NOV3

| | | Smallest | | |
|---|---|---|---|---|
| | | Sum | | |
| | Reading High | Prob | | |
| Sequences producing High-scoring Segment Pairs: | Frame | Score | P (N) | N |
| patp: AAB75567 Gene 16 hum secr prot homol aa Mus mus, 174 aa . . . | +3 | 789 | 1.5e−77 | 1 |

Low density lipoprotein (LDL) particles are the major cholesterol carriers in circulation and their physiological function is to carry cholesterol to the cells. In the process of atherogenesis these particles are modified and they accumulate in the arterial wall. Elevated serum cholesterol bound to low density lipoprotein (LDL) is a characteristic of familial hypercholesterolemia.

By studying cultured fibroblasts from homozygotes, Brown and Goldstein (Proc. Nat. Acad. Sci. 70: 2804-2808, 1973; Proc. Nat. Acad. Sci. 71: 788-792, 1974) showed that the basic defect in patients suffereing from coronary artery disease and/or familial hypercholesterolemia concerns the cell membrane receptor for LDL. Normally, LDL is bound at the cell membrane and taken into the cell ending up in lysosomes where the protein is degraded and the cholesterol is made available for repression of microsomal enzyme 3-hydroxy-3-methylglutaryl coenzyme A (HMG CoA) reductase, the rate-limiting step in cholesterol synthesis. In the disease state, an internalizatin mutant of the LDL receptor binds LDL but is unable to faciliate passage of LDL to the inside of the cell (Goldstein et al., Cell 12: 629-641, 1977). Along with the disease states discussed above, LDL has been implicated in viral infection. Studies indicate that Hepatitis C virus (HCV), the principal viral cause of chronic hepatitis, and other viruses enter cells through the mediation of LDL receptors. The studies demonstate that endocytosis of these viruses correlates with LDL receptor activity (Agnello et al., Proc. Nat. Acad. Sci. 96:12766-71, 1999).

The above defined information for NOV3 suggest that this NOV3 protein may function as a member of a Low Density Lipoprotein B protein family. Therefore, the NOV3 nucleic acids and proteins of the invention are useful in potential therapeutic and diagnostic applications. For example, a cDNA encoding the NOV3 protein may be useful in gene therapy, and the NOV3 protein may be useful when administered to a subject in need thereof. By way of nonlimiting example, the compositions of the present invention will have efficacy for treatment of patients suffering from Familial hypercholesterolemia, hyperlipoproteinemia II phenotype, tendinous xanthomas, corneal arcus, coronary artery disease, planar xanthomas, webbed digits, hypercholesterolemia, fertility, coronary artery disease, diabeties, atherosclerosis, xanthomatosis, Hepatitis C infection, regulation, synthesis, transport, recycling, or turnover of LDL receptors, Cerebral arteriopathy with subcortical infarcts and leukoencephalopathy, Epiphyseal dysplasia, multiple 1, Ichthyosis, nonlamellar and nonerythrodermic, congenital, Leukemia, T-cell acute lymphoblastoid, Pseudoachondroplasia, SCID, autosomal recessive, T-negative/B-positive type, C3 deficiency, Diabetes mellitus, insulin-resistant, with acanthosis nigricans, Glutaricaciduria, type I, Hypothyroidism, congenital, Leprechaunism, Liposarcoma, Mucolipidosis IV, Persistent Mullerian duct syndrome, type I, Rabson-Mendenhall syndrome, Thyroid carcinoma, nonmedullary, with cell oxyphilia, Erythrocytosis, familial, Malaria, cerebral, susceptibility to, Bleeding disorder due to defective thromboxane A2 receptor, Cerebellar ataxia, Cayman type, Convulsions, familial febrile, 2, Cyclic hematopoiesis, Fucosyltransferase-6 deficiency, GAMT deficiency, Von Hippel-Lindau (VHL) syndrome, Cirrhosis, Transplantation, Psoriasis, Actinic keratosis, Tuberous sclerosis, Acne, Hair growth, allopecia, pigmentation disorders and endocrine disorders. The NOV3 nucleic acid encoding Low Density Lipoprotein B-like protein, and the Low Density Lipoprotein B-like protein of the invention, or fragments thereof, may further be useful in diagnostic applications, wherein the presence or amount of the nucleic acid or the protein are to be assessed.

NOV4

A disclosed NOV4 nucleic acid of 1851 nucleotides (designated CuraGen Acc. No. Acc26756-da1) encoding a novel Purinoceptor-like protein is shown in Table 4A. An open reading frame was identified beginning with an ATG initiation codon at nucleotides 347-349 and ending with a TGA codon at nucleotides 1358-1360. Putative untranslated regions upstream from the initiation codon and downstream from the termination codon are underlined in Table 4A, and the start and stop codons are in bold letters.

TABLE 4A
NOV4 Nucleotide Sequence

(SEQ ID NO:7)

CTAGAATTCAGCGGCCGCTGAATTCTAGCAGGCACGCTGGGCGCATGTCCGCCTCGCCGGGGCTGCCAGA
ATCTTGGAATCCCAATCCGTGAGGTTCCTGGGTGTGCTGGCATCAGGACAGCGGTCCACGAACGGTGTGT
TACCCAAATATTGACATCCTGCAGCTAGCCTCAAACAATCACAGCTACTTTCCAATTTCAGAGAAAAAAA
GGCTAAAATTGGTAATCCTGATGAAAATCAACAAAATACACATGAAGAGACAGCACTGAGAGCGAGTTAC
TGCTCATTTGATTCATATTGCCAAACTGAACTCTCTTGTTTTCTTGCAAGATGAAAGGAGACAACCATGA
ATGAGCCACTAGACTATTTAGCAAATGCTTCTGATTTCCCCGATTATGCAGCTGCTTTTGGAAATTGCAC
TGATGAAAACATCCCACTCAAGATGCACTACCTCCCTGTTATTTATGGCATTATCTTCCTCGTGGGATTT
CCAGGCAATGCAGTAGTGATATCCACTTACATTTTCAAAATGAGACCTTGGAAGAGCAGCACCATCATTA
TGCTGAACCTGGCCTGCACAGATCTGCTGTATCTGACCAGCCTCCCCTTCCTGATTCACTACTATGCCAG
TGGCGAAAACTGGATCTTTGGAGATTTCATGTGTAAGTTTATCCGCTTCAGCTTCCATTTCAACCTGTAT
AGCAGCATCCTCTTCCTCACCTGTTTCAGCATCTTCCGCTACTGTGTGATCATTCACCCAATGAGCTGCT
TTTCCATTCACAAAACTCGATGTGCAGTTGTAGCCTGTGCTGTGGTGTGGATCATTTCACTGGTAGCTGT
CATTCCGATGACCTTCTTGATCACATCAACCAACAGGACCAACAGATCAGCCTGTCTCGACCTCACCAGT
TCGGATGAACTCAATACTATTAAGTGGTACAACCTAATTTTGACTGCAACTACTTTCTGCCTCCCCTTGG
TGATAGTGACACTTTGCTATACCACGATTATCCACACTCTGACCCATGGACTGCAAACTGACAGCTGCCT
TAAGCAGAAAGCACGAAGGCTAACCATTCTGCTACTCCTTGCATTTTACGTATGTTTTTTACCCTTCCAT
ATCTTGAGGGTCATTCGGATCGAATCTCGCCTGCTTTCAATCAGTTGTTCCATTGAGAATCAGATCCATG
AAGCTTACATCGTTTCTAGACCATTAGCTGCTCTGAACACCTTTGGTAACCTGTTACTATATGTGGTGGT
CAGCGACAACTTTCAGCAGGCTGTCTGCTCAACAGTGAGATGCAAAGTAAGCGGGAACCTTGAGCAAGCA
AAGAAAATTAGTTACTCAAACAACCCTTGAAATATTTCATTTACTTAACCAAAAACAAATACTTGCTGAT
ACTTTACCTAGCATCCTAAGATGTTCAGGATGTCTCCCTCAATGGAACTCCTGGTAAATACTGTGTATTC
AAGTAATCATGTGCCAAAGCCAGGGCAGAGCTTCTAGTTCTTTGCAATCCCTTTATTGAGCTCCTCCACT
GGGGAGATATAAGAATGGGATGCATGTATATCAGCAAAGTATTCAGACATAGTATTACAAGCTATTGGAA
CTCAGAGGCATCTTAGAGAACATCTGTTCCCACCAACTTACTATATATACACGGAAACCAATTTCTTACC
CTTGCCCTAGATTGCTCAGTAAATTTGTGCCAAGATAGGAGAAAACCAATCTTTTCACTCATCATTTCAT
GCTTCTCTGCACTCTGGGCCTATTTGTATTGAACCATTAGACAATTCAAACCACTACTTGTATCTTTCTT
AATATTTATTTTTTACATCTCAGAGCTCTAC

The nucleic acid sequence of NOV4 has 419 of 717 bases (58%) identical to a Mus musculus P2Y purinoceptor mRNA (gb:GENBANK-ID: MMU22829|acc:U22829) (E=9.8e⁻¹⁹)

A NOV4 polypeptide (SEQ ID NO:8) encoded by SEQ ID NO:7 is 337 amino acid residues and is presented using the one letter code in Table 4B. Signal P, Psort and/or Hydropathy results predict that NOV4 does not contain a signal peptide and is likely to be localized at the plasma membrane with a certainty of 0.6000.

TABLE 4B
NOV4 protein sequence

(SEQ ID NO:8)

MNEPLDYLANASDFPDYAAAFGNCTDENIPLKMHYLPVIYGIIFLVGFPGNAVVISTYIFKMRPWKSSTIIMLN
LACTDLLYLTSLPFLIHYYASGENWIFGDFMCKFIRFSFHFNLYSSILFLTCFSIFRYCVIIHPMSCFSIHKTR
CAVVACAVVWIISLVAVIPMTFLITSTNRTNRSACLDLTSSDELNTIKWYNLILTATTFCLPLVIVTLCYTTII
HTLTHGLQTDSCLKQKARRLTILLLLAFYVCFLPFHILRVIRIESRLLSISCSIENQIHEAYIVSRPLAALNTF
GNLLLYVVVSDNFQQAVCSTVRCKVSGNLEQAKKISYSNNP

The NOV4 amino acid sequence has 112 of 306 amino acid residues (36%) identical to, and 179 of 306 residues (58%) positive with, a Mus musculus 373 amino acid residue P2YI Purinoceptor protein (ATP Receptor) (ptnr:SWISSPROT-ACC:P49650) (E=9.4e−56).

NOV4 is expressed in at least the following tissues corresponding to the 20 original pooled cDNAs it was amplified from: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary grand, pancreas, pituitary gland, placenta, prostate, salivary gland, skeletal muscle, small intestine, spinal cord, spleen, stomach, testis, thyroid, trachea and uterus.

Possible small nucleotide polymorphisms (SNPs) found for GPCR4 are listed in Table 4C. Depth represents the number of clones covering the region of the SNP. The putative allele frequence (PAF) is the fraction of these clones containing the SNP. A dash, when shown, means that a base is not present. The sign “>” means “is changed to.”

TABLE 4C
SNPs

| Consensus | | Base | |
|---|---|---|---|
| Position | Depth | Change | PAF |
| 193 | 39 | T > — | 0.051 |
| 527 | 33 | C > T | 0.061 |
| 591 | 32 | C > T | 0.062 |
| 614 | 38 | C > T | 0.316 |
| 721 | 33 | T > — | 0.061 |
| 823 | 33 | A > G | 0.061 |
| 929 | 33 | G > A | 0.061 |
| 1073 | 33 | A > — | 0.061 |

NOV4 also has homology to the amino acid sequences shown in the BLASTP data listed in Table 4D.

TABLE 4D
BLAST results for NOV4

| Gene Index/ | | Length | Identity | Positives | |
|---|---|---|---|---|---|
| Identifier | Protein/Organism | (aa) | (%) | (%) | Expect |
| gi\|6679193\|ref\|NP_032798.1\| | purinergic receptor | 373 | 109/299 | 176/299 | 1e−51 |
| | P2Y, G-protein | | (36%) | (58%) | |
| | coupled 1; P2Y1 | | | | |
| | receptor [Mus | | | | |
| | musculus] | | | | |
| gi\|4505557\|ref\|NP_002554.1\| | purinergic receptor | 373 | 108/299 | 176/299 | 5e−51 |
| | P2Y, G-protein | | (36%) | (58%) | |
| | coupled, 1 | | | | |
| | [Homo sapiens] | | | | |
| gi\|2829680\|sp\|P79928\|P2Y8_ | P2Y | 537 | 104/283 | 161/283 | 7e−51 |
| XENLA | PURINOCEPTOR | | (36%) | (56%) | |
| | 8 (P2Y8) [Xenopus | | | | |
| | laevis] | | | | |
| gi\|1352693\|sp\|P49652\|P2YR_ | P2Y | 362 | 106/299 | 174/299 | 7e−51 |
| MELGA | PURINOCEPTOR | | (35%) | (57%) | |
| | 1 (ATP receptor) | | | | |
| | (P2Y1) (purinergic | | | | |
| | receptor) (6H1 | | | | |
| | orphan receptor) | | | | |
| | [Meleagris | | | | |
| | gallopavo] | | | | |
| gi\|464327\|sp\|P34996\|P2YR_ | P2Y | 362 | 106/299 | 174/299 | 7e−51 |
| CHICK | PURINOCEPTOR | | (35%) | (57%) | |
| | 1 (ATP receptor) | | | | |
| | (P2Y1) (purinergic | | | | |
| | receptor) | | | | |
| | [Gallus gallus] | | | | |

The homology of these sequences is shown graphically in the ClustalW analysis shown in Table 4E.

[image]

[image]

Table 4F lists the domain description from DOMAIN analysis results against NOV4. This indicates that the NOV4 sequence has properties similar to those of other proteins known to contain these domains.

TABLE 4F
Domain Analysis of NOV4

gnl|Pfam|pfam00001, 7tm_1, 7 transnembrane receptor (rhodopsin
family). (SEQ ID NO:93)
Length = 254 residues, 100.0% aligned
Score = 125 bits (315), Expect = 3e−30

| | | | |
|---|---|---|---|
| NOV4 | 50 | GNAVVISTYIFKMRPWKSSTIIMLNLACTDLLYLTSLPFLIHYYASGENWIFGDFMCKFI | 109 |
| | | \|\| +\|\| + + + \| +\|\|\|\| \|\|\|+\| +\|\| \|\| \| +\|+\|\|\| +\|\| + | |
| 00001 | 1 | GNLLVILVILRTKKLRTPTNIFLLNLAVADLLFLLTLPPWALYYLVGGDWVFGDALCKLV | 60 |
| NOV4 | 110 | RFSFHFNLYSSILFLTCFSIFRYCVIIHPMSCFSIHKTRCAVVACAVVWIISLVAVIPMT | 169 |
| | | \| \| \|+\|\|\| \|\| \|\| \|\| \|+\|\|+ \| \| \| \| +\|\|+++\|+ +\| | |
| 00001 | 61 | GALFVVNGYASILLLTAISIDRYLAIVHPLRYRRIRTPRRAKVLILLVWVLALLLSLPPL | 120 |
| NOV4 | 170 | FLITSTNRTNRSACLDLTSSDELNTIKWYNLILTATTFCLPLVIVTLCYTTIIHTLTHGL | 229 |
| | | + + \| \| + + \| \|+ \| \| \|\|\|+++ +\|\|\| \|+ \|\| | |
| 00001 | 121 | LFSWLRTVEEGNTTVCLIDFPEESVKRSYVLLSTLVGFVLPLLVILVCYTRILRTLRKRA | 180 |
| NOV4 | 230 | QTDSCLK------QKARRLTILLLLAFYVCFLPFHILRVIRIESRLLSISCSIENQIHEA | 283 |
| | | ++ \|\| +\|\| ++ +++++ \| +\|+\|\|+\|\|+ ++ \|\| + \| | |
| 00001 | 181 | RSQRSLKRRSSSERKAAKMLLVVVVVFVLCWLPYHIVLLLDS-----LCLLSIWRVLPTA | 235 |
| N0V4 | 284 | YIVSRPLAALNTFGNLLLY | 302 |
| | | +++ \|\| +\|+ \| ++\| | |
| 00001 | 236 | LLITLWLAYVNSCLNPIIY | 254 |

The amino acid sequence of NOV4 has high homology to other proteins as shown in Table 4G.

TABLE 4G
BLASTX results for NOV4

| | | Smallest | | |
|---|---|---|---|---|
| | | Sum | | |
| | Reading High | Prob | | |
| Sequences producing High-scoring Segment Pairs: | Frame | Score | P(N) | N |
| patp: AAU04584 Human GPCR 3940, Homo Sapiens 337 aa . . . | +2 | 1764 | 7.0e−181 | 1 |
| patp: AA060971 Human nGPCR54 #2 - Homo sapiens 337 aa . . . | +2 | 1601 | 1.3e−163 | 1 |

The above defined information for NOV4 suggests that this NOV4 protein may function as a member of a purinoceptor-like protein family. Therefore, the NOV4 nucleic acids and proteins of the invention are useful in potential therapeutic applications implicated in various diseases and disorders. The NOV4 nucleic acid encoding purinoceptor-like protein, and the purinoceptor-like protein of the invention, or fragments thereof, may further be useful in diagnostic applications, wherein the presence or amount of the nucleic acid or the protein are to be assessed.

NOV5

NOV5 includes two novel CG88411-like proteins disclosed below. The disclosed proteins have been named NOV5a and NOV5b.

NOV5a

A disclosed NOV5a nucleic acid of 3146 nucleotides (also referred to as sggc^{—}draft_d1895c5_{—}2000081_da1) encoding a novel CG8841-like protein is shown in Table 5A. A n open reading frame was identified beginning with an ATG initiation codon at nucleotides 1-3 and ending with a TGA codon at nucleotides 2293-2295. A putative untranslated region downstream from the termination codon are underlined in Table 5A, and the start and stop codons are in bold letters.

TABLE 5A
NOV5a Nucleotide Sequence

(SEQ ID NO:9)

ATGGGGTCGACCGACTCCAAGCTGAACTTCCGGAAGGCGGTGATCCAGCTCACCACCAAGACGCAGCCCGTGG
AAGCCACCGATGATGCCTTTTGGGACCAGTTCTGGGCAGACACAGCCACCTCGGTGCAGGATGTGTTTGCACT
GGTGCCGGCAGCAGAGATCCGGGCCGTGCGGGAAGAGTCACCCTCCAACTTGGCCACCCTGTGCTACAAGGCC
GTTGAGAAGCTGGTGCAGGGAGCTGAGAGTGGCTGCCACTCGGAGAAGGAGAAGCAGATCGTCCTGAACTGCA
GCCGGCTGCTCACCCGCGTGCTGCCCTACATCTTTGAGGACCCCGACTGGAGGGGCTTCTTCTGGTCCACAGT
GCCCCAGCAGGGAGAAGAGGATGATGAGCATGCCAGGCCCCTGGCCGAGTCCCTGCTCCTGGCCATTGCTGAC
CTGCTCTTCTGCCCGGACTTCACGGTTCAGAGCCACCGGAGGAGCACTGTGGACTCGGCAGAGGACGTCCACT
CCCTGGACAGCTGTGAATACATCTGGGAGGCTGGTGTGGGCTTCGCTCACTCCCCCCAGCCTAACTACATCCA
CGATATGAACCGGATGGAGCTGCTGAAACTGCTGCTGACATGCTTCTCCGAGGCCATGTACCTGCCCCCAGCT
CCGGAAAGTGGCAGCACCAACCCATGGGTTCAGTTCTTTTGTTCCACGGAGAACAGACATGCCCTGCCCCTCT
TCACCTCCCTCCTCAACACCGTGTGTGCCTATGACCCTGTGGGCTACGGGATCCCCTACAACCACCTGCTCTT
CTCTGACTACCGGGAACCCCTGGTGGAGGAGGCTGCCCAGGTGCTCATTGTCACTTTGGACCACGACAGTGCC
AGCAGTGCCAGCCCCACTGTGGACGGCACCACCACTGGCACCGCCATGGATGATGCCGATGACTTCCAGTTCA
TCCTCAAGGGTATAGCCCGGCTGCTGTCCAACCCCCTGCTCCAGACCTACCTGCCTAACTCCACCAAGAAGAT
CCAGTTCCACCAGGAGCTGCTAGTTCTCTTCTGGAAGCTCTGCGACTTCAACAAGAAATTCCTCTTCTTCGTG
CTGAAGAGCAGCGACGTCCTAGACATCCTTGTCCCCATCCTCTTCTTCCTCAACGATGCCCGGGCCGATCAGT
CTCGGGTGGGCCTGATGCACATTGGTGTCTTCATCTTGCTGCTTCTGAGCGGGGAGCGGAACTTCGGGGTGCG
GCTGAACAAACCCTACTCAATCCGCGTGCCCATGGACATCCCAGTCTTCACAGGGACCCACGCCGACCTGCTC
ATTGTGGTGTTCCACAAGATCATCACCAGCGGGCACCAGCGGTTGCAGCCCCTCTTCGACTGCCTGCTCACCA
TCGTGGTCAACGTGTCCCCCTACCTCAAGAGCCTGTCCATGGTGACCGCCAACAAGTTGCTGCACCTGCTGGA
GGCCTTCTCCACCACCTGGTTCCTCTTCTCTGCCGCCCAGAACCACCACCTGGTCTTCTTCCTCCTGGAGGTC
TTCAACAACATCATCCAGTACCAGTTTGATGGCAACTCCAACCTGGTCTACGCCATCATCCGCAAGCGCAGCA
TCTTCCACCAGCTGGCCAACCTGCCCACGGACCCGCCCACCATTCACAAGGCCCTGCAGCGGCGCCGGCGGAC
ACCTGAGCCCTTGTCTCGCACCGGCTCCCAGGAGGGCACCTCCATGGAGGGCTCCCGCCCCGCTGCCCCTGCA
GAGCCAGGCACCCTCAAGACCAGTCTGGTGGCTACTCCAGGCATTGACAAGCTGACCGAGAAGTCCCAGGTGT
CAGAGGATGGCACCTTGCGGTCCCTGGAACCTGAGCCCCAGCAGAGCTTGGAGGATGGCAGCCCGGCTAAGGG
GGAGCCCAGCCAGGCATGGAGGGAGCAGCGGCGACCGTCCACCTCATCAGCCAGTGGGCAGTGGAGCCCAACG
CCAGAGTGGGTCCTCTCCTGGAAGTCGAAGCTGCCGCTGCAGACCATCATGAGGCTGCTGCAGGTGCTGGTTC
CGCAGGTGGAGAAGATCTGCATCGACAAGGGCCTGACGGATGAGTCTGAGATCCTGCGGTTCCTGCAGCATGG
CACCCTGGTGGGGCTGCTGCCCGTGCCCCACCCCATCCTCATCCGCAAGTACCAGGCCAACTCGGGCACTGCC
ATGTGGTTCCGCACCTACATGTGGGGCGTCATCTATCTGAGGAATGTGGACCCCCCTGTCTGGTACGACACCG
ACGTGAAGCTGTTTGAGATACAGCGGGTGTGAGGATGAAGCCGACGAGGGGCTCAGTCTAGGGGAAGGCAGGG
CCTTGGTCCCTGAGGCTTCCCCCATCCACCATTCTGAGCTTTAAATTACCACGATCAGGGCCTGGAACAGGCA
GAGTGGCCCTGAGTGTCATGCCCTAGAGACCCCTGTGGCCAGGACAATGTGAACTGGCTCAGATCCCCCTCAA
CCCCTAGGCTGGACTCACAGGAGCCCCATCTCTGGGGCTATGCCCCCACCAGAGACCACTGCCCCCAACACTC
GGACTCCCTCTTTAAGACCTGGCTCAGTGCTGGCCCCTCAGTGCCCACCCACTCCTGTGCTACCCAGCCCCAG
AGGCAGAAGCCAAAATGGGTCACTGTGCCCTAAGGGGTTTGACCAGGGAACCACGGGCTGTCCCTTGAGGTGC
CTGGACAGGGTAAGGGGGTGCTTCCAGCCTCCTAACCCAAAGCCAGCTGTTCCAGGCTCCAGGGGAAAAAGGT
GTGGCCAGGCTGCTCCTCGAGGAGGCTGGGAGCTGGCCGACTGCAAAAGCCAGACTGGGGCACCTCCCGTATC
CTTGGGGCATGGTGTGGGGTGGTGAGGGTCTCCTGCTATATTCTCCTGGATCCATGGAAATAGCCTGGCTCCC
TCTTACCCAGTAATGAGGGGCAGGGAAGGGAACTGGGAGGCAGCCGTTTAGTCCTCCCTGCCCTGCCCACTGC
CTGGATGGGGCGATGCCACCCCTCATCCTTCACCCAGCTCTGGCCTCTGGGTCCCACCACCCAGCCCCCCGTG
TCAGAACAATCTTTGCTCTGTACAATCGGCCTCTTTACAATAAAACCTCCTGCTCCAAAAAAAAAAAAAAAAA
AAAAAAA

The NOV5a nucleic acid was identified on chromosome 17 and has 567 of 571 bases (99%) identical to a Homo sapiens DKFZp43411120 mRNA (gb:GENBANK-ID:HSM802295|acc:AL137556) (E=1.1e⁻²¹⁶)

A disclosed NOV5a polypeptide (SEQ ID NO:10) encoded by SEQ ID NO:9 is 764 amino acid residues and is presented using the one-letter code in Table SB. Signal P, Psort and/or Hydropathy results predict that NOV5a contains a signal peptide and is likely to be localized in the plasma membrane with a certainty of 0.7300 and the microbody (peroxisome) with a certainty of 0.6075. The most likely cleavage site for a NOV5a peptide is between amino acids 49 and 50, at: FAL-VP.

TABLE 5B
Encoded NOV5a protein sequence

(SEQ ID NO:10)

MGSTDSKLNFRKAVIQLTTKTQPVEATDDAFWDQFWADTATSVQDVFALVPAAEIRAVREESPSNLATLCYK
AVEKLVQGAESGCHSEKEKQIVLNCSRLLTRVLPYIFEDPDWRGFFWSTVPQQGEEDDEHARPLAESLLLAI
ADLLFCPDFTVQSHRRSTVDSAEDVHSLDSCEYIWEAGVGFAHSPQPNYIHDMNRMELLKLLLTCFSEAMYL
PPAPESGSTNPWVQFFCSTENRHALPLFTSLLNTVCAYDPVGYGIPYNHLLFSDYREPLVEEAAQVLIVTLD
HDSASSASPTVDGTTTGTAMDDADDFQFILKGIARLLSNPLLQTYLPNSTKKIQFHQELLVLFWKLCDFNKK
FLFFVLKSSDVLDILVPILFFLNDARADQSRVGLMHIGVFILLLLSGERNFGVRLNKPYSIRVPMDIPVFTG
THADLLIVVFHKIITSGHQRLQPLFDCLLTIVVNVSPYLKSLSMVTANKLLHLLEAFSTTWFLFSAAQNHHL
VFFLLEVFNNIIQYQFDGNSNLVYAIIRKRSIFHQLANLPTDPPTIHKALQRRRRTPEPLSRTGSQEGTSME
GSRPAAPAEPGTLKTSLVATPGIDKLTEKSQVSEDGTLRSLEPEPQQSLEDGSPAKGEPSQAWREQRRPSTS
SASGQWSPTPEWVLSWKSKLPLQTIMRLLQVLVPQVEKICIDKGLTDESEILRFLQHGTLVGLLPVPHPILI
RKYQANSGTAMWFRTYMWGVIYLRNVDPPVWYDTDVKLFEIQRV

The NOV5a amino acid sequence has 397 of 638 amino acid residues (62%) identical to, and 478 of 638 amino acid residues (74%) similar to, a Drosophila melanogaster 837 amino acid residue CG8841 protein (ptnr:SPTREMBL-ACC:Q9V695) (E=5.9e⁻²⁷⁰).

NOV5a is expressed in at least the following tissues: adrenal gland/suprarenal gland, amygdala, bone marrow, brain, colon, dermis, duodenum, hippocampus, hypothalamus, kidney, larynx, liver, lung, lymph node, lymphoid tissue, mammary gland/breast, ovary, pancreas, parotid salivary glands, pituitary gland, retina, small Intestine, spinal chord, stomach, substantia nigra, testis, thalamus, tonsils, umbilical vein, uterus, whole organism. This information was derived by determining the tissue sources of the sequences that were included in the invention. In addition, the NOV5A is predicted to be expressed in testis tissue because of the expression pattern of a closely related Homo sapiens DKFZp434I1120 mRNA (gb:GENBANK-ID:HSM802295|acc:AL137556).

NOV5b

A disclosed NOV5b nucleic acid of 3314 nucleotides (also referred to as CG54443-02) encoding a novel CG8841-like protein is shown in Table 5C. An open reading frame was identified beginning with an ATG initiation codon at nucleotides 97-99 and ending with a TGA codon at nucleotides 2461-2463. Putative untranslated regions upstream from the initiation codon and downstream from the termination codon are underlined in Table 5C, and thc start and stop codons are in bold letters.

TABLE 5C
NOV5b Nucleotide Sequence

(SEQ ID NO:11)

GCGAGAGCCGCGGGGGCCGCGGAGCTGGAGCCGGAGCTGAAGCCGGAGCCGGGTTGGAGTCTGGGCGGGG
GCCGGGCCGGAGCGGGCTCCAGAGACATGGGGTCGACCGACTCCAAGCTGAACTTCCGGAAGGCGGTGAT
CCAGCTCACCACCAAGACGCAGCCCGTGGAAGCCACCGATGATGCCTTTTGGGACCAGTTCTGGGCAGAC
ACAGCCACCTCGGTGCAGGATGTGTTTGCACTGGTGCCGGCAGCAGAGATCCGGGCCGTGCGGGAAGAGT
CACCCTCCAACTTGGCCACCCTGTGCTACAAGGCCGTTGAGAAGCTGGTGCAGGGAGCTGAGAGTGGCTG
CCACTCGGAGAAGGAGAAGCAGATCGTCCTGAACTGCAGCCGGCTGCTCACCCGCGTGCTGCCCTACATC
TTTGAGGACCCCGACTGGAGGGGCTTCTTCTGGTCCACAGTGCCCGGGGCAGGGCGAGGAGGGCAGGGAG
AAGAGGATGATGAGCATGCCAGGCCCCTGGCCGAGTCCCTGCTCCTGGCCATTGCTGACCTGCTCTTCTG
CCCGGACTTCACGGTTCAGAGCCACCGGAGGAGCACTGTGGACTCGGCAGAGGACGTCCACTCCCTGGAC
AGCTGTGAATACATCTGGGAGGCTGGTGTGGGCTTCGCTCACTCCCCCCAGCCTAACTACATCCACGATA
TGAACCGGATGGAGCTGCTGAAACTGCTGCTGACATGCTTCTCCGAGGCCATGTACCTGCCCCCAGCTCC
GGAAAGTGGCAGCACCAACCCATGGGTTCAGTTCTTTTGTTCCACGGAGAACAGACATGCCCTGCCCCTC
TTCACCTCCCTCCTCAACACCGTGTGTGCCTATGACCCTGTGGGCTACGGGATCCCCTACAACCACCTGC
TCTTCTCTGACACCGGGGAACCCCTGGTGGAGGAGGCTGCCCAGGTGCTCATTGTCACTTTGGACCACGA
CAGTGCCAGCAGTGCCAGCCCCACTGTGGACGGCACCACCACTGGCACCGCCATGGATGATGCCGATCCT
CCAGGCCCTGAGAACCTGTTTGTGAACTACCTGTCCCGCATCCATCGTGAGGAGGACTTCCAGTTCATCC
TCAAGGGTATAGCCCGGCTGCTGTCCAACCCCCTGCTCCAGACCTACCTGCCTAACTCCACCAAGAAGAT
CCAGTTCCACCAGGAGCTGCTAGTTCTCTTCTGGAAGCTCTGCGACTTCAACAAGAAATTCCTCTTCTTC
GTGCTGAAGAGCAGCGACGTCCTAGACATCCTTGTCCCCATCCTCTTCTTCCTCAACGATGCCCGGGCCG
ATCAGTCTCGGGTGGGCCTGATGCACATTGGTGTCTTCATCTTGCTGCTTCTGAGCGGGGAGCGGAACTT
CGGGGTGCGGCTGAACAAACCCTACTCAATCCGCGTGCCCATGGACATCCCAGTCTTCACAGGGACCCAC
GCCGACCTGCTCATTGTGGTGTTCCACAAGATCATCACCAGCGGGCACCAGCGGTTGCAGCCCCTCTTCG
ACTGCCTGCTCACCATCGTGGTCAACGTGTCCCCCTACCTCAAGAGCCTGTCCATGGTGACCGCCAACAA
GTTGCTGCACCTGCTGGAGGCCTTCTCCACCACCTGGTTCCTCTTCTCTGCCGCCCAGAACCACCACCTG
GTCTTCTTCCTCCTGGAGGTCTTCAACAACATCATCCAGTACCAGTTTGATGGCAACTCCAACCTGGTCT
ACGCCATCATCCGCAAGCGCAGCATCTTCCACCAGCTGGCCAACCTGCCCACGGACCCGCCCACCATTCA
CAAGGCCCTGCAGCGGCGCCGGCGGACACCTGAGCCCTTGTCTCGCACCGGCTCCCAGGAGGGCACCTCC
ATGGAGGGCTCCCGCCCCGCTGCCCCTGCAGAGCCAGGCACCCTCAAGACCAGTCTGGTGGCTACTCCAG
GCATTGACAAGCTGACCGAGAAGTCCCAGGTGTCAGAGGATGGCACCTTGCGGTCCCTGGAACCTGAGCC
CCAGCAGAGCTTGGAGGATGGCAGCCCGGCTAAGGGGGAGCCCAGCCAGGCATGGAGGGAGCAGCGGCGA
CCATCCACCTCATCAGCCAGTGGGCAGTGGAGCCCAACGCCAGAGTGGGTCCTCTCCTGGAAGTCGAAGC
TGCCGCTGCAGACCATCATGAGGCTGCTGCAGGTGCTGGTTCCGCAGGTGGAGAAGATCTGCATCGACAA
GGGCCTGACGGATGAGTCTGAGATCCTGCGGTTCCTGCAGCATGGCACCCTGGTGGGGCTGCTGCCCGTG
CCCCACCCCATCCTCATCCGCAAGTACCAGGCCAACTCGGGCACTGCCATGTGGTTCCGCACCTACATGT
GGGGCGTCATCTATCTGAGGAATGTGGACCCCCCTGTCTGGTACGACACCGACGTGAAGCTGTTTGAGAT
ACAGCGGGTGTGAGGATGAAGCCGACGAGGGGCTCAGTCTAGGGGAAGGCAGGGCCTTGGTCCCTGAGGC
TTCCCCCATCCACCATTCTGAGCTTTAAATTACCACGATCAGGGCCTGGAACAGGCAGAGTGGCCCTGAG
TGTCATGCCCTAGAGACCCCTGTGGCCAGGACAATGTGAACTGGCTCAGATCCCCCTCAACCCCTAGGCT
GGACTCACAGGAGCCCCATCTCTGGGGCTATGCCCCCACCAGAGACCACTGCCCCCAACACTCGGACTCC
CTCTTTAAGACCTGGCTCAGTGCTGGCCCCTCAGTGCCCACCCACTCCTGTGCTACCCAGCCCCAGAGGC
AGAAGCCAAAATGGGTCACTGTGCCCTAAGGGGTTTGACCAGGGAACCACGGGCTGTCCCTTGAGGTGCC
TGGACAGGGTAAGGGGGTGCTTCCAGCCTCCTAACCCAAAGCCAGCTGTTCCAGGCTCCAGGGGAAAAAG
GTGTGGCCAGGCTGCTCCTCGAGGAGGCTGGGAGCTGGCCGACTGCAAAAGCCAGACTGGGGCACCTCCC
GTATCCTTGGGGCATGGTGTGGGGTGGTGAGGGTCTCCTGCTATATTCTCCTGGATCCATGGAAATAGCC
TGGCTCCCTCTTACCCAGTAATGAGGGGCAGGGAAGGGAACTGGGAGGCAGCCGTTTAGTCCTCCCTGCC
CTGCCCACTGCCTGGATGGGGCGATGCCACCCCTCATCCTTCACCCAGCTCTGGCCTCTGGGTCCCACCA
CCCAGCCCCCCGTGTCAGAACAATCTTTGCTCTGTACAATCGGCCTCTTTACAATAAAACCTCCTGCTCC
AAAAAAAAAAAAAAAAAAAAAAAA

The NOV5b nucleic acid was identified on chromosome 17 and has 1155 of 1162 bases (99%) identical to a Homo sapiens DKFZp434I1120 mRNA (gb:GENBANK-ID:HSM802295|acc:AL137556.1) (E=1.2e⁻²⁵⁵)

A disclosed NOV5b polypeptide (SEQ ID NO:12) encoded by SEQ ID NO:11 is 788 amino acid residues and is presented using the one-letter code in Table 5D. Signal P, Psort and/or Hydropathy results predict that NOV5b contains a signal peptide and is likely to be localized to the plasma membrane with a certainty of 0.7300 and to the microbody (peroxisome) with a certainty of 0.6006. The most likely cleavage site for a NOV5b peptide is between amino acids 49 and 50, at: FAL-VP.

TABLE 5D
Encoded NOV5b protein sequence.

(SEQ ID NO:12)

MGSTDSKLNFRKAVIQLTTKTQPVEATDDAFWDQFWADTATSVQDVFALVPAAEIRAVREESPSNLATLCYKAVEK
LVQGAESGCHSEKEKQIVLNCSRLLTRVLPYIFEDPDWRGFFWSTVPGAGRGGQGEEDDEHARPLAESLLLAIADL
LFCPDFTVQSHRRSTVDSAEDVHSLDSCEYIWEAGVGFAHSPQPNYIHDMNRMELLKLLLTCFSEAMYLPPAPESG
STNPWVQFFCSTENRHALPLFTSLLNTVCAYDPVGYGIPYNHLLFSDTGEPLVEEAAQVLIVTLDHDSASSASPTV
DGTTTGTAMDDADPPGPENLFVNYLSRIHREEDFQFILKGIARLLSNPLLQTYLPNSTKKIQFHQELLVLFWKLCD
FNKKFLFFVLKSSDVLDILVPILFFLNDARADQSRVGLMHIGVFILLLLSGERNFGVRLNKPYSIRVPMDIPVFTG
THADLLIVVFHKIITSGHQRLQPLFDCLLTIVVNVSPYLKSLSMVTANKLLHLLEAFSTTWFLFSAAQNHHLVFFL
LEVFNNIIQYQFDGNSNLVYAIIRKRSIFHQLANLPTDPPTIHKALQRRRRTPEPLSRTGSQEGTSMEGSRPAAPA
EPGTLKTSLVATPGIDKLTEKSQVSEDGTLRSLEPEPQQSLEDGSPAKGEPSQAWREQRRPSTSSASGQWSPTPEW
VLSWKSKLPLQTIMRLLQVLVPQVEKICIDKGLTDESEILRFLQHGTLVGLLPVPHPILIRKYQANSGTAMWFRTY
MWGVIYLRNVDPPVWYDTDVKLFEIQRV

The NOV5b amino acid sequence has 409 of 662 amino acid residues (61%) identical to, and 491 of 662 amino acid residues (74%) similar to, a Drosophila melalogaster 837 amino acid residue CG8841 protein (ptnr:SPTREMBL-ACC:Q9V695) (E=1.4e277).

NOV5b is expressed in at least the following tissues: Adrenal Gland/Suprarenal gland, Bone Marrow, Brain, Cartilage, Colon, Dermis, Duodenum, Gall Bladder, Kidney, Larynx, Liver, Lung, Lymph node, Lymphoid tissue, Mammary gland/Breast, Ovary, Pancreas, Parotid Salivary glands, Pituitary Gland, Prostate, Retina, Small Intestine, Spinal Cord, Spleen, Stomach, Testis, Tonsils, Urinary Bladder, Uterus, Vein, Vulva. In addition, this gene was expressed in the following disease states: prostatic adenocarcinoma, ovarian carcinoma, colon carcinoma, uterine carcinoma, pancreatic adenocarcinoma, breast cancer. This information was derived by determining the tissue sources of the sequences that were included in the invention.

NOV5a and NOV5b are very closely homologous as is shown in the amino acid alignment in Table 5E.

[image]

[image]

Homologies to any of the above NOV5 proteins will be shared by the other NOV5 proteins insofar as they are homologous to each other as shown above. Any reference to NOV5 is assumed to refer to both of the NOV5 proteins in general, unless otherwise noted.

NOV5a also has homology to the amino acid sequences shown in the BLASTP data listed in Table 5F.

TABLE 4D
BLAST results for NOV5a

| Gene Index/ | | Length | Identity | Positives | |
|---|---|---|---|---|---|
| Identifier | Protein/Organism | (aa) | (%) | (%) | Expect |
| gi\|7303477\|gb\|AAF58533.1\| | CG8841 gene product | 837 | 519/844 | 600/844 | 0.0 |
| (AE003822) | [Drosophila | | (61%) | (70%) | |
| | melanogaster] | | | | |
| gi\|7505130\|pir\|T16522 | hypothetical protein | 729 | 422/782 | 530/782 | 0.0 |
| | K02E10.2 | | (53%) | (66%) | |
| | [Caenorhabditis | | | | |
| | elegans] | | | | |
| gi\|11360052\|pir\|T46395 | hypothetical protein | 380 | 328/354 | 328/354 | 0.0 |
| | DKFZp434I1120.1 | | (92%) | (92%) | |
| | (fragment) [Homo | | | | |
| | sapiens] | | | | |
| gi\|7106107\|emb\|CAB76033.1\| | conserved hypothetical | 767 | 203/837 | 360/837 | 1e−44 |
| (AL157917) | protein | | (24%) | (42%) | |
| | [Schizosaccharomyces | | | | |
| | pombe] | | | | |
| gi\|6648087\|sp\|O13776\|YE9G_ | hypothetical 104.8 | 925 | 166/679 | 299/679 | 3e−31 |
| SCHPO | KDA protein | | (24%) | (43%) | |
| | C17A5.16 IN | | | | |
| | CHROMOSOME I | | | | |
| | [Schizosaccharomyces | | | | |
| | pombe] | | | | |

The homology of these sequences is shown graphically in the ClustalW analysis shown in Table 5G.

[image]

[image]

[image]

The amino acid sequence of NOV5 has high homology to other proteins as shown in Table 5H.

TABLE 5H
BLASTX results for NOV5

| | | Smallest | | |
|---|---|---|---|---|
| | | Sum | | |
| | Reading High | Prob | | |
| Sequences producing High-scoring Segment Pairs: | Frame | Score | P (N) | N |
| patp: AAY91644 Secreted prot sequ gene 43, Homo Sapi 290 aa . . . | +1 | 1007 | 1.1e−140 | 1 |
| patp: AAY91493 Secreted prot sequ gene 43, Homo Sapi 214 aa . . . | +1 | 614 | 6.3e−97 | 1 |

The above defined information for NOV5 suggests that this NOV5 protein may function as a member of a CG8841-like protein family. Therefore, the NOV5 nucleic acids and proteins of the invention are useful in potential therapeutic applications implicated in various diseases and disorders described below and/or other pathologies. For example, the NOV5 compositions of the present invention will have efficacy for treatment of patients suffering from cancer, trauma, immunological disease, respiratory disease, gastro-intestinal diseases, reproductive health, neurological and neurodegenerative diseases, bone marrow transplantation, metabolic and endocrine diseases, allergy and inflammation, nephrological disorders, hematopoietic disorders or unirary system disorders. The NOV5 nucleic acid encoding CG8841-like protein, and the CG8841-like protein of the invention, or fragments thereof, may further be useful in diagnostic applications, wherein the presence or amount of the nucleic acid or the protein are to be assessed.

NOV6

NOV6 includes two novel Synaptotagmin-like proteins disclosed below. The disclosed proteins have been named NOV6a and NOV6b.

NOV6a

A disclosed NOV6a nucleic acid of 1116 nucleotides (also referred to as SC134912642_da1) encoding a novel Synaptotagmin-like protein is shown in Table 6A. An open reading frame was identified beginning with an ATG initiation codon at nucleotides 1-3 and ending with a TGA codon at nucleotides 1114-1116. The start and stop codons are in bold letters in Table 6A.

TABLE 6A
NOV6a Nucleotide Sequence
(SEQ ID NO:13)

ATGTACCGGGACCCGGAGGCGGCCAGCCCAGGTGCGCCCTCGCGCGACGTCCTGCTGGTCTCTGCCATCATCA
CCGTCAGCCTTAGCGTCACTGTCGTCCTCGCTAGCCGGTGCCACTGGTGTCAGCGCAAACTGGGCAAACGCTA
CAAGAATTCCTTGGAGACGGTGGGCACGCCAGACTCAGGACGTGGGCGCAGTGAGAAGAAGGCTATCAAGTTG
CCTGCAGGAGGGAAGGCGGTGAACACAGCCCCCGTGCCAGGCCAGACACCCCACGATGAGTCCGACCGCCGGA
CCGAGCCACGTTCCTCCTTCTCAGACCTCGTCAACTCCCTCACCAGCGAGATGCTCATGGAGTCCACGCTCAC
CGTGAAGATCATGAAGGCCCAGGAGCTGCCGGCCAAGGACTTCAGCGGCACCAGCGACCCCTTCGTCAAGATC
TACCTGCTGCCCGACAAGAAGCACAAGCTGGAGACCAAGGTGAAGCGGAAGAACCTGAACCCCCACTGGAACG
AGACCTTCCTCTTTGAAGGTTTTCCCTATGAGAAGGTGGTGCAGAGGATCCTCTACCTCCAAGTCCTGGACTA
TGACCGCTTCAGCCGCCACGACCCCATTGGGGAGGTGTCCATCCCCCTTAAACAGGTGGACCTGACCCAGATG
CAGATCTGGAAGGATCTGAAGCCATGCAGCGATGGGAGTGGGAGCCGAGGGGAGCTGCTCTTGTCTCTCTGCT
ACAACCCCTCTGCCAACTCCATCATCGTGAACATCATCAAAGCCCGGAACCTCAAAGCCATGGACATCGGGGG
CACATCAGACCCCTACGTGAAGGTATGGCTGATGTACAAGGACAAGCGGGTGGAGAAGAAGAAGACGGTGACG
ATGAAGAGGAACCTGAACCCCATCTTCAATGAGTCCTTCGCCTTCGATATCCCCACGGAGAAGCTGAGGGAGA
CGACCATCATCATCACTGTCATGGACAAGGACAAGCTCAGCCGCAATGACGTCATCGGCAAGATCTACCTGTC
CTGGAAGAGCGGGCCAGGGGAGGTGAAGCACTGGAAGGACATGATTGCCCGTCCCCGGCAGCCCGTGGCCCAG
TGGCACCAGCTGAAGGCCTGA

The NOV6a nucleic acid was identified on chromosome 11q12.2 and has 709 of 768 bases (92%) identical to a Mus musculus synaptotagmin VII mRNA (gb:GENBANK-208) ID:AB026804|acc:AB026804) (E=1.3e⁻²⁰⁸).

A disclosed NOV6a polypeptide (SEQ ID NO:14) encoded by SEQ ID NO:13 is 371 amino acid residues and is presented using the one-letter code in Table 6B. Signal P, Psort and/or Hydropathy results predict that NOV6a contains a signal peptide and is likely to be localized in the cytoplasm with a certainty of 0.8200. The most likely cleavage site for a NOV6a peptide is between amino acids 35 and 36, at: VLA-SR.

TABLE 6B
Encoded NOV6a protein sequence

(SEQ ID NO:14)

MYRDPEAASPGAPSRDVLLVSAIITVSLSVTVVLASRCHWCQRKLGKRYKNSLETVGTPDSGRGRSEKKAIK
LPAGGKAVNTAPVPGQTPHDESDRRTEPRSSFSDLVNSLTSEMLMESTLTVKIMKAQELPAKDFSGTSDPFV
KIYLLPDKKHKLETKVKRKNLNPHWNETFLFEGFPYEKVVQRILYLQVLDYDRFSRHDPIGEVSIPLKQVDL
TQMQIWKDLKPCSDGSGSRGELLLSLCYNPSANSIIVNIIKARNLKAMDIGGTSDPYVKVWLMYKDKRVEKK
KTVTMKRNLNPIFNESFAFDIPTEKLRETTIIITVMDKDKLSRNDVIGKIYLSWKSGPGEVKHWKDMIARPR
QPVAQWHQLKA

The NOV6a amino acid sequence has 248 of 255 amino acid residues (97%) identical to, and 251 of 255 amino acid residues (98%) similar to, a Rattus norvegicus 403 amino acid residue synaptotagmin VII protein (ptnr:SPTREMBL-ACC:Q62747) (E=1.3⁻¹⁹⁰).

NOV6a is expressed in at least the following tissues: Adrenal Gland/Suprarenal gland, Bone, Brain, Cerebral Medulla/Cerebral white matter, Heart, Hippocampus, Liver, Mammary gland/Breast, Pituitary Gland, Placenta, Salivary Glands, Thalamus. This information was derived by determining the tissue sources of the sequences that were included in the invention including but not limited to SeqCalling sources, Public EST sources, Literature sources, and/or RACE sources.

NOV6b

A disclosed NOV6b nucleic acid of 1212 nucleotides (also referred to as CG56106-01) encoding a novel Synaptotagmin-like protein is shown in Table 6C. An open reading frame was identified beginning with an ATG initiation codon at nucleotides 1-3 and ending with a TGA codon at nucleotides 1210-1212. The start and stop codons are in bold letters in Table 6C.

TABLE 6C
NOV6b Nucleotide Sequence

(SEQ ID NO:15)

ATGTACCGGGACCCGGAGGCGGCCAGCCCAGGGGCGCCCTCGCCCGACGTCCTGCTGGTCTCTGCCATCA
TCACCGTCACCCTTAGCGTCACTGTCGTCCTCTGCCGCCTCTCCCACTOGTGTCAGCGCAAACTGGGCAA
ACGCTACAACAATTCCTTGGAGACGGTGGGCACGCCAGACTCAGGGCGTGGGCGCAGTGAGAAGAAGCCT
ATCAAGTTGCCTGCAGGAGGGAAGGCGGTGAACACAGCCCCCGTGCCAGGCCAGACACCCCACGATGAGT
CCGACCGCCGGACCGAGCCACGTTCCTCCGTCTCAGACCTCGTCAACTCCCTCACCAGCGAGATGCTCAT
GCTCTCCCCAGGCTCCGAGGAGGATGAGGCCCACGACGGTTGCAGCCGAGAGAACCTGGGCCGGATCCAG
TTCAGTGTCGGCTACAACTTCCAGGAGTCCACGCTCACCGTGAAGATCATGAAGGCCCAGGAGCTGCCGG
CCAAGGACTTCAGCGGCACCAGCGACCCCTTCGTCAAGATCTACCTGCTGCCCGACAAGAAGCACAAGCT
GGAGACCAAGGTGAAGCGGAAGAACCTGAACCCCCACTGGAACGAGACCTTCCTCTTTGAAGGTTTTCCC
TATGAGAAGGTGGTGCAGAGGATCCTCTACCTCCAAGTCCTGGACTATGACCGCTTCAGCCGCAACGACC
CCATTCGGGAGGTGTCCATCCCCCTTAACAAGGTGGACCTGACCCAGATGCAGACCTTCTGGAAGGATCT
GAAGCCATGCAGCGATGGGAGTGGGAGCCGAGQGGAGCTGCTCTTGTCTCTCTGCTACAACCCCTCTGCC
AACTCCATCATCGTGAACATCATCAAAGCCCGGAACCTCAAAGCCATGGACATCCGGGCCACATCAGACC
CCTACGTGAAGGTATGGCTGATGTACAAGGACAAGCGGGTGGAGAAGAAGAAGACGGTGACGATGAAGAG
GAACCTGAACCCCATCTTCAATGAGTCCTTCGCCTTCGATATCCCCACGGAGAAGCTGAGGCAGACGACC
ATCATCATCACTGTCATGGACAAGGACAACCTCAGCCGCAATGACGTCATCCGCAAGATCTACCTGTCCT
GGAAGAGCGGGCCAGGGGAGGTGAAGCACTGGAAGGACATGATTGCCCGTCCCCGGCAGCCCGTGGCCCA
GTCGCACCAGCTGAAGGCCTGA

The NOV6b nucleic acid was identified on chromosome 11q2-13.1 and has 1201 of 1212 bases (99%) identical to a Homo sapiens synaptotagmin VII mRNA (gb:GENBANK-ID:AF038535|acc:AF038535.1) (E=5.6e⁻²⁶³)

A disclosed NOV6b polypeptide (SEQ ID NO:16) encoded by SEQ ID NO:15 is 403 amino acid residues and is presented using the one-letter code in Table 6D. Signal P, Psort and/or Hydropathy results predict that NOV6b contains a signal peptide and is likely to be localized in the endoplasmic reticulum (membrane) with a certainty of 0.8200 and the plasma membrane with a certainty of 0.5140. The most likely cleavage site for a NOV6b peptide is between amino acids 46 and 47, at: KLG-KR.

TABLE 6D
Encoded NOV6b protein sequence.

(SEQ ID NO:16)

MYRDPEAASPGAPSRDVLLVSAIITVSLSVTVVLCGLCHWCQRKLGKRYKNSLETVGTPDSGRGRSEKKAIKLPA
GGKAVNTAPVPGQTPHDESDRRTEPRSSVSDLVNSLTSEMLMLSPGSEEDEAHECCSRENLGRIQFSVGYNFQES
TLTVKIMKAQELPAKDPSGTSDPFVKIYLLPDKKHKLETKVKRKNLNPHWNETFLFEGFPYEKVVQRILYLQVLD
YDRFSRNDPIGEVSIPLNKVDLTQMQTFWKDLKPCSDGSGSRGELLLSLCYNPSANSIIVNIIKARNLKAMDIGG
TSDPYVKVWLMYKDKRVEKKKTVTMKRNLNPIFNESFAFDTPTEKLRETTIIITVMDKDKLSRNDVIGKIYLSWK
SCPGEVKHWKDMIARPRQPVAQWHQLKA

The NOV6b amino acid sequence has 398 of 403 amino acid residues (98%) identical to, and 401 of 403 amino acid residues (99%) similar to, a Rattus norvegicus 403 amino acid residue synaptotagmin VII protein (ptnr:SPTREMBL-ACC:Q62747) (E=7.1e⁻²¹⁷).

NOV6b is expressed in at least the following tissues: Adrenal Gland/Suprarenal gland, Bone, Brain, Cerebral Medulla/Cerebral white matter, Heart, Hippocampus, Liver, Mammary gland/Breast, Pituitary Gland, Placenta, Salivary Glands, Thalamus. This information was derived by determining the tissue sources of the sequences that were included in the invention including but not limited to SeqCalling sources, Public EST sources, Literature sources, and/or RACE sources.

NOV6a and NOV6b are very closely homologous as is shown in the amino acid alignment in Table 6E.

[image]

Homologies to any of the above NOV6 proteins will be shared by the other NOV6 proteins insofar as they are homologous to each other as shown above. Any reference to NOV6 is assumed to refer to both of the NOV6 proteins in general, unless otherwise noted.

NOV6a also has homology to the amino acid sequences shown in the BLASTP data listed in Table 6F.

TABLE 4D
BLAST results for NOV6a

| Gene Index/ | | Length | Identity | Positives | |
|---|---|---|---|---|---|
| Identifier | Protein/Organism | (aa) | (%) | (%) | Expect |
| gi\|11067375\|ref\|NP_ | synaptotagmin 7 | 403 | 358/403 | 363/403 | 0.0 |
| 067691.1\| | [Rattus | | (88%) | (89%) | |
| | norvegicus] | | | | |
| gi\|9055364\|ref\|NP_061271.1\| | synaptotagmin 7 | 403 | 356/403 | 362/403 | 0.0 |
| | [Mus musculus] | | (88%) | (89%) | |
| gi\|2724126\|gb\|AAB92667.1\| | synaptotagmin VII | 418 | 350/403 | 356/403 | 0.0 |
| (AF038535) | [Homo sapiens] | | (86%) | (87%) | |
| gi\|12667450\|gb\|AAK01451.1\| | synaptotagmin | 520 | 296/351 | 304/351 | 1e−159 |
| AF336856_1 | VIIa [Rattus | | (84%) | (86%) | |
| (AF336856) | norvegicus] | | | | |
| gi\|12667458\|gb\|AAK01455.1\| | synaptotagmin | 643 | 296/351 | 304/351 | 1e−159 |
| AF336860_1 | VIIe [Rattus | | (84%) | (86%) | |
| (AF336860) | norvegicus] | | | | |

The homology of these sequences is shown graphically in the ClustalW analysis shown in Table 6G.

[image]

[image]

Table 6H-6K lists the domain description from DOMAIN analysis results against NOV6a. This indicates that the NOV6a sequence has properties similar to those of other proteins known to contain this domain.

TABLE 6H
Domain Analysis of NOV6a

gnl|Smart|smart00239, C2, Protein kinase C conserved region 2 (CalB)
Ca2+-binding motif present in phospholipases, protein kinases C, and
synaptotamins (among others). Some do not appear to contain Ca2+-
binding sites. Particular C2s appear to bind phospholipids, inositol
polyphosphates, and intracellular proteins. Unusual occurrence in
perform. Synaptotagmin and PLC C2s are permuted in sequence with
respect to N- and C-terminal beta strands. SMART detects C2 domains
using one or both of two profiles.. (SEQ ID NO:94)
Length = 101 residues, 99.0% aligned
Score = 103 bits (258) , Expect = 1e−23

| | | | |
|---|---|---|---|
| NOV6a | 120 | TLTVKIMKAQELPAKDFSGTSDPFVKIYLLPDKKHKLETKVKRKNLNPHWNETFLPEGPP | 179 |
| | | \|\|\|\|\|\|+\|+\|\|\|\| \|\|\|\|+\|\|+\| \|+\|+\|\|\|+ \|\|\|\|\|\|\|\|\|\| \| | |
| 00239 | 1 | TLTVKIISARNLPPKDKGGKSDPYVKVSLDGDPREKKKTKVVKNTLNPVWNETFEFEVPP | 60 |
| NOV6a | 180 | YEKVVQRILYLQVLDYDRFSRHDPIGEVSIPLKQVDLTQMQIW 222 | |
| | | \| \|++\|\|\|\|\|\|\|\|\|\|\|+\|\|\| +\| | |
| 00239 | 61 | PE---LSELEIEVYDKDRFSRDDFIGRVTIPLSDLLLGGRHEK 100 | |

TABLE 6I
Domain Analysis of NOV6a

gnl|Smart|smart00239 (SEQ ID NO:94)
Length = 101 residues, 96.0% aligned
Score = 91.3 bits (225), Expect = 9e−20

| | | | |
|---|---|---|---|
| NOV6a | 53 | LETVGTPDSGRGRSEKKAIKLPAGGKAVNTAPVPGQTPHDESDRRTEPRSSFSDLVNSLT | 112 |
| | | \|+ \|\| + ++\| +\|\|\|\|\|\|\|\|\|\|\|\|\|\|\|\|\|\|\|\|\|\|\|\|\|\|\|\|\|\|\|\|\|\|\|\|\|\|\| | |
| 00239 | 296 | LQNPGTQQNQNAQGDK---RLPAGGKAVNTAPVPGQTPHDESDRRTEPRSSVSDLVNSLT | 352 |
| NOV6a | 113 | SEMLM-------------------------------ESTLTVKIMKAQELPAKDFSGTSD | 141 |
| | | \|\|\|\|\| \|\|\|\|\|\|\|+\|\|\|\|\|\|\|\|\|\|\|\|\|\|\|\| | |
| 00239 | 353 | SEMLMLSPGSEEDEAHEGCSRENLGRIQFSVGYNFQESTLTVKVMKAQELPAKDFSGTSD | 412 |
| NOV6a | 142 | PFVKIYLLPDKKHKLETKVKRKNLNPHWNETFLFEGFPYEKVVQRILYLQVLDYDRFSRH | 201 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|+ | |
| 00239 | 413 | PFVKIYLLPDKKHKLETKVKRKNLNPHWNETFLFEGFPYEKVVQRILYLQVLDYDRFSRN | 472 |
| NOV6a | 202 | DPIGEVSIPLKQVDLTQMQ-IWKDLKPCSDGSGSRGELLLSLCYNPSANSIIVNIIKARN | 260 |
| | | \|\|\|\|\|\|\|\|\|\|+\|\|\|\|\|\|\| \|\|\|\|\|\|\|\|\|\|\|\|\|\|\|\|\|\|\|\|\|\|\|\|\|\|\|\|\|\|\|\|\|\|\|\|\|\|\| | |
| 00239 | 473 | DPIGEVSIPLNKVDLTQMQTFWKDLKPCSDGSGSRGELLLSLCYNPSANSIIVNIIKARN | 532 |
| NOV6a | 261 | LKAMDIGGTSDPYVKVWLMYKDKRVEKKKTVTMKRNLNPIFNESFAFDIPTEKLRETTII | 320 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 00239 | 533 | LKAMDIGGTSDPYVKVMLMYKDKRVEKKKTVTKKRNLNPIFNESFAFDIPTEKLRETTII | 592 |
| NOV6a | 321 | ITVMDKDKLSRNDVIGKIYLSWKSGPGEVKHWKDMIARPRQPVAQWHQLKA 371 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 00239 | 593 | ITVMDKDKLSRNDVIGKIYLSWKSGPGEVKHWKDMIARPRQPVAQWHQLKA 643 | |

TABLE 6J
Domain Analysis of NOV6a

gnl|Pfam|pfam00168, C2, C2 domain, (SEQ ID NO:95)
Length = 88 residues, 98.9% aligned
Score = 98.6 bits (244), Expect = 6e−22

| | | | |
|---|---|---|---|
| NOV6a | 121 | LTVKIMKAQELPAKDFSGTSDPFVKIYLLPDKK--HKLETKVKRKNLNPHWNETFLFEGF | 178 |
| | | \|\|\|\|++\|+\|\| \|+\|\|\|\|+\|\|+\| \|\| \|+\|\| +\|\|\|\|\|\|\|\|\|+\|\| | |
| 00168 | 1 | LTVKVISARNLPKMDMNGLSDPYVKVDLDGDPKDTKKFKTKTVKKTLNPVWNETFVFEFV | 60 |
| NOV6a | 179 | PYEKVVQRILYLQVLDYDRFSRHDPIGEV 207 | |
| | | \| \| \|\|\|\|\|\|\|\|\|\|+\| | |
| 00168 | 61 | PLPD--LASLRFAVYDEDRFSRDDFIGQV 87 | |

TABLE 6K
Domain Analysis of NOV6a

gnl|Pfam|pfam00168, C2, C2 domain. (SEQ ID NO:95)
Length = 88 residues. 96.6% aligned
Score = 88.6 bits (218), Expect = 6e−19

| | | | |
|---|---|---|---|
| NOV6a | 251 | IIVNIIKARNLKAMDIGGTSDPYVKVWLMYKDKRVEKKKTVTMKRNLNPIFNESFAFD-I | 309 |
| | | +\|+\|\|\|\|\| \|\|+\|\|\|\|\|\|\|\|\| \| +\|\|\|\|+\|+\|\|\|++\|\|+\|\|++ | |
| 00168 | 1 | LTVKVISARNLPKMDMNGLSDPYVKVDLDGDPKDTKKFKTKTVKKTLNPVWNETFVFEKV | 60 |
| NOV6a | 310 | PTEKLRETTIIITVMDKDKLSRNDVIG 336 | |
| | | \| \| + \|\|+\|+\|\|+\|\|\| | |
| 00168 | 61 | PLPDLAS--LRFAVYDEDRFSRDDFIG 85 | |

Synaptotagmins are a family of brain-specific calcium-dependent phospholipid-binding proteins that play a role in synaptic exocytosis and neurotransmitter release. While constructing a transcript map of the human chromosomal 11q13 interval associated with Best vitelliform macular dystrophy, Cooper et al. isolated cDNAs encoding the human homolog of rat synaptotagmin VII (Cooper et al., Genomics 49: 419-429, 1998). The predicted 403-amino acid human and rat proteins are 98% identical. Northern blot analysis revealed that synaptotagmin VII is expressed as 4.4- and 7.5-kb mRNAs in a variety of human adult and fetal tissues, including those from different regions of the brain.

Neurons release neurotransmitters by calcium-dependent exocytosis of synaptic vesicles. Brose et al. reported that synaptotagmin, a highly conserved synaptic vesicle protein, binds calcium at physiological concentrations in a complex with negatively charged phospholipids.(Brose et al., Science 256:1021-1025, 1992). This binding is specific for calcium and involves the cytoplasmic domain of synaptotagmin. Calcium binding is dependent on the intact oligomeric structure of synaptotagmin; it is abolished by proteolytic cleavage at a single site. Brose et al. (1992) interpreted the results as suggesting that synaptotagmin acts as a cooperative calcium receptor in exocytosis. Synaptotagmin contains 2 copies of a sequence that is homologous to the regulatory region of protein kinase C. Perin et al. characterized full-length cDNAs encoding human and Drosophila synaptotagmins (Perin et al., Nature 345:260-263, 1991). Similarity of the phospholipid binding properties of the cytoplasmic domains of rat, human, and Drosophila synaptotagmins and selective conservation of the sequences that are homologous to protein kinase C suggested that these may be involved in phospholipid binding.

The above defined information for NOV6 suggests that NOV6 may function as a member of a synaptotagmin family. Therefore, the NOV6 nucleic acids and proteins of the invention are useful in potential therapeutic applications implicated in various diseases and disorders described below and/or other pathologies. For example, the NOV6 compositions of the present invention will have efficacy for treatment of patients suffering from Atopy; Osteoporosis-pseudoglioma syndrome; Smith-Lemli-Opitz syndrome, type I; Smith-Lemli-Opitz syndrome, type II; Xeroderma pigmentosum, group E, subtype 2; Asthma, atopic, susceptibility to; Diabetes mellitus, insulin-dependent, 4; Susceptibility to IDDM; Angioedema, hereditary; Paraganglioma, familial nonchromaffin, 2; Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection; metabolic disorders and Lambert-Eaton myasthenic syndrome. The NOV6 nucleic acid encoding synaptotagmin-like protein, and the synaptotagmin-like protein of the invention, or fragments thereof, may further be useful in diagnostic applications, wherein the presence or amount of the nucleic acid or the protein are to be assessed.

NOV7

A disclosed NOV7 nucleic acid of 1164 nucleotides (also referred to wugc_draft_h_nh0781m21_{—}20000809_da1) encoding a novel Serine Protease TLSP-like receptor protein is shown in Table 7A. An open reading frame was identified beginning with an ATG initiation codon at nucleotides 113-115 and ending with a TAG codon at nucleotides 854-856. Putative untranslated regions are found upstream from the initiation codon and downstream from the termination codon in Table 7A, and the start and stop codons are in bold letters.

TABLE 7A
NOV7 Nucleotide Sequence

(SEQ ID NO:17)

CTGCCTTGCTCCACACCTGGTCAGGGGAGAGAGGGGAGGAAAGCCAAGGGAAGGGACCTAACTGAAAACAA
ACAAGCTGGGAGAAGCAGGAATCTGCGCTCGGGTTCCGCAGATGCAGAGGTTGAGGTGGCTGCGGGACTGG
AACTCATCGGGCAGAGGTCTCACACCAGCCAAGGAACCTGGGGCCCGCTCCTCCCCCCTCCAGGCCATGAG
GATTCTGCAGTTAATCCTGCTTGCTCTGGCAACAGGGCTTGTAGGGGGAGAGACCAGGATCATCAAGGGGT
TCGAGTGCAAGCCTCACTCCCAGCCCTGGCAGGCAGCCCTGTTCGAGAAGACGCGGCTACTCTGTGGGGCG
ACGCTCATCGCCCCCAGATGGCTCCTGACAGCAGCCCACTGCCTCAAGCCCCTCCCCAACAAAGACCGCCG
CAATGACATCATGCTGGTGAAGATGGCATCGCCAGTCTCCATCACCTGGGCTGTGCGACCCCTCACCCTCT
CCTCACGCTGTGTCACTGCTGGCACCAGCTGCCTCATTTCCGGCTGGGGCAGCACGTCCAGCCCCCAGTTA
CCCCTGCCTCACACCTTGCGATGCCCCAACATCACCATCATTGAGCACCAGAAGTGTGACAACGCCTACCC
CGGCAACATCACAGACACCATGGTGTCTGCCAGCGTGCAGGAAGGGGGCAAGGACTCCTGCCAGGGTGACT
CCGGGGGCCCTCTGGTCTGTAACCACTCTCTTCAAGGCATTATCTCCTGGCGCCAGGATCCGTGTGCGATC
ACCCGAAAGCCTGGTGTCTACACGAAAGTCTGCAAATATGTGGTCTGGATCCAGGAGACGATTAAGAACAA
TTAGGCTGGACCCACCCACCACAGCCCATCACCCTCCATTTCCACTTGGTGTTTGGTTCCTGTTCACTCTG
TTAATAAGAAACCCTAAGCCAAGACCCTCTACGAACATTCTTTGGGCCTCCTGGACTACAGCAGATGCTGT
CACTTAATAATCAACCTGGGGTTCGAAATCAGTGAGACCTGGATTCAAATTCTGCCTTGAAATATTGTGAC
TCTGGGAATGACAACACCTGCTTTGTTTTTTGTTGTATCCCCAGCCCCAAACACAGCTCCTGGCCATATAT
CAAGGTTTCAATAAATATTTGCTAAATG

The disclosed NOV7 nucleic acid sequence, localized to chromosome 19, has 531 of 607 bases (87%) identical to a Homo sapiens trypsin-like serine protease (TLSP) mRNA (gb:GENBANK-ID:AF164623|acc:AF164623) (E=1.3e⁻¹⁶⁵).

A disclosed NOV7 polypeptide (SEQ ID NO:18) encoded by SEQ ID NO:17 is 247 amino acid residues and is presented using the one-letter amino acid code in Table 7B. Signal P, Psort and/or Hydropathy results predict that NOV7 contains a signal peptide and is likely to be localized in the mitochondrial inner membrane with a certainty of 0.6921 and to the plasma membrane with a certainty of 0.6500. The most likely cleavage site for a NOV7 peptide is between amino acids 50 and 51, at: VGG-ET.

TABLE 7B
Encoded NOV7 protein sequence.

(SEQ ID NO:18)

MQRLRWLRDWKSSGRGLTAAKEPGARSSPLQAMRILQLILLALATGLVGGETRIIKGFECKPHSQPWQAAL
FEKTRLLCGATLIAPRWLLTAAHCLKPLPNKDRRNDIMLVKMASPVSITWAVRPLTLSSRCVTAGTSCLIS
GWGSTSSPQLRLPHTLRCANITIIEHQKCENAYPGNITDTMVCASVQEGGKDSCQGDSGGPLVCNQSLQGI
ISWGQDPCAITRKPGVYTKVCKYVVWIQETIKNN

The NOV7 amino acid sequence has 146 of 149 amino acid residues (97%) identical to, and 147 of 149 amino acid residues (98%) similar to the Homo Sapiens 282 amino acid residue serine protease (TLSP) protein (ptnr:SPTREMBL-ACC:075837) (E=5.2e⁻¹³¹).

NOV7 is a spliced isoform of the serine protease (TLSP) from Homo sapiens (GenBank ID: AB012917). It is missing 105 nucleotides between positions 406 and 407. Deletion of this exon resulted in a deletion of 35 amino acid residues between positions 98 and 99 in the protein sequence.

NOV7 is expressed in at least the following tissues: Colon, Heart, Lung, Ovary, Parotid Salivary glands, Prostate, Salivary Glands, Stomach (normal), Stomach (poorly differentiated adenocarcinoma with signet ring cell) Testis and Uterus. In addition, the sequence is predicted to be expressed in the following tissues/cell lines because of the expression pattern of a closely related Homo sapiens trypsin-like serine protease (TLSP) gene homolog (GENBANK-ID: gb:GENBANK-ID: AF164623|acc:AF164623):brain, thymus, spleen, liver and in breast carcinoma cell line BT-474.

NOV7 also has homology to the amino acid sequence shown in the BLASTP data listed in Table 7C.

TABLE 7C
BLAST results for NOV7

| Gene Index/ | | Length | Identity | Positives | |
|---|---|---|---|---|---|
| Identifier | Protein/Organism | (aa) | (%) | (%) | Expect |
| gi\|3649791\|dbj\|BAA33404.1\| | serine protease | 282 | 244/282 | 245/282 | 1e−124 |
| (AB012917) | (TLSP) [Homo | | (86%) | (86%) | |
| | sapiens] | | | | |
| gi\|5803199\|ref\|NP_006844.1\| | kallikrein 11; | 250 | 212/250 | 213/250 | 1e−107 |
| | protease, serine, | | (84%) | (84%) | |
| | trypsin-like; | | | | |
| | protease, serine, | | | | |
| | 20 trypsin-like | | | | |
| | [Homo sapiens] | | | | |
| gi\|6681654\|dbj\|BAA36955.1\| | hippostasin | 276 | 191/282 | 214/282 | 1e−101 |
| (AB016227) | prostate type | | (67%) | (75%) | |
| | [Mus musculus] | | | | |
| gi\|9910298\|ref\|NP_064358.1\| | protease, serine, | 249 | 175/248 | 194/248 | 5e−96 |
| | 20; hippostasin | | (70%) | (77%) | |
| | [Mus musculus] | | | | |
| gi\|9296988\|sp\|Q9UKQ9\| | kallikrein 9 precursor | 250 | 117/242 | 152/242 | 7e−57 |
| KLK9_HUMAN | (kallikrein-like protein | | (48%) | (62%) | |
| | 3) (KLK-L3) [Homo | | | | |
| | sapiens] | | | | |

The homology of these sequences is shown graphically in the ClustalW analysis shown in Table 7D.

[image]

[image]

Tables 7E and 7F list the domain description from DOMAIN analysis results against NOV7. This indicates that the NOV7 sequence has properties similar to those of other proteins known to contain this domain.

TABLE 7E
Domain Analysis of NOV7

gnl|Smart|smart00020, Tryp_SPc, Trypsin-like serine protease; Many of
these are synthesised as inactive precursor zymogena that are cleaved
during limited proteolysis to generate their active forms. A few.
however, are active as single chain molecules, and others are inactive
due to substitutions of the catalytic triad residues. (SEQ ID NO:96)
Length = 230 residues, 100.0% aligned
Score = 210 bits (535), Expect = 7e−56

| | | | |
|---|---|---|---|
| NOV7 | 53 | RIIKGFECKPHSQPWQAALF-EKTRLLCGATLIAPRWLLTAAHCLKPLPNKDRR------ | 105 |
| | | \|\|+\|\| \|\|\|\|+\| \| \|\|+\|\|+\|\|\|+\|\|\|\|\|\|+ \| | |
| 00020 | 1 | RIVGGSEANIGSFPWQVSLQYRGGRHFCGGSLISPRWVLTAAHCVYGSAPSSIRVRLGSH | 60 |
| NOV7 | 106 | ----------------------------NDIMLVKMASPVSITWAVRPLTL--SSRCVTA | 135 |
| | | \|\|\|\|+\|++\|\|+++ \|\|\|+\| \| \|\| | |
| 00020 | 61 | DLSSGEETQTVKVSKVIVHPNYNPSTYDNDIALLKLSEPVTLSDTVRPICLPSSGYNVPA | 120 |
| NOV7 | 136 | GTSCLISGWGSTSSPQLRLPHTLRCANITIIEHQKCENAYPGN--ITDTMVCASVQEGGK | 193 |
| | | \|\|+\|+\|\|\|\|\|\| \|\|\|\|+ \|+\|++ \| \|\|\| \|\|\|\|+\|\| \|\|\|\| | |
| 00020 | 121 | GTTCTVSGWGRTSESSGSLPDTLQEVNVPIVSNATCRRAYSGGPAITDNMLCAGGLEGGK | 180 |
| NOV7 | 194 | DSCQGDSGGPLVCNQS---LQGIISWGQDPCAITRKPGVYTKVCKYVVWI 240 | |
| | | \|+\|\|\|\|\|\|\|\|\|\|\|\| \|\|\|+\|\|\| \|\| \|\|\|\|\|\|+\| \|+\|\| | |
| 00020 | 181 | DACQGDSGGPLVCNDPRWVLVGIVSWGSYGCARPNKPGVYTRVSSYLDWI 230 | |

TABLE 7F
Domain Analysis of NOV7

gnl|Pfam|pfam00089, trypsin, Trypsin. Proteins recognized include all
proteins in families S1, S2A, S29, S2C, and 55 in the classification
of peptidases. Also included are proteins that are clearly members,
but that lack peptidase activity, such as haptoglobin and protein Z
(PRTZ*). (SEQ ID NO:97)
Length = 217 residues, 100.0% aligned
Score = 172 bits (435), Expect = 3e−44

| | | | |
|---|---|---|---|
| NOV7 | 54 | IIKGFECKPHSQPWQAALFEKTRLLCGATLIAPRWLLTAAHCLK---------------- | 97 |
| | | \|+\|\|+ \|\|\|\|+\| + \|\|+\|\|+ \|+\|\|\|\|\|\|+ | |
| 00089 | 1 | IVGGREAQAGSFPWQVSLQVSSGHFCGGSLISENWVLTAAHCVSGASSVRVVLGEHNLGT | 60 |
| NOV7 | 98 | --------------PLPNKD-RRNDIMLVKMASPVSITWAVRPLTLSSRC--VTAGTSCL | 140 |
| | | \|\|+ \|\|\|\|+\|+\|\|\|++ \|\|\|+\|\| + \|\|+\| | |
| 00089 | 61 | TEGTEQKFDVKKIIVHPNYNPDTNDIALLKLKSPVTLGDTVRPICLPSASSDLPVGTTCS | 120 |
| NOV7 | 141 | ISGWGSTSSPQLRLPHTLRCANITIIEHQKCENAYPGNITDTMVCASVQEGGKDSCQGDS | 200 |
| | | +\|\|\|\|\|+ \| \|\|+ +\|+ +\|+\|\|\|+\|\|\|\|+\|\| \|\|\|\|+\|\|\|\|\| | |
| 00089 | 121 | VSGWGRTKN--LGTSDTLQEVVVPIVSRETCRSAYGGTVTDTMICAGAL-GGKDACQGDS | 177 |
| NOV7 | 201 | GGPLVC-NQSLQGIISWGQDPCAITRKPGVYTKVCKYVVWI 240 | |
| | | \|\|\|\|\|\|+ \|\|\|+\|\|\| \|\|+ \|\|\|\|\|+\|+\|+\|\| | |
| 00089 | 178 | GGPLVCSDGELVGIVSWG-YGCAVGNYPGVYTRVSRYLDWI 217 | |

The amino acid sequence of NOV7 has high homology to other proteins as shown in

TABLE 7G
BLASTX results for NOV7

| | | Smallest | | |
|---|---|---|---|---|
| | Reading | Sum | | |
| | High | Prob | | |
| Sequence producing High-scoring Segment Pairs: | Frame | Score | P(N) | N |
| patp:AAY42439 cASB12 amine acid sequence, Homo Sapi 282 aa.. | +2 | 792 | 3.0e−130 | 1 |
| patp:AA811712 Huma serine pretease BSSPG, Homo Sapi 282 aa.. | +2 | 792 | 3.0e−130 | 1 |
| patp.AAY43636 Human prostate-associated serum protease, Home Sapi 282 aa.. | +2 | 792 | 3.0e−130 | 1 |

The trypsin family is almost totally confined to animals, although trypsin-like enzymes are found in actinomycetes of the genera Streptomyces and Saccharopolyspora, and in the fungus Fusarium oxysporum. The enzymes are inherently secreted, being synthesised with a signal peptide that targets them to the secretory pathway. Animal enzymes are either secreted directly, packaged into vesicles for regulated secretion, or are retained in leukocyte granules.

Proteases play a pivotal role in several biologic processes, including tissue remodeling and cell migration. By PCR of human hippocampus cDNA using primers derived from mouse neuropsin cDNA sequences corresponding to conserved regions of serine proteases, a novel serine protease, KLK11, was identified which was named TLSP. The deduced 260-amino acid protein contains a signal peptide, 3 key amino acids essential for serine protease activity, an asp residue in a position that suggests a trypsin-type substrate specificity for basic amino acids at the P1 position, conserved amino acids that can form an oxyanion hole, and a potential N-glycosylation site. KLK11 shares 48% amino acid sequence identity with mouse neuropsin, 43% identity with both human trypsin-1 and human kallikrein, and 38% identity with the mouse nerve growth factor gamma subunit. Western blot analysis of recombinant KLK11 suggested that the protein is secreted and posttranslationally processed.

Proteolytic enzymes have been readily used in traditional medicine and studies have shown that enzyme therapy can reduce the adverse effects caused by radiotherapy and chemotherapy. There is also evidence that, in some types of tumours, survival may be prolonged. The beneficial effect of systemic enzyme therapy seems to be based on its anti-inflammatory potential (Leipner and Saller, Drugs 59(4):769-80, 2000).

The above defined information for NOV7 suggests that this NOV7 protein may function as a member of a Serine Protease TLSP family. Therefore, the NOV7 nucleic acids and proteins of the invention are useful in potential therapeutic applications implicated in various diseases and disorders described below and/or other pathologies. For example, the NOV7 compositions of the present invention will have efficacy for treatment of patients suffering from cancer, neurological disorders, digestive system disorders and all or some of the protease/protease inhibitor deficiency disorders. The NOV7 nucleic acid encoding Serine Protease TLSP-like protein, and the Serine Protease TLSP-like protein of the invention, or fragments thereof, may further be useful in diagnostic applications, wherein the presence or amount of the nucleic acid or the protein are to be assessed.

NOV8

NOV8 includes four novel Glypican-2 Precursor-like proteins disclosed below. The disclosed proteins have been named NOV8a, NOV8b, NOV8c and NOV8d.

NOV8a

A disclosed NOV8a nucleic acid of 1785 nucleotides (also referred to 134913441_EXT) encoding a novel Glypican-2 Precursor-like protein is shown in Table 8A. An open reading frame was identified beginning with an ATG initiation codon at nucleotides 1-3 and ending with a TAA codon at nucleotides 1738-1740. A putitive untranslated region downstream from the termination codon is underlined in Table 8A, and the start and stop codons are in bold letters.

TABLE 8A
NOV8a Nucleotide Sequence

(SEQ ID NO:19)

ATGTCCGCGCTGCGACCTCTCCTGCTTCTGCTGCTGCCTCTGTGTCCCGCTCCTGGTCCCGGACCCGGGAG
CGAGGCAAAGGTCACCCGGAGTTGTGCAGAGACCCGGCAGGTGCTCCCGCCCCGGGGATATAGCTTAAACC
TAATCCCTCCCGCCCTGATCTCAGGTGAGCACCTCCGGGTCTGTCCCCAGGAGTACACCTGCTGTTCCAGT
GAGACAGAGCAGAGGCTGATCAGGGAGACTGAGGCCACCTTCCGACGCCTGGTGGAGGACAGCGGCTCCTT
TCTGGTTCACACACTGGCTGCCAGGCACAGAAAATTTGATGAGTTTTTTCTGGAGATGCTCTCAGTAGCCC
AGCACTCTCTGACCCAGCTCTTCTCCCACTCCTACGGCCGCCTGTATGCCCAGCACGCCCTCATATTCAAT
GGCCTGTTCTCTCGGCTGCGAGACTTCTATGGGGAATCTGGTGAGGGGTTGGATGACACCCTGGCGGATTT
CTGGGCACAGCTCCTGGAGAGAGTGTTCCCGCTGCTGCACCCACACTACAGCTTCCCCCCTGACTACCTGC
TCTGCCTCTCACGCTTGCCCTCATCTACCGATGGCTCTCTGCAGCCCTTTGGGGACTCACCCCGCCGCCTC
CGCCTGCAGATAACCCGGACCCTGGTGGCTGCCCGAGCCTTTGTGCAGGGCCTGGAGACTGGAAGAAATGT
GGTCAGCGAAGCGCTTAAGGTTCCGGTGTCTGAAGGCTGCAGCCACGCTCTGATCCGTCTCATCGGCTGTC
CCCTGTGCCGGGGGGTCCCCTCACTTATGCCCTGCCAGGGCT1CTGCCTCAACGTGGTTCGTGGCTGTCTC
AGCAGCAGGGGACTGGAGCCTGACTGGGGCAACTATCTGGATGGTCTCCTGATCCTGGCTGATAAGCTCCA
GGGCCCCTTTTCCTTTGAGCTGACGGCCGAGTCCATTGGGGTGAAGATCTCGGAGGGTTTGATGTACCTGC
AGGAAAACAGTGCGAAGGTGTCCGCCCAGGTATTTCAGGAGTGCGGCCCCCCCGACCCGGTGCCTGCCCGC
AACCGTCGAGCCCCGCCGCCCCGGGAAGAGGCGGGCCGGCTGTGGTCGATGGTGACCGAGGAGGAGCGGCC
AACGACCGCCGCAGGCACCAACCTGCACCGGCTGGTGTGGGAGCTCCGCGAGCGTCTGGCCCGGATGCGGG
GCTTCTGGGCCCGGCTGTCCCTGACGGTGTGCGGACACTCTCGCATGGCACCGGACGCCTCGCTGGAGGCG
GCGCCCTGCTGGACCGGAGCCGGGCGGGGCCGGTACTTGCCGCCAGTGGTCGGGGGCTCCCCGGCCGAGCA
GGTCAACAACCCCGAGCTCAACGTGGACGCCTCGGGCCCCGATGTCCCGACACGGCCGCGTCGGCTACAGC
TCCGGCCGGCCACGGCCAGAATQAAAACGGCCCCACTGGGACACGACCTGGACCGGCAGGACGCAGATGAG
CATGCCAGCGGCTCTGGAGGGGGACACCAGTATGCAGATGACTGGATGGCTGGGGCTGTGGCTCCCCCAGC
CCGGCCTCCTCGGCCTCCATACCCTCCTAGAAGGGATGGTTCTGGGGGCAAAGGAGGAGGTGGCAGTGCCC
GCTACAACCAGGGCCGGAGCAGGAGTGGGGGGGCATCTATTGGTTTTCACACCCAAACCATCCTCATTCTC
TCCCTCTCAGCCCTGGCCCTGCTTGGACCTCGATAACGGGGAGGGGTGCCCTAGCATCAGAAGGGTTTCAT
GGCCCTTTCC

The disclosed NOV8a nucleic acid sequence, localized to chromosome 7, has 1469 of 1785 bases (82%) identical to a Rattus norvegicus cerebroglycan mRNA (gb:GENBANK-ID:RATCRBGLVC|acc:120468) (E=3.3e⁻²⁶¹).

A disclosed NOV8a polypeptide (SEQ ID NO:20) encoded by SEQ ID NO:19 is 579 amino acid residues and is presented using the one-letter amino acid code in Table 8B. Signal P, Psort and/or Hydropathy results predict that NOV8a contains a signal peptide and is likely to be localized extracellularly with a certainty of 0.4467. The most likely cleavage site for a NOV8a peptide is between amino acids 23 and 24, at: GPG-SE.

TABLE 8B
Encoded NOV8a protein sequence.

(SEQ ID NO:20)

MSALRPLLLLLLPLCPGPGPGPGSEAKVTRSCAETRQVLGARGYSLNLIPPALISGEHLRVCPQEYTCCSS
ETEQRLIRETEATFRGLVEDSGSFLVHTLAARHRKFDEFFLEMLSVAQHSLTQLFSHSYGRLYAQHALIFN
GLFSRLRDFYGESGEGLDDTLADFWAQLLERVFPLLHPQYSFPPDYLLCLSRLASSTDGSLQPFGDSPRRL
RLQITRTLVAARAFVQGLETGRNVVSEALKVPVSEGCSQALMRLIGCPLCRGVPSLMPCQGFCLNVVRGCL
SSRGLEPDWGNYLDGLLILADKLQGPFSFELTAESIGVKISEGLMYLQENSAKVSAQVFQECGPPDPVPAR
NRRAPPPREEAGRLWSMVTEEERPTTAAGTNLHRLVWELRERLARMRGFWARLSLTVCGDSRMAADASLEA
APCWTGAGRGRYLPPVVGGSPAEQVNNPELKVDASGPDVPTRRRRLQLRAATARMKTAALGHDLDGQDADE
DASGSGGGQQYADDWMAGAVAPPARPPRPPYPPRRDGSGGKGGGGSARYNQGRSRSGGASIGFHTQTILIL
SLSALALLGPR

The NOV8a amino acid sequence has 477 of 579 amino acid residues (82%) identical to, and 513 of 579 amino acid residues (88%) similar to, the Rattus norvegicus 579 amino acid residue glypican-2 precursor (cerebroglycan) protein (ptnr:SWISSPROT-ACC:P51653)(E=1.1e−260).

NOV8a is expressed in at least the following tissues: Kidney, Spleen, Brain, Pediatric pre-B cell acute lymphoblastic leukemia. This information was derived by determining the tissue sources of the sequences that were included in the invention. SeqCalling sources: Kidney, Spleen, Brain; PublicEST sources: Pediatric pre-B cell acute lymphoblastic leukemia. In addition, NOV8a is predicted to be expressed in brain tissues because of the expression pattern of a closely related Rattus norvegicus cerebroglycan mRNA homolog (GENBANK-ID: gb:GENBANK-ID:RATCRBGLVC|acc:L20468).

NOV8b

A disclosed NOV8b nucleic acid of 1976 nucleotides (also referred to CG50970-02) encoding a novel Glypican-2 Precursor-like protein is shown in Table 8C. An open reading frame was identified beginning with an ATG initiation codon at nucleotides 54-56 and ending with a TAA codon at nucleotides 1449-1451. Putitive untranslated regions upstream from the intiation codon and downstream from the termination codon is underlined in Table 8C, and the start and stop codons are in bold letters.

TABLE 8C
NOV8b Nucleotide Sequence

| | |
|---|---|
| GGCTCTGCTTTCCTCCTTAGGACCCACTTTGCCGTCCTGGGGTGGCTGCAGTTATGTCCGCGCT | (SEQ ID NO:21) |
| GCGACCTCTCCTGCTTCTGCTGCTGCCTCTGTGTCCCGGTCCTGGTCCCGGACCCGGGAGCGAG | |
| GCAAAGGTCACCCGGAGTTGTGCAGAGACCCGGCAGGTGCTGGGGGCCCGGGGATATAGCTTAA | |
| ACCTAATCCCTCCCGCCCTGATCTCAGGTGAGCACCTCCGGGTCTGTCCCCAGGAGTACACCTG | |
| CTGTTCCAGTGAGACAGAGCAGAGGCTGATCAGGGAGACTGAGGCCACCTTCCGAGGCCTGGTG | |
| GAGGACAGCGGCTCCTTTCTGGTTCACACACTGGCTGCCAGGCACAGAAAATTTGATGAGTTTT | |
| TTCTGGAGATGCTCTCAGTAGCCCAGCACTCTCTGACCCAGCTCTTCTCCCACTCCTACGGCCG | |
| CCTGTATGCCCAGCACGCCCTCATATTCAATGGCCTGTTCTCTCGGCTGCGAGACTTCTATGGG | |
| GAATCTGGTGAGGGGTTGGATGACACCCTGGCGGATTTCTGGGCACAGCTCCTGGAGAGAGTGT | |
| TCCCGCTGCTGCACCCACAGTACAGCTTCCCCCCTGACTACCTGCTCTGCCTCTCACGCTTGGC | |
| CTCATCTACCGATGGCTCTCTGCAGCCCTTTGGGGACTCACCCCGCCGCCTCCGCCTGCAGATA | |
| ACCCGGACCCTGGTGGCTGCCCGAGCCTTTGTGCAGGGCCTGGAGACTGGAAGAAATGTGGTCA | |
| GCGAAGCGCTTAAGGTTCCGGTGTCTGAAGGCTGCAGCCAGGCTCTGATGCGTCTCATCGGCTG | |
| TCCCCTGTGCCGGGGGGTCCCCTCACTTATGCCCTGCCAGGGCTTCTGCCTCAACGTGGTTCGT | |
| GGCTGTCTCAGCAGCAGGGGACTGGAGCCTGACTGGGGCAACTATCTGGATGGTCTCCTGATCC | |
| TGGCTGATAAGCTCCAGGGCCCCTTTTCCTTTGAGCTGACGGCCGAGTCCATTGGGGTGAAGAT | |
| CTCGGAGGGTTTGATGTACCTGCAGGAAAACAGTGCGAAGGTGTCCGCCCAGGTATTTCAGGAG | |
| TGCGGCCCCCCCGACCCGGTGCCTGCCCGCAACCGTCGAGCCCCGCCGCCCCGGGAAGAGGCGG | |
| GCCGGCTGTGGTCGATGGTGACCGAGGAGGAGCGGCCAAGCGCAGATGAGGATGCCAGCGGCTC | |
| TGGAGGGGGACAGCAGTATGCAGATGACTGGATGGCTGGGGCTGTGGCTCCCCCAGCCCGGCCT | |
| CCTCGGCCTCCATACCCTCCTAGAAGGGATGGTTCTGGGGGCAAAGGAGGAGGTGGCAGTGCCC | |
| GCTACAACCAGGGCCGGAGCAGGAGTGGGGGGGCATCTATTGGTTTTCACACCCAAACCATCCT | |
| CATTCTCTCCCTCTCAGCCCTGGCCCTGCTTGGACCTCGATAACGGGGGAGGGGTGCCCTAGCA | |
| TCAGAAGGGTTCATGGCCCTTTCCCCTCCTCCCCCCTCAGCTGGGCCTGGGGAGGAGTCGAAGG | |
| GGGCTGCAGAGAGGGTAGAGAAGGGACTTTGCAGGTGAATGGCTGGGGCCCCAAATCCAGGAGA | |
| TTTTCATCAGAGGTGGGTGGGTGTTCACAATATTTATTTTTTCATTTGGTAATGGGAGGGGGGC | |
| CTGGGGGTATTTATTTAGGAGGGAGTGTGGTTTCCTTAGAAGGTATAGTCTCTAGCCCTCTAAG | |
| GCTGGGGCTGGTGATCAGCCCCAACAGAGAAAATGAGGAGTTTAGAGTTGCAGCTGGGTTCTGT | |
| TGAGTTTTTTCAGTATCAATTTCTTAAACCAAATTTTAAAAAAAACAAGGTGGGGGGGTGCTCA | |
| TCTCGTGACCTCTGCCACCCACATCCTTCACAAACTCCATGTTTCAGTGTTTGAGTCCATGTTT | |
| ATTCTGCAAATAAATGGTAATGTATTAGAAAAAAAAAAAAAAAAAAAAAAAAAAAA | |

The disclosed NOV8b nucleic acid sequence, localized to chromosome 2q35-q37, has 1047 of 1271 bases (82%) identical to a Rattus norvegicus cerebroglycan mRNA (gb:GENBANK-ID:RATCRBGLVC|acc:L20468.1) (E=1.4e-247).

A disclosed NOV8b polypeptide (SEQ ID NO:22) encoded by SEQ ID NO:21 is 465 amino acid residues and is presented using the one-letter amino acid code in Table 8D. Signal P, Psort and/or Hydropathy results predict that NOV8b contains a signal peptide and is likely to be localized extracellularly with a certainty of 0.4467. The most likely cleavage site for a NOV8b peptide is between amino acids 23 and 24, at: GPG-SE.

TABLE 8D
Encoded NOV8b protein sequence.

(SEQ ID NO:22)

MSALRPLLLLLLPLCPGPGPGPGSEAKVTRSCAETRQVLGARGYSLNLIPPALISGEHLRVCPQEYTCCSS
ETEQRLIRETEATFRGLVEDSGSFLVHTLAARHRKFDEFFLEMLSVAQHSLTQLFSHSYGRLYAQHALIFN
GLFSRLRDFYGESGEGLDDTLADFWAQLLERVFPLLHPQYSFPPDYLLCLSRLASSTDGSLQPFGDSPRRL
RLQITRTLVAARAFVQGLETGRNVVSEALKVPVSEGCSQALMRLIGCPLCRGVPSLMPCQGFCLNVVRGCL
SSRGLEPDWGNYLDGLLILADKLQGPFSFELTAESIGVKISEGLMYLQENSAKVSAQVFQECGPPDPVPAR
NRRAPPPREEAGRLWSMVTEEERPSADEDASGSGGGQQYADDWMAGAVAPPARPPRPPYPPRRDGSGGKGG
GGSARYNQGRSRSGGASIGFHTQTILILSLSALALLGPR

The NOV8b amino acid sequence has 322 of 380 amino acid residues (84%) identical to, and 348 of 380 amino acid residues (91%) similar to, the Rattus norvegicus 579 amino acid residue glypican-2 precursor (cerebroglycan) protein (ptnr:SWISSPROT-ACC:P51653) (E=1.5e⁻²¹⁰).

NOV8b is expressed in at least the following tissues: Aorta, Brain, Cartilage, Cervix, Liver, Lung, Oviduct/Uterine Tube/Fallopian tube, Parotid Salivary glands, Placenta, Prostate, Retina, Skeletal Muscle, Stomach, Temporal Lobe, Testis, Vein. This information was derived by determining the tissue sources of the sequences that were included in the invention including but not limited to SeqCalling sources, Public EST sources, Literature sources, and/or RACE sources.

NOV8c

A disclosed NOV8c nucleic acid of 1613 nucleotides (also referred to CG50970-03) encoding a novel Glypican-2 Precursor-like protein is shown in Table 8E. An open reading frame was identified beginning with an ATG initiation codon at nucleotides 1-3 and ending with a TGA codon at nucleotides 1348-1350. A putitive untranslated region downstream from the termination codon is underlined in Table 8E, and the start and stop codons are in bold letters.

TABLE 8E
NOV8c Nucleotide Sequence

| | |
|---|---|
| ATGTCCGCGCTGCGACCTCTCCTGCTTCTGCTGCTGCCTCTGTGTCCCGGTCCTGGTCCCGGAC | (SEQ ID NO:23) |
| CCGGGAGCGAGGCAAAGGTCACCCGGAGTTGTGCAGAGACCCGGCAGGTGCTGGGGGCCCGGGG | |
| ATATAGCTTAAACCTAATCCCTCCCGCCCTGATCTCAGGTGAGCACCTCCGGGTCTGTCCCCAG | |
| GAGTACACCTGCTGTTCCAGTGAGACAGAGCAGAGGCTGATCAGGGAGACTGAGGCCACCTTCC | |
| GAGGCCTGGTGGAGGACAGCGGCTCCTTTCTGGTTCACACACTGGCTGCCAGGCACAGAAAATT | |
| TGATGAGTTTTTTCTGGAGATGCTCTCAGTAGCCCAGCACTCTCTGACCCAGCTCTTCTCCCAC | |
| TCCTACGGCCGCCTGTATGCCCAGCACGCCCTCATATTCAATGGCCTGTTCTCTCGGCTGCGAG | |
| ACTTCTATGGGGAATCTGGTGAGGGGTTGGATGACACCCTGGCGGATTTCTGGGCACAGCTCCT | |
| GGAGAGAGTGTTCCCGCTGCTGCACCCACAGTACAGCTTCCCCCCTGACTACCTGCTCTGCCTC | |
| TCACGCTTGGCCTCATCTACCGATGGCTCTCTGCAGCCCTTTGGGGACTCACCCCGCCGCCTCC | |
| GCCTGCAGATAACCCGGACCCTGGTGGCTGCCCGAGCCTTTGTGCAGGGCCTGGAGACTGGAAG | |
| AAATGTGGTCAGCGAAGCGCTTAAGGTGCCGGTGTCTGAAGGCTGCAGCCAGGCTCTGATGCGT | |
| CTCATCGGCTGTCCCCTGTGCCGGGGGGTCCCCTCACTTATGCCCTGCCAGGGCTTCTGCCTCA | |
| ACGTGGTTCGTGGCTGTCTCAGCAGCAGGGGACTGGAGCCTGACTGGGGCAACTATCTGGATGG | |
| TCTCCTGATCCTGGCTGATAAGCTCCAGGGCCCCTTTTCCTTTGAGCTGACGGCCGAGTCCATT | |
| GGGGTGAAGATCTCGGAGGGTTTGATGTACCTGCAGGAAAACAGTGCGAAGGTGTCCGCCCAGG | |
| TGTTTCAGGAGTGCGGCCCCCCCGACCCGGTGCCTGCCCGCAACCGTCGAGCCCCGCCGCCCCG | |
| GGAAGAGGCGGGCCGGCTGTGGTCGATGGTGACCGAGGAGGAGCGGCCCACGACGGCCGCAGGC | |
| ACCAACCTGCACCGGCTGGTACTTGCCGCCAGTGGTCGGGGGCTCCCCGGCCGAGCAGGTCAAC | |
| AACCCCGAGCTCAAGGTGGACGCCTCGGGCCCCGATGTCCCGACACGGCGGCGTCGGCTACAGC | |
| TCCGGGCGGCCACGGCCAGAATGAAAACGGCCGCACTGGGACACGACCTGGACGGGCAGGACGC | |
| GGATGAGGATGCCAGCGGCTCTGGAGGGGGACAGCAGTATGCAGATGACTGGATGGCTGGGGCT | |
| GTGGCTCCCCCAGCCCGGCCTCCTCGGCCTCCATACCCTCCTAGAAGGGATGGTTCTGGGGGCA | |
| AAGGAGGAGGTGGCAGTGCCCGCTACAACCAGGGCCGGAGCAGGAGTGGGGGGGCATCTATTGG | |
| TTTTCACACCCAAACCATCCTCATTCTCTCCCTCTCAGACCTGGCCCTGCTTGGACCTCGATAA | |
| CGGGGGAGGGGTG | |

The disclosed NOV8c nucleic acid sequence, localized to chromosome 2, has 994 of 1172 bases (84%) identical to a Rattus norvegicus cerebroglycan mRNA (gb:GENBANK-ID:RATCRBGLVC|acc:L20468.1) (E=1.3e⁻²³⁷).

A disclosed NOV8c polypeptide (SEQ ID NO:24) encoded by SEQ ID NO:23 is 449 amino acid residues and is presented using the one-letter amino acid code in Table 8F. Signal P, Psort and/or Hydropathy results predict that NOV8c contains a signal peptide and is likely to be localized extracellularly with a certainty of 0.3700. The most likely cleavage site for a NOV8c peptide is between amino acids 23 and 24, at: GPG-SE.

TABLE 8F
Encoded NOV8c protein sequence.

(SEQ ID NO:24)

MSALRPLLLLLLPLCPGPGPGPGSEAKVTRSCAETRQVLGARGYSLNLIPPALISGEHLRVCPQEYTCCSS
ETEQRLIRETEATFRGLVEDSGSFLVHTLAARHRKFDEFFLEMLSVAQHSLTQLFSHSYGRLYAQHALIFN
GLFSRLRDFYGESGEGLDDTLADFWAQLLERVFPLLHPQYSFPPDYLLCLSRLASSTDGSLQPFGDSPRRL
RLQITRTLVAARAFVQGLETGRNVVSEALKVPVSEGCSQALMRLIGCPLCRGVPSLMPCQGFCLNVVRGCL
SSRGLEPDWGNYLDGLLILADKLQGPFSFELTAESIGVKISEGLMYLQENSAKVSAQVFQECGPPDPVPAR
NRRAPPPREEAGRLWSMVTEEERPTTAAGTNLHRLVLAASGRGLPGRAGQQPRAQGGRLGPRCPDTAASAT
APGGHGQNENGRTGTRPGRAGRG

The NOV8c amino acid sequence has 334 of 391 amino acid residues (85%) identical to, and 359 of 391 amino acid residues (91%) similar to, the Rattus norvegicus 579 amino acid residue glypican-2 precursor (cerebroglycan) protein (ptnr:SWISSPROT-ACC:P51653) (E=1e⁻¹⁸³).

NOV8c is expressed in at least the following tissues: Aorta, Brain, Cartilage, Cervix, Liver, Lung, Oviduct/Uterine Tube/Fallopian tube, Parotid Salivary glands, Placenta, Prostate, Retina, Skeletal Muscle, Stomach, Temporal Lobe, Testis, Vein. Expression information was derived from the tissue sources of the sequences that were included in the derivation of the NOV8c sequence.

NOV8d

A disclosed NOV8d nucleic acid of 725 nucleotides (also referred to CG50970-04) encoding a novel Glypican-2 Precursor-like protein is shown in Table 8G. An open reading frame was identified beginning with an ATG initiation codon at nucleotides 160-162 and ending with a TAA codon at nucleotides 688-690. Putitive untranslated regions upstream from the initiation codon and downstream from the termination codon is underlined in Table 8G, and the start and stop codons are in bold letters.

TABLE 8G
NOV8d Nucleotide Sequence

| | |
|---|---|
| CGCCTGGTCCAGCTATCGTGCTCGGTATTCAGTTTTCCGGAGCAGCGCTCTTTCTCTGGCCCGC | (SEQ ID NO:25) |
| GGAACGGTCCCGCGGCCGAGTACCGGATTCCCGAGTTTGGGAGGCTCTGCTTTCCTCCTTAGGA | |
| CCCACTTTGCCGTCCTGGGGTGGCTGCAGTTATGTCCGCGCTGCGACCTCTCCTGCTTCTGCTG | |
| CTGCCTCTGTGTCCCGGTCCTGGTCCCGGACCCGGGAGCGAGGCAAAGGTCACCCGGAGTTGTG | |
| CAGAGACCCGGCAGGTGCTGGGGGCCCGGGGATATAGCTTAAACCTAATCCCTCCCGCCCTGAT | |
| CTCAGGTGAGCACCTCCGGGTCTGTCCCCAGGAGTACACCTGCTGTTCCAGTGAGACAGAGCAG | |
| AGGCTGATCAGGGAGACTGAGGCCACCTTCCGAGGCCTGGTGGAGGACAGCGGCTCCTTTCTGG | |
| TTCACACACTGGCTGCCAGGCACAGAAAATTTGATGAGTTTTTTCTGGAGATGCTCTCAGTAGC | |
| CCGGCCTCCTCGGCCTCCATACCCTCCTAGAAGGGATGGTTCTGGGGGCAAAGGAGGAGGTGGC | |
| AGTGCCCGCTACAACCAGGGCCGGAGCAGGAGTGGGGGGGCATCTATTGGTTTTCACACCCAAA | |
| CCATCCTCATTCTCTCCCTCTCAGCCCTGGCCTTGCTTGGACCTCGATAACGGGGGAGGGGTGC | |
| CCTAGCATCAGAAGGGTTCAT | |

The disclosed NOV8d nucleic acid sequence, localized to chromosome 2, has 448 of 545 bases (82%) identical to a Rattus norvegicus cerebroglycan mRNA (gb:GENBANK-ID:RATCRBGLVC|acc:L20468.1) (E=4.2e⁻¹⁰¹).

A disclosed NOV8d polypeptide (SEQ ID NO:26) encoded by SEQ ID NO:25 is 176 amino acid residues and is presented using the one-letter amino acid code in Table 8H. Signal P, Psort and/or Hydropathy results predict that NOV8d contains a signal peptide and is likely to be localized extracellularly with a certainty of 0.4467. The most likely cleavage site for a NOV8d peptide is between amino acids 23 and 24, at: GPG-SE.

TABLE 8H
Encoded NOV8d protein sequence.

(SEQ ID NO:26)

MSALRPLLLLLLPLCPGPGPGPGSEAKVTRSCAETRQVLGARGYSLNLIPPALISGEHLRVCPQEYTCCSS
ETEQRLIRETEATFRGLVEDSGSFLVHTLAARHRKFDEFFLEMLSVARPPRPPYPPRRDGSGGKGGGGSAR
YNQGRSRSGGASIGFHTQTILILSLSALALLGPR

The NOV8d amino acid sequence has 103 of 119 amino acid residues (86%) identical to, and 114 of 119 amino acid residues (95%) similar to, the Rattus norvegicus 579 amino acid residue glypican-2 precursor (cerebroglycan) protein (ptnr:SWISSPROT-ACC:P51653) (E=2.6e⁻⁷³).

NOV8d is expressed in at least the following tissues: Aorta, Brain, Cartilage, Cervix, Liver, Lung, Oviduct/Uterine Tube/Fallopian tube, Parotid Salivary glands, Placenta, Prostate, Retina, Skeletal Muscle, Stomach, Temporal Lobe, Testis and Vein. Expression information was derived from the tissue sources of the sequences that were included in the derivation of the NOV8d sequence.

Possible SNPs found for GPCR8d are listed in Table 81.

TABLE 8I
SNPs

| Consensus | | Base | |
|---|---|---|---|
| Position | Depth | Change | PAF |
| 227 | 19 | T > C | 0.105 |
| 482 | 55 | C > T | 0.036 |
| 523 | 55 | A > G | 0.036 |
| 548 | 55 | G > A | 0.036 |
| 573 | 53 | G > A | 0.038 |
| 684 | 28 | T > C | 0.393 |

The NOV8a-NOV8d re very closely homologous as as shown in the alignment in Table 8J.

[image]

[image]

Homologies to either of the above NOV8 proteins will be shared by the other NOV8 protein insofar as they are homologous to each other as shown above. Any reference to NOV8 is assumed to refer to both of the NOV8 proteins in general, unless otherwise noted.

The disclosed NOV8 polypeptide has homology to the amino acid sequences shown in the BLASTP data listed in Table 8K.

TABLE 8K
BLAST results for NOV8a

| Gene Index/ | | Length | Identity | Positives | |
|---|---|---|---|---|---|
| Identifier | Protein/Organism | (aa) | (%) | (%) | Expect |
| gi\|1708021\|sp\|P51653 | glypican-2 | 579 | 476/581 | 512/581 | 0.0 |
| GPC2_RAT | precursor | | (81%) | (87%) | |
| | (cerebroglycan) | | | | |
| | (HSPG M13) [Rattus | | | | |
| | norvegicus] | | | | |
| gi\|7106325\|ref\|NP_ | glypican 6 [Mus | 555 | 226/512 | 332/512 | 1e−124 |
| 035951.1\| | musculus] | | (44%) | (64%) | |
| gi\|5031719\|ref\|NP_ | glypican 6 | 555 | 225/512 | 330/512 | 1e−122 |
| 005699.1\| | precursor [Homo | | (43%) | (63%) | |
| | sapiens] | | | | |
| gi\|6680059\|ref\|NP_ | glypican 4 [Mus | 557 | 208/487 | 314/487 | 1e−114 |
| 032176.1\| | musculus] | | (42%) | (63%) | |
| gi\|13879296\|gb\|AAH0 | glypican 4 [Mus | 557 | 208/487 | 314/487 | 1e−114 |
| 6622.1\|AAH06622 | musculus] | | (42%) | (63%) | |
| (BC006622) | | | | | |

The homology between these and other sequences is shown graphically in the ClustalW analysis shown in Table 8L.

[image]

[image]

Table 8M lists the domain description from DOMAIN analysis results against NOV8a. This indicates that the NOV8a sequence has properties similar to those of other proteins known to contain these domains.

TABLE 8M
Domain Analysis of NOV8a

gnl|Pfan|pfam01153, Glypican. (SEQ ID NO:98)
Length = 554 residues, 86.1% aligned
Score = 536 bits (1380), Expect = 2e−153

| | | | |
|---|---|---|---|
| NOV8a | 24 | SEAKVTRSCAETRQVLGARGYSLNLIPPALISGEHLRVCPQEYTCCSSETEQRLIRETEA | 83 |
| | | +\| +\|\|\|\|\| \|\|+ \|\|+\|+\|\|\| +\| + \|\|\|\|\|\|++\|\|\| \|\|\|\|\|\|\| \|++\| + | |
| 01153 | 17 | AEGSKSRSCAEVRQLFGAKGFSLNDVPQSEISGEHLQICPQGYTCCSSEMEEKLQLKARG | 76 |
| NOV8a | 84 | TFRGLVEDSGSFLVHTLAARHRKFDEFFLEMLSVAQHSLTQLFSHSYGRLYAQHALIFNG | 143 |
| | | \| \|++\|\| \| \| \|\| +\|\| \| \| \|+\|+++++ \| \|\|\| +\|\|\|\|\| \|+\| +\| | |
| 01153 | 77 | DFEQLLQDSSSSLQFLLATNAKKFQEHFEELLNISENYLNALFSKTYGRLYPQNAEMFKD | 136 |
| NOV8a | 144 | LFSRLRDFYGESGEGLDDTLADFWAQLLERVFPLLHPQYSFPPDYLLCLSRLASSTDGSL | 203 |
| | | \|\|+ \|\| +\| \| \|++ \| +\|\|\|+\|\|\|\| \| \|\| \|\| \| \|\|\| \|\| + \| | |
| 01153 | 137 | LFTELRLYYRGSNINLEEALNEFWARLLERAFKQLHGQYDSPDDYLECLRKARE----DL | 192 |
| NOV8a | 204 | QPFGDSPRRLRLQITRTLVAARAFVQGLETGRNVVSEALKVPVSEGCSQALMRLIGCPLC | 263 |
| | | +\|\|\|\| \|\|\|\| \|\|+\|\| \|\|\|\|\| \|+\|\|\| \| \|\|\|+ +\|\|+\|+ \|\|+\|\|+++\| \|\| \| | |
| 01153 | 193 | KPFGDIPRRLMLQVTRALVAARTFLQGLNVGIEVVSKVDQVPLSKECSRALLKMIYCPHC | 252 |
| NOV8a | 264 | RGVPSLMPCQGFCLNVVRGCLSSRG-LEPDWGNYLDGLLILADKLQGPFSFELTAESIGV | 322 |
| | | \|\|+\|\|+ \|\| \|+\|\|\|\|+\|\|\|\|+++ \|+\|+\| \|+\| \| +\|\|\|\|+ \|\|+ \| \|\| | |
| 01153 | 253 | RGLPSVKPCYGYCLNVMRGCLANQADLDPEWRGYIDSLELLADKMLGPYDIENVILSIHT | 312 |
| NOV8a | 323 | KISEGLMYLQENSAKVSAQVFQECGPPDPVPARNRRAPPPREEAGRLWSMVTEEERPTTA | 382 |
| | | \|\|\|\| +\| \|\|\|\| \|++\|+\|\|\| \|\| \| \| \| + \|\|\|\|\|\| | |
| 01153 | 313 | KISEAIMALQENGVKLTAKVFQGCG----TPKPTPYGSASGPEDKRSKRPLKPEERPTTE | 368 |
| NOV8a | 383 | AGTNLHRLVWELRERLARMRGFWARLSLTVCGDSRMAADASLEAAPCWTGAGRGRYLPPV | 442 |
| | | \| \|\|\| \| +\|+\| +++ \|\|+ \| \|+\| \| \|\|\|\| \|+ + \|\|\| \| \| \|\|\|\| \| | |
| 01153 | 369 | T---LERLVVEFKEKLKKVKSFWSTLPGTLCSD-RMAASAA-DDDPCWNGDGVGRYLQEV | 423 |
| NOV8a | 443 | VGGSPAEQVNNPELKVDASGPDVPTRRRRLQLRAATARMKTAALGHDLDGQDADEDASGS | 502 |
| | | \|\| \| \|+\|\|\|\|++\|\| \| \|\|+ \|++ +\|+ \| \|+ \|\| \|+\|+\| \|\|\| +\|+\|\|\| | |
| 01153 | 424 | VGNGLANQINNPEVEVDGSKPDMVIRQQIDKLKHMTNRLLAAASGNDVDFQDASDDSSGS | 483 |
| NOV8a | 503 | GGGQQYADDW | 512 |
| | | \| \| \|\| | |
| 01153 | 484 | GSGDGCGDDD | 493 |

Glypicans are a family of heparan sulfate proteoglycans which are anchored to cell membranes by a glycosylphosphatidylinositol (GPI) linkage. Structurally, these proteins consist of three separate domains: a signal sequence, an extracellular domain of about 500 residues that contains 12 conserved cysteines probably involved in disulfide bonds and which also contains the sites of attachment of the heparan sulfate glycosaminoglycan side chains and a C-terminal hydrophobic region which is post-translationally removed after formation of the GPI-anchor. Glypican-2 Precursor-like The above defined information for NOV8 suggests that NOVS may function as a member of a Glypican-2 Precursor family. Therefore, the NOV8 nucleic acids and proteins of the invention are useful in potential therapeutic applications implicated in various diseases and disorders described below and/or other pathologies. For example, the NOV8 compositions of the present invention will have efficacy for treatment of patients suffering from diabetes, diabetes mellitus non-insulin dependent, autoimmune disease, renal artery stenosis, interstitial nephritis, glomerulonephritis, polycystic kidney disease, systemic lupus erythematosus, renal tubular acidosis, IgA nephropathy, hypercalcemia, Lesch-Nyhan syndrome, hemophilia, hypercoagulation, idiopathic thrombocytopenic purpura, immunodeficiencies, graft versus host disease, Von Hippel-Lindau (VHL) syndrome, Alzheimer's disease, stroke, tuberous sclerosis, Parkinson's disease, Huntington's disease, cerebral palsy, epilepsy, multiple sclerosis, ataxia-telangiectasia, leukodystrophies, behavioral disorders, addiction, anxiety, pain, neurodegeneration, cancer, developmental abnormalities, Acyl-CoA dehydrogenase, deficiency of long chain, Brachydactyly, type A1, Carbamoylphosphate synthetase I deficiency, Cardiomyopathy dilated 1I, Cataract Coppock-like, Cataract crystalline aculeiform, Cataract polymorphic congenital, Cataract variable zonular pulverulent, Cataracts punctate progressive juvenile-onse, Choreoathetosis familial paroxysmal, Craniofacial-deafness-hand syndrome, Ichthyosis lamellar, type 2, Myopathy, desmin-related cardioskeletal, Resistance/susceptibility to TB, Rhabdomyosarcoma alveolar, Waardenburg syndrome type I and type III, Alport syndrome autosomal recessive, Bjornstad syndrome, Hematuria, familial benign, Hyperoxaluria primary, type 1, Syndactyly type 1, Hyperproglucagonemia, Bethlem myopathy, Brachydactyly type E, Brachydactyly-mental retardation syndrome, Finnish lethal neonatal metabolic syndrome, susceptibility to 2, Simpson-Golabi-Behmel syndrome, type 1 and type 2 and Beckwith-Wiedemann syndrome.

The NOV8 nucleic acid encoding Glypican-2 Precursor-like protein, and the Glypican-2 Precursor-like protein of the invention, or fragments thereof, may further be useful in diagnostic applications, wherein the presence or amount of the nucleic acid or the protein are to be assessed.

NOV9

A disclosed NOV9 nucleic acid of 985 nucleotides (also referred to AC011005_da2/139943578) encoding a novel Mitogen Activated Protein Kinase Kinase 2-like protein is shown in Table 9A. An open reading frame was identified beginning with an ATG initiation codon at nucleotides 54-56 and ending with a TGA codon at nucleotides 975-977. The start and stop codons are in bold letters.

TABLE 9A
NOV9 Nucleotide Sequence

(SEQ ID NO:27)

TCCACTACGGGCCCAGGCTAGAGGCGCCGCCGCCGCCGGCCCGCGGAGCCCCGATGCTGGCCCGGAGGAAG
CCGGTGCTGCCGGCGCTCACCATCAACCCTACCATCGCCGAGGGCCCATCCCCTACCAGCGAGGGCGCCTC
CGAGGCAAACCTGGTGGACCTGCAGAAGAAGCTGGAGGAGCTGGAACTTGACGAGCAGCAGAAGAAGCGGC
TGGAAGCCTTTCTCACCCAGAAAGCCAAGGTCGGCGAACTCAAAGACGATGACTTCGAAAGGATCTCAGAG
CTGGGCGCGGGCAACGGCGGGGTGGTCACCAAAGTCCAGCACAGACCCTCGGGCCTCATCATGGCCAGGAA
GCTGATCCACCTTGAGATCAAGCCGGCCATCCGGAACCAGATCATCCGCGAGCTGCAGGTCCTGCACGAAT
GCAACTCGCCGTACATCGTGGGCTTCTACGGGGCCTTCTACAGTGACGGGGAGATCAGCATTTGCATGGAA
CACATGGACGGCGGCTCCCTGGACCAGGTGCTGAAAGAGGCCAAGAGGATTCCCGAGGAGATCCTGGGGAA
AGTCAGCATCGCGGTTCTCCGGGGCTTGGCGTACCTCCGAGAGAAGCACCAGATCATGCACCGAGATGTGA
AGCCCTCCAACATCCTCGTGAACTCTAGAGGGGAGATCAAGCTGTGTGACTTCGGGGTGAGCGGCCAGCTC
ATCGACTCCATGGCCAACTCCTTCGTGGGCACGCGCTCCTACATGGCTCCACCTCCTAAGCTGCCCAACGG
TGTGTTCACCCCCGACTTCCAGGAGTTTGTCAATAAATGCCTCATCAAGAACCCAGCGGAGCGGGCGGACC
TGAAGATGCTCACAAACCACACCTTCATCAAGCGGTCCGAGGTGGAAGAAGTGGATTTTGCCGGCTGGTTG
TGTAAAACCCTGCGGCTGAACCAGCCCGGCACACCCACGCGCACCGCCGTGTGACAGTGGCAA

The disclosed NOV9 nucleic acid sequence has 754 of 759 bases (99%) identical to a Homo sapiens ERK activator kinase (MEK2) mRNA from (gb:GENBANK-ID:HUMMEK2NF|acc:L11285) (E=1.3e⁻²¹¹). The NOV9 nucleic acid sequence contains numerous SNPs which result in various amino acid changes.

A disclosed NOV9 polypeptide (SEQ ID NO:28) encoded by SEQ ID NO:27 is 307 amino acid residues and is presented using the one-letter amino acid code in Table 9B. Signal P, Psort and/or Hydropathy results predict that NOV9 does not contain a signal peptide and is likely to be localized in the cytoplasm with a certainty of 0.5500.

TABLE 9B
Encoded NOV9 protein sequence.

(SEQ ID NO:28)

MLARRKPVLPALTINPTIAEGPSPTSEGASEANLVDLQKKLEELELDEQQKKRLEAFLTQKAKVGELKDDD
FERISELGAGNGGVVTKVQHRPSGLIMARKLIHLEIKPAIRNQIIRELQVLHECNSPYIVGFYGAFYSDGE
ISICMEHMDGGSLDQVLKEAKRIPEEILGKVSIAVLRGLAYLREKHQIMHRDVKPSNILVNSRGEIKLCDF
GVSGQLIDSMANSFVGTRSYMAPPPKLPNGVFTPDFQEFVNKCLIKNPAERADLKMLTNHTFIKRSEVEEV
DFAGWLCKTLRLNQPGTPTRTAV

The NOV9 amino acid sequence has 236 of 236 amino acid residues (100%) identical to, and 236 of 236 amino acid residues (100%) similar to, the Homo sapiens 400 amino acid residue mitogen-activated protein kinase kinase 2 (EC 2.7.1.-) (Map kinase kinase 2) (MAPKK 2) (ERK activator kinase 2 (ptnr:SWISSPROT-ACC:P36507) (E=8.2e⁻¹⁶¹).

NOV9 is expressed in at least the following tissues: Adrenal Gland/Suprarenal gland, Amygdala, Bone, Bone Marrow, Brain, Colon, Coronary Artery, Dermis, Epidermis, Foreskin, Heart, Hypothalamus, Kidney, Liver, Lung, Lymph node, Lymphodid tissue, Mammary gland/Breast, Muscle, Nervous, Ovary, Pancreas, Peripheral Blood, Pituitary Gland, Placenta, Prostate, Retina, Small Intestine, Spleen, Stomach, Testis, Thymus, Tongue, Tonsils, Tumor, Umbilical Vein, Uterus, Whole Organism. This information was derived by determining the tissue sources of the sequences that were included in the invention. In addition, NOV9 is predicted to be expressed in the following tissues because of the expression pattern of a closely related Homo sapiens ERK activator kinase (MEK2) mRNA homolog (GENBANK-ID: gb:GENBANK-ID:HUMMEK2NF|acc:L11285): Lymphoid tissue, Nervous tissue, Gastrointestinal tissue, Peripheral Blood, and Cardiovascular tissue.

NOV9 also has homology to the amino acid sequences shown in the BLASTP data listed in Table 9C.

TABLE 9C
BLAST results for NOV9C

| Gene Index/ | | Length | Identity | Positives | |
|---|---|---|---|---|---|
| Identifier | Protein/Organism | (aa) | (%) | (%) | Expect |
| gi\|13651323\|ref\|XP_ | similar to | 325 | 236/236 | 236/236 | 1e−133 |
| 016871.1\| | mitogen-activated | | (100%) | (100%) | |
| | protein kinase | | | | |
| | kinase 2; protein | | | | |
| | kinase, mitogen- | | | | |
| | activated, kinase | | | | |
| | 2, p45 (MAP | | | | |
| | kinase kinase 2) | | | | |
| | [Homo sapiens] | | | | |
| gi\|13489054\|ref\|NP_ | mitogen-activated | 400 | 236/236 | 236/236 | 1e−131 |
| 109587.1\| | protein kinase | | (100%) | (100%) | |
| | kinase 2; protein | | | | |
| | kinase, mitogen- | | | | |
| | activated, kinase | | | | |
| | 2, p45 (MAP | | | | |
| | kinase kinase 2) | | | | |
| | [Homo sapiens] | | | | |
| gi\|1096928\|prf\| \|2113192A\| | MEK2 protein | 400 | 229/236 | 235/236 | 1e−129 |
| | [Rattus | | (97%) | (99%) | |
| | norvegicus | | | | |
| gi\|12844163\|dbj\|BAB | putative [Mus | 401 | 229/236 | 235/236 | 1e−129 |
| 26261.1\|(AK009392) | musculus] | | (97%) | (99%) | |
| gi\|15990388\|gb\|AAH1 | Unknown (protein | 401 | 229/236 | 235/236 | 1e−129 |
| 4830.1\|AAH14830 | for MGC: 25475) | | (97%) | (99%) | |
| (BC014830) | [Mus musculus] | | | | |

The homology of these sequences is shown graphically in the ClustalW analysis shown in Table 9D.

[image]

Tables 9E and 9F list the domain description from DOMAIN analysis results against NOV9. This indicates that the NOV9 sequence has properties similar to those of other proteins known to contain these domains.

TABLE 9E
Domain Analysis of NOV9

gnl|Smart|smart00220, S_TKc, Serine/Threonine protein kinases,
catalytic domain; Phosphotransferases. Serine or threonine-specific
kinase subfamily. (SEQ ID NO:99)
Length = 256 residues, 100.0% aligned
Score = 184 bits (468), Expect = 5e−48

| | | | |
|---|---|---|---|
| NOV9 | 72 | FERISELGAGNGGVVTKVQHRPSGLIMARKLIHLE-IKPAIRNQIIRELQVLHECNSPYI | 130 |
| | | +\| + \|\| \| \| \| + + +\| ++\| \|+\| \| +\| \| +\|+\|\|+++\| + + \| \| | |
| 00220 | 1 | YELLEVLGKGAFGKVYLARDKKTGKLVAIKVIKKEKLKKKKRERILREIKILKKLDHPNI | 60 |
| NOV9 | 131 | VGFYGAFYSDGEISICMEHMDGGSLDQVLKEAKRIPEEILGKVSIAVLRGLAYLREKHQI | 190 |
| | | \| \| \| \| ++ + \|\|+ +\|\| \| +\|\|+ \|+ \|+ + +\| \| \|\| \| | |
| 00220 | 61 | VKLYDVFEDDDKLYLVMEYCEGGDLFDLLKKRGRLSEDEARFYARQILSALEYL-HSQGI | 119 |
| NOV9 | 191 | MHRDVKPSNILVNSRGEIKLCDFGVSGQLIDS--MANSFVGTRSYMAP------------ | 236 |
| | | +\|\|\|+\|\| \|\|\|++\| \| +\|\| \|\|\|++ \|\| + +\|\|\|\| \|\|\|\| | |
| 00220 | 120 | IHRDLKPENILLDSDGHVKLADFGLAKQLDSGGTLLTTFVGTPEYMAPEVLLGKGYGKAV | 179 |
| NOV9 | 237 | -------------------------------------PPKLPNGVFTPDFQEFVNKCLIK | 259 |
| | | \| \| +\|+ ++ + \| \|+\| | |
| 00220 | 180 | DIWSLGVILYELLTGKPPFPGDDQLLALFKKIGKPPPPFPPPEWKISPEAKDLIKKLLVK | 239 |
| NOV9 | 260 | NPAERADLKMLTNHTFI | 276 |
| | | +\| +\| + \| \| | |
| 00220 | 240 | DPEKRLTAEEALEHPFF | 256 |

TABLE 9F
Domain Analysis of NOV9

gnl|Pfam|pfam00069, pkinase, Protein kinase domain. (SEQ ID NO:100)
Length = 256 residues, 100.0% aligned
Score = 165 bits (418), Expect = 3e−42

| | | | |
|---|---|---|---|
| NOV9 | 72 | FERISELGAGNGGVVTKVQHRPSGLIMARKLIHLEIKPAIRNQIIRELQVLHECNSPYIV | 131 |
| | | +\| +\|\|+\| \| \| \| +\|+ +\| \|+\| \|++ + + +\|\|+\|+\| + \| \|\| | |
| 00069 | 1 | YELGEKLGSGAFGKVYKGKHKDTGEIVAIKILKKRSLSEKKKRFLREIQILRRLSHPNIV | 60 |
| NOV9 | 132 | GFYGAFYSDGEISICMEHMDGGSLDQVLKEAK-RIPEEILGKVSIAVLRGLAYLREKHQI | 190 |
| | | \| \| \| + + \|\|+\|+\|\| \| \|+ + \|+ \|+++ +\|\|\|\| \|\| + \| | |
| 00069 | 61 | RLLGVFEEDDHLYLVMEYMEGGDLFDYLRRNGLLLSEKEAKKIALQILRGLEYLHSRG-I | 119 |
| NOV9 | 191 | MHRDVKPSNILVNSRGEIKLCDFGVSGQL---IDSMANSFVGTRSYMAP----------- | 236 |
| | | +\|\|\|+\|\| \|\|\|++ \| +\|+ \|\|\|++ +\| +\|\|\|\| \|\|\|\| | |
| 00069 | 120 | VHRDLKPENILLDENGTVKIADFGLARKLESSSYEKLTTFVGTPEYMAPEVLEGRGYSSK | 179 |
| NOV9 | 237 | --------------------------------------PPKLPNGVFTPDFQEFVNKCLIK | 259 |
| | | + + ++ + \|\|\| \| | |
| 00069 | 180 | VDVWSLGVILYELLTGKLPFPGIDPLEELFRIKERPRLRLPLPPNCSEELKDLIKKCLNK | 239 |
| NOV9 | 260 | NPAERADLKMLTNHTFI | 276 |
| | | +\| +\| \| + \|\| + | |
| 00069 | 240 | DPEKRPTAKEILNHPWF | 256 |

The amino acid sequence of NOV9 has high homology to other proteins as shown in Table 9G.

TABLE 9G
BLASTX results for NOV9

| | | Smallest | | |
|---|---|---|---|---|
| | | Sum | | |
| | Reading High | Prob | | |
| Sequences producing High-scoring Segment Pairs: | Frame | Score | P(N) | N |
| patp:AAY41652 Human MEK2 protein sequenc, Homo Sap1 400 aa.. | +3 | 1194 | 4.8e−160 | 1 |
| patp:AAW88434 Dis ass prot kinase DAPK-3, Homo Sap1 400 aa. | +3 | 1186 | 3.3e−159 | 1 |

The protein similarity information, expression pattern, and map location for the NOV9 suggest that NOV9 may have important structural and/or physiological functions characteristic of the Mitogen Activated Protein Kinase Kinase 2 protein family. Therefore, the NOV9 nucleic acids and proteins of the invention are useful in potential therapeutic applications implicated in various diseases and disorders described below and/or other pathologies. For example, the NOV9 compositions of the present invention will have efficacy for treatment of patients suffering from atherosclerosis, metabolic diseases, pathogen infections and neurological diseases. The NOV9 nucleic acid encoding Mitogen Activated Protein Kinase Kinase 2-like protein, and the Mitogen Activated Protein Kinase Kinase 2-like protein of the invention, or fragments thereof, may further be useful in diagnostic applications, wherein the presence or amount of the nucleic acid or the protein are to be assessed.

NOV10

A disclosed NOV10 nucleic acid of 1506 nucleotides (also referred to sggc_draft_c333e1_{—}20000804_da2) encoding a zinc finger protein 276 C₂H2 type-like protein is shown in Table 1 OA. An open reading frame was identified beginning with an ATG initiation codon at nucleotides 385-387 and ending with a TGA codon at nucleotides 1504-1506. A putative untranslated region upstream from the intiation codon is underlined in Table 10A, and the start and stop codons are in bold letters.

TABLE 10A
NOV10 Nucleotide Sequence

(SEQ ID NO:29)

CGCTGAGGTTTGAGATCTCGAGAGGGTCCCGTACGACGAGCACTGTGAACCTCCGCCTGCTTGTCCGGCTC
ATGGCCACACTGATCCTTTGCAGGGTCGGTGCCCAGCCCCCCACAGGGGCAGAGGAGGGAGCGTGTCTGGG
TGAGTCCTCCCCCGGTGGAGGGTGGGCTGGGTGCCGACCAGCCGTGGATCTGACATCTCTGTTGACTCTCT
GCAGTGGATCTGATCACATCCAGCCCCCAGTGCCTGCACGGCTTGGTGGGGTGGGTGCATGGACATGCGGC
CAGCTGCGGGGCCCTACCCCACCTTCAGAGGACACTGTCCTCCGAGTACTGCGGCGTCATCCAGGTCGTGT
GGGGCTGCGACCAGGGCCACGACTACACCATGGATACCAGCTCCAGCTGCAAGGCCTTCTTGCTGGACAGT
GCGCTGGCAGTCAAGTGGCCATGGGACAAAGAGACGGCGCCACGGCTGCCCCAGCACCGAGGGTGGAACCC
TGGGGATGCCCCTCAGACCTCCCAGGGTAGAGGGACAGGGACCCCAGTTGGGGCTGAGACCAAGACCCTGC
CCAGCACGGATGTGGCCCAGCCTCCTTCGGACAGCGACGCGGTGGGGCCCAGGTCGGGCTTCCCACCTCAG
CCAAGCCTGCCCCTTTGCAGGGCCCCAGGGCAGTTGGGTGAGAAGCAGCTTCCATCTTCAACCTCGGATGA
TCGGGTAAAAGACGAGTTCAGTGACCTTTCTGAGGGAGACGTCTTGAGTGAAGATGAAAATGACAAGAAGC
AAAATGCCCAGTCTTCGGACGAGTCCTTTGAGCCTTACCCAGAAAGGAAAGTCTCTGGTAAGAAGAGTGAA
AGCAAAGAAGCCAAGAAGTCTGAAGAACCAAGAATTCGGAAGAAGCCGGGACCCAAGCCCGGATGGAAGAA
GAAGCTTCGTTGTGAGAGGGAGGAGCTTCCCACCATCTACAAGTGTCCTTACCAGGGCTGCACGGCCGTGT
ACCGAGGCGCTGACGGCATGAAGAAGCACATCAAGGAGCACCACGAGGAGGTCCGGGAGCGGCCCTGCCCC
CACCCTGGCTGCAACAAGGTTTTCATGATCGACCGCTACCTGCAGCGCCACGTGAAGCTCATCCACACAGA
GGTGCGGAACTATATCTGTGACGAATGTGGACAAACCTTCAAGCAGCGGAAGCACCTTCTCGTCCACCAAA
TGCGACATTCGGGAGCCAAGCCTTTGCAGTGTGAGGTCTGTGGGTTCCAGTGCAGGCAGCGGGCATCCCTC
AAGTACCACATGACCAAACACAAGGCTGAGACTGAGCTGGACTTTGCCTGTGACCAGTGTGGCCGGCGGTT
TGAGAAGGCCCACAACCTCAATGTACACATGTCCATGGTGCACCCGCTGACACAGACCCAGGACAAGGCCC
TGCCCCTGGAGGCGGAACCACCACCTGGGCCACCGAGCCCCTCTGTGACCACAGAGGGCCAGGCGGTGAAG
CCCGAACCCACCTGA

The disclosed NOV10 nucleic acid sequence, localized to chromosome 16, has 271 of 271 bases (100%) identical Homo sapiens Fanconi anaemia group A gene, exons 39, 40, 41, 42 and 43 mRNA (gb:GENBANK-ID:HSZ83095|acc:Z83095) (E=9.4e⁻⁷⁷). 5 A disclosed NOV10 polypeptide (SEQ ID NO:30) encoded by SEQ ID NO:29 is 373 amino acid residues and is presented using the one-letter amino acid code in Table 10B. Signal P, Psort and/or Hydropathy results predict that NOV10 does not contain a signal peptide and is likely to be localized at the mitochondrial matrix space with a certainty of 0.5517.

TABLE 10B
Encoded NOV10 protein sequence.

(SEQ ID NO:30)

MDTSSSCKAFLLDSALAVKWPWDKETAPRLPQHRGWNPGDAPQTSQGRGTGTPVGAETKTLPSTDVAQPP
SDSDAVGPRSGFPPQPSLPLCRAPGQLGEKQLPSSTSDDRVKDEFSDLSEGDVLSEDENDKKQNAQSSDE
SFEPYPERKVSGKKSESKEAKKSEEPRIRKKPGPKPGWKKKLRCEREELPTIYKCPYQGCTAVYRGADGM
KKHIKEHHEEVRERPCPHPGCNKVFMIDRYLQRHVKLIHTEVRNYICDECGQTFKQRKHLLVHQMRHSGA
KPLQCEVCGFQCRQRASLKYHMTKHKAETELDFACDQCGRRFEKAHNLNVHMSMVHPLTQTQDKALPLEA
EPPPGPPSPSVTTEGQAVKPEPT

The NOV10 amino acid sequence has 310 of 373 amino acid residues (83%) identical to, and 325 of 373 amino acid residues (87%) similar to, the Mus musculus 372 amino acid residue zinc finger protein 276 C₂H2 type (ptnr:TREMBLNEW-ACC:AAGO 1634)(E=6.3e^{169).}

NOV10 is expressed in at least the following tissues: bone marrow, brain, cervix, Icolon, coronary artery, heart, hypothalamus, kidney, lymph node, lung, ovary, peripheral blood, prostate, testis, thyroid, tonsils, uterus and whole organism.

The disclosed NOV10 polypeptide has homology to the amino acid sequences shown in the BLASTP data listed in Table 10C.

TABLE 10C
BLAST results for NOV10

| Gene Index/ | | Length | Identity | Positives | |
|---|---|---|---|---|---|
| Identifier | Protein/Organism | (aa) | (%) | (%) | Expect |
| gi\|10048420\|ref\|NP_ | zinc finger protein | 372 | 310/374 | 325/374 | 1e−155 |
| 065243.1\| | (C2H2 type) | | (82%) | (86%) | |
| | 276 [Mus | | | | |
| | musculus] | | | | |
| gi\|11611571\|dbj\|BAB | hypothetical protein | 298 | 251/253 | 252/253 | 1e−121 |
| 19000.1\|(AB052145) | [Macaca fascicularis] | | (99%) | (99%) | |
| gi\|14776742\|ref\|XP | hypothetical | 400 | 253/253 | 253/253 | 1e−120 |
| 047520.1\| | protein XP_047520 | | (100%) | (100%) | |
| | [Homo sapiens] | | | | |
| gi\|11611570\|dbj\|BAB | hypothetical | 280 | 104/110 | 106/110 | 8e−53 |
| 18999.1\|(AB052145) | protein [Macaca | | (94%) | (95%) | |
| | fascicularis] | | | | |
| gi\|15559662\|gb\|AAH1 | Unknown (protein | 615 | 86/226 | 127/226 | 7e−38 |
| 4187.1\|AAH14187 | for MGC: 20975) | | (38%) | (56%) | |
| (BC014187) | [Homo sapiens] | | | | |

The homology between these and other sequences is shown graphically in the ClustalW analysis shown in Table 10D.

[image]

[image]

Table 10E lists the domain description from DOMAIN analysis results against NOV10. This indicates that the NOV10 sequence has properties similar to those of other proteins known to contain these domains.

TABLE 10E
Domain Analysis of NOV10

gnl|Pfam|pfam00096, zf-C2H2, Zinc finger, C2H2
type. The C2H2 zinc finger is the classical zinc
finger domain. The two conserved cysteines and
histidines co-ordinate a zinc ion. The following
pattern describes the zinc finger. #-X-C-X(1-5)-C-
X3-#-X5-#-X2-H-X(3-6)-[H/C] Where X can be any
amino acid, and numbers in brackets indicate the
number of residues. The positions marked # are
those that are important for the stable fold of
the zinc finger. The final position can be either
his or cys. The C2H2 zinc finger is composed of
two short beta strands followed by an alpha helix.
The amino terminal part of the helix binds the
major groove in DNA binding zinc fingers.
(SEQ ID NO:101)
Length = 23 residues, 100.0% aligned
Score = 35.8 bits (81), Expect = 0.004

| | | | |
|---|---|---|---|
| NOV10 | 255 | YICDECGQTFKQRKHLLVHQMRH | 277 |
| | | \| \| +\|\|++\| ++ +\| \| \| | |
| 00096 | 1 | YKCPDCGKSFSRKSNLKRHLRTH | 23 |

The protein similarity information, expression pattern, and map location for the NOV10 suggest that NOV10 may have important structural and/or physiological functions characteristic of the zinc finger protein 276 C₂H2 protein family. Therefore, the NOV10 nucleic acids and proteins of the invention are useful in potential therapeutic applications implicated in various diseases and disorders described below and/or other pathologies. For example, the NOV10 compositions of the present invention will have efficacy for treatment of patients suffering from cancer, trauma, immunological disease, respiratory disease, heart disease, gastro-intestinal diseases, reproductive health, neurological and neurodegenerative diseases, bone marrow transplantation, metabolic and endocrine diseases, allergy and inflammation, nephrological disorders, hematopoietic disorders and urinary system disorders. The NOV10 nucleic acid encoding zinc finger protein 276 C₂H2 type-like protein, and the zinc finger protein 276 C₂H2 type-like protein of the invention, or fragments thereof, may further be useful in diagnostic applications, wherein the presence or amount of the nucleic acid or the protein are to be assessed.

NOV11

NOV11 includes in vivo novel Thymosin beta-10-like proteins disclosed below. The disclosed proteins have been named NOV11a and NOV11b.

NOV11a

A disclosed NOV11a nucleic acid of 129 nucleotides (also referred to GMAC079400_A) encoding a novel Thymosin beta-10-like protein is shown in Table 11A. An open reading frame was identified beginning with an ATG initiation codon at nucleotides 28-30 and ending with a TAA codon at nucleotides 157-159. Putative untranslated regions upstream from the initiation codon and downstream from the termination codon are underlined in Table 11A, and the start and stop codons are in bold letters.

TABLE 11A
NOV11a Nucleotide Sequence

(SEQ ID NO:31)

ACGGATGGTACCGATTGTTTTAAGAAAATGGCAGACAAACCAGACGTGGGGGGAATCGCCAGCTTCAATA
GGGCCAAGCTGAAGAAAACGGAGACGCAGGAGAAGAACACCCTGCCGACCAAAGAGACCACTGGGCAGAA
GCGGAGTGAAATTTCCTAAGAGCCCGGAGGATTTCCTGCCCTCGTC

The disclosed NOV11a nucleic acid sequence has 172 of 190 bases (90%) identical to a Homo sapiens Thymosin beta-10 mRNA (GENBANK-ID: S54005) (E=3.1e⁻²⁸).

A disclosed NOV11a polypeptide (SEQ ID NO:32) encoded by SEQ ID NO:31 is 43 amino acid residues and is presented using the one-letter amino acid code in Table 11B. Signal P, Psort and/or Hydropathy results predict that NOV1 la does not contain a signal peptide and is likely to be localized to the nucleus with a certainty of 0.5426

TABLE 11B
Encoded NOV11a protein sequence.

(SEQ ID NO:32)

MADKPDVGGIASFNRAKLKKTETQEKNTLPTKETTGQKRSEIS

The NOV11a amino acid sequence has 37 of 44 amino acid residues (84%) identical to, and 40 of 44 amino acid residues (90%) similar to, the Rattus norvegicus 44 amino acid residue Thymosin beta-10 protein (A27266) (E=2.4e⁻¹²). The global sequence homology is 88.372% amino acid homology and 86.047% amino acid identity.

NOV11a is predicted to be expressed in the Metastatic Melanoma tissues because of the expression pattern of a closely related Homo sapiens Thymosin beta-10 homolog (GENBANK-ID: S54005).

NOV11b

A disclosed NOV11b nucleic acid of 173 nucleotides (also referred to CG109754-01) encoding a novel Thymosin beta-10-like protein is shown in Table 11C. An open reading frame was identified beginning with an ATG initiation codon at nucleotides 27-29 and ending with a TAA codon at nucleotides 156-158. Putative untranslated regions upstream from the initiation codon and downstream from the termination codon are underlined in Table 11C, and the start and stop codons are in bold letters.

TABLE 11C
NOV11b Nucleotide Sequence

(SEQ ID NO:33)

CGGATGGTACCGATTGTTTTAAGAAAATGGCAGACAAACCAGACGTGGGGGGAATCGCCAGCTTCAATAGG
GCCAAGCTGAAGAAAACGGAGACGCAGGAGAAGAACACCCTGCCGACCAAAGAGACCACTGGGCAGAAGCG
GAGTGAAATTTCCTAAGAGCCCGGAGGATTT

The disclosed NOV11b nucleic acid sequence, localized to chromosome 2, has 155 of 168 bases (92%) identical to a Homo sapiens Thymosin beta-10 mRNA (gb:GENBANK-.ID:HUMTHMBX|acc:M92381.1) (E=4.1⁻²⁵).

A disclosed NOV11b polypeptide (SEQ ID NO:34) encoded by SEQ ID NO:33 is 43 amino acid residues and is presented using the one-letter amino acid code in Table 11D. Signal P, Psort and/or Hydropathy results predict that NOV11b does not contain a signal peptide and is likely to be localized to the nucleus with a certainty of 0.5426 Although PSORT suggests the NOV11b polypeptide may be localized in the nucleus, the NOV11b protein is similar to the Thymosin family, some members of which are released extracellularly. Therefore it is likely that this novel Thymosin Beta 10-like protein is localized to the extracellular space.

TABLE 11D
Encoded NOV11b protein sequence.

(SEQ ID NO:34)

MADKPDVGGIASFNRAKLKKTETQEKNTLPTKETTGQKRSEIS

The NOV11b amino acid sequence has 36 of 43 amino acid residues (83%) identical to, and 39 of 43 amino acid residues (90%) similar to, the Homo sapiens 43 amino acid residue Thymosin beta-10 protein (ptnr:SWISSNEW-ACC:P13472) (E=1.7e⁻²). NOV11b protein is 43 amino acids long, which is the same length as public protein P13472. NOV11b protein differs at eight amino acid positions. NOV11b begins with a methionine that the public GenBank submission is lacking. In addition to this, there are six single amino acid changes (M6V, E8G, D14N, K15R, 134T, E35G) and a single amino acid deletion (E37-). This number of changes in such a short peptide indicates that NOV11b protein is derived from a different gene than the public protein.

NOV11b is predicted to be expressed in brain and neuroblastoma tissues because of the expression pattern of a closely related Homo sapiens Thymosin beta-10 homolog (GENBANK-ID: gb:GENBANK-ID:HUMTHMBX|acc:M92381.1).

NOV11a and NOV11b are very closely homologous as is shown in the amino acid alignment in Table 11E.

[image]

Homologies to any of the above NOV11 proteins will be shared by the other NOV11 proteins insofar as they are homologous to each other as shown above. Any reference to NOV11 is assumed to refer to both of the NOV11 proteins in general, unless otherwise noted.

NOV11a also has homology to the amino acid sequences shown in the BLASTP data listed in Table 11F.

TABLE 11F
BLAST results for NOV11a

| Gene Index/ | | Length | Identity | | |
|---|---|---|---|---|---|
| Identifier | Protein/Organism | (aa) | (%) | Positives (%) | Expect |
| gi\|339697\|gb\|AAA36746.1\| | thymosin beta-10 | 49 | 37/44 | 40/44 | 4e−04 |
| (M92383) | [Homo sapiens] | | (84%) | (90%) | |
| gi\|10863895\|ref\|NP_ | thymosin, beta 10 | 42 | 34/42 | 39/42 | 0.002 |
| 066926.1\| | [Homo sapiens] | | (80%) | (91%) | |
| gi\|223789\|prf\| \|0912169A | thymosin | 44 | 37/44 | 40/44 | 0.005 |
| | beta10,Arg | | (84%) | (90%) | |
| | [Oryctolagus | | | | |
| | cuniculus] | | | | |
| gi\|2143995\|pir\| \|I52084 | thymosin beta-4 | 43 | 36/43 | 39/43 | 0.019 |
| | precursor | | (83%) | (89%) | |
| | (fragment) | | | | |
| | [Rattus | | | | |
| | norvegicus] | | | | |

The homology of these sequences is shown graphically in the ClustalW analysis shown in Table 11G.

[image]

The amino acid sequence of NOV 1I has high homology to other proteins as shown in

TABLE 11H
BLASTX results for NOV11

| | | Smallest | | |
|---|---|---|---|---|
| | | Sum | | |
| | Reading High | Prob | | |
| Sequences producing High-scoring Segment Pairs: | Frame | Score | P(N) | N |
| ptkm1 patp:AAY80267 Thymosin beta 4 peptide isoform Tbeta10, Unknown 43 aa.. | +1 | 169 | 7.2e-12 | 1 |

The protein similarity information, expression pattern, and map location for the NOV11 protein and nucleic acid suggest that NOV11 may have important structural and/or physiological functions characteristic of the Thymosin beta 10 family. Therefore, the NOV11 nucleic acids and proteins of the invention are useful in potential therapeutic applications implicated in various diseases and disorders described below and/or other pathologies. For example, the NOV11 compositions of the present invention will have efficacy for treatment of patients suffering from prostate cancer, immunological and autoimmune disorders (ie hyperthyroidism), angiogenesis and wound healing, modulation of apoptosis, neurodegenerative and neuropsychiatric disorders, age-related disorders, pathological disorders involving spleen, thymus, lung, and peritoneal macrophages and/or other pathologies and disorders. The NOV11 nucleic acid encoding Thymosin beta 10-like protein, and the Thymosin beta 10-like protein of the invention, or fragments thereof, may further be useful in diagnostic applications, wherein the presence or amount of the nucleic acid or the protein are to be assessed.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

An NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, I kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence SEQ ID NOS:1, 3, 5, 7, 9, 11, 13, 15, 17, 19, 21, 23, 25, 27, 29, 31 and 33, or a complement of this aforementioned nucleotide sequence, can be isolated using standard molecular biology techniques and the sequence information provided herein. Using all or a portion of the nucleic acid sequence of SEQ ID NOS:1, 3, 5, 7, 9, 11, 13, 15, 17, 19, 21, 23, 25, 27, 29, 31 and 33 as a hybridization probe, NOVX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oliigonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides SEQ ID NOS:1, 3, 5, 7, 9, 11, 13, 15, 17, 19, 21, 23, 25, 27, 29, 31 and 33, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NOS:1, 3, 5, 7, 9, 11, 13, 15, 17, 19, 21, 23, 25, 27, 29, 31 and 33, or a portion of this nucleotide sequence (e.g., a fragment that can be used as a probe or primer or a fragment encoding a biologically-active portion of an NOVX polypeptide). A nucleic acid molecule that is complementary to the nucleotide sequence shown NOS:1, 3, 5, 7, 9, 11, 13, 15, 17, 19, 21, 23, 25, 27, 29, 31 or 33 is one that is sufficiently complementary to the nucleotide sequence shown NOS:1, 3, 5, 7, 9, 11, 13, 15, 17, 19, 21, 23, 25, 27, 29, 31 or 33 that it can hydrogen bond with little or no mismatches to the nucleotide sequence shown SEQ ID NOS:1, 3, 5, 7, 9, 11, 13, 15, 17, 19, 21, 23, 25, 27, 29, 31 and 33, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for an NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NOS:1, 3, 5, 7, 9, 11, 13, 15, 17, 19, 21, 23, 25, 27, 29, 31 and 33, as well as a polypeptide possessing NOVX biological activity. Various biological activities of the NOVX proteins are described below.

An NOVX polypeptide is encoded by the open reading frame (“ORF”) of an NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with without a start codon, a stop codon, or both. For an ORF to be considered as a good candidate for coding for a bona fide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence SEQ ID NOS:1, 3, 5, 7, 9, 11, 13, 15, 17, 19, 21, 23, 25, 27, 29, 31 and 33; or an anti-sense strand nucleotide sequence of SEQ ID NOS:1, 3, 5, 7, 9, 11, 13, 15, 17, 19, 21, 23, 25, 27, 29, 31 and 33; or of a naturally occurring mutant of SEQ ID NOS:1, 3, 5, 7, 9, 11, 13, 15, 17, 19, 21, 23, 25, 27, 29, 31 and 33.

Probes based on the human NOVX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express an NOVX protein, such as by measuring a level of an NOVX-encoding nucleic acid in a sample of cells from a subject e.g., detecting NOVX mRNA levels or determining whether a genomic NOVX gene has been mutated or deleted.

“A polypeptide having a biologically-active portion of an NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion SEQ ID NOS:1, 3, 5, 7, 9, 11, 13, 15, 17, 19, 21, 23, 25, 27, 29, 31 and 33, that encodes a polypeptide having an NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences shown in SEQ ID NOS:1, 3, 5, 7, 9, 11, 13, 15, 17, 19, 21, 23, 25, 27, 29, 31 and 33 due to degeneracy of the genetic code and thus encode the same NOVX proteins as that encoded by the nucleotide sequences shown in SEQ ID NOS:1, 3, 5, 7, 9, 11, 13, 15, 17, 19, 21, 23, 25, 27, 29, 31 and 33. In another embodiment, an isolated nucleic acid molecule of the invention has a nucleotide sequence encoding a protein having an amino acid sequence shown in SEQ ID NOS:2, 4, 6, 8, 10, 12, 14, 16, 18, 20, 22, 24, 26, 28, 30, 32 and 34.

In addition to the human NOVX nucleotide sequences shown in SEQ ID NOS:1, 3, 5, 7, 9, 11, 13, 15, 17, 19, 21, 23, 25, 27, 29, 31 and 33, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding an NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from the human SEQ ID NOS:1, 3, 5, 7, 9, 11, 13, 15, 17, 19, 21, 23, 25, 27, 29, 31 and 33 are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NOS:1, 3, 5, 7, 9, 11, 13, 15, 17, 19, 21, 23, 25, 27, 29, 31 and 33. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to the sequences SEQ ID NOS:1, 3, 5, 7, 9, 11, 13, 15, 17, 19, 21, 23, 25, 27, 29, 31 and 33, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NOS:1, 3, 5, 7, 9, 11, 13, 15, 17, 19, 21, 23, 25, 27, 29, 31 and 33, or fragments, analogs or derivatives thereof, under conditions of moderate stringency is provided. A non-limiting example of moderate stringency hybridization conditions are hybridization in 6×SSC, 5× Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al (eds.), 1993, CURRENT PROTOCOLS IN MOLECULAR BIOLOGY, John Wiley & Sons, NY, and Kriegler, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences SEQ ID NOS:1, 3, 5, 7, 9, 11, 13, 15, 17, 19, 21, 23, 25, 27, 29, 31 and 33, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences SEQ ID NOS:1, 3, 5, 7, 9, 11, 13, 15, 17, 19, 21, 23, 25, 27, 29, 31 and 33, thereby leading to changes in the amino acid sequences of the encoded NOVX proteins, without altering the functional ability of said NOVX proteins. For example, nucleotide substitutions leading to amino acid substitutions at “non-essential” amino acid residues can be made in the sequence SEQ ID NOS:2, 4, 6, 8, 10, 12, 14, 16, 18, 20, 22, 24, 26, 28, 30, 32 and 34. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NOS:1, 3, 5, 7, 9, 11, 13, 15, 17, 19, 21, 23, 25, 27, 29, 31 and 33 yet retain biological activity. In one embodiment, the isolated nucleic acid molecule comprises a nucleotide sequence encoding a protein, wherein the protein comprises an amino acid sequence at least about 45% homologous to the amino acid sequences SEQ ID NOS:2, 4, 6, 8, 10, 12, 14, 16, 18, 20, 22, 24, 26, 28, 30, 32 and 34. Preferably, the protein encoded by the nucleic acid molecule is at least about 60% homologous to SEQ ID NOS:2, 4, 6, 8, 10, 12, 14, 16, 18, 20, 22, 24, 26, 28, 30, 32 and 34; more preferably at least about 70% homologous SEQ ID NOS:2, 4, 6, 8, 10, 12, 14, 16, 18, 20, 22, 24, 26, 28, 30, 32 and 34; still more preferably at least about 80% homologous to SEQ ID NOS:2, 4, 6, 8, 10, 12, 14, 16, 18, 20, 22, 24, 26, 28, 30, 32 and 34; even more preferably at least about 90% homologous to SEQ ID NOS:2, 4, 6, 8, 10, 12, 14, 16, 18, 20, 22, 24, 26, 28, 30, 32 and 34; and most preferably at least about 95% homologous to SEQ ID NOS:2, 4, 6, 8, 10, 12, 14, 16, 18, 20, 22, 24, 26, 28, 30, 32 and 34.

An isolated nucleic acid molecule encoding an NOVX protein homologous to the protein of SEQ ID NOS:2, 4, 6, 8, 10, 12, 14, 16, 18, 20, 22, 24, 26, 28, 30, 32 and 34 can be created by introducing one or more nucleotide substitutions, additions or deletions into the nucleotide sequence of SEQ ID NOS:1, 3, 5, 7, 9, 11, 13, 15, 17, 19, 21, 23, 25, 27, 29, 31 and 33, such that one or more amino acid substitutions, additions or deletions are introduced into the encoded protein.

Mutations can be introduced into SEQ ID NOS:1, 3, 5, 7, 9, 11, 13, 15, 17, 19, 21, 23, 25, 27, 29, 31 and 33 by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of an NOVX coding sequence, such as by saturation mutagenesis, and the resultant mutants can be screened for NOVX biological activity to identify mutants that retain activity. Following mutagenesis SEQ ID NOS:1, 3, 5, 7, 9, 11, 13, 15, 17, 19, 21, 23, 25, 27, 29, 31 and 33, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, VLIM, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and an NOVX ligand; or (iii) the ability of a mutant NOVX protein to bind to an intracellular target protein or biologically-active portion thereof; (e.g. avidin proteins).

In yet another embodiment, a mutant NOVX protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NOS:1, 3, 5, 7, 9, 11, 13, 15, 17, 19, 21, 23, 25, 27, 29, 31 and 33, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of an NOVX protein of SEQ ID NOS:2, 4, 6, 8, 10, 12, 14, 16, 18, 20, 22, 24, 26, 28, 30, 32 and 34, or antisense nucleic acids complementary to an NOVX nucleic acid sequence of SEQ ID NOS:1, 3, 5, 7, 9, 11, 13, 15, 17, 19, 21, 23, 25, 27, 29, 31 and 33, are additionally provided.

In one embodiment, an antisense nucleic acid molecule is antisense to a “coding region” of the coding strand of a nucleotide sequence encoding an NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated ini situ such that they hybridize with or bind to cellular mRNA and/or genomic DNA encoding an NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual α-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for an NOVX-encoding nucleic acid can be designed based upon the nucleotide sequence of an NOVX cDNA disclosed herein (i.e., SEQ ID NOS:1, 3, 5, 7, 9, 11, 13, 15, 17, 19, 21, 23, 25, 27, 29, 31 and 33). For example, a derivative of a Tetrahymena L-19 IVS RNA can be constructed in which the nucleotide sequence of the active site is complementary to the nucleotide sequence to be cleaved in an NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. NY. Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g, DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996.supra); or as probes or primers for DNA sequence and hybridization (See, Hyrup, et al., 1996, supra; Perry-O'Keefe, et al., 1996. supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Clien. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in SEQ ID NOS:2, 4, 6, 8, 10, 12, 14, 16, 18, 20, 22, 24, 26, 28, 30, 32 and 34. The invention also includes a mutant or variant protein any of whose residues may be changed from the corresponding residues shown in SEQ ID NOS:2, 4, 6, 8, 10, 12, 14, 16, 18, 20, 22, 24, 26, 28, 30, 32 and 34 while still encoding a protein that maintains its NOVX activities and physiological functions, or a functional fragment thereof.

In general, an NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, an NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence shown in SEQ ID NOS:2, 4, 6, 8, 10, 12, 14, 16, 18, 20, 22, 24, 26, 28, 30, 32 and 34) that include fewer amino acids than the full-length NOVX proteins, and exhibit at least one activity of an NOVX protein. Typically, biologically-active portions comprise a domain or motif with at least one activity of the NOVX protein. A biologically-active portion of an NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence shown SEQ ID NOS:2, 4, 6, 8, 10, 12, 14, 16, 18, 20, 22, 24, 26, 28, 30, 32 and 34. In other embodiments, the NOVX protein is substantially homologous to SEQ ID NOS:2, 4, 6, 8, 10, 12, 14, 16, 18, 20, 22, 24, 26, 28, 30, 32 and 34, and retains the functional activity of the protein of SEQ ID NOS:2, 4, 6, 8, 10, 12, 14, 16, 18, 20, 22, 24, 26, 28, 30, 32 and 34, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence SEQ ID NOS:2, 4, 6, 8, 10, 12, 14, 16, 18, 20, 22, 24, 26, 28, 30, 32 and 34, and retains the functional activity of the NOVX proteins of SEQ ID NOS:2, 4, 6, 8, 10, 12, 14, 16, 18, 20, 22, 24, 26, 28, 30, 32 and 34.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOS:1, 3, 5, 7, 9, 11, 13, 15, 17, 19, 21, 23, 25, 27, 29, 31 and 33.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, an NOVX “chimeric protein” or “fusion protein” comprises an NOVX polypeptide operatively-linked to a non-NOVX polypeptide. An “NOVX polypeptide” refers to a polypeptide having an amino acid sequence corresponding to an NOVX protein SEQ ID NOS:2, 4, 6, 8, 10, 12, 14, 16, 18, 20, 22, 24, 26, 28, 30, 32 and 34, whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within an NOVX fusion protein the NOVX polypeptide can correspond to all or a portion of an NOVX protein. In one embodiment, an NOVX fusion protein comprises at least one biologically-active portion of an NOVX protein. In another embodiment, an NOVX fusion protein comprises at least two biologically-active portions of an NOVX protein. In yet another embodiment, an NOVX fusion protein comprises at least three biologically-active portions of an NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is an NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is an NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between an NOVX ligand and an NOVX protein on the surface of a cell, to thereby suppress NOVX-mediated signal transduction in vivo. The NOVX-immunoglobulin fusion proteins can be used to affect the bioavailability of an NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with an NOVX ligand.

An NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). An NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahiedi-osl 39: 3; Itakura, et al., 1984. Annu. Rev. Biochem. 53: 323; Itakura, et al., 1984. Science 198: 1056; Ike, et al., 1983. Nucl. Acids Res. 11:477.

Polypeptide Libraries

In addition, libraries of fragments of the NOVX protein coding sequences can be used to generate a variegated population of NOVX fragments for screening and subsequent selection of variants of an NOVX protein. In one embodiment, a library of coding sequence fragments can be generated by treating a double stranded PCR fragment of an NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S1 nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

Anti-NOVX Antibodies

Also included in the invention are antibodies to NOVX proteins, or fragments of NOVX proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, Fab, F_{ab′}and F_{(ab′)2} fragments, and an F_{ab} expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated NOVX-related protein of the invention may be intended to serve as an antigen, or a portion or fragment thereof, and additionally can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX-related protein that is located on the surface of the protein, e.g., a hydrophilic region. A hydrophobicity analysis of the human NOVX-related protein sequence will indicate which regions of a NOVX-related protein are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each of which is incorporated herein by reference in its entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow and Lane, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, MONOCLONAL ANTIBODIES: PRINCIPLES AND PRACTICE, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., MONOCLONAL ANTIBODY PRODUCTION TECHNIQUES AND APPLICATIONS, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Preferably, antibodies having a high degree of specificity and a high binding affinity for the target antigen are isolated.

After the desired hybridoma cells are identified, the clones can be subcloned by limiting dilution procedures and grown by standard methods. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies relate to antibody molecules in which essentially the entire sequences of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al, (Nature Biotechnology 14, 845-51 (1996)); Neuberger (Nature Biotechnology 14, 826 (1996)); and Lonberg and Huszar (Intern. Rev. Immunol. 1365-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of Fab expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., 1991 EMBO J., 10:3655-3659.

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5): 1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγR11 (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of an NOVX protein is facilitated by generation of hybridomas that bind to the fragment of an NOVX protein possessing such a domain. Thus, antibodies that are specific for a desired domain within an NOVX protein, or derivatives, fragments, analogs or homologs thereof, are also provided herein.

Anti-NOVX antibodies may be used in methods known within the art relating to the localization and/or quantitation of an NOVX protein (e.g., for use in measuring levels of the NOVX protein within appropriate physiological samples, for use in diagnostic methods, for use in imaging the protein, and the like). In a given embodiment, antibodies for NOVX proteins, or derivatives, fragments, analogs or homologs thereof, that contain the antibody derived binding domain, are utilized as pharmacologically-active compounds (hereinafter “Therapeutics”).

An anti-NOVX antibody (e.g., monoclonal antibody) can be used to isolate an NOVX polypeptide by standard techniques, such as affinity chromatography or immunoprecipitation. An anti-NOVX antibody can facilitate the purification of natural NOVX polypeptide from cells and of recombinantly-produced NOVX polypeptide expressed in host cells. Moreover, an anti-NOVX antibody can be used to detect NOVX protein (e.g., in a cellular lysate or cell supernatant) in order to evaluate the abundance and pattern of expression of the NOVX protein. Anti-NOVX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, P-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol, examples of bioluminescent materials include luciferase, luciferin, and aequorin, and examples of suitable radioactive material include ¹²⁵I, ¹³¹I, ³⁵S or ³H.

NOVX Recombinant Expression Vectors and Host Cells

Another aspect of the invention pertains to vectors, preferably expression vectors, containing a nucleic acid encoding an NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an ill vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated ill vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews—Trends in Genetics, Vol. 1(1)1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences SEQ ID NOS:1, 3, 5, 7, 9, 11, 13, 15, 17, 19, 21, 23, 25, 27, 29, 31 and 33 can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of an NOVX gene into which a deletion, addition or substitution has been introduced to thereby alter, e.g., functionally disrupt, the NOVX gene. The NOVX gene can be a human gene (e.g., the cDNA of SEQ ID NOS:1, 3, 5, 7, 9, 11, 13, 15, 17, 19, 21, 23, 25, 27, 29, 31 and 33), but more preferably, is a non-human homologue of a human NOVX gene. For example, a mouse homologue of human NOVX gene of SEQ ID NOS:1, 3, 5, 7, 9, 11, 13, 15, 17, 19, 21, 23, 25, 27, 29, 31 and 33 can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P 1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermnal (i.e., topical), transmucosal, and rectal administration. Solutions or suspensions used for parenteral, intradermnal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustmnent of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in an NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease (possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of an NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. US.A. 90: 6909; Erb, et al., 1994. Proc. Natl. Acad. Sci. US.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Client. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to an NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with an NOVX target molecule. As used herein, a “target molecule” is a molecule with which an NOVX protein binds or interacts in nature, for example, a molecule on the surface of a cell which expresses an NOVX interacting protein, a molecule on the surface of a second cell, a molecule in the extracellular milieu, a molecule associated with the internal surface of a cell membrane or a cytoplasmic molecule. An NOVX target molecule can be a non-NOVX molecule or an NOVX protein or polypeptide of the invention. In one embodiment, an NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with an NOVX target molecule can be accomplished by one of the methods described above for determining direct binding. In one embodiment, determining the ability of the NOVX protein to bind to or interact with an NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting an NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to an NOVX target molecule by one of the methods described above for determining direct binding. In an alternative embodiment, determining the ability of the test compound to modulate the activity of NOVX protein can be accomplished by determining the ability of the NOVX protein further modulate an NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with an NOVX protein, wherein determining the ability of the test compound to interact with an NOVX protein comprises determining the ability of the NOVX protein to preferentially bind to or modulate the activity of an NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether),, N-dodecyl-N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also likely to be involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences, SEQ ID NOS:1, 3, 5, 7, 9, 11, 13, 15, 17, 19, 21, 23, 25, 27, 29, 31 and 33, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually.

The FISH technique can be used with a DNA sequence as short as 500 or 600 bases.

However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NOS:1, 3, 5, 7, 9, 11, 13, 15, 17, 19, 21, 23, 25, 27, 29, 31 and 33 are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in an NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., dnrgs) for therapeutic or prophylactic treatment of an individual based on the genotype of the individual (e.g., the genotype of the individual examined to determine the ability of the individual to respond to a particular agent.) Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NOS:1, 3, 5, 7, 9, 11, 13, 15, 17, 19, 21, 23, 25, 27, 29, 31 and 33, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, iil vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in an NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an NOVX-protein, or the misexpression of the NOVX gene. For example, such genetic lesions can be detected by ascertaining the existence of at least one of: (i) a deletion of one or more nucleotides from an NOVX gene; (ii) an addition of one or more nucleotides to an NOVX gene; (iii) a substitution of one or more nucleotides of an NOVX gene, (iv) a chromosomal rearrangement of an NOVX gene; (v) an alteration in the level of a messenger RNA transcript of an NOVX gene, (vi) aberrant modification of an NOVX gene, such as of the methylation pattern of the genomic DNA, (vii) the presence of a non-wild-type splicing pattern of a messenger RNA transcript of an NOVX gene, (viii) a non-wild-type level of an NOVX protein, (ix) allelic loss of an NOVX gene, and (x) inappropriate post-translational modification of an NOVX protein. As described herein, there are a large number of assay techniques known in the art which can be used for detecting lesions in an NOVX gene. A preferred biological sample is a peripheral blood leukocyte sample isolated by conventional means from a subject. However, any biological sample containing nucleated cells may be used, including, for example buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucelic acid sample with one or more primers that specifically hybridize to an NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of nucleic acid molecules if such molecules are present in very low numbers.

In an alternative embodiment, mutations in an NOVX gene from a sample cell can be identified by alterations in restriction enzyme cleavage patterns. For example, sample and control DNA is isloated, amplified (optionally), digested with one or more restriction endonucleases, and fragment lenght sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It i also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by Laze on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on an NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g. NOVX gene expression), as identified by a screening assay described herein can be administered to individuals to treat (prophylactically or therapeutically) disorders (The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.) In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C 19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C 19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, and other diseases, disorders and conditions of the like.

These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof, or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, ill situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, an NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of an NOVX protein, a peptide, an NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed ill vitro (e.g., by culturing the cell with the agent) or, alternatively, ill vivo (e.g., by administering the agent to a subject). As such, the invention provides methods of treating an individual afflicted with a disease or disorder characterized by aberrant expression or activity of an NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering an NOVX protein or nucleic acid molecule as therapy to compensate for reduced or aberrant NOVX expression or activity.

Stimulation of NOVX activity is desirable ill situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for ill vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders including, but not limited to: metabolic disorders, diabetes, obesity, infectious disease, anorexia, cancer-associated cancer, neurodegenerative disorders, Alzheimer's Disease, Parkinson's Disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

The invention will be further described in the following examples, which do not limit the scope of the invention described in the claims.

### EXAMPLES

### Example 1

### Identification of NOVX Nucleic Acids

TblastN using CuraGen Corporation's sequence file for polypeptides or homologs was run against the Genomic Daily Files made available by GenBank or from files downloaded from the individual sequencing centers. Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

The novel NOVX target sequences identified in the present invention were subjected to the exon linking process to confirm the sequence. PCR primers were designed by starting at the most upstream sequence available, for the forward primer, and at the most downstream sequence available for the reverse primer. PCR primer sequences were used for obtaining different clones. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

Physical clone: Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

### Example 2

### Identification of Single Nucleotide Polymorphisms in NOVX Nucleic Acid Sequences

Variant sequences are also included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Examples include alteration in temporal expression, physiological response regulation, cell type expression regulation, intensity of expression, and stability of transcribed message.

SeqCalling assemblies produced by the exon linking process were selected and extended using the following criteria. Genomic clones having regions with 98% identity to all or part of the initial or extended sequence were identified by BLASTN searches using the relevant sequence to query human genomic databases. The genomic clones that resulted were selected for further analysis because this identity indicates that these clones contain the genomic locus for these SeqCalling assemblies. These sequences were analyzed for putative coding regions as well as for similarity to the known DNA and protein sequences. Programs used for these analyses include Grail, Genscan, BLAST, HMMER, FASTA, Hybrid and other relevant programs.

Some additional genomic regions may have also been identified because selected SeqCalling assemblies map to those regions. Such SeqCalling sequences may have overlapped with regions defined by homology or exon prediction. They may also be included because the location of the fragment was in the vicinity of genomic regions identified by similarity or exon prediction that had been included in the original predicted sequence. The sequence so identified was manually assembled and then may have been extended using one or more additional sequences taken from CuraGen Corporation's human SeqCalling database. SeqCalling fragments suitable for inclusion were identified by the CuraTools™ program SeqExtend or by identifying SeqCalling fragments mapping to the appropriate regions of the genomic clones analyzed.

The regions defined by the procedures described above were then manually integrated and corrected for apparent inconsistencies that may have arisen, for example, from miscalled bases in the original fragments or from discrepancies between predicted exon junctions, EST locations and regions of sequence similarity, to derive the final sequence disclosed herein. When necessary, the process to identify and analyze SeqCalling assemblies and genomic clones was reiterated to derive the full length sequence.

### Example 3

### Quantitative Expression Analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR). RTQ PCR was performed on a Perkin-Elmer Biosystems ABI PRISM® 7700 Sequence Detection System. Various collections of samples are assembled on the plates, and referred to as Panel I (containing normal tissues and cancer cell lines), Panel 2 (containing samples derived from tissues from normal and cancer sources), Panel 3 (containing cancer cell lines), Panel 4 (containing cells and cell lines from normal tissues and cells related to inflammatory conditions), Panel SD/5I (containing human tissues and cell lines with an emphasis on metabolic diseases), AI_comprehensive_panel (containing normal tissue and samples from autoinflammatory diseases), Panel CNSD.01 (containing samples from normal and diseased brains) and CNS_neurodegeneration₁₃ panel (containing samples from normal and Alzheimer's diseased brains).

RNA integrity from all samples is controlled for quality by visual assessment of agarose gel electropherograms using 28S and 18S ribosomal RNA staining intensity ratio as a guide (2:1 to 2.5:128s: 18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

First, the RNA samples were normalized to reference nucleic acids such as constitutively expressed genes (for example, β-actin and GAPDH). Normalized RNA (5 μl) was converted to cDNA and analyzed by RTQ-PCR using One Step RT-PCR Master Mix Reagents (PE Biosystems; Catalog No. 4309169) and gene-specific primers according to the manufacturer's instructions. Probes and primers were designed for each assay according to Perkin Elmer Biosystem's Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tₘ) range=58°-60° C., primer optimal Tm=59° C., maximum primer difference=2° C., probe does not have 5 G, probe Tₘ must be 10° C. greater than primer Tₘ, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5 and 3′ ends of the probe, respectively. Their final concentrations were: forward and reverse primers, 900 nM each, and probe, 200 nM.

PCR conditions: Normalized RNA from each tissue and each cell line was spotted in each well of a 96 well PCR plate (Perkin Elmer Biosystems). PCR cocktails including two probes (a probe specific for the target clone and another gene-specific probe multiplexed with the target probe) were set up using 1×TaqMan™ PCR Master Mix for the PE Biosystems 7700, with 5 mM MgCl2, dNTPs (dA, G, C, U at 1:1:1:2 ratios), 0.25 U/ml AmpliTaq Gold™ (PE Biosystems), and 0.4 U/μl RNase inhibitor, and 0.25 U/μl reverse transcriptase. Reverse transcription was performed at 48° C. for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

Panels 1, 1.1, 1.2, and 1.3D

The plates for Panels 1, 1.1, 1.2 and 1.3D include 2 control wells (genomic DNA control and chemistry control) and 94 wells containing cDNA from various samples. The samples in these panels are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in these panels are widely available through the American Type Culture Collection (ATCC), a repository for cultured cell lines, and were cultured using the conditions recommended by the ATCC. The normal tissues found on these panels are comprised of samples derived from all major organ systems from single adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

In the results for Panels 1, 1.11, 1.2 and 1.3D, the following abbreviations are used:

ca. =carcinoma,

*=established from metastasis,

met=metastasis,

s cell var=small cell variant,

non-s=non-sm=non-small,

squam=squamous,

pl. eff=pl effusion=pleural effusion,

glio=glioma,

astro=astrocytoma, and

neuro=neuroblastoma.

General_screening_panel_v1.4

The plates for Panel 1.4 include 2 control wells (genomic DNA control and chemistry control) and 94 wells containing cDNA from various samples. The samples in Panel 1.4 are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in Panel 1.4 are widely available through the American Type Culture Collection (ATCC), a repository for cultured cell lines, and were cultured using the conditions recommended by the ATCC. The normal tissues found on Panel 1.4 are comprised of pools of samples derived from all major organ systems from 2 to 5 different adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, utenis, placenta, prostate, testis and adipose.

Panels 2D and 2.2

The plates for Panels 2D and 2.2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologists at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen.

Panel 3D

The plates of Panel 3D are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D and 1.3D are of the most common cell lines used in the scientific literature.

Panels 4D, 4R, and 4.1D

Panel 4 includes samples on a 96 well plate (2 control wells, 94 test samples) composed of RNA (Panel 4R) or cDNA (Panels 4D/4. I D) isolated from various human cell lines or tissues related to inflammatory conditions. Total RNA from control normal tissues such as colon and lung (Stratagene, La Jolla, Calif.) and thymus and kidney (Clontech) were employed. Total RNA from liver tissue from cirrhosis patients and kidney from lupus patients was obtained from BioChain (Biochain Institute, Inc., Hayvard, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵ M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) with PHA (phytohemagglutinin) or PWM (pokeweed mitogen) at approximately 51 g/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 AM non essential amino acids (Gibco), 1 mM sodium pyruvate (Gibco), mercaptoethanol (5.5×10⁻⁵ M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵ M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. Then CD45RO beads were used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3 μg/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2 for 4-6 days before RNA was prepared.

To obtain B cells, tonsils were procured from NDR1. The tonsil was cut up with sterile dissecting scissors and then passed through a sieve. Tonsil cells were then spun down and resupended at 10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24, 48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (4 ng/ml). IL-12 (5 ng/ml) and anti-IL4 (1 μg/ml) were used to direct to Th1, while IL-4 (5 ng/ml) and anti-IFN gamma (1 μg/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (1 ng/ml). Following this, the activated Th 1, Th2 and Tr1 lymphocytes were re-stimulated for 5 days with anti-CD28/OKT3 and cytokines as described above, but with the addition of anti-CD95L (1 pg/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100,M non essential amino acids (Gibco), 1 mM sodium pyruvate (Gibco), mercaptoethanol 5.5×10⁻⁵ M (Gibco), 10 mM Hepes (Gibco). RNA was either prepared from resting cells or cells activated with PMA at 10 ng/ml and ionomycin at 1 pg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). CCDI 106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20 degrees C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37 degrees C. for 30 minutes to remove contaminating genomic DNA, extracted once with phenol chloroform and re-precipitated with 1/10 volume of 3 M sodium acetate and 2 volumes of 100% ethanol. The RNA was spun down and placed in RNAse free water. RNA was stored at −80 degrees C.

AI_comprehensive_panel_v1.0

The plates for AI_comprehensive panel_v1.0 include two control wells and 89 test samples comprised of cDNA isolated from surgical and postmortem human tissues obtained from the Backus Hospital and Clinomics (Frederick, Md.). Total RNA was extracted from tissue samples from the Backus Hospital in the Facility at CuraGen. Total RNA from other tissues was obtained from Clinomics.

Joint tissues including synovial fluid, synovium, bone and cartilage were obtained from patients undergoing total knee or hip replacement surgery at the Backus Hospital. Tissue samples were immediately snap frozen in liquid nitrogen to ensure that isolated RNA was of optimal quality and not degraded. Additional samples of osteoarthritis and rheumatoid arthritis joint tissues were obtained from Clinomics. Normal control tissues were supplied by Clinomics and were obtained during autopsy of trauma victims.

Surgical specimens of psoriatic tissues and adjacent matched tissues were provided as total RNA by Clinomics. Two male and two female patients were selected between the ages of 25 and 47. None of the patients were taking prescription drugs at the time samples were isolated.

Surgical specimens of diseased colon from patients with ulcerative colitis and Crohns disease and adjacent matched tissues were obtained from Clinomics. Bowel tissue from three female and three male Crohn's patients between the ages of 41-69 were used. Two patients were not on prescription medication while the others were taking dexamethasone, phenobarbital, or tylenol. Ulcerative colitis tissue was from three male and four female patients. Four of the patients were taking lebvid and two were on phenobarbital.

Total RNA from post mortem lung tissue from trauma victims was purchased from Clinomics. Emphysema patients ranged in age from 40-70 and all were smokers, this age range was chosen to focus on patients with cigarette-linked emphysema and to avoid those patients with alpha-i anti-trypsin deficiencies. Asthma patients ranged in age from 36-75, and excluded smokers to prevent those patients that could also have COPD. COPD patients ranged in age from 35-80 and included both smokers and non-smokers. Most patients were taking corticosteroids, and bronchodilators.

In the labels employed to identify tissues in the AI_comprehensive panel_v1.0 panel, the following abbreviations are used:

Syn=Synovial

Normal=No apparent disease

Rep22/Rep20=individual patients

RA=Rheumatoid arthritis

Backus=From Backus Hospital

OA=Osteoarthritis

(SS) (BA) (MF)=Individual patients

Adj=Adjacent tissue

Match control =adjacent tissues

-M=Male

-F=Female

COPD=Chronic obstructive pulmonary disease

Panels 5D and 5I

The plates for Panel 5D and 5I include two control wells and a variety of cDNAs isolated from human tissues and cell lines with an emphasis on metabolic diseases. Metabolic tissues were obtained from patients enrolled in the Gestational Diabetes study. Cells were obtained during different stages in the differentiation of adipocytes from human mesenchymal stem cells. Human pancreatic islets were also obtained.

In the Gestational Diabetes study subjects are young (18-40 years), otherwise healthy women with and without gestational diabetes undergoing routine (elective) Caesarean section. After delivery of the infant, when the surgical incisions were being repaired/closed, the obstetrician removed a small sample (<1 cc) of the exposed metabolic tissues during the closure of each surgical level. The biopsy material was rinsed in sterile saline, blotted and fast frozen within 5 minutes from the time of removal. The tissue was then flash frozen in liquid nitrogen and stored, individually, in sterile screw-top tubes and kept on dry ice for shipment to or to be picked up by CuraGen. The metabolic tissues of interest include uterine wall (smooth muscle), visceral adipose, skeletal muscle (rectus) and subcutaneous adipose. Patient descriptions are as follows:

| | |
|---|---|
| Patient 2 | Diabetic Hispanic, overweight, not on insulin |
| Patient 7-9 | Nondiabetic Caucasian and obese (BMI > 30) |
| Patient 10 | Diabetic Hispanic, overweight, on insulin |
| Patient 11 | Nondiabetic African American and overweight |
| Patient 12 | Diabetic Hispanic on insulin |

Adiocyte differentiation was induced in donor progenitor cells obtained from Osirus (a division of Clonetics/BioWhittaker) in triplicate except for Donor 3U which had only two replicates. Scientists at Clonetics isolated, grew and differentiated human mesenchymal stem cells (HuMSCs) for CuraGen based on the published protocol found in Mark F. Pittenger, et al., Multilineage Potential of Adult Human Mesenchymal Stem Cells Science Apr. 2, 1999: 143-147. Clonetics provided Trizol lysates or frozen pellets suitable for mRNA isolation and ds cDNA production. A general description of each donor is as follows:

| | | |
|---|---|---|
| Donor 2 and 3 U | Mesenchymal | Undifferentiated Adipose |
| | Stem cells | |
| Donor 2 and 3 AM | Adipose | AdiposeMidway Differentiated |
| Donor 2 and 3 AD | Adipose | Adipose Differentiated |

Human cell lines were generally obtained from ATCC (American Type Culture Collection), NCI or the German tumor cell bank and fall into the following tissue groups: kidney proximal convoluted tubule, uterine smooth muscle cells, small intestine, liver HepG2 cancer cells, heart primary stromal cells, and adrenal cortical adenoma cells. These cells are all cultured under standard recommended conditions and RNA extracted using the standard procedures. All samples were processed at CuraGen to produce single stranded cDNA.

Panel 5I contains all samples previously described with the addition of pancreatic islets from a 58 year old female patient obtained from the Diabetes Research Institute at the University of Miami School of Medicine. Islet tissue was processed to total RNA at an outside source and delivered to CuraGen for addition to panel 5I.

In the labels employed to identify tissues in the 5D and 5I panels, the following abbreviations are used:

GO Adipose=Greater Omentum Adipose

SK=Skeletal Muscle

UT=Uterus

PL=Placenta

AD=Adipose Differentiated

AM=Adipose Midway Differentiated

U=Undifferentiated Stem Cells

Panel CNSD.01

The plates for Panel CNSD.01 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease, Parkinson's disease, Huntington's disease, Progressive Supernuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex), Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

In the labels employed to identify tissues in the CNS panel, the following abbreviations are used:

PSP=Progressive supranuclear palsy

Sub Nigra=Substantia nigra

Glob Palladus=Globus palladus

Temp Pole=Temporal pole

Cing Gyr=Cingulate gyrus

BA 4=Brodman Area 4

Panel CNS_Neurodegeneration_V1.0

The plates for Panel CNS_Neurodegeneration_V1.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains six brains from Alzheimer's disease (AD) pateins, and eight brains from “Normal controls” who showed no evidence of dementia prior to death. The eight normal control brains are divided into two categories: Controls with no dementia and no Alzheimer's like pathology (Controls) and controls with no dementia but evidence of severe Alzheimer's like pathology, (specifically senile plaque load rated as level 3 on a scale of 0-3; 0=no evidence of plaques, 3=severe AD senile plaque load). Within each of these brains, the following regions are represented: hippocampus, temporal cortex (Broddmann Area 21), parietal cortex (Broddmann area 7), and occipital cortex (Brodmann area 17). These regions were chosen to encompass all levels of neurodegeneration in AD. The hippocampus is a region of early and severe neuronal loss in AD; the temporal cortex is known to show neurodegeneration in AD after the hippocampus; the parietal cortex shows moderate neuronal death in the late stages of the disease; the occipital cortex is spared in AD and therefore acts as a “control” region within AD patients. Not all brain regions are represented in all cases.

In the labels employed to identify tissues in the CNS_Neurodegeneration-V 1.0 panel, the following abbreviations are used:

AD=Alzheimer's disease brain; patient was demented and showed AD-like pathology upon autopsy

Control=Control brains; patient not demented, showing no neuropathology

Control (Path)=Control brains; pateint not demented but showing sever AD-like pathology

SupTemporal Ctx=Superior Temporal Cortex

Inf Temporal Ctx=Inferior Temporal Cortex

NOV1: Calpain-Like

Expression of the NOV1 gene (also referred to as 3352274) was assessed using the primer-probe set Ag2003 described in Table 12 Results from RTQ-PCR runs are shown in Tables 13 and 14.

TABLE 12
Probe Name Ag2003

| | | | | Start | SEQ ID |
|---|---|---|---|---|---|
| Primers | Sequences | TM | Length | Position | NO: |
| Forward | 5′-CAGCCTAATGCTGAAACCTTCT-3′ | 59.9 | 22 | 1117 | 102 |
| Probe | TET-5′-ATCCTCAGTTCCGTTTAACGCTCCTG-3′-TAMRA | 69.2 | 26 | 1145 | 103 |
| Reverse | 5′-ATCCTCGTCATCCTCCTCAT-3′ | 58.5 | 20 | 1178 | 104 |

TABLE 13
Panel 1.3D

| | Relative | | Relative |
|---|---|---|---|
| | Expression(%) | | Expression(%) |
| | 1.3dx4tm5423t_{—} | | 1.3dx4tm5423t_{—} |
| Tissue Name | ag2003_b2 | Tissue Name | ag2003_b2 |
| Liver adenocarcinoma | 0.0 | Kidney (fetal) | 0.0 |
| Pancreas | 0.0 | Renal ca. 786-0 | 0.0 |
| Pancreatic ca. CAPAN 2 | 0.0 | Renal ca A498 | 0.0 |
| Adrenal gland | 0.0 | Renal ca. RXF 393 | 0.0 |
| Thyroid | 0.7 | Renal ca. ACHN | 0.0 |
| Salivary gland | 0.0 | Renal ca. UO-31 | 0.0 |
| Pituitary gland | 0.0 | Renal ca TK-10 | 9.2 |
| Brain (fetal) | 0.0 | Liver | 100.0 |
| Brain (whole) | 2.0 | Liver (fetal) | 34.5 |
| Brain (amygdala) | 0.0 | Liver ca. (hepatoblast) HepG2 | 33.7 |
| Brain (cerebellum) | 0.0 | Lung | 17.5 |
| Brain (hippocampus) | 0.0 | Lung (fetal) | 0.0 |
| Brain (substantia nigra) | 3.0 | Lung ca. (small cell) LX-1 | 6.3 |
| Brain (thalamus) | 0.0 | Lung ca. (small cell) NCI-H69 | 0.0 |
| Cerebral Cortex | 0.0 | Lung ca. (s.cell var.) SHP-77 | 0.0 |
| Spinal cord | 0.0 | Lung ca. (large cell) NCI-H460 | 0.0 |
| CNS ca. (glio/astro) U87-MG | 0.0 | Lung ca. (non-sm.cell) A549 | 0.0 |
| CNS ca. (glio/astro) U-118-MG | 0.0 | Lung ca. (non-s.cell) NCI-H23 | 0.0 |
| CNS ca. (astro) SW1783 | 0.0 | Lung ca (non-s.cell) HOP-62 | 0.0 |
| CNS ca.* (neuro; met ) SK-N-AS | 0.0 | Lung ca. (non-s.cl) NCI-H522 | 0.0 |
| CNS ca. (astro) SF-539 | 0.0 | Lung ca. (squam.) SW 900 | 0.0 |
| CNS ca. (astro) SNB-75 | 0.0 | Lung ca. (squam.) NCI-H596 | 0.0 |
| CNS ca. (glio) SNB-19 | 0.0 | Mammary gland | 0.0 |
| CNS ca. (glio) U251 | 0.0 | Breast ca.* (pl. effusion) MCF-7 | 7.5 |
| CNS ca. (glio) SF-295 | 0.0 | Breast ca.* (pl. ef) MDA-MB-231 | 0.0 |
| Heart (fetal) | 0.0 | Breast ca.* (pl. effusion) T47D | 0.0 |
| Heart | 6.3 | Breast ca. BT-549 | 0.0 |
| Fetal Skeletal | 0.0 | Breast ca. MDA-N | 0.0 |
| Skeletal muscle | 0.0 | Ovary | 0.0 |
| Bone marrow | 4.4 | Ovarian ca. OVCAR-3 | 0.6 |
| Thymus | 0.0 | Ovarian ca. OVCAR-4 | 0.0 |
| Spleen | 9.7 | Ovarian ca. OVCAR-5 | 0.0 |
| Lymph node | 0.0 | Ovarian ca. OVCAR-8 | 0.0 |
| Colorectal | 0.0 | Ovarian ca. IGROV-1 | 0.0 |
| Stomach | 0.0 | Ovarian ca.* (ascites) SK-OV-3 | 0.0 |
| Small intestine | 0.0 | Uterus | 5.1 |
| Colon ca. SW480 | 0.0 | Placenta | 7.1 |
| Colon ca.* (SW480 met)SW620 | 0.0 | Prostate | 0.0 |
| Colon ca. HT29 | 2.9 | Prostate ca.* (bone met)PC-3 | 0.0 |
| Colon ca. HCT-116 | 0.0 | Testis | 11.8 |
| Colon ca. CaCo-2 | 0.0 | Melanoma Hs688(A).T | 0.0 |
| 83219 CC Well to Mod Diff | 22.9 | Melanoma* (met) Hs688(B).T | 0.0 |
| (ODO3866) | | | |
| Colon ca. HCC-2998 | 1.4 | Melanoma UACC-62 | 0.0 |
| Gastric ca.* (liver met) NCI-N87 | 0.0 | Melanoma M14 | 0.0 |
| Bladder | 0.0 | Melanoma LOX IMVI | 0.0 |
| Trachea | 5.5 | Melanoma* (met) SK-MEL-5 | 0.0 |
| Kidney | 0.0 | Adipose | 0.6 |

TABLE 14
Panel 4D

| | Relative | | Relative |
|---|---|---|---|
| | Expression(%) | | Expression(%) |
| | 4dx4tm5530t_{—} | | 4dx4tm5530t_{—} |
| Tissue Name | ag2003_b2 | Tissue Name | ag2003_b2 |
| 93768_Secondary Th1_anti- | 7.1 | 93100_HUVEC (Endothelial)_IL- | 0.0 |
| CD28/anti-CD3 | | 1b | |
| 93769_Secondary Th2_anti- | 0.0 | 93779_HUVEC (Endothelial)_IFN | 0.0 |
| CD28/anti-CD3 | | gamma | |
| 93770_Secondary Tr1_anti- | 0.0 | 93102_HUVEC | 0.0 |
| CD28/anti-CD3 | | (Endothelial)_TNF alpha + IFN | |
| | | gamma | |
| 93573_Secondary Th1_resting day | 0.0 | 93101 _HUVEC | 0.0 |
| 4-6 in IL-2 | | (Endothelial)_TNF alpha + IL4 | |
| 93572_Secondary Th2_resting day | 9.1 | 93781 _HUVEC (Endothelial)_IL- | 0.0 |
| 4-6 in IL-2 | | 11 | |
| 93571_Secondary Tr1_resting day | 0.0 | 93583_Lung Microvascular | 0.0 |
| 4-6 in IL-2 | | Endothelial Cells_none | |
| 93568_primary Th1_anti- | 0.0 | 93584_Lung Microvascular | 0.0 |
| CD28/anti-CD3 | | Endothelial Cells_TNFa (4 ng/ml) | |
| | | and IL1b (1 ng/ml) | |
| 93569_primary Th2_anti- | 0.0 | 92662_Microvascular Dermal | 0.0 |
| CD28/anti-CD3 | | endothelium_none | |
| 93570_primary Tr1_anti- | 0.0 | 92663_Microsvasular Dermal | 0.0 |
| CD28/anti-CD3 | | endothelium_TNFa (4 ng/ml) and | |
| | | IL1b (1 ng/ml) | |
| 93565_primary Th1_resting dy 4-6 | 17.0 | 93773_Bronchial | 98.9 |
| in IL-2 | | epithelium_TNFa (4 ng/ml) and | |
| | | IL1b (1 ng/ml)** | |
| 93566_primary Th2_resting dy 4-6 | 0.0 | 93347_Small Airway | 0.0 |
| in IL-2 | | Epithelium_none | |
| 93567_primary Tr1_resting dy 4-6 | 0.0 | 93348_Small Airway | 24.9 |
| in IL-2 | | Epithelium_TNFa (4 ng/ml) and | |
| | | IL1b (1 ng/ml) | |
| 93351_CD45RA CD4 | 1.7 | 92668_Coronery Artery | 0.0 |
| lymphocyte_anti-CD28/anti-CD3 | | SMC_resting | |
| 93352_CD45RO CD4 | 0.0 | 92669_Coronery Artery | 0.0 |
| lymphocyte_anti-CD28/anti-CD3 | | SMC_TNFa (4 ng/ml) and IL1b (1 ng/ml) | |
| 93251_CD8 Lymphocytes_anti- | 0.0 | 93107_astrocytes_resting | 0.0 |
| CD28/anti-CD3 | | | |
| 93353_chronic CD8 Lymphocytes | 0.0 | 93108_astrocytes_TNFa (4 ng/ml) | 6.5 |
| 2ry_resting dy 4-6 in IL-2 | | and IL1b (1 ng/ml) | |
| 93574_chronic CD8 Lymphocytes | 0.0 | 92666_KU-812 (Basophil)_resting | 0.0 |
| 2ry_activated CD3/CD28 | | | |
| 93354_CD4_none | 0.0 | 92667_KU-812 | 0.0 |
| | | (Basophil)_PMA/ionoycin | |
| 93252_Secondary | 4.0 | 93579_CCD1106 | 5.9 |
| Th1/Th2/Tr1_anti-CD95 CH11 | | (Keratinocytes)_none | |
| 93103_LAK cells_resting | 0.0 | 93580_CCD1106 | 28.9 |
| | | (Keratinocytes)_TNFa and IFNg** | |
| 93788_LAK cells_IL-2 | 6.5 | 93791 _Liver Cirrhosis | 100.0 |
| 93787_LAK cells_IL-2 + IL-12 | 0.0 | 93792_Lupus Kidney | 30.2 |
| 93789_LAK cells_IL-2 + IFN | 0.0 | 93577_NCI-H292 | 0.0 |
| gamma | | | |
| 93790_LAK cells_IL-2 + IL-18 | 0.0 | 93358_NCI-H292_IL-4 | 0.0 |
| 93104_LAK | 0.0 | 93360_NCI-H292_IL-9 | 1.1 |
| cells_PMA/ionomycin and IL-18 | | | |
| 93578_NK Cells IL-2_resting | 0.0 | 93359_NCI-H292_IL-13 | 0.0 |
| 93109_Mixed Lymphocyte | 0.0 | 93357_NCI-H292_IFN gamma | 0.0 |
| Reaction_Two Way MLR | | | |
| 93110_Mixed Lymphocyte | 0.0 | 93777_HPAEC_- | 0.0 |
| Reaction_Two Way MLR | | | |
| 93111_Mixed Lymphocyte | 0.0 | 93778_HPAEC_IL-1 beta/TNA | 0.0 |
| Reaction_Two Way MLR | | alpha | |
| 93112_Mononuclear Cells | 0.0 | 93254_Normal Human Lung | 0.0 |
| (PBMCs)_resting | | Fibroblast_none | |
| 93113_Mononuclear Cells | 0.0 | 93253_Normal Human Lung | 0.0 |
| (PBMCs)_PWM | | Fibroblast_TNFa (4 ng/ml) and IL- | |
| | | 1b (1 ng/ml) | |
| 93114_Mononuclear Cells | 0.0 | 93257_Normal Human Lung | 0.0 |
| (PBMCs)_PHA-L | | Fibroblast_IL-4 | |
| 93249_Ramos (B cell)_none | 0.0 | 93256_Normal Human Lung | 0.0 |
| | | Fibroblast_IL-9 | |
| 93250_Ramos (B cell)_ionomycin | 0.0 | 93255_Normal Human Lung | 0.0 |
| | | Fibroblast IL-13 | |
| 93349_B lymphocytes_PWM | 5.6 | 93258_Normal Human Lung | 0.0 |
| | | Fibroblast_IFN gamma | |
| 93350_B lymphocytes_CD40L and | 1.0 | 93106_Dermal Fibroblasts | 0.0 |
| IL-4 | | CCD1070_resting | |
| 92665_EOL-1 | 0.0 | 93361 _Dermal Fibroblasts | 0.0 |
| (Eosinophil)_dbcAMP | | CCD1070_TNF alpha 4 ng/ml | |
| differentiated | | | |
| 93248_EOL-1 | 3.1 | 93105_Dermal Fibroblasts | 0.0 |
| (Eosinophil)_dbcAMP/PMAionomycin | | CCD1070_IL-1 beta 1 ng/ml | |
| 93356_Dendritic Cells_none | 0.0 | 93772_dermal fibroblast_IFN | 0.0 |
| | | gamma | |
| 93355_Dendritic Cells_LPS 100 ng/ml | 0.0 | 93771_dermal fibroblast_IL-4 | 0.0 |
| 93775_Dendritic Cells_anti-CD40 | 0.0 | 93260_IBD Colitis 2 | 0.0 |
| 93774_Monocytes_resting | 6.3 | 93261 _IBD Crohns | 0.0 |
| 93776_Monocytes_LPS 50 ng/ml | 0.0 | 735010_Colon_normal | 14.7 |
| 93581_Macrophages_resting | 0.0 | 735019_Lung_none | 0.0 |
| 93582_Macrophages_LPS 100 ng/ml | 0.0 | 64028-1_Thymus_none | 36.1 |
| 93098_HUVEC | 0.0 | 64030-1_Kidney_none | 0.0 |
| (Endothelial)_none | | | |
| 93099_HUVEC | 0.0 | | |
| (Endothelial)_starved | | | |

Panel 1.3D Summary Expression of the NOV1 gene appears to be specific to the liver, with the highest expression in normal liver tissue (CT=32.1), and significant expression detected in fetal liver and a liver cancer cell line as well. Since the expression of the NOV1 gene appears to be associated with the liver, it could potentially be used to differentiate between tissues derived from the liver and other tissues. Furthermore, therapeutic modulation of the NOV1 gene may be beneficial in the treatment of liver related disorders, Such as liver cirrhosis.

Panel 4D Summary Expression of the NOV1 gene is in this panel is restricted to a few samples, with highest expression detected in liver cirrhosis (CT=33.2). This result is in concordance with the liver specific expression seen in Panel 1.3D. Expression of the gene is also detected at low but significant levels in the thymus and TNF-alpha and IL-1 beta treated bronchial epithelium. The protein encoded by the NOV1 gene has homology to calcium-activated neutral proteases (calpain). Calpains have been identified in the trachea and in the lung, and may be involved in tissue destruction. Therapeutic drugs designed with the protein encoded for by the NOV1 gene may be important for the treatment of asthma, emphysema, and liver cirrhosis (Dear et al., A new subfamily of vertebrate calpains lacking a calmodulin-like domain: implications for calpain regulation and evolution. Genomics. 45:175-84, 1997).

NOV2: Epsin-Like

Expression of the NOV2 gene (also referred to as 21421174) was assessed using the primer-probe set Ag3088 described in Table BA Results from RTQ-PCR runs are shown in Tables 15, 16, 17, 18 and 19.

TABLE 15
Probe Name Ag3088

| | | | | Start | |
|---|---|---|---|---|---|
| Primers | Sequences | TM | Length | Position | SEQ ID NO: |
| Forward | 5′-CACGTTTACAAGGCCATGAC-3′ | 59 | 20 | 256 | 105 |
| Probe | FAM-5′-ATGGAGTACCTCATCAAGACCGGCTC-3′-TAMRA | 68.6 | 26 | 280 | 106 |
| Reverse | 5′-ATGTTCTCCTTGCACTGCTG-3′ | 59 | 20 | 319 | 107 |

TABLE 16
Panel 1.3D

| | Relative | | Relative |
|---|---|---|---|
| | Expression(%) | | Expression(%) |
| | 1.3dx4tm5430f_{—} | | 1.3dx4tm5430f_{—} |
| Tissue Name | ag3088_b1 | Tissue Name | ag3088_b1 |
| Liver adenocarcinoma | 37.2 | Kidney (fetal) | 11.7 |
| Pancreas | 19.0 | Renal ca. 786-0 | 11.6 |
| Pancreatic ca. CAPAN 2 | 31.2 | Renal ca. A498 | 64.4 |
| Adrenal gland | 15.5 | Renal ca. RXF 393 | 44.2 |
| Thyroid | 15.5 | Renal ca. ACHN | 25.7 |
| Salivary gland | 11.7 | Renal ca. UO-31 | 36.5 |
| Pituitary gland | 10.3 | Renal ca. TK-10 | 9.9 |
| Brain (fetal) | 46.5 | Liver | 12.1 |
| Brain (whole) | 70.5 | Liver (fetal) | 21.9 |
| Brain (amygdala) | 64.8 | Liver ca. (hepatoblast) HepG2 | 52.1 |
| Brain (cerebellum) | 53.4 | Lung | 17.2 |
| Brain (hippocampus) | 77.3 | Lung (fetal) | 18.6 |
| Brain (substantia nigra) | 29.2 | Lung ca. (small cell) LX-1 | 12.4 |
| Brain (thalamus) | 55.5 | Lung ca. (small cell) NCI-H69 | 19.1 |
| Cerebral Cortex | 84.6 | Lung ca. (s.cell var.) SHP-77 | 24.9 |
| Spinal cord | 19.2 | Lung ca. (large cell)NCI-H460 | 18.1 |
| CNS ca. (glio/astro) U87-MG | 43.5 | Lung ca. (non-sm. cell) A549 | 24.0 |
| CNS ca. (glio/astro) U-118-MG | 100.0 | Lung ca. (non-s.cell) NCI-H23 | 6.0 |
| CNS ca. (astro) SW1783 | 38.8 | Lung ca. (non-s.cell) HOP-62 | 11.6 |
| CNS ca.* (neuro; met) SK-N-AS | 65.9 | Lung ca. (non-s.cl) NCI-H522 | 6.2 |
| CNS ca. (astro) SF-539 | 21.7 | Lung ca. (squam.) SW 900 | 18.0 |
| CNS ca. (astro) SNB-75 | 64.9 | Lung ca. (squam.) NCI-H596 | 32.0 |
| CNS ca. (glio) SNB-19 | 40.3 | Mammary gland | 16.8 |
| CNS ca. (glio) U251 | 40.6 | Breast ca.* (pl. effusion) MCF-7 | 19.7 |
| CNS ca. (glio) SF-295 | 32.1 | Breast ca.* (pl.ef) MDA-MB-231 | 80.4 |
| Heart (fetal) | 36.8 | Breast ca.* (pl. effusion) T47D | 11.9 |
| Heart | 22.0 | Breast ca. BT-549 | 44.8 |
| Fetal Skeletal | 14.4 | Breast ca. MDA-N | 12.6 |
| Skeletal muscle | 84.5 | Ovary | 22.2 |
| Bone marrow | 12.1 | Ovarian ca. OVCAR-3 | 19.1 |
| Thymus | 6.7 | Ovarian ca. OVCAR-4 | 85.5 |
| Spleen | 23.1 | Ovarian ca. OVCAR-5 | 21.0 |
| Lymph node | 18.7 | Ovarian ca. OVCAR-8 | 9.6 |
| Colorectal | 7.7 | Ovarian ca. IGROV-1 | 5.7 |
| Stomach | 58.5 | Ovarian ca.* (ascites) SK-OV-3 | 41.0 |
| Small intestine | 44.4 | Uterus | 19.9 |
| Colon ca. SW480 | 19.0 | Placenta | 9.8 |
| Colon ca.* (SW480 met)SW620 | 13.5 | Prostate | 16.7 |
| Colon ca. HT29 | 12.1 | Prostate ca.* (bone met)PC-3 | 87.5 |
| Colon ca. HCT-116 | 19.1 | Testis | 23.8 |
| Colon ca. CaCo-2 | 21.9 | Melanoma Hs688(A).T | 15.0 |
| 83219 CC Well to Mod Diff | 16.3 | Melanoma* (met) Hs688(B).T | 12.5 |
| (ODO3866) | | | |
| Colon ca. HCC-2998 | 9.6 | Melanoma UACC-62 | 31.6 |
| Gastric ca.* (liver met) NCI-N87 | 41.3 | Melanoma M14 | 36.6 |
| Bladder | 22.4 | Melanoma LOX IMVI | 24.1 |
| Trachea | 21.9 | Melanoma* (met) SK-MEL-5 | 15.5 |
| Kidney | 24.0 | Adipose | 8.5 |

TABLE 17
Panel 2.2

| | Relative | | Relative |
|---|---|---|---|
| | Expression(%) | | Expression(%) |
| | 2.2x4tm6408f_{—} | | 2.2x4tm6408f_{—} |
| Tissue Name | ag3088_b1 | Tissue Name | ag3088_b1 |
| Normal Colon GENPAK 061003 | 26.7 | 83793 Kidney NAT (OD04348) | 94.6 |
| 97759 Colon cancer (OD06064) | 14.9 | 98938 Kidney malignant cancer | 12.2 |
| | | (OD06204B) | |
| 97760 Colon cancer NAT | 14.7 | 98939 Kidney normal adjacent | 29.1 |
| (OD06064) | | tissue (OD06204E) | |
| 97778 Colon cancer (OD06159) | 16.3 | 85973 Kidney Cancer (OD04450- | 43.1 |
| | | 01) | |
| 97779 Colon cancer NAT | 25.3 | 85974 Kidney NAT (OD04450-03) | 40.6 |
| (OD06159) | | | |
| 98861 Colon cancer (OD06297-04) | 11.5 | Kidney Cancer Clontech 8120613 | 5.7 |
| 98862 Colon cancer NAT | 15.8 | Kidney NAT Clontech 8120614 | 52.1 |
| (OD06297-015) | | | |
| 83237 CC Gr.2 ascend colon | 9.0 | Kidney Cancer Clontech 9010320 | 15.5 |
| (ODO3921) | | | |
| 83238 CC NAT (ODO3921) | 10.8 | Kidney NAT Clontech 9010321 | 22.4 |
| 97766 Colon cancer metastasis | 5.8 | Kidney Cancer Clontech 8120607 | 83.0 |
| (OD06104) | | | |
| 97767 Lung NAT (OD06104) | 17.5 | Kidney NAT Clontech 8120608 | 35.5 |
| 87472 Colon mets to lung | 23.2 | Normal Uterus GENPAK 061018 | 13.0 |
| (OD04451-01) | | | |
| 87473 Lung NAT (OD04451-02) | 19.2 | Uterus Cancer GENPAK 064011 | 12.3 |
| Normal Prostate Clontech A+ | 22.4 | Normal Thyroid Clontech A+ | 7.5 |
| 6546-1 (8090438) | | 6570-1 (7080817) | |
| 84140 Prostate Cancer (OD04410) | 7.8 | Thyroid Cancer GENPAK 064010 | 12.9 |
| 84141 Prostate NAT (OD04410) | 7.5 | Thyroid Cancer INVITROGEN | 28.0 |
| | | A302152 | |
| Normal Ovary Res. Gen. | 48.3 | Thyroid NAT INVITROGEN | 7.1 |
| | | A302153 | |
| 98863 Ovarian cancer (OD06283- | 10.4 | Normal Breast GENPAK 061019 | 14.9 |
| 03) | | | |
| 98865 Ovarian cancer | 7.6 | 84877 Breast Cancer (OD04566) | 14.5 |
| NAT/fallopian tube (OD06283-07) | | | |
| Ovarian Cancer GENPAK 064008 | 11.9 | Breast Cancer Res. Gen. 1024 | 30.7 |
| 97773 Ovarian cancer (OD06145) | 11.7 | 85975 Breast Cancer (OD04590- | 60.4 |
| | | 01) | |
| 97775 Ovarian cancer NAT | 19.8 | 85976 Breast Cancer Mets | 25.3 |
| (OD06145) | | (OD04590-03) | |
| 98853 Ovarian cancer (OD06455- | 14.2 | 87070 Breast Cancer Metastasis | 55.0 |
| 03) | | (OD04655-05) | |
| 98854 Ovarian NAT (OD06455- | 1.9 | GENPAK Breast Cancer 064006 | 20.4 |
| 07) Fallopian tube | | | |
| Normal Lung GENPAK 061010 | 13.2 | Breast Cancer Clontech 9100266 | 16.3 |
| 92337 Invasive poor diff. lung | 22.1 | Breast NAT Clontech 9100265 | 7.2 |
| adeno (ODO4945-01) | | | |
| 92338 Lung NAT (ODO4945-03) | 13.5 | Breast Cancer INVITROGEN | 7.7 |
| | | A209073 | |
| 84136 Lung Malignant Cancer | 12.2 | Breast NAT INVITROGEN | 17.9 |
| (OD03126) | | A2090734 | |
| 84137 Lung NAT (OD03126) | 5.6 | 97763 Breast cancer (OD06083) | 29.5 |
| 90372 Lung Cancer (OD05014A) | 15.3 | 97764 Breast cancer node | 30.2 |
| | | metastasis (OD06083) | |
| 90373 Lung NAT (OD05014B) | 19.8 | Normal Liver GENPAK 061009 | 50.3 |
| 97761 Lung cancer (OD06081) | 21.2 | Liver Cancer Research Genetics | 27.7 |
| | | RNA 1026 | |
| 97762 Lung cancer NAT | 12.8 | Liver Cancer Research Genetics | 80.2 |
| (OD06081) | | RNA 1025 | |
| 85950 Lung Cancer (OD04237-01) | 5.5 | Paired Liver Cancer Tissue | 51.3 |
| | | Research Genetics RNA 6004-T | |
| 85970 Lung NAT (OD04237-02) | 23.6 | Paired Liver Tissue Research | 7.0 |
| | | Genetics RNA 6004-N | |
| 83255 Ocular Mel Met to Liver | 16.4 | Paired Liver Cancer Tissue | 54.3 |
| (ODO4310) | | Research Genetics RNA 6005-T | |
| 83256 Liver NAT (ODO4310) | 19.3 | Paired Liver Tissue Research | 100.0 |
| | | Genetics RNA 6005-N | |
| 84139 Melanoma Mets to Lung | 21.4 | Liver Cancer GENPAK 064003 | 62.4 |
| (OD04321) | | | |
| 84138 Lung NAT (OD04321) | 7.0 | Normal Bladder GENPAK 061001 | 19.8 |
| Normal Kidney GENPAK 061008 | 12.8 | Bladder Cancer Research Genetics | 10.0 |
| | | RNA 1023 | |
| 83786 Kidney Ca, Nuclear grade 2 | 59.3 | Bladder Cancer INVITROGEN | 24.4 |
| (OD04338) | | A302173 | |
| 83787 Kidney NAT (OD04338) | 18.1 | Normal Stomach GENPAK | 98.0 |
| | | 061017 | |
| 83788 Kidney Ca Nuclear grade | 55.8 | Gastric Cancer Clontech 9060397 | 13.5 |
| 1/2 (OD04339) | | | |
| 83789 Kidney NAT (OD04339) | 26.5 | NAT Stomach Clontech 9060396 | 41.4 |
| 83790 Kidney Ca, Clear cell type | 13.5 | Gastric Cancer Clontech 9060395 | 26.0 |
| (OD04340) | | | |
| 83791 Kidney NAT (OD04340) | 29.0 | NAT Stomach Clontech 9060394 | 37.4 |
| 83792 Kidney Ca, Nuclear grade 3 | 12.1 | Gastric Cancer GENPAK 064005 | 30.9 |
| (OD04348) | | | |

TABLE 18
Panel 4D

| | Relative | | Relative |
|---|---|---|---|
| | Expression (%) | | Expression (%) |
| | 4dx4tm5510f_{—} | | 4dx4tm5510f_{—} |
| Tissue Name | ag3088_b2 | Tissue Name | ag3088_b2 |
| 93768_Secondary Th1_anti- | 8.7 | 93100_HUVEC (Endothelial)_IL- | 6.4 |
| CD28/anti-CD3 | | 1b | |
| 93769_Secondary Th2_anti- | 7.7 | 93779_HUVEC (Endothelial)_IFN | 17.7 |
| CD28/anti-CD3 | | gamma | |
| 93770_Secondary Tr1_anti- | 9.0 | 93102_HUVEC | 12.1 |
| CD28/anti-CD3 | | (Endothelial)_TNF alpha + IFN | |
| | | gamma | |
| 93573_Secondary Th1_resting day | 5.8 | 93101_HUVEC | 16.3 |
| 4-6 in IL-2 | | (Endothelial)_TNF alpha + IL4 | |
| 93572_Secondary Th2_resting day | 4.1 | 93781_HUVEC (Endothelial)_IL- | 14.0 |
| 4-6 in IL-2 | | 11 | |
| 93571_Secondary Tr1_resting day | 4.3 | 93583_Lung Microvascular | 18.4 |
| 4-6 in IL-2 | | Endothelial Cells_none | |
| 93568_primary Th1_anti- | 4.3 | 93584_Lung Microvascular | 12.4 |
| CD28/anti-CD3 | | Endothelial Cells_TNFa (4 ng/ml) | |
| | | and IL1b (1 ng/ml) | |
| 93569_primary Th2_anti- | 8.4 | 92662_Microvascular Dermal | 23.4 |
| CD28/anti-CD3 | | endothelium_none | |
| 93570_primary Tr1_anti- | 11.7 | 92663_Microsvasular Dermal | 13.0 |
| CD28/anti-CD3 | | endothelium_TNFa (4 ng/ml) and | |
| | | IL1b (1 ng/ml) | |
| 93565_primary Th1_resting dy 4-6 | 21.0 | 93773_Bronchial | 11.9 |
| in IL-2 | | epithelium_TNFa (4 ng/ml) and | |
| | | IL1b (1 ng/ml)** | |
| 93566_primary Th2_resting dy 4-6 | 10.3 | 93347_Small Airway | 14.4 |
| in IL-2 | | Epithelium_none | |
| 93567_primary Tr1_resting dy 4-6 | 7.3 | 93348_Small Airway | 44.3 |
| in IL-2 | | Epithelium_TNFa (4 ng/ml) and | |
| | | IL1b (1 ng/ml) | |
| 93351_CD45RA CD4 | 12.7 | 92668_Coronery Artery | 24.4 |
| lymphocyte_anti-CD28/anti-CD3 | | SMC_resting | |
| 93352_CD45RO CD4 | 10.3 | 92669_Coronery Artery | 12.3 |
| lymphocyte_anti-CD28/anti-CD3 | | SMC_TNFa (4 ng/ml) and IL1b (1 ng/ml) | |
| 93251_CD8 Lymphocytes_anti- | 7.2 | 93107_astrocytes_resting | 19.9 |
| CD28/anti-CD3 | | | |
| 93353_chronic CD8 Lymphocytes | 9.4 | 93108_astrocytes_TNFa (4 ng/ml) | 36.7 |
| 2ry_resting dy 4-6 in IL-2 | | and IL1b (1 ng/ml) | |
| 93574_chronic CD8 Lymphocytes | 8.5 | 92666_KU-812 (Basophil)_resting | 15.8 |
| 2ry_activated CD3/CD28 | | | |
| 93354_CD4_none | 3.1 | 92667_KU-812 | 27.8 |
| | | (Basophil)_PMA/ionoycin | |
| 93252_Secondary | 8.1 | 93579_CCD1106 | 14.8 |
| Th1/Th2/Tr1_anti-CD95 CH11 | | (Keratinocytes)_none | |
| 93103_LAK cells_resting | 4.9 | 93580_CCD1106 | 49.4 |
| | | (Keratinocytes)_TNFa and IFNg** | |
| 93788_LAK cells_IL-2 | 11.3 | 93791_Liver Cirrhosis | 11.6 |
| 93787_LAK cells_IL-2 + IL-12 | 14.4 | 93792_Lupus Kidney | 11.6 |
| 93789_LAK cells_IL-2 + IFN | 12.8 | 93577_NCI-H292 | 33.5 |
| gamma | | | |
| 93790_LAK cells_IL-2+ IL-18 | 9.0 | 93358_NCI-H292_IL-4 | 68.3 |
| 93104_LAK | 3.2 | 93360_NCI-H292_IL-9 | 41.9 |
| cells_PMA/ionomycin and IL-18 | | | |
| 93578_NK Cells IL-2_resting | 4.7 | 93359_NCI-H292_IL-13 | 27.9 |
| 93109_Mixed Lymphocyte | 7.1 | 93357_NCI-H292_IFN gamma | 21.6 |
| Reaction_Two Way MLR | | | |
| 93110_Mixed Lymphocyte | 6.0 | 93777_HPAEC_- | 13.8 |
| Reaction_Two Way MLR | | | |
| 93111_Mixed Lymphocyte | 4.9 | 93778_HPAEC_IL-1 beta/TNA | 16.4 |
| Reaction_Two Way MLR | | alpha | |
| 93112_Mononuclear Cells | 5.0 | 93254_Normal Human Lung | 38.7 |
| (PBMCs)_resting | | Fibroblast_none | |
| 93113_Mononuclear Cells | 5.8 | 93253_Normal Human Lung | 46.6 |
| (PBMCs)_PWM | | Fibroblast_TNFa (4 ng/ml) and IL- | |
| | | 1b (1 ng/ml) | |
| 93114_Mononuclear Cells | 4.1 | 93257_Normal Human Lung | 34.6 |
| (PBMCs)_PHA-L | | Fibroblast_IL-4 | |
| 93249_Ramos (B cell)_none | 23.0 | 93256_Normal Human Lung | 20.3 |
| | | Fibroblast_IL-9 | |
| 93250_Ramos (B cell)_ionomycin | 16.8 | 93255_Normal Human Lung | 19.8 |
| | | Fibroblast_IL-13 | |
| 93349_B lymphocytes_PWM | 7.4 | 93258_Normal Human Lung | 32.4 |
| | | Fibroblast_IFN gamma | |
| 93350_B lymphoytes_CD40L and | 5.4 | 93106_Dermal Fibroblasts | 48.1 |
| IL-4 | | CCD1070_resting | |
| 92665_EOL-1 | 12.2 | 93361_Dermal Fibroblasts | 39.6 |
| (Eosinophil)_dbcAMP | | CCD1070_TNF alpha 4 ng/ml | |
| differentiated | | | |
| 93248_EOL-1 | 10.6 | 93105_Dermal Fibroblasts | 23.7 |
| (Eosinophil)_dbcAMP/PMAionomycin | | CCD1070_IL-1 beta 1 ng/ml | |
| 93356_Dendritic Cells_none | 10.0 | 93772_dermal fibroblast_IFN | 11.6 |
| | | gamma | |
| 93355_Dendritic Cells_LPS 100 ng/ml | 8.8 | 93771_dermal fibroblast_IL-4 | 23.5 |
| 93775_Dendritic Cells_anti-CD40 | 11.9 | 93260_IBD Colitis 2 | 5.0 |
| 93774_Monocytes_resting | 13.5 | 93261_IBD Crohns | 12.2 |
| 93776_Monocytes_LPS 50 ng/ml | 8.5 | 735010_Colon_normal | 100.0 |
| 93581_Macrophages_resting | 11.6 | 735019_Lung_none | 11.4 |
| 93582_Macrophages_LPS 100 ng/ml | 7.4 | 64028-1_Thymus_none | 25.3 |
| 93098_HUVEC | 43.0 | 64030-1_Kidney_none | 8.4 |
| (Endothelial)_none | | | |
| 93099_HUVEC | 43.9 | | |
| (Endothelial)_starved | | | |

TABLE 19
Panel CNS_neurodegeneration_v1.0

| | Relative | | Relative |
|---|---|---|---|
| | Expression (%) | | Expression (%) |
| | tm7048f_{—} | | tm7048f_{—} |
| Tissue Name | ag3088_a2_s1 | Tissue Name | ag3088_a2_s1 |
| AD 1 Hippo | 19.7 | Control (Path) 3 Temporal Ctx | 14.0 |
| AD 2 Hippo | 35.6 | Control (Path) 4 Temporal Ctx | 44.4 |
| AD 3 Hippo | 17.9 | AD 1 Occipital Ctx | 27.8 |
| AD 4 Hippo | 17.4 | AD 2 Occipital Ctx (Missing) | 0.0 |
| AD 5 Hippo | 100.0 | AD 3 Occipital Ctx | 15.6 |
| AD 6 Hippo | 61.7 | AD 4 Occipital Ctx | 92.3 |
| Control 2 Hippo | 58.0 | AD 5 Occipital Ctx | 70.8 |
| Control 4 Hippo | 15.2 | AD 6 Occipital Ctx | 25.9 |
| Control (Path) 3 Hippo | 13.4 | Control 1 Occipital Ctx | 8.7 |
| AD 1 Temporal Ctx | 29.1 | Control 2 Occipital Ctx | 77.7 |
| AD 2 Temporal Ctx | 47.3 | Control 3 Occipital Ctx | 29.6 |
| AD 3 Temporal Ctx | 14.7 | Control 4 Occipital Ctx | 9.8 |
| AD 4 Temporal Ctx | 34.1 | Control (Path) 1 Occipital Ctx | 69.0 |
| AD 5 Inf Temporal Ctx | 84.2 | Control (Path) 2 Occipital Ctx | 16.7 |
| AD 5 Sup Temporal Ctx | 47.4 | Control (Path) 3 Occipital Ctx | 7.0 |
| AD 6 Inf Temporal Ctx | 65.5 | Control (Path) 4 Occipital Ctx | 24.5 |
| AD 6 Sup Temporal Ctx | 60.2 | Control 1 Parietal Ctx | 13.8 |
| Control 1 Temporal Ctx | 10.0 | Control 2 Parietal Ctx | 66.5 |
| Control 2 Temporal Ctx | 69.1 | Control 3 Parietal Ctx | 19.8 |
| Control 3 Temporal Ctx | 33.8 | Control (Path) 1 Parietal Ctx | 63.4 |
| Control 3 Temporal Ctx | 17.8 | Control (Path) 2 Parietal Ctx | 33.1 |
| Control (Path) 1 Temporal Ctx | 67.6 | Control (Path) 3 Parietal Ctx | 8.6 |
| Control (Path) 2 Temporal Ctx | 52.5 | Control (Path) 4 Parietal Ctx | 59.2 |

Panel 1.3D Summary The NOV2 gene is widely expressed in many of the samples in this panel, with highest expression in a brain cancer cell line (CT=26). The NOV2 gene is also highly expressed in all the normal tissues originating in the central nervous system, including the amygdala, cerebellum, hippocampus, substantia nigra, thalamus, cerebral cortex and spinal cord. The protein encoded by the NOV2 gene is a homolog of epsin, Which is involved in the phagocytosis of macromolecules, and interacts with Huntingtin-interacting protein. Therefore, this gene may play a critical role in the endocytosis of Huntingtin protein and the etiology of Huntington's disease. Downregulation of this gene or its protein product may be of therapeutic benefit in the treatment of Huntington's disease.

The NOV2 gene is also expressed in many tissues with metabolic function, including adipose, the pancreas, the adrenal, thyroid, and pituitary glands, and skeletal muscle, heart and liver from both fetal and adult sources. Thus, this gene product may be important in the pathogenesis and/or treatment of disease in any or all of these tissues, including obesity and diabetes.

The NOV2 gene is highly expressed in renal, breast, brain, ovarian, lung, colon, kidney, pancreatic and prostate cancer cell lines, when compared to normal kidney, breast, ovary, and protate tissues, and thus may play a role in cancer of these tissues. The gene may also play a role in metastasis of melanoma as one cell line expresses this gene at a higher level compared to other melanoma cell lines. Based on this expression profile, the expression of the NOV2 gene could be of use as a marker for different grades/types of these cancers. Furthermore, since this gene is expressed in multiple fetal tissues and cancer cell lines, Panel 2.2 Summary Highest expression of the NOV2 gene is detected in liver tissue adjacent to a liver tumor (CT 27.3). In addition, the level of expression in some lung, breast, liver and kidney cancer tissue samples appears to be increased when compared to the matched normal tissue. The reverse appears to be true for colon, ovary and stomach tissue, where expression is slightly higher in normal tissue than the matched cancer tissues. Thus, based upon its profile, the expression of the NOV2 gene could be of use as a marker for distinguishing some cancers from the normal adjacent tissue or as a marker for different grades/types of cancer.

Panel 4D Summary The NOV2 gene is most highly expressed in colon (CT=22). Significant expression is also detected in a variety of tissues including fibroblasts, endothelial and epithelial cells, keratinocytes, leukocytes and smooth muscle cells. The protein encoded by the NOV2 gene is a homolog of an EH-domain binding like protein, epsin, thought to be involved in endocytosis. Members of the epsin family have been shown to play an important role in wound healing Since the NOV2 gene is expressed in several cell types, therapeutics designed with the protein encoded for by this gene may serve important roles in regulating the cellular uptake of bio-therapeutic molecules in general, and specifically in enhancing wound healing.

Panel_CNS_neurodegeneration_v1.0 Summary Highest expression of the NOV2 gene is detected in the hippocampus of a patient with Alzheimer's disease (CT=25.6). However, there is also widespread expression in all the samples in this panel and no specific association between the expression of this gene and the presence of Alzheimer's disease is observed from these results. These results do however confirm expression of the NOV2 gene in the brains of an additional set of individuals. Please see Panel 1.3D for a discussion of potential utility of this gene in the central nervous system (Rosenthal et al., The epsins define a family of proteins that interact with components of the clathrin coat and contain a new protein module. J. Biol. Chem. 274:33959-65, 1999; Mishra et al., Clathrin- and AP-2-binding sites in HIP I uncover a general assembly role for endocytic accessory proteins. J Biol Chem, 2001; Spradling et al., Epsin 3 is a novel extracellular matrix-induced transcript specific to wounded epithelia. J. Biol. Chem. 276:29257-67, 2001).

NOV3: Low Density Lipoprotein B-Like

Expression of the NOV3 gene (also referred to as AC025263_da1) was assessed using the primer-probe sets Ag2002 and Ag2452 described in Tables 20 and 21. Results from RTQ-PCR runs are shown in Tables 22, 23, 24 and 25.

TABLE 20
Probe Name Ag2002

| | | | | Start | SEQ ID |
|---|---|---|---|---|---|
| Primers | Sequences | TM | Length | Position | NO: |
| Forward | 5′-GCCAGAAAGGCAACTATTCAG-3′ | 59 | 21 | 727 | 108 |
| Probe | FAM-5′-AACTTCTCAACCAGCCACACCATGGT-3′-TAMRA | 69.7 | 26 | 749 | 109 |
| Reverse | 5′-ACCAACTCCACTAATGAGCAAA-3′ | 59 | 22 | 794 | 110 |

TABLE 21
Probe Name Ag2452

| | | | | Start | SEQ ID |
|---|---|---|---|---|---|
| Primers | Sequences | TM | Length | Position | NO: |
| Forward | 5′-AGCAGTGCAGTTGTGAAAGTTT-3′ | 59.1 | 22 | 2053 | 111 |
| Probe | TET-5′-TGATTCATGGATTCACCCAGTCATTA-3′-TAMRA | 65.5 | 26 | 2075 112 | |
| Reverse | S′-CAGAACTGAGCCAGCATCAT-3′ | 59 | 20 | 2108 | 113 |

TABLE 22
Panel 1.3D

| | Relative | Relative |
|---|---|---|
| | Expression (%) | Expression (%) |
| | 1.3Dtm3824t_{—} | 1.3Dtm2811f_{—} |
| Tissue Name | ag2452 | ag2002 |
| Liver adenocarcinoma | 6.1 | 15.9 |
| Pancreas | 3.1 | 5.0 |
| Pancreatic ca. CAPAN 2 | 1.7 | 3.8 |
| Adrenal gland | 7.7 | 12.7 |
| Thyroid | 6.7 | 13.6 |
| Salivary gland | 4.9 | 7.3 |
| Pituitary gland | 24.8 | 23.8 |
| Brain (fetal) | 8.9 | 8.5 |
| Brain (whole) | 18.9 | 33.9 |
| Brain (amygdala) | 28.9 | 19.6 |
| Brain (cerebellum) | 9.1 | 8.5 |
| Brain (hippocampus) | 100.0 | 48.6 |
| Brain (substantia nigra) | 4.3 | 5.3 |
| Brain (thalamus) | 13.1 | 15.4 |
| Cerebral Cortex | 41.2 | 100.0 |
| Spinal cord | 5.3 | 8.5 |
| CNS ca. (glio/astro) U87-MG | 5.3 | 14.4 |
| CNS ca. (glio/astro) U-118-MG | 20.0 | 39.5 |
| CNS ca. (astro) SW1783 | 10.3 | 25.5 |
| CNS ca.* (neuro; met) SK-N-AS | 34.6 | 36.9 |
| CNS ca. (astro) SF-539 | 3.8 | 12.0 |
| CNS ca. (astro) SNB-75 | 6.7 | 33.7 |
| CNS ca. (glio) SNB-19 | 3.9 | 16.0 |
| CNS ca. (glio) U251 | 3.9 | 0.0 |
| CNS ca. (glio) SF-295 | 8.2 | 28.9 |
| Heart (fetal) | 9.6 | 55.1 |
| Heart | 2.7 | 7.2 |
| Fetal Skeletal | 24.8 | 84.1 |
| Skeletal muscle | 3.8 | 8.4 |
| Bone marrow | 4.5 | 3.1 |
| Thymus | 3.3 | 7.5 |
| Spleen | 9.0 | 12.7 |
| Lymph node | 4.1 | 12.9 |
| Colorectal | 5.9 | 18.7 |
| Stomach | 5.2 | 17.7 |
| Small intestine | 10.4 | 10.4 |
| Colon ca. SW480 | 7.8 | 34.2 |
| Colon ca.* (SW480 met)SW620 | 6.0 | 17.1 |
| Colon ca. HT29 | 3.8 | 10.2 |
| Colon ca. HCT-116 | 5.8 | 9.2 |
| Colon ca. CaCo-2 | 5.4 | 22.2 |
| 83219 CC Well to Mod Diff | 5.3 | 15.7 |
| (ODO3866) | | |
| Colon ca. HCC-2998 | 10.7 | 14.9 |
| Gastric ca.* (liver met) NCI-N87 | 10.7 | 31.6 |
| Bladder | 3.8 | 5.3 |
| Trachea | 13.2 | 14.0 |
| Kidney | 2.3 | 3.3 |
| Kidney (fetal) | 5.9 | 9.7 |
| Renal ca. 786-0 | 2.7 | 6.8 |
| Renal ca. A498 | 14.8 | 34.9 |
| Renal ca. RXF 393 | 1.3 | 6.9 |
| Renal ca. ACHN | 1.4 | 24.5 |
| Renal ca. UO-31 | 3.7 | 15.5 |
| Renal ca. TK-10 | 4.6 | 14.9 |
| Liver | 2.9 | 2.8 |
| Liver (fetal) | 7.1 | 7.9 |
| Liver ca. (hepatoblast) HepG2 | 5.8 | 28.1 |
| Lung | 11.7 | 7.5 |
| Lung (fetal) | 7.6 | 14.6 |
| Lung ca. (small cell) LX-1 | 3.1 | 16.6 |
| Lung ca. (small cell) NCI-H69 | 14.7 | 36.1 |
| Lung ca. (s.cell var.) SHP-77 | 15.6 | 30.6 |
| Lung ca. (large cell)NCI-H460 | 2.2 | 4.5 |
| Lung ca. (non-sm. cell) A549 | 8.2 | 12.0 |
| Lung ca. (non-s.cell) NCI-H23 | 3.8 | 15.4 |
| Lung ca. (non-s.cell) HOP-62 | 5.1 | 21.8 |
| Lung ca. (non-s.cl) NCI-H522 | 5.5 | 18.3 |
| Lung ca. (squam) SW 900 | 4.0 | 9.8 |
| Lung ca. (squam.) NCI-H596 | 3.1 | 14.7 |
| Mammary gland | 11.2 | 27.5 |
| Breast ca.* (pl. effusion) MCF-7 | 7.3 | 23.7 |
| Breast ca.* (pl.ef) MDA-MB-231 | 23.7 | 39.8 |
| Breast ca.* (pl. effusion) T47D | 8.4 | 37.1 |
| Breast ca. BT-549 | 11.0 | 16.4 |
| Breast ca. MDA-N | 8.7 | 20.6 |
| Ovary | 17.6 | 52.5 |
| Ovarian ca. OVCAR-3 | 4.9 | 19.9 |
| Ovarian ca. OVCAR-4 | 0.9 | 3.3 |
| Ovarian ca. OVCAR-5 | 7.0 | 32.5 |
| Ovarian ca. OVCAR-8 | 5.4 | 14.4 |
| Ovarian ca. IGROV-1 | 1.9 | 3.8 |
| Ovarian ca.* (ascites) SK-OV-3 | 4.4 | 12.0 |
| Uterus | 7.6 | 14.2 |
| Placenta | 7.9 | 13.2 |
| Prostate | 6.0 | 6.8 |
| Prostate ca.* (bone met)PC-3 | 8.1 | 18.4 |
| Testis | 10.6 | 19.6 |
| Melanoma Hs688(A).T | 3.7 | 28.9 |
| Melanoma* (met) Hs688(B)T | 2.3 | 45.7 |
| Melanoma UACC-62 | 1.1 | 3.3 |
| Melanoma M14 | 1.2 | 3.5 |
| Melanoma LOX IMVI | 9.1 | 6.7 |
| Melanoma* (met) SK-MEL-5 | 12.9 | 13.7 |
| Adipose | 2.6 | 4.6 |

TABLE 23
Panel 2D

| | Relative | | Relative |
|---|---|---|---|
| | Expression (%) | | Expression (%) |
| | 2Dtm3825t_{—} | | 2Dtm3825t_{—} |
| Tissue Name | ag2452 | Tissue Name | ag2452 |
| Normal Colon GENPAK 061003 | 100.0 | Kidney NAT Clontech 8120608 | 24.5 |
| 83219 CC Well to Mod Diff | 18.2 | Kidney Cancer Clontech 8120613 | 51.1 |
| (ODO3866) | | | |
| 83220 CC NAT (ODO3866) | 19.1 | Kidney NAT Clontech 8120614 | 29.1 |
| 83221 CC Gr.2 rectosigmoid | 8.0 | Kidney Cancer Clontech 9010320 | 19.3 |
| (ODO3868) | | | |
| 83222 CC NAT (ODO3868) | 6.0 | Kidney NAT Clontech 9010321 | 31.4 |
| 83235 CC Mod Diff (ODO3920) | 23.3 | Normal Uterus GENPAK 061018 | 6.8 |
| 83236 CC NAT (ODO3920) | 24.0 | Uterus Cancer GENPAK 064011 | 32.1 |
| 83237 CC Gr.2 ascend colon | 91.4 | Normal Thyroid Clontech A+ | 29.9 |
| (ODO3921) | | 6570-1 | |
| 83238 CC NAT (ODO3921) | 19.8 | Thyroid Cancer GENPAK 064010 | 28.3 |
| 83241 CC from Partial | 66.4 | Thyroid Cancer INVITROGEN | 17.1 |
| Hepatectomy (ODO4309) | | A302152 | |
| 83242 Liver NAT (ODO4309) | 21.2 | Thyroid NAT INVITROGEN | 29.9 |
| | | A302153 | |
| 87472 Colon mets to lung | 24.3 | Normal Breast GENPAK 061019 | 25.7 |
| OD04451-01) | | | |
| 87473 Lung NAT (OD04451-02) | 14.7 | 84877 Breast Cancer (OD04566) | 15.3 |
| Normal Prostate Clontech A+ | 33.9 | 85975 Breast Cancer (OD04590- | 76.8 |
| 6546-1 | | 01) | |
| 84140 Prostate Cancer (OD04410) | 38.7 | 85976 Breast Cancer Mets | 68.3 |
| | | (OD04590-03) | |
| 84141 Prostate NAT (OD04410) | 35.8 | 87070 Breast Cancer Metastasis | 77.9 |
| | | (OD04655-05) | |
| 87073 Prostate Cancer (OD04720- | 52.5 | GENPAK Breast Cancer 064006 | 14.2 |
| 01) | | | |
| 87074 Prostate NAT OD04720-02) | 68.3 | Breast Cancer Res. Gen. 1024 | 24.3 |
| Normal Lung GENPAK 061010 | 35.8 | Breast Cancer Clontech 9100266 | 68.3 |
| 83239 Lung Met to Muscle | 28.3 | Breast NAT Clontech 9100265 | 31.6 |
| (ODO4286) | | | |
| 83240 Muscle NAT (ODO4286) | 17.8 | Breast Cancer INVITROGEN | 35.4 |
| | | A209073 | |
| 84136 Lung Malignant Cancer | 35.6 | Breast NAT INVITROGEN | 22.8 |
| (OD03126) | | A2090734 | |
| 84137 Lung NAT (OD03126) | 45.1 | Normal Liver GENPAK 061009 | 9.6 |
| 84871 Lung Cancer (OD04404) | 17.9 | Liver Cancer GENPAK 064003 | 9.3 |
| 84872 Lung NAT (OD04404) | 18.0 | Liver Cancer Research Genetics | 9.7 |
| | | RNA 1025 | |
| 84875 Lung Cancer (OD04565) | 6.9 | Liver Cancer Research Genetics | 9.6 |
| | | RNA 1026 | |
| 84876 Lung NAT (OD04565) | 8.2 | Paired Liver Cancer Tissue | 13.6 |
| | | Research Genetics RNA 6004-T | |
| 85950 Lung Cancer (OD04237-01) | 50.0 | Paired Liver Tissue Research | 18.8 |
| | | Genetics RNA 6004-N | |
| 85970 Lung NAT (OD04237-02) | 16.8 | Paired Liver Cancer Tissue | 10.3 |
| | | Research Genetics RNA 6005-T | |
| 83255 Ocular Mel Met to Liver | 19.9 | Paired Liver Tissue Research | 1.7 |
| (ODO4310) | | Genetics RNA 6005-N | |
| 83256 Liver NAT (ODO4310) | 18.6 | Normal Bladder GENPAK 061001 | 53.2 |
| 84139 Melanoma Mets to Lung | 35.1 | Bladder Cancer Research Genetics | 37.1 |
| (OD04321) | | RNA 1023 | |
| 84138 Lung NAT (OD04321) | 35.1 | Bladder Cancer INVITROGEN | 26.6 |
| | | A302173 | |
| Normal Kidney GENPAK 061008 | 57.4 | 87071 Bladder Cancer (OD04718 | 46.3 |
| | | -01) | |
| 83786 Kidney Ca, Nuclear grade 2 | 58.2 | 87072 Bladder Normal Adjacent | 24.8 |
| (OD04338) | | (OD04718-03) | |
| 83787 Kidney NAT (OD04338) | 30.1 | Normal Ovary Res. Gen. | 41.8 |
| 83788 Kidney Ca Nuclear grade | 26.4 | Ovarian Cancer GENPAK 064008 | 54.0 |
| 1/2 (OD04339) | | | |
| 83789 Kidney NAT (OD04339) | 35.4 | 87492 Ovary Cancer (OD04768- | 76.8 |
| | | 07) | |
| 83790 Kidney Ca, Clear cell type | 38.7 | 87493 Ovary NAT (OD04768-08) | 10.5 |
| (OD04340) | | | |
| 83791 Kidney NAT (OD04340) | 28.7 | Normal Stomach GENPAK | 33.0 |
| | | 061017 | |
| 83792 Kidney Ca, Nuclear grade 3 | 18.3 | Gastric Cancer Clontech 9060358 | 11.5 |
| (OD04348) | | | |
| 83793 Kidney NAT (OD04348) | 25.7 | NAT Stomach Clontech 9060359 | 28.5 |
| 87474 Kidney Cancer (OD04622- | 18.4 | Gastric Cancer Clontech 9060395 | 35.1 |
| 01) | | | |
| 87475 Kidney NAT (OD04622-03) | 7.0 | NAT Stomach Clontech 9060394 | 40.3 |
| 85973 Kidney Cancer (OD04450- | 25.7 | Gastric Cancer Clontech 9060397 | 71.7 |
| 01) | | | |
| 85974 Kidney NAT (OD04450-03) | 24.1 | NAT Stomach Clontech 9060396 | 18.2 |
| Kidney Cancer Clontech 8120607 | 13.2 | Gastric Cancer GENPAK 064005 | 35.1 |

TABLE 24
Panel 4D

| | Relative | Relative |
|---|---|---|
| | Expression (%) | Expression (%) |
| | 4dx4tm5532f_{—} | 4Dtm3826t_{—} |
| Tissue Name | ag2002_a2 | ag2452 |
| 93768_Secondary Th1_anti-CD28/anti-CD3 | 25.4 | 27.4 |
| 93769_Secondary Th2_anti-CD28/anti-CD3 | 35.8 | 16.7 |
| 93770_Secondary Tr1_anti-CD28/anti-CD3 | 36.9 | 46.3 |
| 93573_Secondary Th1_resting day 4-6 in IL-2 | 25.8 | 13.4 |
| 93572_Secondary Th2_resting day 4-6 in IL-2 | 18.9 | 17.8 |
| 93571_Secondary Tr1_resting day 4-6 in IL-2 | 23.0 | 17.0 |
| 93568_primary Th1_anti-CD28/anti-CD3 | 15.9 | 31.0 |
| 93569_primary Th2_anti-CD28/anti-CD3 | 30.9 | 26.8 |
| 93570_primary Tr1_anti-CD28/anti-CD3 | 30.2 | 35.8 |
| 93565_primary Th1_resting dy 4-6 in IL-2 | 77.0 | 83.5 |
| 93566_primary Th2_resting dy 4-6 in IL-2 | 33.8 | 39.2 |
| 93567_primary Tr1_resting dy 4-6 in IL-2 | 29.2 | 23.2 |
| 93351_CD45RA CD4 lymphocyte_anti-CD28/anti-CD3 | 26.1 | 30.1 |
| 93352_CD45RO CD4 lymphocyte_anti-CD28/anti-CD3 | 37.1 | 34.9 |
| 93251_CD8 Lymphocytes_anti-CD28/anti-CD3 | 21.8 | 16.8 |
| 93353_chronic CD8 Lymphocytes 2ry_resting dy 4-6 in IL-2 | 26.8 | 24.3 |
| 93574_chronic CD8 Lymphocytes 2ry_activated CD3/CD28 | 23.1 | 28.9 |
| 93354_CD4_none | 22.9 | 19.6 |
| 93252_Secondary Th1/Th2/Tr1_anti-CD95 CH11 | 29.2 | 22.5 |
| 93103_LAK cells_resting | 16.9 | 21.5 |
| 93788_LAK cells_IL-2 | 33.2 | 22.8 |
| 93787_LAK cells_IL-2 + IL-12 | 33.1 | 18.4 |
| 93789_LAK cells_IL-2 + IFN gamma | 35.0 | 37.9 |
| 93790_LAK cells_IL-2 + IL-18 | 30.1 | 35.6 |
| 93104_LAK cells_PMA/ionomycin and IL-18 | 5.6 | 6.0 |
| 93578_NK Cells IL-2_resting | 24.2 | 19.3 |
| 93109_Mixed Lymphocyte Reaction_Two Way MLR | 29.3 | 28.9 |
| 93110_Mixed Lymphocyte Reaction_Two Way MLR | 22.3 | 15.3 |
| 93111_Mixed Lymphocyte Reaction_Two Way MLR | 21.6 | 12.1 |
| 93112_Mononuclear Cells (PBMCs)_resting | 14.6 | 12.4 |
| 93113_Mononuclear Cells (PBMCs)_PWM | 22.3 | 57.8 |
| 93114_Mononuclear Cells (PBMCs)_PHA-L | 11.5 | 28.7 |
| 93249_Ramos (B cell)_none | 27.1 | 21.5 |
| 93250_Ramos (B cell)_ionomycin | 16.9 | 66.9 |
| 93349_B lymphocytes_PWM | 23.6 | 65.5 |
| 93350_B lymphoytes_CD40L and IL-4 | 23.6 | 27.5 |
| 92665_EOL-1 (Eosinophil)_dbcAMP differentiated | 8.8 | 6.0 |
| 93248_EOL-1 (Eosinophil)_dbcAMP/PMAionomycin | 8.3 | 6.6 |
| 93356_Dendritic Cells_none | 14.0 | 10.1 |
| 93355_Dendritic Cells_LPS 100 ng/ml | 14.0 | 10.4 |
| 93775_Dendritic Cells_anti-CD40 | 19.4 | 15.0 |
| 93774_Monocytes_resting | 25.3 | 22.5 |
| 93776_Monocytes_LPS 50 ng/ml | 25.4 | 20.0 |
| 93581_Macrophages_resting | 21.6 | 24.8 |
| 93582_Macrophages_LPS 100 ng/ml | 16.5 | 13.7 |
| 93098_HUVEC (Endothelial)_none | 31.1 | 36.9 |
| 93099_HUVEC (Endothelial)_starved | 45.2 | 55.9 |
| 93100_HUVEC (Endothelial)_IL-1b | 15.8 | 24.7 |
| 93779_HUVEC (Endothelial)_IFN gamma | 37.6 | 45.4 |
| 93102_HUVEC (Endothelial)_TNF alpha + IFN gamma | 29.3 | 27.4 |
| 93101_HUVEC (Endothelial)_TNF alpha + IL4 | 27.6 | 20.4 |
| 93781_HUVEC (Endothelial)_IL-11 | 13.9 | 11.7 |
| 93583_Lung Microvascular Endothelial Cells_none | 21.2 | 26.4 |
| 93584_Lung Microvascular Endothelial Cells_TNFa (4 ng/ml) and | 29.0 | 34.9 |
| IL1b (1 ng/ml) | | |
| 92662_Microvascular Dermal endothelium_none | 25.5 | 36.3 |
| 92663_Microsvasular Dermal endothelium_TNFa (4 ng/ml) and IL1b | 25.7 | 28.7 |
| (1 ng/ml) | | |
| 93773_Bronchial epithelium_TNFa (4 ng/ml) and IL1b (1 ng/ml)** | 22.9 | 6.2 |
| 93347_Small Airway Epithelium_none | 18.8 | 17.7 |
| 93348_Small Airway Epithelium_TNFa (4 ng/ml) and IL1b (1 ng/ml) | 44.4 | 51.1 |
| 92668_Coronery Artery SMC_resting | 28.0 | 45.4 |
| 92669_Coronery Artery SMC_TNFa (4 ng/ml) and IL1b (1 ng/ml) | 22.3 | 25.0 |
| 93107_astrocytes_resting | 41.0 | 24.0 |
| 93108_astrocytes_TNFa (4 ng/ml) and IL1b (1 ng/ml) | 58.4 | 17.4 |
| 92666_KU-812 (Basophil)_resting | 28.6 | 31.0 |
| 92667_KU-812 (Basophil)_PMA/ionoycin | 63.5 | 65.1 |
| 93579_CCD1106 (Keratinocytes)_none | 17.0 | 18.9 |
| 93580_CCD1106 (Keratinocytes)_TNFa and IFNg** | 79.9 | 2.6 |
| 93791_Liver Cirrhosis | 16.7 | 3.3 |
| 93792_Lupus Kidney | 21.9 | 5.1 |
| 93577_NCI-H292 | 24.0 | 46.3 |
| 93358_NCI-H292_IL-4 | 24.3 | 42.6 |
| 93360_NCI-H292_IL-9 | 25.2 | 58.6 |
| 93359_NCI-H292_IL-13 | 11.7 | 32.3 |
| 93357_NCI-H292_IFN gamma | 14.7 | 37.9 |
| 93777_HPAEC_- | 23.1 | 25.7 |
| 93778_HPAEC_IL-1 beta/TNA alpha | 39.3 | 44.4 |
| 93254_Normal Human Lung Fibroblast_none | 40.6 | 26.2 |
| 93253_Normal Human Lung Fibroblast_TNFa (4 ng/ml) and IL-1b (1 ng/ml) | 71.3 | 31.6 |
| 93257_Normal Human Lung Fibroblast_IL-4 | 52.7 | 66.4 |
| 93256_Normal Human Lung Fibroblast_IL-9 | 29.9 | 67.8 |
| 93255_Normal Human Lung Fibroblast_IL-13 | 33.0 | 35.1 |
| 93258_Normal Human Lung Fibroblast_IFN gamma | 45.0 | 77.4 |
| 93106_Dermal Fibroblasts CCD1070_resting | 56.3 | 83.5 |
| 93361_Dermal Fibroblasts CCD1070_TNF alpha 4 ng/ml | 84.6 | 100.0 |
| 93105_Dermal Fibroblasts CCD1070_IL-1 beta 1 ng/ml | 39.7 | 45.4 |
| 93772_dermal fibroblast_IFN gamma | 15.5 | 19.5 |
| 93771_dermal fibroblast_IL-4 | 29.2 | 42.3 |
| 93260_IBD Colitis 2 | 4.7 | 2.2 |
| 93261_IBD Crohns | 7.2 | 4.7 |
| 735010_Colon_normal | 100.0 | 37.9 |
| 735019_Lung_none | 15.0 | 26.6 |
| 64028-1_Thymus_none | 39.9 | 55.1 |
| 64030-1_Kidney_none | 35.3 | 67.8 |

TABLE 25
Panel CNS_neurodegeneration_v1.0

| | Relative | | Relative |
|---|---|---|---|
| | Expression (%) | | Expression (%) |
| | tm6902t_{—} | | tm6902t_{—} |
| Tissue Name | ag2452_a2s2 | Tissue Name | ag2452_a2s2 |
| AD 1 Hippo | 8.0 | Control (Path) 3 Temporal Ctx | 4.2 |
| AD 2 Hippo | 36.4 | Control (Path) 4 Temporal Ctx | 35.8 |
| AD 3 Hippo | 2.9 | AD 1 Occipital Ctx | 5.2 |
| AD 4 Hippo | 8.3 | AD 2 Occipital Ctx (Missing) | 0.0 |
| AD 5 hippo | 52.3 | AD 3 Occipital Ctx | 2.1 |
| AD 6 Hippo | 47.1 | AD 4 Occipital Ctx | 28.1 |
| Control 2 Hippo | 37.4 | AD 5 Occipital Ctx | 15.8 |
| Control 4 Hippo | 7.7 | AD 6 Occipital Ctx | 49.3 |
| Control (Path) 3 Hippo | 3.6 | Control 1 Occipital Ctx | 2.2 |
| AD 1 Temporal Ctx | 7.9 | Control 2 Occipital Ctx | 65.3 |
| AD 2 Temporal Ctx | 49.1 | Control 3 Occipital Ctx | 10.0 |
| AD 3 Temporal Ctx | 4.1 | Control 4 Occipital Ctx | 5.3 |
| AD 4 Temporal Ctx | 28.4 | Control (Path) 1 Occipital Ctx | 87.2 |
| AD 5 Inf Temporal Ctx | 76.5 | Control (Path) 2 Occipital Ctx | 9.0 |
| AD 5 SupTemporal Ctx | 32.8 | Control (Path) 3 Occipital Ctx | 1.5 |
| AD 6 Inf Temporal Ctx | 46.7 | Control (Path) 4 Occipital Ctx | 12.0 |
| AD 6 Sup Temporal Ctx | 42.1 | Control 1 Parietal Ctx | 5.0 |
| Control 1 Temporal Ctx | 3.7 | Control 2 Parietal Ctx | 28.9 |
| Control 2 Temporal Ctx | 51.3 | Control 3 Parietal Ctx | 15.5 |
| Control 3 Temporal Ctx | 14.0 | Control (Path) 1 Parietal Ctx | 87.4 |
| Control 4 Temporal Ctx | 7.6 | Control (Path) 2 Parietal Ctx | 24.7 |
| Control (Path) 1 Temporal Ctx | 100.0 | Control (Path) 3 Parietal Ctx | 1.3 |
| Control (Path) 2 Temporal Ctx | 45.4 | Control (Path) 4 Parietal Ctx | 41.9 |

Panel 1.3D Summary Ag2002/Ag2452 Two experiments with two different probe/primer sets produce results that are in very good agreement, with highest expression in both runs occurring in regions of the brain. Expression of the NOV3 gene is highest in the cerebral cortex (CTs=26) in one run and the hippocampus in the other (CT=27) with significant expression also detected in the amygdala. This expression pattern indicates a functional role for the NOV3 gene product in Alzheimer's disease (AD), since the gene, a low density lipoprotein homolog, is expressed in the regions of the brain important to AD pathology. Increased expression of apolipoprotein B in the serum of Alzheimer's disease, and evidence that LRP contributes to the pathogenesis of Alzheimer's disease suggest a pathological role for the protein encoded by the NOV3 gene. Therefore, the AC024263_A gene product may be a promising antibody or small molecule target for the treatment of Alzheimer's disease.

High levels of expression are also detected in cell lines derived from brain cancer, breast cancer, lung cancer, kidney cancer and melanoma. In addition, the expression in normal ovary seems to be higher than in cell lines derived from ovarian cancer tissues. Thus, the expression of this gene could be of use as a marker or as a therapeutic for these cancers.

The NOV3 gene is widely expressed in tissues with metabolic function and significantly, is expressed at higher levels in fetal skeletal muscle (CTs=27-30) than in adult skeletal muscle (CTs=30-33). This difference in expression suggests that the NOV3 protein product could be involved in muscular growth or development in the fetus and therefore could act in a regenerative capacity in an adult. Thus, therapeutic modulation of the NOV3 gene could be useful in the treatment of muscle related diseases and treatment with the protein product could restore muscle mass or function to weak or dystrophic muscle.

Panel 2D Summary A 2452 Highest expression of the NOV3 gene occurs in colon (CT=29.7). High levels of expression are also detectable in breast cancer, prostate cancer, ovarian cancer, and colon cancer when compared to their normal adjacent tissue. Thus, expression of the NOV3 gene could be used as a marker to detect the presence of these cancers.

Panel 4D Summary Ag2002/Ag2452 Two experiments with two different probe and primer sets show highest expression of the NOV3 gene in normal colon (CT=26.2) and dermal fibroblasts treated with TNF-alpha (CT=29.2). Significant expression is also seen in fibroblasts, endothelial and epithelial cells, keratinocytes, leukocytes, smooth muscle cells and normal kidney. The NOV3 gene is expressed at much lower levels in colon from a patient with inflammatory bowel disease (IBD) when compared to expression in normal colon. Similarly, expression in lupus kidney is much lower than normal kidney. Thus, the protein encoded by the NOV3 gene may be involved in normal tissue/cellular functions and at least in the kidney and colon, downregulation of this protein may serve as a diagnostic marker for lupus or IBD.

Panel CNS_neurodegeneration_v1.0 Summary Ag2452 The NOV3 gene is expressed in most of the samples in this panel with highest expression detected in the temporal cortex of a control patient (CT=29.4). While no clear disease association emerged for the gene expression in this neurodegeneration panel, based on its homology to a low density lipoprotein and its expression profile in Panel 1.3D, the NOV3 gene product remains a promising antibody or small molecule target for the treatment of Alzheimer's disease (Caramelli et al., Increased apolipoprotein B serum concentration in Alzheimer's disease. Acta Neurol Scand. 100:61-3, 1999 and Ulery et al., Modulation of beta-amyloid precursor protein processing by the low density lipoprotein receptor-related protein (LRP). Evidence that LRP contributes to the pathogenesis of Alzheimer's disease. J Biol Chem 275(10):7410-5, 2000).

NOV4: Purinoceptor-Like

Expression of NOV4 gene (also referred to as AC026756_da1) was assessed using the primer-probe sets Ag1905 and Ag2504 described in Tables 26 and 27. Results from RTQ-PCR runs are shown in Tables 28, 29, and 30.

TABLE 26
Probe Name Ag1905

| | | | | Start | SEQ ID |
|---|---|---|---|---|---|
| Primers | Sequences | TM | Length | Position | NO: |
| Forward | 5′-TGAGAATCAGATCCATGAAGCT-3′ | 58.9 | 22 | 1174 | 114 |
| Probe | TET-5′-CCATTAGCTGCTCTGAACACCTTTCG-3′-TAMRA | 67.9 | 26 | 1211 | 115 |
| Reverse | 5′-GTCCCTGACCACCACATATAGT-3′ | 59 | 22 | 1246 | 116 |

TABLE 27
Probe Name Ag2504

| | | | | Start | SEQ ID |
|---|---|---|---|---|---|
| Primers | Sequences | TM | Length | Position | NO: |
| Forward | 5′-CTGAGAGCGAGTTACTGCTCAT-3′ | 58.9 | 22 | 272 | 117 |
| Probe | TET-5′-TGATTCATATTGCCAAACTGAACTCTCTTG-3′-TAMRA | 67.1 | 30 | 295 | 118 |
| Reverse | 5′-TGTCTCCTTTCATCTTGCAAGA-3′ | 60 | 22 | 328 | 119 |

TABLE 28
Panel 1.3D

| | Relative Expression (%) | |
|---|---|---|
| | 1.3Dtm2783t_{—} | 1.3Dtm2834t_{—} |
| Tissue Name | ag1905 | ag1905 |
| Liver adenocarcinoma | 0.0 | 0.0 |
| Pancreas | 1.3 | 3.2 |
| Pancreatic ca. CAPAN 2 | 0.0 | 0.0 |
| Adrenal gland | 0.0 | 0.5 |
| Thyroid | 1.9 | 1.1 |
| Salivary gland | 2.1 | 1.2 |
| Pituitary gland | 0.0 | 0.5 |
| Brain (fetal) | 2.7 | 1.3 |
| Brain (whole) | 7.5 | 9.9 |
| Brain (amygdala) | 4.2 | 6.7 |
| Brain (cerebellum) | 0.0 | 0.0 |
| Brain (hippocampus) | 4.5 | 10.7 |
| Brain (substantia nigra) | 0.7 | 0.4 |
| Brain (thalamus) | 15.1 | 9.2 |
| Cerebral Cortex | 14.2 | 17.3 |
| Spinal cord | 4.8 | 1.0 |
| CNS ca. (glio/astro) U87-MG | 0.0 | 0.0 |
| CNS ca. (glio/astro) U-118-MG | 0.4 | 0.9 |
| CNS ca. (astro) SW1783 | 0.0 | 0.4 |
| CNS ca.* (neuro. met) SK-N-AS | 3.4 | 1.4 |
| CNS ca. (astro) SF-539 | 0.0 | 0.0 |
| CNS ca. (astro) SNB-75 | 0.0 | 0.0 |
| CNS ca. (glio) SNB-19 | 0.0 | 0.0 |
| CNS ca. (glio) U251 | 0.0 | 0.0 |
| CNS ca. (glio) SF-295 | 0.0 | 0.0 |
| Heart (fetal) | 0.0 | 0.0 |
| Heart | 0.5 | 0.4 |
| Fetal Skeletal | 2.5 | 3.7 |
| Skeletal muscle | 0.0 | 0.0 |
| Bone marrow | 0.4 | 0.0 |
| Thymus | 0.0 | 0.0 |
| Spleen | 0.9 | 1.6 |
| Lymph node | 0.6 | 1.2 |
| Colorectal | 3.5 | 4.4 |
| Stomach | 1.5 | 1.1 |
| Small intestine | 0.3 | 1.3 |
| Colon ca. SW480 | 15.2 | 18.8 |
| Colon ca.* (SW480 met)SW620 | 5.1 | 8.8 |
| Colon ca. HT29 | 0.0 | 0.0 |
| Colon ca. HCT-116 | 0.0 | 0.5 |
| Colon ca. CaCo-2 | 0.0 | 1.0 |
| 83219 CC Well to Mod Diff | 30.1 | 38.2 |
| (ODO3866) | | |
| Colon ca. HCC-2998 | 1.0 | 0.5 |
| Gastric ca.* (liver met) NCI-N87 | 0.9 | 0.0 |
| Bladder | 0.0 | 0.0 |
| Trachea | 100.0 | 61.1 |
| Kidney | 5.3 | 3.7 |
| Kidney (fetal) | 1.7 | 1.9 |
| Renal ca. 786-0 | 0.0 | 0.0 |
| Renal ca. A498 | 0.0 | 0.0 |
| Renal ca. RXF 393 | 0.0 | 0.0 |
| Renal ca. ACHN | 0.0 | 0.0 |
| Renal ca. UO-31 | 0.0 | 0.0 |
| Renal ca. TK-10 | 0.0 | 0.0 |
| Liver | 0.0 | 0.0 |
| Liver (fetal) | 0.0 | 0.0 |
| Liver ca. (hepatoblast) HepG2 | 0.0 | 0.0 |
| Lung | 1.9 | 1.1 |
| Lung (fetal) | 3.3 | 3.8 |
| Lung ca. (small cell) LX-1 | 3.1 | 2.1 |
| Lung ca. (small cell) NCI-H69 | 1.0 | 0.3 |
| Lung ca. (s cell var.) SHP-77 | 84.1 | 100.0 |
| Lung ca. (large cell) NCI-H460 | 0.0 | 0.0 |
| Lung ca. (non-sm. cell) A549 | 0.0 | 0.0 |
| Lung ca. (non-s.cell) NCI-H23 | 1.0 | 0.0 |
| Lung ca. (non-s.cell) HOP-62 | 0.4 | 0.0 |
| Lung ca. (non-s.cl) NCI-H522 | 0.0 | 0.0 |
| Lung ca. (squam.) SW 900 | 0.0 | 0.0 |
| Lung ca. (squam.) NCI-H596 | 0.5 | 1.0 |
| Mammary gland | 10.4 | 15.4 |
| Breast ca.* (pl. effusion) MCF-7 | 0.0 | 0.4 |
| Breast ca.* (pl. ef) MDA-MB-231 | 0.0 | 0.0 |
| Breast ca.* (pl. effusion) T47D | 1.0 | 0.5 |
| Breast ca. BT-549 | 0.5 | 1.0 |
| Breast ca. MDA-N | 0.0 | 0.0 |
| Ovary | 0.0 | 1.0 |
| Ovarian ca. OVCAR-3 | 7.9 | 9.9 |
| Ovarian ca. OVCAR-4 | 0.0 | 0.0 |
| Ovarian ca. OVCAR-5 | 0.0 | 0.0 |
| Ovarian ca. OVCAR-8 | 10.1 | 7.9 |
| Ovarian ca. IGROV-1 | 0.0 | 0.5 |
| Ovarian ca.* (ascites) SK-OV-3 | 0.0 | 0.0 |
| Uterus | 2.1 | 3.9 |
| Placenta | 12.1 | 13.6 |
| Prostate | 0.6 | 0.5 |
| Prostate ca.* (bone met)PC-3 | 0.0 | 0.0 |
| Testis | 1.7 | 1.4 |
| Melanoma Hs688(A).T | 0.0 | 0.0 |
| Melanoma* (met) Hs688(B).T | 0.0 | 0.0 |
| Melanoma UACC-62 | 0.0 | 0.0 |
| Melanoma M14 | 0.0 | 0.0 |
| Melanoma LOX IMVI | 0.2 | 0.5 |
| Melanoma* (met) SK-MEL-5 | 0.0 | 0.0 |
| Adipose | 0.0 | 1.1 |

TABLE 29
Panel 2D

| | Relative | | Relative |
|---|---|---|---|
| | Expression (%) | | Expression (%) |
| | 2Dtm3014t_{—} | | 2Dtm3014t_{—} |
| Tissue Name | ag1905 | Tissue Name | ag1905 |
| Normal Colon GENPAK 061003 | 21.6 | Kidney NAT Clontech 8120608 | 0.6 |
| 83219 CC Well to Mod Diff | 33.9 | Kidney Cancer Clontech 8120613 | 44.1 |
| (ODO3866) | | | |
| 83220 CC NAT (ODO3866) | 7.5 | Kidney NAT Clontech 8120614 | 2.3 |
| 83221 CC Gr.2 rectosigmoid | 6.6 | Kidney Cancer Clontech 9010320 | 0.5 |
| (ODO3868) | | | |
| 83222 CC NAT (ODO3868) | 0.3 | Kidney NAT Clontech 9010321 | 2.8 |
| 83235 CC Mod Diff (ODO3920) | 37.1 | Normal Uterus GENPAK 061018 | 2.2 |
| 83236 CC NAT (ODO3920) | 2.9 | Uterus Cancer GENPAK 064011 | 8.1 |
| 83237 CC Gr.2 ascend colon | 100.0 | Normal Thyroid Clontech A+ | 2.3 |
| (ODO3921) | | 6570-1 | |
| 83238 CC NAT (ODO3921) | 11.8 | Thyroid Cancer GENPAK 064010 | 0.9 |
| 83241 CC from Partial | 22.2 | Thyroid Cancer INVITROGEN | 1.0 |
| Hepatectomy (ODO4309) | | A302152 | |
| 83242 Liver NAT (ODO4309) | 0.0 | Thyroid NAT INVITROGEN | 2.3 |
| | | A302153 | |
| 87472 Colon mets to lung | 12.9 | Normal Breast GENPAK 061019 | 4.5 |
| (OD04451-01) | | | |
| 87473 Lung NAT (OD04451-02) | 2.3 | 84877 Breast Cancer (OD04566) | 0.3 |
| Normal Prostate Clontech A+ | 3.9 | 85975 Breast Cancer (OD04590- | 0.0 |
| 6546-1 | | 01) | |
| 84140 Prostate Cancer (OD04410) | 1.0 | 85976 Breast Cancer Mets | 0.6 |
| | | (OD04590-03) | |
| 84141 Prostate NAT (OD04410) | 2.5 | 87070 Breast Cancer Metastasis | 0.8 |
| | | (OD04655-05) | |
| 87073 Prostate Cancer (OD04720- | 4.2 | GENPAK Breast Cancer 064006 | 6.9 |
| 01) | | | |
| 87074 Prostate NAT (OD04720- | 4.0 | Breast Cancer Res. Gen 1024 | 14.1 |
| 02) | | | |
| Normal Lung GENPAK 061010 | 16.6 | Breast Cancer Clontech 9100266 | 1.0 |
| 83239 Lung Met to Muscle | 0.0 | Breast NAT Clontech 9100265 | 0.4 |
| (ODO4286) | | | |
| 83240 Muscle NAT (ODO4286) | 0.0 | Breast Cancer INVITROGEN | 6.7 |
| | | A209073 | |
| 84136 Lung Malignant Cancer | 8.8 | Breast NAT INVITROGEN | 11.3 |
| (OD03126) | | A2090734 | |
| 84137 Lung NAT (OD03126) | 4.7 | Normal Liver GENPAK 061009 | 0.0 |
| 84871 Lung Cancer (OD04404) | 3.3 | Liver Cancer GENPAK 064003 | 0.0 |
| 84872 Lung NAT (OD04404) | 3.9 | Liver Cancer Research Genetics | 0.5 |
| | | RNA 1025 | |
| 84875 Lung Cancer (OD04565) | 0.0 | Liver Cancer Research Genetics | 0.0 |
| | | RNA 1026 | |
| 84876 Lung NAT (OD04565) | 0.6 | Paired Liver Cancer Tissue | 0.0 |
| | | Research Genetics RNA 6004-T | |
| 85950 Lung Cancer (OD04237-01) | 10.7 | Paired Liver Tissue Research | 0.6 |
| | | Genetics RNA 6004-N | |
| 85970 Lung NAT (OD04237-02) | 3.2 | Paired Liver Cancer Tissue | 0.6 |
| | | Research Genetics RNA 6005-T | |
| 83255 Ocular Mel Met to Liver | 0.0 | Paired Liver Tissue Research | 0.0 |
| (ODO4310) | | Genetics RNA 6005-N | |
| 83256 Liver NAT (ODO4310) | 0.5 | Normal Bladder GENPAK 061001 | 0.0 |
| 84139 Melanoma Mets to Lung | 0.0 | Bladder Cancer Research Genetics | 0.0 |
| (OD04321) | | RNA 1023 | |
| 84138 Lung NAT (OD04321) | 2.9 | Bladder Cancer INVITROGEN | 6.3 |
| | | A302173 | |
| Normal Kidney GENPAK 061008 | 66.4 | 87071 Bladder Cancer (OD04718- | 2.1 |
| | | 01) | |
| 83786 Kidney Ca, Nuclear grade 2 | 5.8 | 87072 Bladder Normal Adjacent | 2.3 |
| (OD04338) | | (OD04718-03) | |
| 83787 Kidney NAT (OD04338) | 49.3 | Normal Ovary Res Gen. | 0.0 |
| 83788 Kidney Ca Nuclear grade | 0.0 | Ovarian Cancer GENPAK 064008 | 16.4 |
| 1/2 (OD04339) | | | |
| 83789 Kidney NAT (OD04339) | 28.1 | 87492 Ovary Cancer (OD04768- | 0.5 |
| | | 07) | |
| 83790 Kidney Ca, Clear cell type | 1.5 | 87493 Ovary NAT (OD04768-08) | 0.0 |
| (OD04340) | | | |
| 83791 Kidney NAT (OD04340) | 54.7 | Normal Stomach GENPAK | 0.5 |
| | | 061017 | |
| 83792 Kidney Ca, Nuclear grade 3 | 0.0 | Gastric Cancer Clontech 9060358 | 1.7 |
| (OD04348) | | | |
| 83793 Kidney NAT (OD04348) | 12.5 | NAT Stomach Clontech 9060359 | 1.4 |
| 87474 Kidney Cancer (OD04622- | 0.0 | Gastric Cancer Clontech 9060395 | 0.5 |
| 01) | | | |
| 87475 Kidney NAT (OD04622-03) | 1.4 | NAT Stomach Clontech 9060394 | 0.0 |
| 85973 Kidney Cancer (OD04450- | 0.0 | Gastric Cancer Clontech 9060397 | 0.7 |
| 01) | | | |
| 85974 Kidney NAT (OD04450-03) | 71.2 | NAT Stomach Clontech 9060396 | 0.0 |
| Kidney Cancer Clontech 8120607 | 0.0 | Gastric Cancer GENPAK 064005 | 1.0 |

TABLE 30
Panel 4D

| | Relative | | Relative |
|---|---|---|---|
| | Expression (%) | | Expression (%) |
| | 4Dtm3015t_{—} | | 4Dtm3015t_{—} |
| Tissue Name | ag1905 | Tissue Name | ag1905 |
| 93768_Secondary Th1_anti- | 0.0 | 93100_HUVEC (Endothelial)_IL- | 0.0 |
| CD28/anti-CD3 | | 1b | |
| 93769_Secondary Th2_anti- | 0.0 | 93779_HUVEC (Endothelial)_IFN | 0.0 |
| CD28/anti-CD3 | | gamma | |
| 93770_Secondary Tr1_anti- | 0.0 | 93102_HUVEC | 0.0 |
| CD28/anti-CD3 | | (Endothelial)_TNF alpha + IFN | |
| | | gamma | |
| 93573_Secondary Th1_resting day | 0.0 | 93101_HUVEC | 0.0 |
| 4-6 in IL-2 | | (Endothelial)_TNF alpha + IL4 | |
| 93572_Secondary Th2_resting day | 0.0 | 93781_HUVEC (Endothelial)_IL- | 0.0 |
| 4-6 in IL-2 | | 11 | |
| 93571_Secondary Tr1_resting day | 0.0 | 93583_Lung Microvascular | 0.0 |
| 4-6 in IL-2 | | Endothelial Cells_none | |
| 93568_primary Th1_anti- | 0.0 | 93584_Lung Microvascular | 0.0 |
| CD28/anti-CD3 | | Endothelial Cells_TNFa (4 ng/ml) | |
| | | and IL1b (1 ng/ml) | |
| 93569_primary Th2_anti- | 0.0 | 92662_Microvascular Dermal | 1.3 |
| CD28/anti-CD3 | | endothelium_none | |
| 93570_primary Tr1_anti- | 0.0 | 92663_Microsvasular Dermal | 0.0 |
| CD28/anti-CD3 | | endothelium_TNFa (4 ng/ml) and | |
| | | IL1b (1 ng/ml) | |
| 93565_primary Th1_resting dy 4-6 | 0.0 | 93773_Bronchial | 0.0 |
| in IL-2 | | epithelium_TNFa (4 ng/ml) and | |
| | | IL1b (1 ng/ml)** | |
| 93566_primary Th2_resting dy 4-6 | 0.0 | 93347_Small Airway | 0.0 |
| in IL-2 | | Epithelium_none | |
| 93567_primary Tr1_resting dy 4-6 | 0.0 | 93348_Small Airway | 0.0 |
| in IL-2 | | Epithelium_TNFa (4 ng/ml) and | |
| | | IL1b (1 ng/ml) | |
| 93351_CD45RA CD4 | 0.0 | 92668_Coronery Artery | 0.0 |
| lymphocyte_anti-CD28/anti-CD3 | | SMC_resting | |
| 93352_CD45RO CD4 | 0.0 | 92669_Coronery Artery | 0.0 |
| lymphocyte_anti-CD28/anti-CD3 | | SMC_TNFa (4 ng/ml) and IL1b (1 ng/ml) | |
| 93251_CD8 Lymphocytes_anti- | 0.0 | 93107_astrocytes_resting | 0.0 |
| CD28/anti-CD3 | | | |
| 93353_chronic CD8 Lymphocytes | 0.0 | 93108_astrocytes_TNFa (4 ng/ml) | 0.0 |
| 2ry_resting dy 4-6 in IL-2 | | and IL1b (1 ng/ml) | |
| 93574_chronic CD8 Lymphocytes | 0.0 | 92666_KU-812 (Basophil)_resting | 0.7 |
| 2ry_activated CD3/CD28 | | | |
| 93354_CD4_none | 0.0 | 92667_KU-812 | 1.4 |
| | | (Basophil)_PMA/ionoycin | |
| 93252_Secondary | 0.0 | 93579_CCD1106 | 1.0 |
| Th1/Th2/Tr1_anti-CD95 CH11 | | (Keratinocytes)_none | |
| 93103_LAK cells_resting | 0.0 | 93580_CCD1106 | 0.0 |
| | | (Keratinocytes)_TNFa and IFNg** | |
| 93788_LAK cells_IL-2 | 0.0 | 93791_Liver Cirrhosis | 5.6 |
| 93787_LAK cells_IL-2 + IL-12 | 0.0 | 93792_Lupus Kidney | 9.0 |
| 93789_LAK cells_IL-2 + IFN | 0.0 | 93577_NCI-H292 | 0.0 |
| gamma | | | |
| 93790_LAK cells_IL-2+ IL-18 | 0.0 | 93358_NCI-H292_IL-4 | 0.0 |
| 93104_LAK | 0.0 | 93360_NCI-H292_IL-9 | 0.0 |
| cells_PMA/ionomycin and IL-18 | | | |
| 93578_NK Cells IL-2_resting | 0.0 | 93359_NCI-H292_IL-13 | 0.0 |
| 93109_Mixed Lymphocyte | 0.0 | 93357_NCI-H292_IFN gamma | 0.0 |
| Reaction_Two Way MLR | | | |
| 93110_Mixed Lymphocyte | 0.0 | 93777_HPAEC_- | 0.0 |
| Reaction_Two Way MLR | | | |
| 93111_Mixed Lymphocyte | 0.0 | 93778_HPAEC_IL-1 beta/TNA | 0.0 |
| Reaction_Two Way MLR | | alpha | |
| 93112_Mononuclear Cells | 1.4 | 93254_Normal Human Lung | 0.0 |
| (PBMCs)_resting | | Fibroblast_none | |
| 93113_Mononuclear Cells | 0.0 | 93253_Normal Human Lung | 0.0 |
| (PBMCs)_PWM | | Fibroblast_TNFa (4 ng/ml) and IL- | |
| | | 1b (1 ng/ml) | |
| 93114_Mononuclear Cells | 0.0 | 93257_Normal Human Lung | 1.2 |
| (PBMCs)_PHA-L | | Fibroblast_IL-4 | |
| 93249_Ramos (B cell)_none | 0.0 | 93256_Normal Human Lung | 0.0 |
| | | Fibroblast_IL-9 | |
| 93250_Ramos (B cell)_ionomycin | 0.0 | 93255_Normal Human Lung | 0.0 |
| | | Fibroblast_IL-13 | |
| 93349_B lymphocytes_PWM | 0.0 | 93258_Normal Human Lung | 0.0 |
| | | Fibroblast_IFN gamma | |
| 93350_B lymphoytes_CD40L and | 0.0 | 93106_Dermal Fibroblasts | 0.0 |
| IL-4 | | CCD1070_resting | |
| 92665_EOL-1 | 0.0 | 93361_Dermal Fibroblasts | 0.0 |
| (Eosinophil)_dbcAMP | | CCD1070_TNF alpha 4 ng/ml | |
| differentiated | | | |
| 93248_EOL-1 | 0.0 | 93105_Dermal Fibroblasts | 0.0 |
| (Eosinophil)_dbcAMP/PMAionomycin | | CCD1070_IL-1 beta 1 ng/ml | |
| 93356_Dendritic Cells_none | 0.0 | 93772_dermal fibroblast_IFN | 0.0 |
| | | gamma | |
| 93355_Dendritic Cells_LPS 100 ng/ml | 0.0 | 93771_dermal fibroblast_IL-4 | 0.0 |
| 93775_Dendritic Cells_anti-CD40 | 0.0 | 93260_IBD Colitis 2 | 0.0 |
| 93774_Monocytes_resting | 1.3 | 93261_IBD Crohns | 0.0 |
| 93776_Monocytes_LPS 50 ng/ml | 0.0 | 735010_Colon_normal | 9.6 |
| 93581_Macrophages_resting | 0.0 | 735019_Lung_none | 5.6 |
| 93582_Macrophages_LPS 100 ng/ml | 0.0 | 64028-1_Thymus_none | 100.0 |
| 93098_HUVEC | 0.0 | 64030-1_Kidney_none | 0.6 |
| (Endothelial)_none | | | |
| 93099_HUVEC | 0.0 | | |
| (Endothelial)_starved | | | |

Panel 1.3D Summary Ag1905 Two experiments with the same probe and primer set produce results that are in good agreement with highest expression in the lung cancer cell line SHP-77 (CTs=30) and the trachea (CTs=30-31). There is also significant expression of the NOV4 gene in cell lines derived from the colon and ovary. This gene may play a role in different types of lung, ovary and colon cancer as it is more highly expressed in cell lines derived from these cancers compared to the normal tissues. Furthermore, expression in normal brain and pancreas seems to be higher than cancer cell lines derived from these tissues. Thus, expression of the NOV4 gene could be used as a marker or as a therapeutic for colon, ovarian, brain, lung, and pancreatic cancer. In addition, therapeutic modulation of the product of this gene, through the use of peptides, chimeric molecules or small molecule drugs, may be useful in the therapy of these cancers.

There is also significant expression of the NOV4 gene in tissues involved in the central nervous system including the amygdala, hippocampus, thalamus, cerebral cortex, and spinal cord.

Purinoceptors found in GDNF sensitive sensory neurons mediate nociceptor function. Since the NOV4 gene product is a homolog of a purinoceptor, agents that block the action of this receptor may have utility in treating pain, either acting as analgesics or inhibiting the establishment of chronic pain. In addition, since adenosine plays a significant neuromodulatory role in brain regions such as the hippocampus, cortex, basal ganglia, and thalamus, the NOV4 purinoceptor-homolog is localized in a position to participate with the action of adenosine in these brain regions. The protein encoded by the NOV4 gene is most homologous to P2Y4 and P2Y6 purinoceptors, suggesting that its function may be similar to the PLC-mediated Ca2+ mobilization induced by these receptors. Ca2+ mobilization is an important component of the molecular process leading to neurotransmitter release. Adenosine modulates the release of glutamate in the brain, which is the main excitatory amino acid neurotransmitter. Glutamate exerts excitotoxic neuronal damage and death in a number of pathological conditions, including stroke. Agonists of A1 adenosine receptors attenuate this damage via G protein-coupled inhibition of glutamate release. Antagonists of A2 receptors also attenuate glutamate induced excitoxicity. Therefore, agents that inhibit or stimulate the protein encoded by the NOV4 gene are likely to affect glutamate release in the brain and the subsequent action of glutamate in these regions. If the NOV4 gene product functions similarly to the A1 receptor with respect to glutamate release, then agonists of the putative receptor are likely to have utility in the treatment of stroke. If the NOV4 gene product functions similarly to the A2 receptor, then antagonists of the putative receptor are likely to have utility in the treatment of stroke. Furthermore, antagonists of the A2a purinoceptor are antidepressants. Therefore, antagonists of the NOV4 gene product may be useful antidepressants. A2a receptor antagonists also counter parkinsonian-like symptoms in mice, suggesting that the NOV4 gene product antagonists may also have utility in the treatment of Parkinson's disease.

Ag2504 Expression of the NOV4 gene is low/undetectable (Ct values >35) in all samples in Panel 1.3D (data not shown)

Panel 2.2 Summary Ag2504 Expression of the NOV4 gene is low/undetectable (Ct values >35) in all samples in Panel 2.2 (data not shown).

Panel 2D Summary Ag1905 Highest expression of the NOV4 gene is detected in a colon cancer (CT=30.4). Furthermore, expression of this gene appears to be overexpressed in colon cancer when compared to normal adjacent tissue in all six matched tissue pairs present in this panel. Thus, expression of the AC025756_da1 gene could be used to differentiate between colon cancer and normal tissue. Furthermore, therapeutic modulation of the function or activity of the NOV4 gene product could be effective in the treatment of colon cancer. The NOV4 gene also shows a reverse association in the kidney, with overexpression of the gene present in normal kidney when compared to the corresponding cancerous tissue. Thus, expression of the gene could also be used to differentiate between normal and cancerous kidney tissue and therapeutic modulation of the gene product could be effective in the treatment of renal cancer.

Panel 4D Summary Ag1905 Expression of the NOV4 gene is limited to the thymus (CT=31.9). The putative GPCR encoded by this gene could be important in T cell development since purinoreceptors have been demonstrated in thymocytes. Immunomodulatory, therapeutic drugs designed with the protein encoded for by the NOV4 gene may regulate T cell production in the thymus and be important in preventing tissue rejection, treating autoimmune disorders and treating viral diseases such as AIDS. In addition, the transcript or antibodies designed against the protein encoded for by the transcript could be used as diagnostic markers for identifying subsets of thymocytes at specific developmental stages.

Ag2504 Expression of the NOV4 gene is low/undetectable (Ct values >34.5) in all samples in Panel 4D (data not shown).

Panel CNS_neurodegeneration_v1.0 Summary Ag2504 Expression of the NOV4 gene is low/undetectable (Ct values >35) in all samples in Panel CNS_neurodegeneration_v1.0 (data not shown). (Nagy et al., Apoptosis of murine thymocytes induced by extracellular ATP is dose- and cytosolic pH-dependent. Immunol Lett. 72:23-30, 2000; Liu et al., P2Y purinoceptor activation mobilizes intracellular Ca2+ and induces a membrane current in rat intracardiac neurones. J. Physiol. 526 Pt 2:287-98, 2000; Ongini et al., Selective adenosine A2A receptor antagonists. Farmaco. 56(1-2):87-90, 2001; Chen et al., Neuroprotection by caffeine and A(2A) adenosine receptor inactivation in a model of Parkinson's disease. J. Neurosci. 21:RC143, 2001; Wardas et al., SCH 58261, an A(2A) adenosine receptor antagonist, counteracts parkinsonian-like muscle rigidity in rats. Synapse. 41:160-71, 2001; Driessen et al., Depression of C fiber-evoked activity by intrathecally administered reactive red 2 in rat thalamic neurons. Brain Res. 796 (12):284-90, 1998; El Yacoubi et al., Adenosine A2A receptor antagonists are potential antidepressants: evidence based on pharmacology and A2A receptor knockout mice. Br J. Pharmacol. 134:68-77, 2001).

NOV5: CG8841-Like

Expression of NOV5 gene (also referred to as AC026756 da1) was assessed using the primer-probe set Ag2000 described in Table 31. Results from RTQ-PCR runs are shown in Tables 32, 33, and 34.

TABLE 31
Probe Name Ag2000

| | | | | Start | SEQ ID |
|---|---|---|---|---|---|
| Primers | Sequences | TM | Length | Position | NO: |
| Forward | 5′-ACTCCACCAAGAAGATCCAGTT-3′ | 59.1 | 22 | 1007 | 120 |
| Probe | FAM-5′-TCTCTTCTGGAAGCTCTGCGACTTCA-3′-TAMRA | 68.2 | 26 | 1047 | 121 |
| Reverse | 5′-GCACGAAGAAGAGGAATTTCTT-3′ | 59 | 22 | 1075 | 122 |

TABLE 32
Panel 1.3D

| | Relative | | Relative |
|---|---|---|---|
| | Expression (%) | | Expression (%) |
| | 1.3Dtm2809f_{—} | | 1.3Dtm2809f_{—} |
| Tissue Name | ag2000 | Tissue Name | ag2000 |
| Liver adenocarcinoma | 9.8 | Kidney (fetal) | 6.0 |
| Pancreas | 24.8 | Renal ca. 786-0 | 0.0 |
| Pancreatic ca. CAPAN 2 | 1.3 | Renal ca. A498 | 1.0 |
| Adrenal gland | 3.3 | Renal ca. RXF 393 | 0.0 |
| Thyroid | 11.0 | Renal ca. ACHN | 1.5 |
| Salivary gland | 30.6 | Renal ca. UO-31 | 1.1 |
| Pituitary gland | 30.4 | Renal ca. TK-10 | 2.4 |
| Brain (fetal) | 13.0 | Liver | 0.7 |
| Brain (whole) | 39.2 | Liver (fetal) | 2.5 |
| Brain (amygdala) | 23.7 | Liver ca. (hepatoblast) HepG2 | 8.8 |
| Brain (cerebellum) | 21.0 | Lung | 12.9 |
| Brain (hippocampus) | 46.7 | Lung (fetal) | 30.4 |
| Brain (substantia nigra) | 10.4 | Lung ca. (small cell) LX-1 | 8.7 |
| Brain (thalamus) | 33.2 | Lung ca. (small cell) NCI-H69 | 29.5 |
| Cerebral Cortex | 100.0 | Lung ca. (s.cell var.) SHP-77 | 33.0 |
| Spinal cord | 14.6 | Lung ca. (large cell)NCI-H460 | 0.9 |
| CNS ca. (glio/astro) U87-MG | 0.1 | Lung ca. (non-sm. cell) A549 | 15.9 |
| CNS ca. (glio/astro) U-118-MG | 0.3 | Lung ca. (non-s.cell) NCI-H23 | 2.3 |
| CNS ca. (astro) SW1783 | 0.0 | Lung ca (non-s cell) HOP-62 | 3.3 |
| CNS ca.* (neuro; met) SK-N-AS | 4.3 | Lung ca. (non-s.cl) NCI-H522 | 1.8 |
| CNS ca. (astro) SF-539 | 0.0 | Lung ca. (squam.) SW 900 | 20.2 |
| CNS ca. (astro) SNB-75 | 35.6 | Lung ca. (squam.) NCI-H596 | 3.3 |
| CNS ca. (glio) SNB-19 | 5.7 | Mammary gland | 40.1 |
| CNS ca. (glio) U251 | 2.1 | Breast ca.* (pl. effusion) MCF-7 | 42.0 |
| CNS ca. (glio) SF-295 | 2.6 | Breast ca.* (pl.ef) MDA-MB-231 | 6.3 |
| Heart (fetal) | 44.4 | Breast ca.* (pl. effusion) T47D | 73.2 |
| Heart | 3.6 | Breast ca. BT-549 | 0.0 |
| Fetal Skeletal | 69.3 | Breast ca. MDA-N | 0.2 |
| Skeletal muscle | 0.6 | Ovary | 17.6 |
| Bone marrow | 1.8 | Ovarian ca. OVCAR-3 | 23.5 |
| Thymus | 2.9 | Ovarian ca. OVCAR-4 | 9.2 |
| Spleen | 14.8 | Ovarian ca. OVCAR-5 | 13.0 |
| Lymph node | 8.6 | Ovarian ca. OVCAR-8 | 2.8 |
| Colorectal | 18.9 | Ovarian ca. IGROV-1 | 1.9 |
| Stomach | 68.3 | Ovarian ca.* (ascites) SK-OV-3 | 2.7 |
| Small intestine | 21.9 | Uterus | 9.9 |
| Colon ca. SW480 | 10.0 | Placenta | 27.2 |
| Colon ca.* (SW480 met)SW620 | 2.9 | Prostate | 25.9 |
| Colon ca. HT29 | 16.8 | Prostate ca.* (bone met)PC-3 | 18.7 |
| Colon ca. HCT-116 | 5.5 | Testis | 7.4 |
| Colon ca. CaCo-2 | 11.6 | Melanoma Hs688(A).T | 0.0 |
| 83219 CC Well to Mod Diff | 27.0 | Melanoma* (met) Hs688(B)T | 0.1 |
| (ODO3866) | | | |
| Colon ca. HCC-2998 | 17.2 | Melanoma UACC-62 | 0.0 |
| Gastric ca.* (liver met) NCI-N87 | 48.6 | Melanoma M14 | 0.0 |
| Bladder | 10.7 | Melanoma LOX IMVI | 0.0 |
| Trachea | 36.1 | Melanoma* (met) SK-MEL-5 | 0.7 |
| Kidney | 1.9 | Adipose | 4.3 |

TABLE 33
Panel 2.2

| | Relative | | Relative |
|---|---|---|---|
| | Expression (%) | | Expression (%) |
| | 2.2x4tm6394 | | 2.2x4tm6394 |
| Tissue Name | f_ag2000_a1 | Tissue Name | f_ag2000_a1 |
| Normal Colon GENPAK 061003 | 13.9 | 83793 Kidney NAT (OD04348) | 10.7 |
| 97759 Colon cancer (OD06064) | 21.3 | 98938 Kidney malignant cancer | 29.6 |
| | | (OD06204B) | |
| 97760 Colon cancer NAT | 24.4 | 98939 Kidney normal adjacent | 3.8 |
| (OD06064) | | tissue (OD06204E) | |
| 97778 Colon cancer (OD06159) | 7.0 | 85973 Kidney Cancer (OD04450- | 4.1 |
| | | 01) | |
| 97779 Colon cancer NAT | 11.0 | 85974 Kidney NAT (OD04450-03) | 5.0 |
| (OD06159) | | | |
| 98861 Colon cancer (OD06297-04) | 8.7 | Kidney Cancer Clontech 8120613 | 1.3 |
| 98862 Colon cancer NAT | 14.1 | Kidney NAT Clontech 8120614 | 7.6 |
| (OD06297-015) | | | |
| 83237 CC Gr.2 ascend colon | 9.4 | Kidney Cancer Clontech 9010320 | 2.7 |
| (ODO3921) | | | |
| 83238 CC NAT (ODO3921) | 4.8 | Kidney NAT Clontech 9010321 | 2.9 |
| 97766 Colon cancer metastasis | 3.2 | Kidney Cancer Clontech 8120607 | 8.9 |
| (OD06104) | | | |
| 97767 Lung NAT (OD06104) | 10.2 | Kidney NAT Clontech 8120608 | 3.0 |
| 87472 Colon mets to lung | 10.8 | Normal Uterus GENPAK 061018 | 9.0 |
| (OD04451-01) | | | |
| 87473 Lung NAT (OD04451-02) | 8.3 | Uterus Cancer GENPAK 064011 | 4.9 |
| Normal Prostate Clontech A+ | 43.0 | Normal Thyroid Clontech A+ | 5.4 |
| 6546-1 (8090438) | | 6570-1 (7080817) | |
| 84140 Prostate Cancer (OD04410) | 17.2 | Thyroid Cancer GENPAK 064010 | 2.8 |
| 84141 Prostate NAT (OD04410) | 10.4 | Thyroid Cancer INVITROGEN | 6.3 |
| | | A302152 | |
| Normal Ovary Res Gen. | 7.6 | Thyroid NAT INVITROGEN | 4.6 |
| | | A302153 | |
| 98863 Ovarian cancer (OD06283- | 9.5 | Normal Breast GENPAK 061019 | 19.6 |
| 03) | | | |
| 98865 Ovarian cancer | 4.7 | 84877 Breast Cancer (OD04566) | 15.8 |
| NAT/fallopian tube (OD06283-07) | | | |
| Ovarian Cancer GENPAK 064008 | 7.3 | Breast Cancer Res. Gen. 1024 | 22.3 |
| 97773 Ovarian cancer (OD06145) | 0.4 | 85975 Breast Cancer (OD04590- | 47.6 |
| | | 01) | |
| 97775 Ovarian cancer NAT | 7.3 | 85976 Breast Cancer Mets | 41.3 |
| (OD06145) | | (OD04590-03) | |
| 98853 Ovarian cancer (OD06455- | 18.0 | 87070 Breast Cancer Metastasis | 100.0 |
| 03) | | (OD04655-05) | |
| 98854 Ovarian NAT (OD06455- | 2.4 | GENPAK Breast Cancer 064006 | 11.1 |
| 07) Fallopian tube | | | |
| Normal Lung GENPAK 061010 | 18.6 | Breast Cancer Clontech 9100266 | 49.1 |
| 92337 Invasive poor diff. lung | 10.0 | Breast NAT Clontech 9100265 | 20.7 |
| adeno (ODO4945-01 | | | |
| 92338 Lung NAT (ODO4945-03) | 5.7 | Breast Cancer INVITROGEN | 18.6 |
| | | A209073 | |
| 84136 Lung Malignant Cancer | 17.6 | Breast NAT INVITROGEN | 21.5 |
| (OD03126) | | A2090734 | |
| 84137 Lung NAT (OD03126) | 3.9 | 97763 Breast cancer (OD06083) | 81.2 |
| 90372 Lung Cancer (OD05014A) | 11.4 | 97764 Breast cancer node | 65.8 |
| | | metastasis (OD06083) | |
| 90373 Lung NAT (OD05014B) | 0.2 | Normal Liver GENPAK 061009 | 2.4 |
| 97761 Lung cancer (OD06081) | 4.2 | Liver Cancer Research Genetics | 4.4 |
| | | RNA 1026 | |
| 97762 Lung cancer NAT | 6.2 | Liver Cancer Research Genetics | 4.6 |
| (OD06081) | | RNA 1025 | |
| 85950 Lung Cancer (OD04237-01) | 4.6 | Paired Liver Cancer Tissue | 3.8 |
| | | Research Genetics RNA 6004-T | |
| 85970 Lung NAT (OD04237-02) | 9.1 | Paired Liver Tissue Research | 1.5 |
| | | Genetics RNA 6004-N | |
| 83255 Ocular Mel Met to Liver | 0.7 | Paired Liver Cancer Tissue | 12.1 |
| (ODO4310) | | Research Genetics RNA 6005-T | |
| 83256 Liver NAT (ODO4310) | 2.8 | Paired Liver Tissue Research | 9.6 |
| | | Genetics RNA 6005-N | |
| 84139 Melanoma Mets to Lung | 0.3 | Liver Cancer GENPAK 064003 | 1.5 |
| (OD04321) | | | |
| 84138 Lung NAT (OD04321) | 9.2 | Normal Bladder GENPAK 061001 | 19.6 |
| Normal Kidney GENPAK 061008 | 2.4 | Bladder Cancer Research Genetics | 6.3 |
| | | RNA 1023 | |
| 83786 Kidney Ca, Nuclear grade 2 | 9.7 | Bladder Cancer INVITROGEN | 8.6 |
| (OD04338) | | A302173 | |
| 83787 Kidney NAT (OD04338) | 1.7 | Normal Stomach GENPAK | 62.5 |
| | | 061017 | |
| 83788 Kidney Ca Nuclear grade | 4.2 | Gastric Cancer Clontech 9060397 | 5.1 |
| 1/2 (OD04339) | | | |
| 83789 Kidney NAT (OD04339) | 4.1 | NAT Stomach Clontech 9060396 | 38.5 |
| 83790 Kidney Ca, Clear cell type | 2.7 | Gastric Cancer Clontech 9060395 | 21.5 |
| (OD04340) | | | |
| 83791 Kidney NAT (OD04340) | 6.7 | NAT Stomach Clontech 9060394 | 43.5 |
| 83792 Kidney Ca, Nuclear grade 3 | 0.6 | Gastric Cancer GENPAK 064005 | 11.4 |
| (OD04348) | | | |

TABLE 34
Panel 4D

| | Relative | | Relative |
|---|---|---|---|
| | Expression (%) | | Expression (%) |
| | 4dx4tm5534f_{—} | | 4dx4tm5534f_{—} |
| Tissue Name | ag2000_a1 | Tissue Name | ag2000_a1 |
| 93768_Secondary Th1_anti- | 0.2 | 93100_HUVEC (Endothelial)_IL- | 4.8 |
| CD28/anti-CD3 | | 1b | |
| 93769_Secondary Th2_anti- | 0.3 | 93779_HUVEC (Endothelial)_IFN | 14.7 |
| CD28/anti-CD3 | | gamma | |
| 93770_Secondary Tr1_anti- | 0.6 | 93102_HUVEC | 1.9 |
| CD28/anti-CD3 | | (Endothelial)_TNF alpha + IFN | |
| | | gamma | |
| 93573_Secondary Th1_resting day | 0.1 | 93101_HUVEC | 4.0 |
| 4-6 in IL-2 | | (Endothelial)_TNF alpha + IL4 | |
| 93572_Secondary Th2_resting day | 0.7 | 93781_HUVEC (Endothelial)_IL- | 15.7 |
| 4-6 in IL-2 | | 11 | |
| 93571_Secondary Tr1_resting day | 0.3 | 93583_Lung Microvascular | 14.4 |
| 4-6 in IL-2 | | Endothelial Cells_none | |
| 93568_primary Th1_anti- | 0.1 | 93584_Lung Microvascular | 6.3 |
| CD28/anti-CD3 | | Endothelial Cells_TNFa (4 ng/ml) | |
| | | and IL1b (1 ng/ml) | |
| 93569_primary Th2_anti- | 0.2 | 92662_Microvascular Dermal | 15.5 |
| CD28/anti-CD3 | | endothelium_none | |
| 93570_primary Tr1_anti- | 0.1 | 92663_Microsvasular Dermal | 5.2 |
| CD28/anti-CD3 | | endothelium_TNFa (4 ng/ml) and | |
| | | IL1b (1 ng/ml) | |
| 93565_primary Th1_resting dy 4-6 | 0.4 | 93773_Bronchial | 2.6 |
| in IL-2 | | epithelium_TNFa (4 ng/ml) and | |
| | | IL1b (1 ng/ml)** | |
| 93566_primary Th2_resting dy 4-6 | 0.2 | 93347_Small Airway | 0.8 |
| in IL-2 | | Epithelium_none | |
| 93567_primary Tr1_resting dy 4-6 | 0.1 | 93348_Small Airway | 3.4 |
| in IL-2 | | Epithelium_TNFa (4 ng/ml) and | |
| | | IL1b (1 ng/ml) | |
| 93351_CD45RA CD4 | 0.3 | 92668_Coronery Artery | 0.1 |
| lymphocyte_anti-CD28/anti-CD3 | | SMC_resting | |
| 93352_CD45RO CD4 | 0.7 | 92669_Coronery Artery | 0.3 |
| lymphocyte_anti-CD28/anti-CD3 | | SMC_TNFa (4 ng/ml) and IL1b (1 ng/ml) | |
| 93251_CD8 Lymphocytes_anti- | 0.8 | 93107_astrocytes_resting | 3.6 |
| CD28/anti-CD3 | | | |
| 93353_chronic CD8 Lymphocytes | 0.9 | 93108_astrocytes_TNFa (4 ng/ml) | 6.9 |
| 2ry_resting dy 4-6 in IL-2 | | and IL1b (1 ng/ml) | |
| 93574_chronic CD8 Lymphocytes | 0.0 | 92666_KU-812 (Basophil)_resting | 0.0 |
| 2ry_activated CD3/CD28 | | | |
| 93354_CD4_none | 2.7 | 92667_KU-812 | 0.0 |
| | | (Basophil)_PMA/ionoycin | |
| 93252_Secondary | 0.0 | 93579_CCD1106 | 0.3 |
| Th1/Th2/Tr1_anti-CD95 CH11 | | (Keratinocytes)_none | |
| 93103_LAK cells_resting | 1.5 | 93580_CCD1106 | 1.5 |
| | | (Keratinocytes)_TNFa and IFNg** | |
| 93788_LAK cells_IL-2 | 2.6 | 93791_Liver Cirrhosis | 11.3 |
| 93787_LAK cells_IL-2 + IL-12 | 1.4 | 93792_Lupus Kidney | 9.2 |
| 93789_LAK cells_IL-2 + IFN | 1.2 | 93577_NCI-H292 | 20.3 |
| gamma | | | |
| 93790_LAK cells_IL-2 + IL-18 | 1.6 | 93358_NCI-H292_IL-4 | 17.5 |
| 93104_LAK | 0.3 | 93360_NCI-H292_IL-9 | 21.6 |
| cells_PMA/ionomycin and IL-18 | | | |
| 93578_NK Cells IL-2_resting | 0.4 | 93359_NCI-H292_IL-13 | 9.5 |
| 93109_Mixed Lymphocyte | 1.2 | 93357_NCI-H292_IFN gamma | 10.3 |
| Reaction_Two Way MLR | | | |
| 93110_Mixed Lymphocyte | 0.4 | 93777_HPAEC_- | 13.7 |
| Reaction_Two Way MLR | | | |
| 93111_Mixed Lymphocyte | 0.0 | 93778_HPAEC_IL-1 beta/TNA | 9.2 |
| Reaction_Two Way MLR | | alpha | |
| 93112_Mononuclear Cells | 0.8 | 93254_Normal Human Lung | 0.2 |
| (PBMCs)_resting | | Fibroblast_none | |
| 93113_Mononuclear Cells | 0.2 | 93253_Normal Human Lung | 0.8 |
| (PBMCs)_PWM | | Fibroblast_TNFa (4 ng/ml) and IL- | |
| | | 1b (1 ng/ml) | |
| 93114_Mononuclear Cells | 0.3 | 93257_Normal Human Lung | 0.1 |
| (PBMCs)_PHA-L | | Fibroblast_IL-4 | |
| 93249_Ramos (B cell)_none | 0.5 | 93256_Normal Human Lung | 0.2 |
| | | Fibroblast_IL-9 | |
| 93250_Ramos (B cell)_ionomycin | 0.7 | 93255_Normal Human Lung | 0.2 |
| | | Fibroblast_IL-13 | |
| 93349_B lymphocytes_PWM | 0.8 | 93258_Normal Human Lung | 0.3 |
| | | Fibroblast_IFN gamma | |
| 93350_B lymphocytes_CD40L and | 5.8 | 93106_Dermal Fibroblasts | 0.1 |
| IL-4 | | CCD1070_resting | |
| 92665_EOL-1 | 0.0 | 93361_Dermal Fibroblasts | 0.0 |
| (Eosinophil)_dbcAMP | | CCD1070_TNF alpha 4 ng/ml | |
| differentiated | | | |
| 93248_EOL-1 | 0.0 | 93105_Dermal Fibroblasts | 0.1 |
| (Eosinophil)_dbcAMP/PMAionomycin | | CCD1070_IL-1 beta 1 ng/ml | |
| 93356_Dendritic Cells_none | 0.2 | 93772_dermal fibroblast_IFN | 0.1 |
| | | gamma | |
| 93355_Dendritic Cells_LPS 100 ng/ml | 0.0 | 93771_dermal fibroblast_IL-4 | 0.1 |
| 93775_Dendritic Cells_anti-CD40 | 0.0 | 93260_IBD Colitis 2 | 2.9 |
| 93774_Monocytes_resting | 0.0 | 93261_IBD Crohns | 9.3 |
| 93776_Monocytes_LPS 50 ng/ml | 0.0 | 735010_Colon_normal | 100.0 |
| 93581_Macrophages_resting | 0.1 | 735019_Lung_none | 19.4 |
| 93582_Macrophages_LPS 100 ng/ml | 0.0 | 64028-1_Thymus_none | 11.2 |
| 93098_HUVEC | 7.4 | 64030-1_Kidney_none | 6.4 |
| (Endothelial)_none | | | |
| 93099_HUVEC | 17.8 | | |
| (Endothelial)_starved | | | |

Panel 1.3D Summary Highest expression of the NOV5 gene, a homolog of a transmembrane multi-pass protein, is seen in the cerebral cortex (CT=26.8), with moderate expression detectable across all regions of the brain. Because this gene shows a large down-regulation in brain cancers, its absence would be an excellent marker to determine if brain tissue was pre-cancerous in the examining and classifying of postmortem tissue Expression of the NOV5 gene is also widespread among tissues with metabolic relevance, including adipose, pancreas, adult and fetal heart, adult and fetal liver, adult and fetal skeletal muscle, and the adrenal, pituitary, and thyroid glands. The NOV5 gene is expressed at much higher levels in fetal heart and skeletal muscle (CTs=28) than in adult heart and skeletal muscle (CTs=31-34). This differential expression pattern suggests that NOV5 gene expression could be used to differentiate between the two tissue sources for heart and skeletal muscle. Furthermore, the significantly higher level of expression of the gene in fetal skeletal muscle suggests that the NOV5 gene product may be involved in muscular growth or development in the fetus and could potentially act in a regenerative capacity in an adult. Therefore, therapeutic modulation of the NOV5 gene could be useful in the treatment of muscle related diseases and the treatment of week or dystrophic muscle.

The NOV5 gene is also expressed at significant levels in cell lines derived from ovarian, breast, lung, gastric, prostate and colon cancers compared to the normal tissues. Thus, the expression of this gene could be of use as a marker or as a therapeutic for ovarian, breast, lung, gastric, prostate and colon. In addition, therapeutic modulation of the product of this gene, through the use of peptides, chimeric molecules or small molecule drugs, may be useful in the treatment of these cancers.

Panel 2.2 Summary Highest expression of the NOV5 gene is seen in breast cancer (CT=28) as is seen in Panel 1.3D. In addition, there is significant overexpression of the NOV5 gene in a cluster of breast, lung, and ovarian cancer samples when compared to corresponding normal tissues. Thus, expression of the NOV5 gene could be used to differentiate breast, ovarian and lung cancers from normal tissue and as a marker for the presence of these cancers. Furthermore, therapeutic modulation of the protein product of the NOV5 gene could be beneficial in the treatment of breast, ovarian and lung cancers. The expression of this gene also shows a reverse association with some normal stomach samples when compared to the matched gastric cancer tissue. This suggests that the NOV5 gene could be used to distinguish between normal and cancerous gastric tissue and that therapeutic modulation of the gene product may be useful in the treatment of gastric cancer.

Panel 4D Summary The highest expression of the NOV5 gene is found in the colon (CT=26.2), with modest expression detectable in the muco-epidremoid cell line H292, and the lung. It is also expressed at moderate levels on HUVEC and lung microvasculature regardless of their activation status. The protein encoded by the NOV5 gene is homologous to an epidermal growth factor related protein (fibropellin like) and could be used as a marker of lung muco-epidermoid cells, colon or vasculature. The putative protein encoded by the transcript may also play an important role in the normal homeostasis of these tissues. Small molecule or antibody therapeutics designed with the NOV5 gene product could be important for maintaining or restoring normal function to these organs during inflammation associated with asthma and emphysema.

NOV6: Synaptotagmin-Like

Expression of NOV6 gene (also referred to as SCI34912642-da1) was assessed using the primer-probe set Ag2056 described in Table 35. Results from RTQ-PCR runs are shown in Tables 36, 37, 38, 39 and 40.

TABLE 35
Probe Name Ag2056

| | | | | Start | SEQ ID |
|---|---|---|---|---|---|
| Primers | Sequences | TM | Length | Position | NO: |
| Forward | 5′-CTGGTCTCTGCCATCATCAC-3′ | 59.2 | 20 | 55 | 123 |
| Probe | TET-5′-CTTAGCGTCACTGTCGTCCTCGCTAG-3′-TAMRA | 68.4 | 26 | 82 | 124 |
| Reverse | 5′-TGTAGCGTTTGCCCAGTTT-3′ | 59.3 | 19 | 130 | 125 |

TABLE 36
Panel 1.3D

| | Relative | | Relative |
|---|---|---|---|
| | Expression (%) | | Expression (%) |
| | 1.3Dtm2580t_{—} | | 1.3Dtm2580t_{—} |
| Tissue Name | ag2056 | Tissue Name | ag2056 |
| Liver adenocarcinoma | 2.4 | Kidney (fetal) | 1.9 |
| Pancreas | 1.8 | Renal ca. 786-0 | 0.2 |
| Pancreatic ca. CAPAN 2 | 2.3 | Renal ca. A498 | 6.0 |
| Adrenal gland | 0.8 | Renal ca. RXF 393 | 0.8 |
| Thyroid | 1.3 | Renal ca. ACHN | 0.0 |
| Salivary gland | 7.9 | Renal ca. UO-31 | 0.0 |
| Pituitary gland | 16.3 | Renal ca. TK-10 | 0.6 |
| Brain (fetal) | 4.8 | Liver | 0.3 |
| Brain (whole) | 26.8 | Liver (fetal) | 1.1 |
| Brain (amygdala) | 24.0 | Liver ca (hepatoblast) HepG2 | 1.8 |
| Brain (cerebellum) | 8.8 | Lung | 0.6 |
| Brain (hippocampus) | 56.3 | Lung (fetal) | 0.9 |
| Brain (substantia nigra) | 2.9 | Lung ca. (small cell) LX-1 | 7.3 |
| Brain (thalamus) | 23.0 | Lung ca. (small cell) NCI-H69 | 16.2 |
| Cerebral Cortex | 100.0 | Lung ca. (s.cell var.) SHP-77 | 20.6 |
| Spinal cord | 0.6 | Lung ca. (large cell)NCI-H460 | 0.1 |
| CNS ca. (glio/astro) U87-MG | 0.4 | Lung ca. (non-sm. cell) A549 | 0.1 |
| CNS ca. (glio/astro) U-118-MG | 19.1 | Lung ca. (non-s.cell) NCI-H23 | 0.6 |
| CNS ca. (astro) SW1783 | 1.8 | Lung ca (non-s.cell) HOP-62 | 0.3 |
| CNS ca.* (neuro; met) SK-N-AS | 0.6 | Lung ca. (non-s.cl) NCI-H522 | 2.0 |
| CNS ca. (astro) SF-539 | 0.5 | Lung ca. (squam.) SW 900 | 1.1 |
| CNS ca. (astro) SNB-75 | 17.0 | Lung ca. (squam.) NCI-H596 | 13.3 |
| CNS ca. (glio) SNB-19 | 0.0 | Mammary gland | 12.9 |
| CNS ca. (glio) U251 | 0.1 | Breast ca.* (pl. effusion) MCF-7 | 4.7 |
| CNS ca. (glio) SF-295 | 0.6 | Breast ca.* (pl.ef) MDA-MB-231 | 0.1 |
| Heart (fetal) | 2.8 | Breast ca.* (pl. effusion) T47D | 17.1 |
| Heart | 1.7 | Breast ca. BT-549 | 0.0 |
| Fetal Skeletal | 9.4 | Breast ca. MDA-N | 0.1 |
| Skeletal muscle | 0.1 | Ovary | 0.9 |
| Bone marrow | 0.0 | Ovarian ca. OVCAR-3 | 2.5 |
| Thymus | 0.1 | Ovarian ca. OVCAR-4 | 1.1 |
| Spleen | 1.1 | Ovarian ca. OVCAR-5 | 3.9 |
| Lymph node | 0.1 | Ovarian ca. OVCAR-8 | 2.3 |
| Colorectal | 3.2 | Ovarian ca. IGROV-1 | 0.0 |
| Stomach | 1.9 | Ovarian ca.* (ascites) SK-OV-3 | 3.5 |
| Small intestine | 0.3 | Uterus | 1.3 |
| Colon ca. SW480 | 6.7 | Placenta | 18.0 |
| Colon ca.* (SW480 met)SW620 | 0.3 | Prostate | 18.8 |
| Colon ca. HT29 | 1.5 | Prostate ca.* (bone met)PC-3 | 4.5 |
| Colon ca. HCT-116 | 4.5 | Testis | 2.3 |
| Colon ca. CaCo-2 | 18.8 | Melanoma Hs688(A).T | 0.0 |
| 83219 CC Well to Mod Diff | 14.7 | Melanoma* (met) Hs688(B).T | 1.6 |
| (ODO3866) | | | |
| Colon ca. HCC-2998 | 10.6 | Melanoma UACC-62 | 0.1 |
| Gastric ca.* (liver met) NCI-N87 | 10.5 | Melanoma M14 | 0.0 |
| Bladder | 0.9 | Melanoma LOX IMVI | 0.6 |
| Trachea | 3.5 | Melanoma* (met) SK-MEL-5 | 1.5 |
| Kidney | 0.5 | Adipose | 1.0 |

TABLE 37
Panel 2.2

| | Relative | | Relative |
|---|---|---|---|
| | Expression (%) | | Expression (%) |
| | 2.2x4tm6379 | | 2.2x4tm6379 |
| Tissue Name | t_ag2056_a1 | Tissue Name | t_ag2056_a1 |
| Normal Colon GENPAK 061003 | 5.3 | 83793 Kidney NAT (OD04348) | 10.3 |
| 97759 Colon cancer (OD06064) | 0.0 | 98938 Kidney malignant cancer | 1.3 |
| | | (OD06204B) | |
| 97760 Colon cancer NAT | 0.7 | 98939 Kidney normal adjacent | 0.8 |
| (OD06064) | | tissue (OD06204E) | |
| 97778 Colon cancer (OD06159) | 1.1 | 85973 Kidney Cancer (OD04450- | 0.0 |
| | | 01) | |
| 97779 Colon cancer NAT | 1.7 | 85974 Kidney NAT (OD04450-03) | 2.7 |
| (OD06159) | | | |
| 98861 Colon cancer (OD06297-04) | 2.9 | Kidney Cancer Clontech 8120613 | 2.1 |
| 98862 Colon cancer NAT | 4.2 | Kidney NAT Clontech 8120614 | 3.0 |
| (OD06297-015) | | | |
| 83237 CC Gr.2 ascend colon | 2.3 | Kidney Cancer Clontech 9010320 | 0.2 |
| (ODO3921) | | | |
| 83238 CC NAT (ODO3921) | 1.3 | Kidney NAT Clontech 9010321 | 0.6 |
| 97766 Colon cancer metastasis | 0.0 | Kidney Cancer Clontech 8120607 | 0.5 |
| (OD06104) | | | |
| 97767 Lung NAT (OD06104) | 0.0 | Kidney NAT Clontech 8120608 | 1.3 |
| 87472 Colon mets to lung | 4.1 | Normal Uterus GENPAK 061018 | 1.0 |
| (OD04451-01) | | | |
| 87473 Lung NAT (OD04451-02) | 0.0 | Uterus Cancer GENPAK 064011 | 0.6 |
| Normal Prostate Clontech A+ | 11.8 | Normal Thyroid Clontech A+ | 0.0 |
| 6546-1 (8090438) | | 6570-1 (7080817) | |
| 84140 Prostate Cancer (OD04410) | 14.1 | Thyroid Cancer GENPAK 064010 | 0.0 |
| 84141 Prostate NAT (OD04410) | 21.1 | Thyroid Cancer INVITROGEN | 1.4 |
| | | A302152 | |
| Normal Ovary Res. Gen. | 0.0 | Thyroid NAT INVITROGEN | 0.3 |
| | | A302153 | |
| 98863 Ovarian cancer (OD06283- | 0.3 | Normal Breast GENPAK 061019 | 2.9 |
| 03) | | | |
| 98865 Ovarian cancer | 0.3 | 84877 Breast Cancer (OD04566) | 2.0 |
| NAT/fallopian tube (OD06283-07) | | | |
| Ovarian Cancer GENPAK 064008 | 0.6 | Breast Cancer Res. Gen. 1024 | 8.5 |
| 97773 Ovarian cancer (OD06145) | 0.2 | 85975 Breast Cancer (OD04590- | 39.2 |
| | | 01) | |
| 97775 Ovarian cancer NAT | 0.8 | 85976 Breast Cancer Mets | 22.1 |
| (OD06145) | | (OD04590-03) | |
| 98853 Ovarian cancer (OD06455- | 3.5 | 87070 Breast Cancer Metastasis | 100.0 |
| 03) | | (OD04655-05) | |
| 98854 Ovarian NAT (OD06455- | 0.3 | GENPAK Breast Cancer 064006 | 5.7 |
| 07) Fallopian tube | | | |
| Normal Lung GENPAK 061010 | 0.2 | Breast Cancer Clontech 9100266 | 3.6 |
| 92337 Invasive poor diff. lung | 1.3 | Breast NAT Clontech 9100265 | 5.5 |
| adeno (ODO4945-01) | | | |
| 92338 Lung NAT (ODO4945-03) | 0.4 | Breast Cancer INVITROGEN | 1.6 |
| | | A209073 | |
| 84136 Lung Malignant Cancer | 6.9 | Breast NAT INVITROGEN | 5.5 |
| (OD03126) | | A2090734 | |
| 84137 Lung NAT (OD03126) | 0.0 | 97763 Breast cancer (OD06083) | 5.0 |
| 90372 Lung Cancer (OD05014A) | 4.6 | 97764 Breast cancer node | 11.7 |
| | | metastasis (OD06083) | |
| 90373 Lung NAT (OD05014B) | 0.7 | Normal Liver GENPAK 061009 | 2.5 |
| 97761 Lung cancer (OD06081) | 1.0 | Liver Cancer Research Genetics | 0.8 |
| | | RNA 1026 | |
| 97762 Lung cancer NAT | 0.0 | Liver Cancer Research Genetics | 2.7 |
| (OD06081) | | RNA 1025 | |
| 85950 Lung Cancer (OD04237-01) | 0.7 | Paired Liver Cancer Tissue | 2.0 |
| | | Research Genetics RNA 6004-T | |
| 85970 Lung NAT (OD04237-02) | 0.3 | Paired Liver Tissue Research | 2.0 |
| | | Genetics RNA 6004-N | |
| 83255 Ocular Mel Met to Liver | 3.7 | Paired Liver Cancer Tissue | 1.1 |
| (ODO4310) | | Research Genetics RNA 6005-T | |
| 83256 Liver NAT (ODO4310) | 0.5 | Paired Liver Tissue Research | 0.5 |
| | | Genetics RNA 6005-N | |
| 84139 Melanoma Mets to Lung | 0.0 | Liver Cancer GENPAK 064003 | 0.4 |
| (OD04321) | | | |
| 84138 Lung NAT (OD04321) | 0.5 | Normal Bladder GENPAK 061001 | 1.4 |
| Normal Kidney GENPAK 061008 | 0.8 | Bladder Cancer Research Genetics | 0.5 |
| | | RNA 1023 | |
| 83786 Kidney Ca. Nuclear grade 2 | 9.6 | Bladder Cancer INVITROGEN | 0.9 |
| (OD04338) | | A302173 | |
| 83787 Kidney NAT (OD04338) | 0.3 | Normal Stomach GENPAK | 0.6 |
| | | 061017 | |
| 83788 Kidney Ca Nuclear grade | 0.0 | Gastric Cancer Clontech 9060397 | 2.0 |
| 1/2 (OD04339) | | | |
| 83789 Kidney NAT (OD04339) | 0.9 | NAT Stomach Clontech 9060396 | 0.0 |
| 83790 Kidney Ca. Clear cell type | 0.0 | Gastric Cancer Clontech 9060395 | 0.2 |
| (OD04340) | | | |
| 83791 Kidney NAT (OD04340) | 1.2 | NAT Stomach Clontech 9060394 | 2.3 |
| 83792 Kidney Ca. Nuclear grade 3 | 1.1 | Gastric Cancer GENPAK 064005 | 1.4 |
| (OD04348) | | | |

TABLE 38
Panel 4D

| | Relative Expression (%) | |
|---|---|---|
| | 4dx4tm4455t_{—} | 4dx4tm4982t_{—} |
| Tissue Name | ag2056_a1 | ag2056_a1 |
| 93768_Secondary Th1_anti-CD28/anti-CD3 | 0.0 | 0.0 |
| 93769_Secondary Th2_anti-CD28/anti-CD3 | 0.3 | 0.5 |
| 93770_Secondary Tr1_anti-CD28/anti-CD3 | 0.0 | 0.0 |
| 93573_Secondary Th1_resting day 4-6 in IL-2 | 0.0 | 0.0 |
| 93572_Secondary Th2_resting day 4-6 in IL-2 | 0.0 | 0.0 |
| 93571_Secondary Tr1_resting day 4-6 in IL-2 | 0.0 | 0.0 |
| 93568_primary Th1_anti-CD28/anti-CD3 | 0.4 | 0.0 |
| 93569_primary Th2_anti-CD28/anti-CD3 | 0.0 | 0.0 |
| 93570_primary Tr1_anti-CD28/anti-CD3 | 0.4 | 0.0 |
| 93565_primary Th1_resting dy 4-6 in IL-2 | 0.0 | 0.0 |
| 93566_primary Th2_resting dy 4-6 in IL-2 | 0.0 | 0.0 |
| 93567_primary Tr1_resting dy 4-6 in IL-2 | 0.0 | 0.0 |
| 93351_CD45RA CD4 lymphocyte_anti-CD28/anti-CD3 | 0.0 | 0.0 |
| 93352_CD45RO CD4 lymphocyte_anti-CD28/anti-CD3 | 0.0 | 0.4 |
| 93251_CD8 Lymphocytes_anti-CD28/anti-CD3 | 0.0 | 0.0 |
| 93353_chronic CD8 Lymphocytes 2ry_resting dy 4-6 in IL-2 | 0.0 | 0.0 |
| 93574_chronic CD8 Lymphocytes 2ry_activated CD3/CD28 | 0.3 | 0.7 |
| 93354_CD4_none | 0.0 | 0.0 |
| 93252_Secondary Th1/Th2/Tr1_anti-CD95 CH11 | 0.0 | 0.0 |
| 93103_LAK cells_resting | 0.4 | 1.7 |
| 93788_LAK cells_IL-2 | 0.0 | 0.0 |
| 93787_LAK cells_IL-2 + IL-12 | 0.3 | 0.0 |
| 93789_LAK cells_IL-2 + IFN gamma | 0.4 | 0.0 |
| 93790_LAK cells_IL-2 + IL-18 | 0.0 | 0.0 |
| 93104_LAK cells_PMA/ionomycin and IL-18 | 0.2 | 0.3 |
| 93578_NK Cells IL-2_resting | 0.0 | 0.0 |
| 93109_Mixed Lymphocyte Reaction_Two Way MLR | 0.3 | 0.0 |
| 93110_Mixed Lymphocyte Reaction_Two Way MLR | 0.5 | 1.7 |
| 93111_Mixed Lymphocyte Reaction_Two Way MLR | 0.4 | 0.6 |
| 93112_Mononuclear Cells (PBMCs)_resting | 0.4 | 0.0 |
| 93113_Mononuclear Cells (PBMCs)_PWM | 0.0 | 0.0 |
| 93114_Mononuclear Cells (PBMCs)_PHA-L | 0.0 | 0.0 |
| 93249_Ramos (B cell)_none | 0.0 | 0.0 |
| 93250_Ramos (B cell)_ionomycin | 0.0 | 0.0 |
| 93349_B lymphocytes_PWM | 0.0 | 0.0 |
| 93350_B lymphoytes_CD40L and IL-4 | 0.0 | 0.0 |
| 92665_EOL-1 (Eosinophil)_dbcAMP differentiated | 0.0 | 0.0 |
| 93248_EOL-1 (Eosinophil)_dbcAMP/PMAionomycin | 0.0 | 0.0 |
| 93356_Dendritic Cells_none | 0.0 | 0.6 |
| 93355_Dendritic Cells_LPS 100 ng/ml | 0.3 | 0.7 |
| 93775_Dendritic Cells_anti-CD40 | 0.0 | 0.0 |
| 93774_Monocytes_resting | 0.0 | 0.0 |
| 93776_Monocytes_LPS 50 ng/ml | 0.0 | 0.0 |
| 93581_Macrophages_resting | 1.1 | 0.6 |
| 93582_Macrophages_LPS 100 ng/ml | 4.8 | 4.4 |
| 93098_HUVEC (Endothelial)_none | 0.0 | 0.0 |
| 93099_HUVEC (Endothelial)_starved | 0.0 | 0.7 |
| 93100_HUVEC (Endothelial)_IL-1b | 0.0 | 0.0 |
| 93779_HUVEC (Endothelial)_IFN gamma | 0.0 | 0.5 |
| 93102_HUVEC (Endothelial)_TNF alpha + IFN gamma | 0.0 | 0.0 |
| 93101_HUVEC (Endothelial)_TNF alpha + IL4 | 0.0 | 0.0 |
| 93781_HUVEC (Endothelial)_IL-11 | 0.8 | 0.5 |
| 93583_Lung Microvascular Endothelial Cells_none | 0.0 | 0.0 |
| 93584_Lung Microvascular Endothelial Cells_TNFa (4 ng/ml) and | 0.0 | 0.0 |
| IL1b (1 ng/ml) | | |
| 92662_Microvascular Dermal endothelium_none | 0.7 | 0.0 |
| 92663_Microsvasular Dermal endothelium_TNFa (4 ng/ml) | 0.4 | 0.0 |
| and IL1b (1 ng/ml) | | |
| 93773_Bronchial epithelium_TNFa (4 ng/ml) and IL1b (1 ng/ml)** | 4.3 | 44.0 |
| 93347_Small Airway Epithelium_none | 1.6 | 6.1 |
| 93348_Small Airway Epithelium_TNFa (4 ng/ml) | 17.8 | 18.0 |
| and IL1b (1 ng/ml) | | |
| 92668_Coronery Artery SMC_resting | 0.0 | 1.3 |
| 92669_Coronery Artery SMC_TNFa (4 ng/ml) and IL1b (1 ng/ml) | 1.0 | 1.1 |
| 93107_astrocytes_resting | 0.0 | 0.0 |
| 93108_astrocytes_TNFa (4 ng/ml) and IL1b (1 ng/ml) | 0.4 | 0.0 |
| 92666_KU-812 (Basophil)_resting | 0.8 | 0.4 |
| 92667_KU-812 (Basophil)_PMA/ionoycin | 0.4 | 1.1 |
| 93579_CCD1106 (Keratinocytes)_none | 6.4 | 9.7 |
| 93580_CCD1106 (Keratinocytes)_TNFa and iFNg** | 0.0 | 13.3 |
| 93791_Liver Cirrhosis | 0.1 | 1.4 |
| 93792_Lupus Kidney | 1.6 | 1.7 |
| 93577_NCI-H292 | 37.5 | 35.2 |
| 93358_NCI-H292_IL-4 | 19.8 | 19.1 |
| 93360_NCI-H292_IL-9 | 31.1 | 31.3 |
| 93359_NCI-H292_IL-13 | 8.6 | 9.7 |
| 93357_NCI-H292_IFN gamma | 9.6 | 10.7 |
| 93777_HPAEC_- | 0.0 | 0.0 |
| 93778_HPAEC_IL-1 beta/TNA alpha | 0.0 | 0.0 |
| 93254_Normal Human Lung Fibroblast_none | 33.5 | 41.6 |
| 93253_Normal Human Lung Fibroblast_TNFa | 16.3 | 25.0 |
| (4 ng/ml) and IL-1b (1 ng/ml) | | |
| 93257_Normal Human Lung Fibroblast_IL-4 | 77.1 | 77.1 |
| 93256_Normal Human Lung Fibroblast_IL-9 | 59.5 | 68.4 |
| 93255_Normal Human Lung Fibroblast_IL-13 | 51.1 | 69.2 |
| 93258_Normal Human Lung Fibroblast_IFN gamma | 100.0 | 100.0 |
| 93106_Dermal Fibroblasts CCD1070_resting | 0.0 | 1.4 |
| 93361_Dermal Fibroblasts CCD1070_TNF alpha 4 ng/ml | 0.0 | 0.0 |
| 93105_Dermal Fibroblasts CCD1070_IL-1 beta 1 ng/ml | 0.0 | 1.4 |
| 93772_dermal fibroblast_IFN gamma | 8.7 | 8.4 |
| 93771_dermal fibroblast_IL-4 | 16.4 | 25.3 |
| 93260_IBD Colitis 2 | 0.8 | 0.0 |
| 93261_IBD Crohns | 2.1 | 0.0 |
| 735010_Colon_normal | 11.9 | 13.2 |
| 735019_Lung_none | 8.1 | 5.2 |
| 64028-1_Thymus_none | 16.6 | 20.6 |
| 64030-1_Kidney_none | 0.9 | 1.2 |

TABLE 39
Panel CNS_1

| | Relative | | Relative |
|---|---|---|---|
| | Expression (%) | | Expression (%) |
| | cns1x4tm6169t_{—} | | cns1x4tm6169t_{—} |
| Tissue Name | ag2056_a2 | Tissue Name | ag2056_a2 |
| 102633_BA4 Control | 17.6 | 102605_BA17 PSP | 31.8 |
| 102641_BA4 Control2 | 36.2 | 102612_BA17 PSP2 | 7.4 |
| 102625_BA4 Alzheimer's2 | 15.0 | 102637_Sub Nigra Control | 9.2 |
| 102649_BA4 Parkinson's | 72.0 | 102645_Sub Nigra Control2 | 17.2 |
| 102656_BA4 Parkinson's2 | 93.7 | 102629_Sub Nigra Alzheimer's2 | 4.8 |
| 102664_BA4 Huntington's | 28.3 | 102660_Sub Nigra Parkinson's2 | 13.6 |
| 102671_BA4 Huntington's2 | 11.5 | 102667_Sub Nigra Huntington's | 15.1 |
| 102603_BA4 PSP | 12.3 | 102674_Sub Nigra Huntington's2 | 14.4 |
| 102610_BA4 PSP2 | 22.4 | 102614_Sub Nigra PSP2 | 0.9 |
| 102588_BA4 Depression | 21.3 | 102592_Sub Nigra Depression | 0.3 |
| 102596_BA4 Depression2 | 9.2 | 102599_Sub Nigra Depression2 | 2.9 |
| 102634_BA7 Control | 57.0 | 102636_Glob Palladus Control | 3.7 |
| 102642_BA7 Control2 | 53.1 | 102644_Glob Palladus Control2 | 7.3 |
| 102626_BA7 Alzheimer's2 | 12.0 | 102620_Glob Palladus Alzheimer's | 2.1 |
| 102650_BA7 Parkinson's | 32.7 | 102628_Glob Palladus | 8.1 |
| | | Alzheimer's2 | |
| 102657_BA7 Parkinson's2 | 59.7 | 102652_Glob Palladus Parkinson's | 72.2 |
| 102665_BA7 Huntington's | 58.6 | 102659_Glob Palladus | 8.6 |
| | | Parkinson's2 | |
| 102672_BA7 Huntington's2 | 55.5 | 102606_Glob Palladus PSP | 0.8 |
| 102604_BA7 PSP | 38.8 | 102613_Glob Palladus PSP2 | 5.3 |
| 102611_BA7 PSP2 | 18.4 | 102591_Glob Palladus Depression | 1.1 |
| 102589_BA7 Depression | 13.5 | 102638_Temp Pole Control | 10.8 |
| 102632_BA9 Control | 33.7 | 102646_Temp Pole Control2 | 34.8 |
| 102640_BA9 Control2 | 83.4 | 102622_Temp Pole Alzheimer's | 7.8 |
| 102617_BA9 Alzheimer's | 4.7 | 102630_Temp Pole Alzheimer's2 | 7.9 |
| 102624_BA9 Alzheimer's2 | 30.0 | 102653_Temp Pole Parkinson's | 43.0 |
| 102648_BA9 Parkinson's | 68.7 | 102661_Temp Pole Parkinson's2 | 51.6 |
| 102655_BA9 Parkinson's2 | 51.0 | 102668_Temp Pole Huntington's | 46.9 |
| 102663_BA9 Huntington's | 50.7 | 102607_Temp Pole PSP | 11.8 |
| 102670_BA9 Huntington's2 | 25.1 | 102615_Temp Pole PSP2 | 12.9 |
| 102602_BA9 PSP | 23.7 | 102600_Temp Pole Depression2 | 14.1 |
| 102609_BA9 PSP2 | 6.3 | 102639_Cing Gyr Control | 56.7 |
| 102587_BA9 Depression | 9.5 | 102647_Cing Gyr Control2 | 63.0 |
| 102595_BA9 Depression2 | 17.0 | 102623_Cing Gyr Alzheimer's | 11.5 |
| 102635_BA17 Control | 63.0 | 102631_Cing Gyr Alzheimer's2 | 12.7 |
| 102643_BA17 Control2 | 61.8 | 102654_Cing Gyr Parkinson's | 25.6 |
| 102627_BA17 Alzheimer's2 | 14.8 | 102662_Cing Gyr Parkinson's2 | 19.8 |
| 102651_BA17 Parkinson's | 64.3 | 102669_Cing Gyr Huntington's | 35.6 |
| 102658_BA17 Parkinson's2 | 100.0 | 102676_Cing Gyr Huntington's2 | 17.3 |
| 102666_BA17 Huntington's | 53.9 | 102608_Cing Gyr PSP | 12.4 |
| 102673_BA17 Huntington's2 | 22.0 | 102616_Cing Gyr PSP2 | 3.9 |
| 102590_BA17 Depression | 9.8 | 102594_Cing Gyr Depression | 10.5 |
| 102597_BA17 Depression2 | 40.5 | 102601_Cing Gyr Depression2 | 13.9 |

TABLE 40
Panel CNS_neurodegeneration_v1.0

| | Relative | | Relative |
|---|---|---|---|
| | Expression (%) | | Expression (%) |
| | tm7005t_{—} | | tm7005t_{—} |
| Tissue Name | ag2056_b1_s2 | Tissue Name | ag2056_b1_s2 |
| AD 1 Hippo | 9.7 | Control (Path) 3 Temporal Ctx | 2.6 |
| AD 2 Hippo | 19.2 | Control (Path) 4 Temporal Ctx | 31.2 |
| AD 3 Hippo | 3.8 | AD 1 Occipital Ctx | 9.8 |
| AD 4 Hippo | 6.1 | AD 2 Occipital Ctx (Missing) | 0.0 |
| AD 5 hippo | 100.0 | AD 3 Occipital Ctx | 3.6 |
| AD 6 Hippo | 26.9 | AD 4 Occipital Ctx | 12.5 |
| Control 2 Hippo | 14.3 | AD 5 Occipital Ctx | 11.1 |
| Control 4 Hippo | 4.7 | AD 6 Occipital Ctx | 27.6 |
| Control (Path) 3 Hippo | 3.2 | Control 1 Occipital Ctx | 1.4 |
| AD 1 Temporal Ctx | 6.6 | Control 2 Occipital Ctx | 44.9 |
| AD 2 Temporal Ctx | 23.5 | Control 3 Occipital Ctx | 19.6 |
| AD 3 Temporal Ctx | 9.0 | Control 4 Occipital Ctx | 3.6 |
| AD 4 Temporal Ctx | 19.4 | Control (Path) 1 Occipital Ctx | 62.4 |
| AD 5 Inf Temporal Ctx | 74.9 | Control (Path) 2 Occipital Ctx | 16.9 |
| AD 5 SupTemporal Ctx | 38.2 | Control (Path) 3 Occipital Ctx | 1.4 |
| AD 6 Inf Temporal Ctx | 28.5 | Control (Path) 4 Occipital Ctx | 24.9 |
| AD 6 Sup Temporal Ctx | 26.6 | Control 1 Parietal Ctx | 4.8 |
| Control 1 Temporal Ctx | 3.7 | Control 2 Parietal Ctx | 29.0 |
| Control 2 Temporal Ctx | 24.9 | Control 3 Parietal Ctx | 16.5 |
| Control 3 Temporal Ctx | 10.4 | Control (Path) 1 Parietal Ctx | 54.5 |
| Control 4 Temporal Ctx | 9.7 | Control (Path) 2 Parietal Ctx | 14.2 |
| Control (Path) 1 Temporal Ctx | 37.8 | Control (Path) 3 Parietal Ctx | 2.3 |
| Control (Path) 2 Temporal Ctx | 27.2 | Control (Path) 4 Parietal Ctx | 45.9 |

Panel 1.3D Summary The NOV6 gene is a homolog of synaptotagmin, and shows moderate to high expression across all brain regions with highest expression in the cerebral cortex (CT=27.6) Synaptotagmin is a presynaptic protein involved in synaptic vesicle release, making this an ideal drug target for diseases such as epilepsy, in which reduction of neurotransmission is beneficial. Selective inhibition of this gene or its protein product may therefore be useful in the treatment of seizure disorders. Furthermore, selective inhibition of neural transmission through antagonism of the protein encoded by the NOV6 gene may show therapeutic benefit in psychiatric diseases where it is believed that inappropriate neural connections have been established, such as schizophrenia and bipolar disorder. In addition, antibodies against synaptotagmin may cause Lambert-Eaton myasthenic syndrome. Therefore, peptide fragments of the protein encoded by the NOV6 gene may serve to block the action of these antibodies and treat Lambert-Eaton myasthenic syndrome.

The NOV6 gene also shows low but significant expression in many metabolic tissues including adipose, adult and fetal heart, adult and fetal liver, pancreas, and the adrenal, pituitary and thyroid glands. This gene product appears to be expressed at much higher levels in fetal skeletal muscle (CT value=31) when compared to adult skeletal muscle (CT value=37), and may be useful for the differentiation of the adult from the fetal phenotype in this tissue.

The NOV6 gene is significantly expressed in a cluster of cell lines derived from lung, gastric, colon and ovarian cancer compared to the normal tissues. The expression of this gene also shows an association with some normal brain and prostate samples when compared to the cell lines derived from cancers of these tissues. Thus, based upon its profile, the expression of this gene could be of use as a marker or as a therapeutic for lung, gastric, colon and ovarian cancers. In addition, therapeutic modulation of the product of this gene, through the use of peptides, antibodies, chimeric molecules or small molecule drugs, may be useful in the treatment of these cancers.

Panel 2.2 Summary Expression of the NOV6 gene is highest in a breast cancer metastasis (CT=27.8) and appears to be highly expressed in samples derived from breast cancer when compared to normal adjacent tissue. The expression of this gene also shows an association with some normal kidney, prostate and lung samples when compared to the matched kidney, prostate and lung cancer tissue. Thus, based upon its profile, absence/presence of expression of this gene could be of use as a marker for breast, kidney, prostate and lung cancer. Therapeutic modulation of the product of this gene, through the use of peptides, antibodies, chimeric molecules or small molecule drugs, may be useful in the therapy of lung, kidney, prostate and breast cancers.

Panel 4D Summary Results from two experiments with the same probe and primer set show that the NOV6 gene is selectively expressed, at moderate levels, in lung related tissues. Expression of the gene is found on normal human lung fibroblast and is up regulated in these cells following treatment with IFNg, IL4, ILI 3 and IL-9, with highest expression in IFNg treated cells (CTs=30). The protein encoded by the NOV6 gene is also up regulated in small airway epithelium treated with TNF-a and IL-1b and downregulated in the muco-epidemoid cell line H292 upon treatment with IL-13 and IFNg. The NOV6 gene is a homolog of synaptotagmin, whose ubiquitously expressed isoform, synaptotagmin VII, regulates exocytosis of lysosomes. Synaptotagmin VII has recently been implicated in fibroblast plasma membrane repair along with lysosomes which act as Ca(2+)-regulated exocytic compartments responsible for the plasma membrane repair. Therefore, therapeutic modulation of the expression or function of this gene or gene product, through the use of antibodies or small molecule drugs, might be beneficial for treating lung diseases such as asthma, emphysema, and viral and bacterial lung infection associated with cellular stress due to the local production of inflammatory cytokines.

Panel CNS_{—}1 Summary Highest expression of the NOV6 gene is seen in the brain of a patient with Parkinson's disease (CT=29.6). Please see Panel 10.3D for a discussion of potential utility in the central nervous system.

Panel CNS_neurodegeneration_v1.0 Summary Expression of the NOV6 gene is ubiquitous throughout the samples in this panel, with highest expression in the hippocampus of a patient with Alzheimer's disease (CT=25.8). While no association between the expression of this gene and the presence of Alzheimer's disease is detected in this panel, these results confirm the expression of this gene in areas that degenerate in Alzheimer's disease, including the cortex, hippocampus, amygdala and thalamus. Synaptotagmin expression is altered in the brain of Alzheimer's patients, possibly explaining impaired synaptogenesis and/or synaptosomal loss secondary to neuronal loss observed in the neurodegenerative disorder. It may also represent, reflect or account for the impaired neuronal transmission in Alzheimer's disease (AD), caused by deterioration of the exocytic machinery. Since the NOV6 gene is a homolog of synaptotagmin, agents that potentiate the expression or function of the protein encoded by the NOV6 gene may be useful in the treatment of Alzheimer's disease. (Reddy et al., Plasma membrane repair is mediated by Ca(2+)-regulated exocytosis of lysosomes. Cell 106:157-69, 2001; Takamori et al., Antibodies to calcium channel and synaptotagmin in Lambert-Eaton myasthenic syndrome. Am J Med Sci. 319:204-8, 2000; Sze et al., Selective regional loss of exocytotic presynaptic vesicle proteins in Alzheimer's disease brains. J Neurol Sci. 175:81-90, 2000; Sokolov et al., Levels of mRNAs encoding synaptic vesicle and synaptic plasma membrane proteins in the temporal cortex of elderly schizophrenic patients. Biol Psychiatry. 48:184-96, 2000; Masliah et al., Altered expression of synaptic proteins occurs early during progression of Alzheimer's disease. Neurology 56:127-9, 2001; Yoo et al., Synaptosomal proteins, beta-soluble N-ethylmaleimide-sensitive factor attachment protein (beta-SNAP), gamma-SNAP and synaptotagmin I in brain of patients with Down syndrome and Alzheimer's disease. Dement Geriatr Cogn Disord. 12:219-25, 2001).

NOV8: Glypican 2 Precursor-Like

Expression of the NOV8a gene (134913441 _EXT) and variants NOV8b (CG50970-O₂) and NOV8c (CG50970-03) was assessed using the primer-probe sets Ag1309 and Ag2251 described in Tables 41 and 42. Results from RTQ-PCR runs are shown in Tables 43, 44, 45, and 46.

TABLE 41
Probe Name Ag1309

| | | | | Start | SEQ ID |
|---|---|---|---|---|---|
| Primers | Sequences | TM | Length | Position | NO: |
| Forward | 5′-ACTCTCTGACCCAGCTCTTCTC-3′ | 59.3 | 22 | 412 | 126 |
| Probe | TET-5′-CCACTCCTACGGCCGCCTGTATG-3′-TAMRA | 70.6 | 23 | 434 | 127 |
| Reverse | 5′-GAGAACAGGCCATTGAATATGA-3′ | 59 | 22 | 469 | 128 |

TABLE 42
Probe Name Ag2251

| | | | | Start | SEQ ID |
|---|---|---|---|---|---|
| Primers | Sequences | TM | Length | Position | NO: |
| Forward | 5′-ACTCTCTGACCCAGCTCTTCTC-3′ | 59.3 | 22 | 359 | 129 |
| Probe | TET-5′-CCACTCCTACGGCCGCCTGTATG-3′-TAMRA | 70.6 | 23 | 381 | 130 |
| Reverse | 5′-GAGAACAGGCCATTGAATATGA-3′ | 59 | 22 | 416 | 131 |

TABLE 43
Panel 1.3D

| | Relative | | Relative |
|---|---|---|---|
| | Expression (%) | | Expression (%) |
| | 1.3dtm4197t_{—} | | 1.3dtm417t_{—} |
| Tissue Name | ag2251 | Tissue Name | ag2251 |
| Liver adenocarcinoma | 0.9 | Kidney (fetal) | 1.9 |
| Pancreas | 0.4 | Renal ca. 786-0 | 1.0 |
| Pancreatic ca. CAPAN 2 | 0.4 | Renal ca. A498 | 4.5 |
| Adrenal gland | 0.6 | Renal ca. RXF 393 | 0.0 |
| Thyroid | 0.4 | Renal ca. ACHN | 0.3 |
| Salivary gland | 1.2 | Renal ca. UO-31 | 2.8 |
| Pituitary gland | 0.7 | Renal ca. TK-10 | 3.8 |
| Brain (fetal) | 73.7 | Liver | 0.0 |
| Brain (whole) | 4.6 | Liver (fetal) | 1.7 |
| Brain (amygdala) | 6.4 | Liver ca. (hepatoblast) HepG2 | 1.8 |
| Brain (cerebellum) | 1.8 | Lung | 0.0 |
| Brain (hippocampus) | 22.2 | Lung (fetal) | 3.1 |
| Brain (substantia nigra) | 2.1 | Lung ca (small cell) LX-1 | 4.5 |
| Brain (thalamus) | 4.5 | Lung ca. (small cell) NCI-H69 | 8.7 |
| Cerebral Cortex | 3.5 | Lung ca. (s.cell var.) SHP-77 | 25.7 |
| Spinal cord | 3.2 | Lung ca. (large cell)NCI-H460 | 2.5 |
| CNS ca. (glio/astro) U87-MG | 4.3 | Lung ca. (non-sm. cell) A549 | 2.8 |
| CNS ca. (glio/astro) U-118-MG | 2.2 | Lung ca. (non-s.cell) NCI-H23 | 12.4 |
| CNS ca. (astro) SW1783 | 14.3 | Lung ca. (non-s.cell) HOP-62 | 1.7 |
| CNS ca.* (neuro; met) SK-N-AS | 100.0 | Lung ca. (non-s.cl) NCI-H522 | 28.1 |
| CNS ca. (astro) SF-539 | 0.5 | Lung ca. (squam.) SW 900 | 2.1 |
| CNS ca. (astro) SNB-75 | 13.0 | Lung ca. (squam.) NCI-H596 | 0.7 |
| CNS ca. (glio) SNB-19 | 14.7 | Mammary gland | 1.0 |
| CNS ca. (glio) U251 | 3.6 | Breast ca.* (pl. effusion) MCF-7 | 4.0 |
| CNS ca. (glio) SF-295 | 3.6 | Breast ca.* (pl.ef) MDA-MB-231 | 1.1 |
| Heart (fetal) | 3.4 | Breast ca.* (pl. effusion) T47D | 1.1 |
| Heart | 0.0 | Breast ca. BT-549 | 16.3 |
| Fetal Skeletal | 15.2 | Breast ca. MDA-N | 6.4 |
| Skeletal muscle | 0.0 | Ovary | 3.2 |
| Bone marrow | 1.8 | Ovarian ca. OVCAR-3 | 1.7 |
| Thymus | 21.2 | Ovarian ca. OVCAR-4 | 0.8 |
| Spleen | 0.8 | Ovarian ca. OVCAR-5 | 2.3 |
| Lymph node | 1.1 | Ovarian ca. OVCAR-8 | 7.3 |
| Colorectal | 0.8 | Ovarian ca. IGROV-1 | 2.4 |
| Stomach | 0.6 | Ovarian ca.* (ascites) SK-OV-3 | 0.6 |
| Small intestine | 2.6 | Uterus | 0.8 |
| Colon ca. SW480 | 2.5 | Placenta | 0.8 |
| Colon ca.* (SW480 met)SW620 | 1.5 | Prostate | 1.1 |
| Colon ca HT29 | 1.7 | Prostate ca.* (bone met)PC-3 | 3.2 |
| Colon ca. HCT-116 | 2.4 | Testis | 69.7 |
| Colon ca. CaCo-2 | 2.5 | Melanoma Hs688(A) T | 0.0 |
| 83219 CC Well to Mod Diff | 2.2 | Melanoma* (met) Hs688(B).T | 0.0 |
| (ODO3866) | | | |
| Colon ca. HCC-2998 | 2.0 | Melanoma UACC-62 | 0.4 |
| Gastric ca.* (liver met) NCI-N87 | 0.8 | Melanoma M14 | 2.6 |
| Bladder | 1.0 | Melanoma LOX IMVI | 0.7 |
| Trachea | 1.8 | Melanoma* (met) SK-MEL-5 | 5.6 |
| Kidney | 0.7 | Adipose | 0.0 |

TABLE 44
Panel 2D

| | Relative | | Relative |
|---|---|---|---|
| | Expression (%) | | Expression (%) |
| | 2dtm4198t_{—} | | 2dtm4198t_{—} |
| Tissue Name | ag2251 | Tissue Name | ag2251 |
| Normal Colon GENPAK 061003 | 5.5 | Kidney NAT Clontech 8120608 | 0.0 |
| 83219 CC Well to Mod Diff | 4.5 | Kidney Cancer Clontech 8120613 | 0.0 |
| (ODO3866) | | | |
| 83220 CC NAT (ODO3866) | 2.6 | Kidney NAT Clontech 8120614 | 0.6 |
| 83221 CC Gr 2 rectosigmoid | 1.2 | Kidney Cancer Clontech 9010320 | 0.0 |
| (ODO3868) | | | |
| 83222 CC NAT (ODO3868) | 1.1 | Kidney NAT Clontech 9010321 | 1.3 |
| 83235 CC Mod Diff (ODO3920) | 5.8 | Normal Uterus GENPAK 061018 | 1.1 |
| 83236 CC NAT (ODO3920) | 2.3 | Uterus Cancer GENPAK 064011 | 3.0 |
| 83237 CC Gr.2 ascend colon | 4.1 | Normal Thyroid Clontech A+ | 0.6 |
| (ODO3921) | | 6570-1 | |
| 83238 CC NAT (ODO3921) | 0.0 | Thyroid Cancer GENPAK 064010 | 0.6 |
| 83241 CC from Partial | 1.3 | Thyroid Cancer INVITROGEN | 0.4 |
| Hepatectomy (ODO4309) | | A302152 | |
| 83242 Liver NAT (ODO4309) | 0.0 | Thyriod NAT INVITROGEN | 2.3 |
| | | A302153 | |
| 87472 Colon mets to lung | 4.3 | Normal Breast GENPAK 061019 | 4.4 |
| (OD04451-01) | | | |
| 87473 Lung NAT (OD04451-02) | 0.0 | 84877 Breast Cancer (OD04566) | 1.2 |
| Normal Prostate Clontech A+ | 0.0 | 85975 Breast Cancer (OD04590- | 100.0 |
| 6546-1 | | 01) | |
| 84140 Prostate Cancer (OD04410) | 3.4 | 85976 Breast Cancer Mets | 1.5 |
| | | (OD04590-03) | |
| 84141 Prostate NAT (OD04410) | 0.0 | 87070 Breast Cancer Metastasis | 3.7 |
| | | (OD04655-05) | |
| 87073 Prostate Cancer (OD04720- | 0.6 | GENPAK Breast Cancer 064006 | 6.8 |
| 01) | | | |
| 87074 Prostate NAT (OD04720- | 1.8 | Breast Cancer Res. Gen. 1024 | 10.4 |
| 02) | | | |
| Normal Lung GENPAK 061010 | 5.1 | Breast Cancer Clontech 9100266 | 6.6 |
| 83239 Lung Met to Muscle | 0.0 | Breast NAT Clontech 9100265 | 3.4 |
| (ODO4286) | | | |
| 83240 Muscle NAT (ODO4286) | 0.6 | Breast Cancer INVITROGEN | 7.9 |
| | | A209073 | |
| 84136 Lung Malignant Cancer | 3.9 | Breast NAT INVITROGEN | 2.5 |
| (OD03126) | | A2090734 | |
| 84137 Lung NAT (OD03126) | 0.0 | Normal Liver GENPAK 061009 | 0.0 |
| 84871 Lung Cancer (OD04404) | 0.0 | Liver Cancer GENPAK 064003 | 0.6 |
| 84872 Lung NAT (OD04404) | 0.6 | Liver Cancer Research Genetics | 0.0 |
| | | RNA 1025 | |
| 84875 Lung Cancer (OD04565) | 0.6 | Liver Cancer Research Genetics | 0.6 |
| | | RNA 1026 | |
| 84876 Lung NAT (OD04565) | 0.0 | Paired Liver Cancer Tissue | 0.0 |
| | | Research Genetics RNA 6004-T | |
| 85950 Lung Cancer (OD04237-01) | 99.3 | Paired Liver Tissue Research | 0.6 |
| | | Genetics RNA 6004-N | |
| 85970 Lung NAT (OD04237-02) | 2.4 | Paired Liver Cancer Tissue | 1.1 |
| | | Research Genetics RNA 6005-T | |
| 83255 Ocular Mel Met to Liver | 0.7 | Paired Liver Tissue Research | 0.0 |
| (ODO4310) | | Genetics RNA 6005-N | |
| 83256 Liver NAT (ODO4310) | 0.0 | Normal Bladder GENPAK 061001 | 1.8 |
| 84139 Melanoma Mets to Lung | 18.0 | Bladder Cancer Research Genetics | 2.8 |
| (OD04321) | | RNA 1023 | |
| 84138 Lung NAT (OD04321) | 0.6 | Bladder Cancer INVITROGEN | 13.2 |
| | | A302173 | |
| Normal Kidney GENPAK 061008 | 1.4 | 87071 Bladder Cancer | 0.0 |
| | | (OD04718-01) | |
| 83786 Kidney Ca, Nuclear grade 2 | 8.0 | 87072 Bladder Normal Adjacent | 1.3 |
| (OD04338) | | (OD04718-03) | |
| 83787 Kidney NAT (OD04338) | 0.0 | Normal Ovary Res. Gen. | 2.8 |
| 83788 Kidney Ca Nuclear grade | 2.4 | Ovarian Cancer GENPAK 064008 | 4.3 |
| 1/2 (OD04339) | | | |
| 83789 Kidney NAT (OD04339) | 0.0 | 87492 Ovary Cancer (OD04768- | 4.0 |
| | | 07) | |
| 83790 Kidney Ca, Clear cell type | 1.2 | 87493 Ovary NAT (OD04768-08) | 0.0 |
| (OD04340) | | | |
| 83791 Kidney NAT (OD04340) | 1.0 | Normal Stomach GENPAK | 0.8 |
| | | 061017 | |
| 83792 Kidney Ca, Nuclear grade 3 | 0.0 | Gastric Cancer Clontech 9060358 | 0.3 |
| (OD04348) | | | |
| 83793 Kidney NAT (OD04348) | 0.8 | NAT Stomach Clontech 9060359 | 1.2 |
| 87474 Kidney Cancer (OD04622- | 1.1 | Gastric Cancer Clontech 9060395 | 0.0 |
| 01) | | | |
| 87475 Kidney NAT (OD04622-03) | 0.0 | NAT Stomach Clontech 9060394 | 1.5 |
| 85973 Kidney Cancer (OD04450- | 4.6 | Gastric Cancer Clontech 9060397 | 6.8 |
| 01) | | | |
| 85974 Kidney NAT (OD04450-03) | 0.6 | NAT Stomach Clontech 9060396 | 0.0 |
| Kidney Cancer Clontech 8120607 | 0.6 | Gastric Cancer GENPAK 064005 | 2.5 |

TABLE 45
Panel 4D

| | Relative | Relative |
|---|---|---|
| | Expression (%) | Expression (%) |
| | 4dtm4199t_{—} | 4Dtm1886t_{—} |
| Tissue Name | ag2251 | ag1309 |
| 93768_Secondary Th1_anti-CD28/anti-CD3 | 1.6 | 1.5 |
| 93769_Secondary Th2_anti-CD28/anti-CD3 | 1.2 | 1.0 |
| 93770_Secondary Tr1_anti-CD28/anti-CD3 | 1.7 | 2.0 |
| 93573_Secondary Th1_resting day 4-6 in IL-2 | 0.5 | 1.7 |
| 93572_Secondary Th2_resting day 4-6 in IL-2 | 0.6 | 1.4 |
| 93571_Secondary Tr1_resting day 4-6 in IL-2 | 1.2 | 1.4 |
| 93568_primary Th1_anti-CD28/anti-CD3 | 2.7 | 1.7 |
| 93569_primary Th2_anti-CD28/anti-CD3 | 1.9 | 3.4 |
| 93570_primary Tr1_anti-CD28/anti-CD3 | 1.2 | 5.9 |
| 93565_primary Th1_resting dy 4-6 in IL-2 | 17.1 | 12.5 |
| 93566_primary Th2_resting dy 4-6 in IL-2 | 8.6 | 6.5 |
| 93567_primary Tr1_resting dy 4-6 in IL-2 | 2.4 | 3.7 |
| 93351_CD45RA CD4 lymphocyte_anti-CD28/anti-CD3 | 1.2 | 3.2 |
| 93352_CD45RO CD4 lymphocyte_anti-CD28/anti-CD3 | 2.7 | 4.3 |
| 93251_CD8 Lymphocytes_anti-CD28/anti-CD3 | 1.9 | 1.1 |
| 93353_chronic CD8 Lymphocytes 2ry_resting dy 4-6 in IL-2 | 0.9 | 1.7 |
| 93574_chronic CD8 Lymphocytes 2ry_activated CD3/CD28 | 1.0 | 1.1 |
| 93354_CD4_none | 0.5 | 1.4 |
| 93252_Secondary Th1/Th2/Tr1_anti-CD95 CH11 | 1.7 | 4.5 |
| 93103_LAK cells_resting | 1.6 | 1.2 |
| 93788_LAK cells_IL-2 | 1.8 | 3.1 |
| 93787_LAK cells_IL-2 + IL-12 | 0.7 | 1.8 |
| 93789_LAK cells_IL-2 + IFN gamma | 1.3 | 1.7 |
| 93790_LAK cells_IL-2 + IL-18 | 1.4 | 1.5 |
| 93104_LAK cells_PMA/ionomycin and IL-18 | 0.0 | 0.8 |
| 93578_NK Cells IL-2_resting | 1.1 | 0.9 |
| 93109_Mixed Lymphocyte Reaction_Two Way MLR | 2.3 | 1.6 |
| 93110_Mixed Lymphocyte Reaction_Two Way MLR | 0.3 | 1.7 |
| 93111_Mixed Lymphocyte Reaction_Two Way MLR | 0.4 | 0.8 |
| 93112_Mononuclear Cells (PBMCs)_resting | 0.0 | 0.4 |
| 93113_Mononuclear Cells (PBMCs)_PWM | 2.1 | 6.1 |
| 93114_Mononuclear Cells (PBMCs)_PHA-L | 5.7 | 9.9 |
| 93249_Ramos (B cell)_none | 6.2 | 13.6 |
| 93250_Ramos (B cell)_ionomycin | 24.3 | 34.9 |
| 93349_B lymphocytes_PWM | 7.3 | 7.4 |
| 93350_B lymphoytes_CD40L and IL-4 | 4.4 | 2.7 |
| 92665_EOL-1 (Eosinophil)_dbcAMP differentiated | 2.3 | 2.6 |
| 93248_EOL-1 (Eosinophil)_dbcAMP/PMAionomycin | 1.3 | 0.3 |
| 93356_Dendritic Cells_none | 0.8 | 0.5 |
| 93355_Dendritic Cells_LPS 100 ng/ml | 0.0 | 0.0 |
| 93775_Dendritic Cells_anti-CD40 | 0.0 | 0.3 |
| 93774_Monocytes_resting | 0.0 | 0.3 |
| 93776_Monocytes_LPS 50 ng/ml | 0.3 | 1.1 |
| 93581_Macrophages_resting | 1.3 | 0.6 |
| 93582_Macrophages_LPS 100 ng/ml | 0.0 | 0.0 |
| 93098_HUVEC (Endothelial)_none | 3.5 | 5.3 |
| 93099_HUVEC (Endothelial)_starved | 12.4 | 12.7 |
| 93100_HUVEC (Endothelial)_IL-1b | 1.6 | 1.3 |
| 93779_HUVEC (Endothelial)_IFN gamma | 2.5 | 2.9 |
| 93102_HUVEC (Endothelial)_TNF alpha + IFN gamma | 0.1 | 1.5 |
| 93101_HUVEC (Endothelial)_TNF alpha + IL4 | 2.6 | 3.5 |
| 93781_HUVEC (Endothelial)_IL-11 | 1.4 | 4.4 |
| 93583_Lung Microvascular Endothelial Cells_none | 2.3 | 1.3 |
| 93584_Lung Microvascular Endothelial Cells_TNFa (4 ng/ml) and | 1.7 | 2.1 |
| IL1b (1 ng/ml) | | |
| 92662_Microvascular Dermal endothelium_none | 2.6 | 6.1 |
| 92663_Microsvasular Dermal endothelium_TNFa (4 ng/ml) and IL1b | 1.3 | 2.0 |
| (1 ng/ml) | | |
| 93773_Bronchial epithelium_TNFa (4 ng/ml) and IL1b (1 ng/ml)** | 1.6 | 2.9 |
| 93347_Small Airway Epithelium_none | 0.4 | 0.8 |
| 93348_Small Airway Epithelium_TNFa (4 ng/ml) and IL1b (1 ng/ml) | 1.5 | 3.1 |
| 92668_Coronery Artery SMC_resting | 1.1 | 1.3 |
| 92669_Coronery Artery SMC_TNFa (4 ng/ml) and IL1b (1 ng/ml) | 2.0 | 1.4 |
| 93107_astrocytes_resting | 22.5 | 17.8 |
| 93108_astrocytes_TNFa (4 ng/ml) and IL1b (1 ng/ml) | 4.7 | 6.2 |
| 92666_KU-812 (Basophil)_resting | 0.2 | 0.3 |
| 92667_KU-812 (Basophil)_PMA/ionoycin | 0.3 | 1.2 |
| 93579_CCD1106 (Keratinocytes)_none | 3.9 | 3.9 |
| 93580_CCD1106 (Keratinocytes)_TNFa and IFNg** | 3.1 | 19.5 |
| 93791_Liver Cirrhosis | 2.6 | 2.0 |
| 93792_Lupus Kidney | 0.0 | 0.3 |
| 93577_NCI-H292 | 0.4 | 0.7 |
| 93358_NCI-H292_IL-4 | 0.4 | 1.7 |
| 93360_NCI-H292_IL-9 | 1.6 | 0.0 |
| 93359_NCI-H292_IL-13 | 1.6 | 0.6 |
| 93357_NCI-H292_IFN gamma | 0.3 | 0.0 |
| 93777_HPAEC_- | 2.0 | 3.3 |
| 93778_HPAEC_IL-1 beta/TNA alpha | 0.6 | 1.6 |
| 93254_Normal Human Lung Fibroblast_none | 3.4 | 3.7 |
| 93253_Normal Human Lung Fibroblast_TNFa (4 ng/ml) | 1.5 | 1.6 |
| and IL-1b (1 ng/ml) | | |
| 93257_Normal Human Lung Fibroblast_IL-4 | 2.8 | 3.6 |
| 93256_Normal Human Lung Fibroblast_IL-9 | 3.2 | 2.6 |
| 93255_Normal Human Lung Fibroblast_IL-13 | 2.8 | 2.7 |
| 93258_Normal Human Lung Fibroblast_IFN gamma | 1.9 | 0.5 |
| 93106_Dermal Fibroblasts CCD1070_resting | 3.7 | 4.2 |
| 93361_Dermal Fibroblasts CCD1070_TNF alpha 4 ng/ml | 4.2 | 2.4 |
| 93105_Dermal Fibroblasts CCD1070_IL-1 beta 1 ng/ml | 2.5 | 1.3 |
| 93772_dermal fibroblast_IFN gamma | 0.2 | 0.7 |
| 93771_dermal fibroblast_IL-4 | 0.8 | 0.7 |
| 93260_IBD Colitis 2 | 0.0 | 0.2 |
| 93261_IBD Crohns | 0.0 | 0.0 |
| 735010_Colon_normal | 4.2 | 3.1 |
| 735019_Lung_none | 1.3 | 1.5 |
| 64028-1_Thymus_none | 0.3 | 1.6 |
| 64030-1_Kidney_none | 100.0 | 100.0 |

TABLE 46
Panel CNS_neurodegeneration_v1.0

| | Relative | | Relative |
|---|---|---|---|
| | Expression (%) | | Expression (%) |
| | tm6901t_{—} | | tm6901t_{—} |
| Tissue Name | ag2251_a2s2 | Tissue Name | ag2251_a2s2 |
| 106655_4951 Hippo | 19.7 | 106677_4624 BA21 | 3.4 |
| 106657_4986 Hippo | 35.8 | 106681_4640 BA21 | 34.1 |
| 106652_4933 Hippo | 7.2 | 106654_4951 BA17 | 19.5 |
| 106649_4901 Hippo | 9.7 | cns_water | 0.0 |
| 110138_3087 hippo | 59.0 | 106651_4933 BA17 | 17.3 |
| 110121_3027 Hippo | 97.7 | 106648_4901 BA17 | 19.1 |
| 106670_4971 Hippo | 37.0 | 110123_3027 Occ Ctx | 52.8 |
| 106666_4867 Hippo | 34.8 | 110140_3087 occ ctx | 41.9 |
| 106680_4624 Hippo | 17.2 | 106659_4595 BA17 | 6.3 |
| 106653_4951 BA21 | 20.5 | 106668_4971 BA17 | 51.5 |
| 106656_4986 BA21 | 33.7 | 106662_4737 BA17 | 22.9 |
| 106650_4933 BA21 | 9.8 | 106665_4867 BA17 | 6.6 |
| 106647_4901 BA21 | 36.0 | 106675_3975 BA17 | 73.6 |
| 110136_3087 inf temp ctx | 76.8 | 106672_3954 BA17 | 16.7 |
| 110137_3087 sup temp ctx | 97.7 | 106678_4624 BA17 | 11.8 |
| 110118_3027 Inf Temp Ctx | 59.8 | 106682_4640 BA17 | 28.1 |
| 110119_3027 Sup Temp Ctx | 100.0 | 106660_4595 BA7 | 12.0 |
| 106658_4595 BA21 | 9.8 | 113670_106669 pool | 62.2 |
| 106667_4971 BA21 | 29.9 | 106663_4737 BA7 | 20.3 |
| 106661_4737 BA21 | 10.5 | 106676_3975 BA7 | 43.7 |
| 106664_4867 BA21 | 34.1 | 106673_3954 BA7 | 14.9 |
| 106674_3975 BA21 | 63.8 | 106679_4624 BA7 | 7.8 |
| 106671_3954 BA21 | 13.7 | 106683_4640 BA7 | 29.8 |

Panel 1.3D Summary Ag2251 The highest level of expression of the NOVS gene is seen in a CNS cancer cell line SK-N-AS (CT=29.6). The gene is also expressed at higher levels in cell lines derived from lung, prostate, and breast cancers compared to the normal tissues and may play a role in these cancers. Thus, expression of the NOV8 gene could be used as a marker or as a therapeutic for lung, prostate and breast cancer. In addition, therapeutic modulation of the activity of the product of this gene, through the use of peptides, antibodies, chimeric molecules or small molecule drugs, may be useful in the treatment of these cancers.

The NOV8 gene is also expressed at higher levels in fetal liver, lung, skeletal muscle, and heart (CTs=32-35) when compared to the expression in adult tissues (CTs=40). These results suggest that expression of the NOV8 gene could potentially be used to distinguish between the adult and fetal phenotypes of these tissues. Furthermore, the difference in expression in fetal and adult tissue may also indicate an involvement of the gene product in the differentiation processes leading to the formation of the adult organs. Therefore, the protein encoded by the NOV8 gene could potentially play a role in the regeneration of these tissues in the adult.

The NOV8 gene, a glypican homolog, is expressed at moderate to low levels across many regions of the brain. These regions include the hippocampus, amygdala, thalamus and cerebral cortex, all of which are key regions subject to Alzheimer's disease neurodegeneration. Furthermore, glypican is expressed in senile plaques and neurofibrillary tangles, also indicating a role in Alzheimer's disease. Therefore, the expression profile of the NOV8 gene suggests that antibodies against the protein encoded by the NOV8 gene can be used to distinguish neurodegenerative disease in the human brain. Furthermore, since NOV8 gene-product-like substances are components of senile plaques which are thought to give rise to the dementia pathology of Alzheimer's disease, agents that target this gene and disrupt its role in senile plaques may have utility in treating the cause and symptoms or Alzheimer's disease as well as other neurodegenerative diseases that involve this glypican.

Panel 2D Summary A-2251 The highest expression of NOV8 gene is seen in a breast cancer sample (CT=30.3). The expression of this gene appears to show an association with samples derived from colon, lung, kidney, breast, bladder and gastric cancers when compared to the matched normal tissue. Thus, expression of the NOV8 gene could be used as a marker for these cancers. In addition, therapeutic activity of the product of this gene, through the use of peptides, antibodies, chimeric molecules or small molecule drugs, may be useful in the treatment of colon, lung, kidney, breast, bladder and gastric cancers.

Panel 4D Summary Ag2251/Ag1309 Two experiments using two different probe and primer sets produce results that are in very good agreement, with highest expression seen in the kidney (CTs=28-29). This high level of expression in the kidney suggests that expression of the NOV8 gene can serve as a marker for kidney tissue. The NOV8 gene is also expressed at low level in activated Ramos B cell line, in activated primary B cells, Th1 T cells, activated HUVEC and keratinocytes. The NOV8 gene encodes for a protein that is a homolog of a glypican molecule, which belongs to the family of HSPG (heparan sulfate proteoglycans). Glypicans can regulate the activism of a wide variety of growth and survival factors. Therefore, therapeutic modulation of the expression or function of this gene or gene product, through the use of antibody drugs could potentially prevent T and B cell activation in the treatment of autoimmune mediated diseases such as insulin-dependent diabetes mellitus, rheumatoid arthritis, Crohn's disease, allergies delayed type hypersensitivity, asthma, and psoriasis.

Panel CNS_neurodegeneration_v1.0 Summary Ag2251 Highest expression of the NOV8 gene in this panel is detected in the cerebral cortex of an Alzheimer's patient (CT=32.7). While no association between the expression of this gene and the presence of Alzheimer's disease is detected in this panel, these results confirm the expression of this gene in areas that degenerate in Alzheimer's disease. Please see Panel 1.3D for a discussion of potential utility of this gene in the central nervous system. (Verbeek et al., Agrin is a major heparan sulfate proteoglycan accumulating in Alzheimer's disease brain. Am J Pathol. 155:2115-25, 1999).

NOV9: Mitogen-Activtivated-Protein Kinase Kinase 2-like

Expression of NOV9 gene (also referred to as AC011005_da2) %,as assessed using the primer-probe set Ag2022 described in Tables 47. Results from RTQ-PCR runs are shown in Tables 48, 49, and 50.

TABLE 47
Probe Name Ag2022

| | | | | Start | SEQ ID |
|---|---|---|---|---|---|
| Primers | Sequences | TM | Length | Position | NO: |
| Forward | 5′-CCAGGAGTTTGTCAATAAATGC-3′ | 586 | 22 | 800 | 132 |
| Probe | FAM-5′-CTCATCAAGAACCCAGCGGAGCG-3′-TAMRA | 712 | 23 | 822 | 133 |
| Reverse | 5′-TTGATGAAGCTGTGGTTTGTG-3′ | 59.5 | 21 | 863 | 134 |

TABLE 48
Panel 1.3D

| | Relative Expression (%) | |
|---|---|---|
| | 1.3dx4tm5437 | 1.3dx4tm5441 |
| Tissue Name | f_ag2022_b1 | f_ag2022_a1 |
| Liver adenocarcinoma | 23.1 | 15.9 |
| Pancreas | 9.6 | 4.3 |
| Pancreatic ca. CAPAN 2 | 4.1 | 4.4 |
| Adrenal gland | 7.9 | 10.3 |
| Thyroid | 12.1 | 9.7 |
| Salivary gland | 10.9 | 5.9 |
| Pituitary gland | 12.0 | 9.6 |
| Brain (fetal) | 13.6 | 7.6 |
| Brain (whole) | 47.3 | 25.1 |
| Brain (amygdala) | 33.7 | 19.9 |
| Brain (cerebellum) | 33.2 | 16.3 |
| Brain (hippocampus) | 42.8 | 21.7 |
| Brain (substantia nigra) | 30.6 | 13.8 |
| Brain (thalamus) | 50.3 | 24.6 |
| Cerebral Cortex | 36.5 | 31.4 |
| Spinal cord | 16.9 | 8.7 |
| CNS ca. (glio/astro) U87-MG | 17.6 | 18.5 |
| CNS ca. (glia/astro) U-118-MG | 54.6 | 38.5 |
| CNS ca. (astro) SW1783 | 13.5 | 12.8 |
| CNS ca.* (neuro; met) SK-N-AS | 15.4 | 12.9 |
| CNS ca. (astro) SF-539 | 14.3 | 9.5 |
| CNS ca. (astro) SNB-75 | 29.6 | 25.8 |
| CNS ca. (glio) SNB-19 | 23.7 | 17.9 |
| CNS ca. (glio) U251 | 38.4 | 34.5 |
| CNS ca. (glio) SF-295 | 18.5 | 17.2 |
| Heart (fetal) | 17.1 | 16.3 |
| Heart | 25.8 | 10.3 |
| Fetal Skeletal | 12.2 | 12.9 |
| Skeletal muscle | 100.0 | 100.0 |
| Bone marrow | 15.0 | 14.5 |
| Thymus | 7.6 | 8.1 |
| Spleen | 14.5 | 11.4 |
| Lymph node | 25.7 | 19.2 |
| Colorectal | 6.7 | 4.7 |
| Stomach | 14.4 | 10.1 |
| Small intestine | 30.2 | 32.3 |
| Colon ca. SW480 | 9.2 | 6.7 |
| Colon ca.* (SW480 met)SW620 | 3.1 | 4.1 |
| Colon ca. HT29 | 1.4 | 2.6 |
| Colon ca. HCT-116 | 8.5 | 9.1 |
| Colon ca. CaCo-2 | 5.6 | 7.1 |
| 83219 CC Well to Mod | 11.8 | 11.5 |
| Diff (ODO3866) | | |
| Colon ca. HCC-2998 | 4.6 | 7.2 |
| Gastric ca.* (liver met) NCI-N87 | 13.1 | 9.3 |
| Bladder | 3.4 | 4.2 |
| Trachea | 13.7 | 10.5 |
| Kidney | 14.6 | 6.4 |
| Kidney (fetal) | 9.2 | 4.2 |
| Renal ca. 786-0 | 9.5 | 7.3 |
| Renal ca. A498 | 23.2 | 19.4 |
| Renal ca. RXF 393 | 16.9 | 16.0 |
| Renal ca. ACHN | 14.4 | 10.5 |
| Renal ca. UO-31 | 11.2 | 8.1 |
| Renal ca. TK-10 | 5.4 | 4.8 |
| Liver | 11.2 | 3.4 |
| Liver (fetal) | 24.1 | 18.8 |
| Liver ca. (hepatoblast) HepG2 | 12.8 | 10.0 |
| Lung | 11.4 | 11.9 |
| Lung (fetal) | 11.8 | 8.9 |
| Lung ca. (small cell) LX-1 | 12.4 | 8.4 |
| Lung ca. (small cell) NCI-H69 | 15.8 | 17.0 |
| Lung ca. (s. cell var.) SHP-77 | 11.2 | 12.7 |
| Lung ca. (large cell)NCI-H460 | 30.6 | 28.4 |
| Lung ca. (non-sm. cell) A549 | 5.7 | 6.2 |
| Lung ca. (non-s. cell) NCI-H23 | 6.3 | 7.0 |
| Lung ca. (non-s. cell) HOP-62 | 13.1 | 12.0 |
| Lung ca. (non-s. cl) NCI-H522 | 5.7 | 4.6 |
| Lung ca. (squam.) SW 900 | 3.6 | 4.1 |
| Lung ca. (squam.) NCI-H596 | 12.2 | 11.1 |
| Mammary gland | 7.2 | 8.9 |
| Breast ca.* (pl. effusion) MCF-7 | 9.6 | 9.1 |
| Breast ca.* (pl. ef) MDA-MB-231 | 46.9 | 56.9 |
| Breast ca.* (pl. effusion) T47D | 4.7 | 4.6 |
| Breast ca. BT-549 | 19.6 | 20.7 |
| Breast ca. MDA-N | 6.3 | 6.2 |
| Ovary | 7.3 | 6.5 |
| Ovarian ca. OVCAR-3 | 7.4 | 5.6 |
| Ovarian ca. OVCAR-4 | 28.0 | 20.7 |
| Ovarian ca. OVCAR-5 | 7.0 | 7.0 |
| Ovarian ca. OVCAR-8 | 11.7 | 9.8 |
| Ovarian ca. IGROV-1 | 3.5 | 2.3 |
| Ovarian ca.* (ascites) SK-OV-3 | 23.7 | 17.0 |
| Uterus | 25.9 | 18.7 |
| Placenta | 10.9 | 6.3 |
| Prostate | 10.5 | 10.5 |
| Prostate ca.* (bone met)PC-3 | 20.5 | 18.1 |
| Testis | 27.2 | 19.5 |
| Melanoma Hs688(A).T | 6.6 | 5.1 |
| Melanoma* (met) Hs688(B).T | 10.7 | 8.5 |
| Melanoma UACC-62 | 43.5 | 36.3 |
| Melanoma M14 | 42.1 | 37.0 |
| Melanoma LOX IMVI | 7.9 | 9.1 |
| Melanoma* (met) SK-MEL-5 | 16.0 | 14.2 |
| Adipose | 4.8 | 3.8 |

TABLE 49
Panel 2.2

| | Relative | | Relative |
|---|---|---|---|
| | Expression (%) | | Expression (%) |
| | 2.2x4tm6395f_{—} | | 2.2x4tm6395f_{—} |
| Tissue Name | ag2022_b1 | Tissue Name | ag2022_b1 |
| Normal Colon GENPAK 061003 | 24.5 | 83793 Kidney NAT (OD04348) | 70.6 |
| 97759 Colon cancer (OD06064) | 14.3 | 98938 Kidney malignant cancer | 22.0 |
| | | (OD06204B) | |
| 97760 Colon cancer NAT | 18.0 | 98939 Kidney normal adjacent | 18.7 |
| (OD06064) | | tissue (OD06204E) | |
| 97778 Colon cancer (OD06159) | 12.2 | 85973 Kidney Cancer (OD04450- | 48.2 |
| | | 01) | |
| 97779 Colon cancer NAT | 18.1 | 85974 Kidney NAT (OD04450-03) | 16.6 |
| (OD06159) | | | |
| 98861 Colon cancer (OD06297-04) | 15.5 | Kidney Cancer Clontech 8120613 | 14.1 |
| 98862 Colon cancer NAT | 23.7 | Kidney NAT Clontech 8120614 | 40.5 |
| (OD06297-015) | | | |
| 83237 CC Gr.2 ascend colon | 25.7 | Kidney Cancer Clontech 9010320 | 20.5 |
| (ODO3921) | | | |
| 83238 CC NAT (ODO3921) | 21.9 | Kidney NAT Clontech 9010321 | 12.9 |
| 97766 Colon cancer metastasis | 6.0 | Kidney Cancer Clontech 8120607 | 48.0 |
| (OD06104) | | | |
| 97767 Lung NAT (OD06104) | 19.5 | Kidney NAT Clontech 8120608 | 37.8 |
| 87472 Colon mets to lung | 30.2 | Normal Uterus GENPAK 061018 | 11.9 |
| (OD04451-01) | | | |
| 87473 Lung NAT (OD04451-02) | 11.4 | Uterus Cancer GENPAK 064011 | 16.7 |
| Normal Prostate Clontech A+ | 21.9 | Normal Thyroid Clontech A+ | 9.6 |
| 6546-1 (8090438) | | 6570-1 (7080817) | |
| 84140 Prostate Cancer (OD04410) | 9.1 | Thyroid Cancer GENPAK 064010 | 19.0 |
| 84141 Prostate NAT (OD04410) | 11.2 | Thyroid Cancer INVITROGEN | 28.3 |
| | | A302152 | |
| Normal Ovary Res. Gen. | 58.3 | Thyroid NAT INVITROGEN | 11.8 |
| | | A302153 | |
| 98863 Ovarian cancer (OD06283- | 12.9 | Normal Breast GENPAK 061019 | 12.3 |
| 03) | | | |
| 98865 Ovarian cancer | 8.5 | 84877 Breast Cancer (OD04566) | 12.9 |
| NAT/fallopian tube (OD06283-07) | | | |
| Ovarian Cancer GENPAK 064008 | 15.8 | Breast Cancer Res Gen. 1024 | 23.1 |
| 97773 Ovarian cancer (OD06145) | 19.1 | 85975 Breast Cancer (OD04590- | 45.8 |
| | | 01) | |
| 97775 Ovarian cancer NAT | 34.4 | 85976 Breast Cancer Mets | 35.7 |
| (OD06145) | | (OD04590-03) | |
| 98853 Ovarian cancer (OD06455- | 11.0 | 87070 Breast Cancer Metastasis | 100.0 |
| 03) | | (OD04655-05) | |
| 98854 Ovarian NAT (OD06455- | 2.8 | GENPAK Breast Cancer 064006 | 19.3 |
| 07) Fallopian tube | | | |
| Normal Lung GENPAK 061010 | 11.2 | Breast Cancer Clontech 9100266 | 8.3 |
| 92337 Invasive poor diff lung | 19.1 | Breast NAT Clontech 9100265 | 9.3 |
| adeno (ODO4945-01 | | | |
| 92338 Lung NAT (ODO4945-03) | 9.9 | Breast Cancer INVITROGEN | 3.6 |
| | | A209073 | |
| 84136 Lung Malignant Cancer | 22.4 | Breast NAT INVITROGEN | 22.0 |
| (OD03126) | | A2090734 | |
| 84137 Lung NAT (OD03126) | 8.6 | 97763 Breast cancer (OD06083) | 47.4 |
| 90372 Lung Cancer (OD05014A) | 24.5 | 97764 Breast cancer node | 48.8 |
| | | metastasis (OD06083) | |
| 90373 Lung NAT (OD05014B) | 13.8 | Normal Liver GENPAK 061009 | 29.2 |
| 97761 Lung cancer (OD06081) | 12.4 | Liver Cancer Research Genetics | 31.1 |
| | | RNA 1026 | |
| 97762 Lung cancer NAT | 2.6 | Liver Cancer Research Genetics | 39.1 |
| (OD06081) | | RNA 1025 | |
| 85950 Lung Cancer (OD04237-01) | 14.4 | Paired Liver Cancer Tissue | 35.9 |
| | | Research Genetics RNA 6004-T | |
| 85970 Lung NAT (OD04237-02) | 29.6 | Paired Liver Tissue Research | 8.1 |
| | | Genetics RNA 6004-N | |
| 83255 Ocular Mel Met to Liver | 44.3 | Paired Liver Cancer Tissue | 74.1 |
| (ODO4310) | | Research Genetics RNA 6005-T | |
| 83256 Liver NAT (ODO4310) | 12.2 | Paired Liver Tissue Research | 74.3 |
| | | Genetics RNA 6005-N | |
| 84139 Melanoma Mets to Lung | 32.4 | Liver Cancer GENPAK 064003 | 45.7 |
| (OD04321) | | | |
| 84138 Lung NAT (OD04321) | 13.2 | Normal Bladder GENPAK 061001 | 19.3 |
| Normal Kidney GENPAK 061008 | 14.8 | Bladder Cancer Research Genetics | 29.9 |
| | | RNA 1023 | |
| 83786 Kidney Ca, Nuclear grade 2 | 40.8 | Bladder Cancer INVITROGEN | 30.8 |
| (OD04338) | | A302173 | |
| 83787 Kidney NAT (OD04338) | 12.3 | Normal Stomach GENPAK | 48.5 |
| | | 061017 | |
| 83788 Kidney Ca Nuclear grade | 49.2 | Gastric Cancer Clontech 9060397 | 16.4 |
| 1/2 (OD04339) | | | |
| 83789 Kidney NAT (OD04339) | 17.9 | NAT Stomach Clontech 9060396 | 33.1 |
| 83790 Kidney Ca, Clear cell type | 21.0 | Gastric Cancer Clontech 9060395 | 14.5 |
| (OD04340) | | | |
| 83791 Kidney NAT (OD04340) | 18.0 | NAT Stomach Clontech 9060394 | 34.4 |
| 83792 Kidney Ca, Nuclear grade 3 | 16.6 | Gastric Cancer GENPAK 064005 | 25.3 |
| (OD04348) | | | |

TABLE 50
Panel 4D

| | Relative | | Relative |
|---|---|---|---|
| | Expression (%) | | Expression (%) |
| | 4dx4tm4449f_{—} | | 4dx4tm4449f_{—} |
| Tissue Name | ag2022_b1 | Tissue Name | ag2022_b1 |
| 93768_Secondary Th1_anti- | 21.3 | 93100_HUVEC (Endothelial)_IL- | 3.8 |
| CD28/anti-CD3 | | 1b | |
| 93769_Secondary Th2_anti- | 16.3 | 93779_HUVEC (Endothelial)_IFN | 15.3 |
| CD28/anti-CD3 | | gamma | |
| 93770_Secondary Tr1_anti- | 0.0 | 93102_HUVEC | 13.4 |
| CD28/anti-CD3 | | (Endothelial)_TNF alpha + IFN | |
| | | gamma | |
| 93573_Secondary Th1_resting day | 5.2 | 93101_HUVEC | 13.7 |
| 4-6 in IL-2 | | (Endothelial)_TNF alpha + IL4 | |
| 93572_Secondary Th2_resting day | 6.7 | 93781_HUVEC (Endothelial)_IL- | 13.7 |
| 4-6 in IL-2 | | 11 | |
| 93571_Secondary Tr1_resting day | 6.3 | 93583_Lung Microvascular | 14.8 |
| 4-6 in IL-2 | | Endothelial Cells_none | |
| 93568_primary Th1_anti- | 15.6 | 93584_Lung Microvascular | 18.0 |
| CD28/anti-CD3 | | Endothelial Cells_TNFa (4 ng/ml) | |
| | | and IL1b (1 ng/ml) | |
| 93569_primary Th2_anti- | 12.9 | 92662_Microvascular Dermal | 22.2 |
| CD28/anti-CD3 | | endothelium_none | |
| 93570_primary Tr1_anti- | 20.1 | 92663_Microsvasular Dermal | 18.8 |
| CD28/anti-CD3 | | endothelium_TNFa (4 ng/ml) and | |
| | | IL1b (1 ng/ml) | |
| 93565_primary Th1_resting dy 4-6 | 23.0 | 93773_Bronchial | 5.8 |
| in IL-2 | | epithelium_TNFa (4 ng/ml) and | |
| | | IL1b (1 ng/ml)** | |
| 93566_primary Th2_resting dy 4-6 | 11.4 | 93347_Small Airway | 6.9 |
| in IL-2 | | Epithelium_none | |
| 93567_primary Tr1_resting dy 4-6 | 1.6 | 93348_Small Airway | 25.1 |
| in IL-2 | | Epithelium_TNFa (4 ng/ml) and | |
| | | IL1b (1 ng/ml) | |
| 93351_CD45RA CD4 | 12.7 | 92668_Coronery Artery | 18.5 |
| lymphocyte_anti-CD28/anti-CD3 | | SMC_resting | |
| 93352_CD45RO CD4 | 11.4 | 92669_Coronery Artery | 14.0 |
| lymphocyte_anti-CD28/anti-CD3 | | SMC_TNFa (4 ng/ml) and IL1b (1 ng/ml) | |
| 93251_CD8 Lymphocytes_anti- | 12.4 | 93107_astrocytes_resting | 12.6 |
| CD28/anti-CD3 | | | |
| 93353_chronic CD8 Lymphocytes | 11.9 | 93108_astrocytes_TNFa (4 ng/ml) | 15.4 |
| 2ry_resting dy 4-6 in IL-2 | | and IL1b (1 ng/ml) | |
| 93574_chronic CD8 Lymphocytes | 11.4 | 92666_KU-812 (Basophil)_resting | 58.7 |
| 2ry_activated CD3/CD28 | | | |
| 93354_CD-4_none | 3.2 | 92667_KU-812 | 100.0 |
| | | (Basophil)_PMA/ionoycin | |
| 93252_Secondary | 9.0 | 93579_CCD1106 | 14.2 |
| Th1/Th2/Tr1_anti-CD95 CH11 | | (Keratinocytes)_none | |
| 93103_LAK cells_resting | 10.6 | 93580_CCD1106 | 1.7 |
| | | (Keratinocytes)_TNFa and IFNg** | |
| 93788_LAK cells_IL-2 | 12.8 | 93791_Liver Cirrhosis | 1.6 |
| 93787_LAK cells_IL-2 + IL-12 | 9.3 | 93792_Lupus Kidney | 1.4 |
| 93789_LAK cells_IL-2 + IFN | 11.3 | 93577_NCI-H292 | 19.8 |
| gamma | | | |
| 93790_LAK cells_IL-2 + IL-18 | 9.3 | 93358_NCI-H292_IL-4 | 20.4 |
| 93104_LAK | 5.7 | 93360_NCI-H292_IL-9 | 22.3 |
| cells_PMA/ionomycin and IL-18 | | | |
| 93578_NK Cells IL-2_resting | 9.7 | 93359_NCI-H292_IL-13 | 12.5 |
| 93109_Mixed Lymphocyte | 9.2 | 93357_NCI-H292_IFN gamma | 12.9 |
| Reaction_Two Way MLR | | | |
| 93110_Mixed Lymphocyte | 11.5 | 93777_HPAEC_- | 16.3 |
| Reaction_Two Way MLR | | | |
| 93111 _Mixed Lymphocyte | 7.5 | 93778_HPAEC_IL-1beta/TNA | 17.5 |
| Reaction_Two Way MLR | | alpha | |
| 93112_Mononuclear Cells | 3.9 | 93254_Normal Human Lung | 23.0 |
| (PBMCs)_resting | | Fibroblast_none | |
| 93113_Mononuclear Cells | 24.7 | 93253_Normal Human Lung | 14.3 |
| (PBMCs)_PWM | | Fibroblast_TNFa (4 ng/ml) and IL- | |
| | | 1b (1 ng/ml) | |
| 93114_Mononuclear Cells | 12.6 | 93257_Normal Human Lung | 30.8 |
| (PBMCs)_PHA-L | | Fibroblast_IL-4 | |
| 93249_Ramos (B cell)_none | 13.6 | 93256_Normal Human Lung | 27.0 |
| | | Fibroblast_IL-9 | |
| 93250_Ramos (B cell)_ionomycin | 32.2 | 93255_Normal Human Lung | 19.5 |
| | | Fibroblast_IL-13 | |
| 93349_B lymphocytes_PWM | 49.8 | 93258_Normal Human Lung | 27.4 |
| | | Fibroblast_IFN gamma | |
| 93350_B lymphoytes_CD40L and | 25.5 | 93106_Dermal Fibroblasts | 24.5 |
| IL-4 | | CCD1070_resting | |
| 92665_EOL-1 | 18.0 | 93361_Dermal Fibroblasts | 47.3 |
| (Eosinophil)_dbcAMP | | CCD1070_TNF alpha 4 ng/ml | |
| differentiated | | | |
| 93248_EOL-1 | 27.5 | 93105_Dermal Fibroblasts | 21.4 |
| (Eosinophil)_dbcAMP/PMAionomycin | | CCD1070_IL-1beta 1 ng/ml | |
| 93356_Dendritic Cells_none | 10.0 | 93772_dermal fibroblast_IFN | 12.4 |
| | | gamma | |
| 93355_Dendritic Cells_LPS 100 ng/ml | 6.9 | 93771_dermal fibroblast_IL-4 | 22.5 |
| 93775_Dendritic Cells_anti-CD40 | 8.9 | 93260_IBD Colitis 2 | 0.8 |
| 93774_Monocytes_resting | 11.1 | 93261_IBD Crohns | 1.3 |
| 93776_Monocytes_LPS 50 ng/ml | 7.0 | 735010_Colon_normal | 13.7 |
| 93581_Macrophages_resting | 12.4 | 735019_Lung_none | 8.6 |
| 93582_Macrophages_LPS 100 ng/ml | 5.8 | 64028-1_Thymus_none | 9.9 |
| 93098_HUVEC | 22.1 | 64030-1_Kidney_none | 18.6 |
| (Endothelial)_none | | | |
| 93099_HUVEC | 22.3 | | |
| (Endothelial)_starved | | | |

Panel 1.3D Summary Two results using the same probe and primer set show results that are in excellent agreement, with highest expression of the NOV9 gene in adult skeletal muscle (CTs=27). The NOV9 gene also shows moderate expression in other tissues with metabolic function including adipose, adult and fetal heart and liver, adult skeletal muscle, pancreas, and the adrenal, thyroid, and pituitary glands. Expression is much lower in fetal skeletal muscle (CTs=30) relative to the adult tissue (CTs=27), which may implicate the expression of this gene in differentiation of skeletal muscle and thus suggests that expression of the NOV9 gene could be used to differentiate between the adult and fetal phenotypes of this tissue. The pathway mediated by MAP kinase kinase (MAPKK) has been shown to influence myoblast proliferation and both insulin and exercise stimulate signaling via this pathway in skeletal muscle. Insulin resistance in obese and diabetic subjects may in part be due to tumor necrosis factor alpha, whose effects are mediated through interference with the normal activation of MAPKK by insulin. In addition, exercise training significantly improves insulin-induced MAPKK activity in obese Zucker rats. This indicates that an activator of this kinase may be an effective pharmaceutical agent in the treatment of diabetes. Furthermore, activation of the MAPKK pathway is involved in adipocyte differentiation from preadipocytes in androgen deficiency. Therefore, a MAPKK antagonist may be a suitable pharmacological agent in the treatment of obesity in some cases.

The NOV9 gene is expressed at higher levels in cell lines derived from melanoma, and kidney and lung cancers compared to the normal tissues and may play a role in cancers in these tissues. Thus, the expression of this gene could be useful as a marker or as a therapeutic for lung and kidney cancer as well as melanomas. In addition, therapeutic modulation of the activity of the gene product, through the use of peptides, chimeric molecules or small molecule drugs, may be useful in the therapy of these cancers. The NOV9 gene, a homolog of Mitogen Activated Protein Kinase Kinase, is expressed at high to moderate levels across the brain, with highest expression in the central nervous seen in the thalamus (CT=28.4). Mitogen Activated Protein Kinase Kinase is activated by Valproic acid, a drug that is used to treat both seizure disorders and bipolar depression. Valproic acid is believed to work by increasing neuronal production of GABA, the major inhibitory neurotransmitter in the brain. Selective activation of this kinase may therefore have therapeutic benefit in the treatment of seizure disorders, bipolar disorder, or in any other neurological/psychiatric condition believed to be caused by a GABA deficit (schizophrenia).

Panel 2.2 Summary Highest expression of the NOV9 gene in this panel is seen in a breast cancer sample (CT=29.0). The expression of this gene shows an association with samples derived from breast and kidney cancers when compared to the matched normal tissue.

Thus, expression of the NOV9 gene could be useful as a marker for breast and kidney cancers.

Furthermore, therapeutic activity of the product of this gene, through the use of peptides, chimeric molecules or small molecule drugs, may be useful in the treatment of breast and kidney cancers.

Panel 4D Summary Expression of the NOV9 gene is ubiquitous throughout this panel. Highest expression of this gene is found in the basophil cell line, KU-812, upon activation with PMA/ionomycin (CT=26.2), compared to non-activated cells. High expression of the NOV9 gene is also found on activated B cells, a B cell line, and dermal fibroblasts. The NOV9 gene is homologous to a Mitogen Activated Protein Kinase Kinase 2 (MAPKK2), a serine threonine kinase which functions downstream of Raf in the signaling pathway that affects proliferation and differentiation. The high expression of this kinase on basophiles suggests a role for this kinase in mast cell/basophile signal transduction. Activated mast/basophile cells have been associated with many atopic diseases, including asthma, atopic contact dermatitis, allergies, and rhinitis. Therefore, therapeutic modulation of the expression or function of the NOV9 gene product, through the use of small molecule drugs, might be beneficial in the treatment of these diseases. In addition, the high expression of this kinase in activated B cells suggests that the use of small molecule drugs designed to the NOV9 gene product could prevent B cell hyperproliferative disorders such as autoimmune diseases and lymphomas (Yuan et al., The mood stabilizer valproic acid activates mitogen-activated protein kinases and promotes neurite growth. J. Biol. Chem. 276:31674-83, 2001; Bullet and Hsieh, MEK inhibitors block BDNF-dependent and -independent expression of GABA(A) receptor subunit mRNAs in cultured mouse cerebellar granule neurons. Brain Res Dev Brain Res. 119: 1-10, 2000; Jones et al., ERKI/2 is required for myoblast proliferation but is dispensable for muscle gene expression and cell fusion. J Cell Physiol. 186:104-15, 2001; Wojtaszewski et al., Differential regulation of MAP kinase by contraction and insulin in skeletal muscle: metabolic implications. Am J. Physiol. 277(4 Pt 1):E724-32, 1999; Begum et al., Effect of tumor necrosis factor-alpha on insulin-stimulated mitogen-activated protein kinase cascade in cultured rat skeletal muscle cells. Eur J. Biochem. 238:214-20, 1996; Osman et al., Exercise training increases ERK2 activity in skeletal muscle of obese Zucker rats. J Appl Physiol. 90:454-60, 2001; Lacasa et al., Site-related specificities of the control by androgenic status of adipogenesis and mitogen-activated protein kinase cascade/c-fos signaling pathways in rat preadipocytes. Endocrinology 138:3181-6, 1997).

NOV11: Thymosin beta 10-Like

Expression of the NOV11a gene (GMAC079400_A) and variant NOV11b (CG109754-01) was assessed using the primer-probe sets Ag2431 and Ag3087 described in Tables 51. Results from RTQ-PCR mins are shown in Tables 52, 53, 54, and 55.

TABLE 51
Probe Name Ag2431

| | | | | Start | SEQ ID |
|---|---|---|---|---|---|
| Primers | Sequences | TM | Length | Position | NO: |
| Forward | 5′-AGAAAATGGCAGACAAACCA-3′ | 58.2 | 20 | 23 | 135 |
| Probe | TET-5′-AATCGCCAGCTTCAATAGGGCCAAG-3′-TAMRA | 70.7 | 25 | 54 | 136 |
| Reverse | 5′-GCGTCTCCCTTTTCTTCAG-3′ | 58.6 | 19 | 79 | 137 |

TABLE 52
Panel 1.3D

| | Relative | | Relative |
|---|---|---|---|
| | Expression (%) | | Expression (%) |
| | 1.3dtm4242t_{—} | | 1.3dtm4242t_{—} |
| Tissue Name | ag2431 | Tissue Name | ag2431 |
| Liver adenocarcinoma | 13.7 | Kidney (fetal) | 3.6 |
| Pancreas | 0.9 | Renal ca. 786-0 | 2.4 |
| Pancreatic ca. CAPAN 2 | 0.4 | Renal ca. A498 | 3.6 |
| Adrenal gland | 3.0 | Renal ca. RXF 393 | 0.5 |
| Thyroid | 0.9 | Renal ca. ACHN | 0.1 |
| Salivary gland | 1.2 | Renal ca. UO-31 | 0.2 |
| Pituitary gland | 1.5 | Renal ca. TK-10 | 0.3 |
| Brain (fetal) | 28.1 | Liver | 1.6 |
| Brain (whole) | 2.2 | Liver (fetal) | 3.6 |
| Brain (amygdala) | 24.1 | Liver ca. (hepatoblast) HepG2 | 0.8 |
| Brain (cerebellum) | 1.5 | Lung | 8.6 |
| Brain (hippocampus) | 100.0 | Lung (fetal) | 5.0 |
| Brain (substantia nigra) | 1.9 | Lung ca. (small cell) LX-1 | 1.4 |
| Brain (thalamus) | 6.3 | Lung ca. (small cell) NCI-H69 | 1.1 |
| Cerebral Cortex | 20.6 | Lung ca.(s. cell var.) SHP-77 | 1.4 |
| Spinal cord | 1.0 | Lung ca. (large cell)NCI-H460 | 1.0 |
| CNS ca. (glio/astro) U87-MG | 9.4 | Lung ca.(non-sm. cell) A549 | 1.7 |
| CNS ca. (glio/astro) U-118-MG | 6.5 | Lung ca.(non-s. cell) NCI-H23 | 1.4 |
| CNS ca. (astro) SW1783 | 3.5 | Lung ca.(non-s. cell) HOP-62 | 0.4 |
| CNS ca.* (neuro; met) SK-N-AS | 5.6 | Lung ca. (non-s. cl) NCI-H522 | 0.8 |
| CNS ca. (astro) SF-539 | 1.5 | Lung ca. (squam.) SW 900 | 1.6 |
| CNS ca. (astro) SNB-75 | 3.9 | Lung ca. (squam.) NCI-H596 | 0.5 |
| CNS ca. (glio) SNB-19 | 1.8 | Mammary gland | 3.3 |
| CNS ca. (glio) U251 | 0.6 | Breast ca.* (pl. effusion) MCF-7 | 3.8 |
| CNS ca. (glio) SF-295 | 0.4 | Breast ca.* (pl. ef) MDA-MB-231 | 32.3 |
| Heart (fetal) | 3.1 | Breast ca.* (pl. effusion) T47D | 0.2 |
| Heart | 1.5 | Breast ca. BT-549 | 25.3 |
| Fetal Skeletal | 6.8 | Breast ca. MDA-N | 1.0 |
| Skeletal muscle | 0.5 | Ovary | 4.5 |
| Bone marrow | 7.9 | Ovarian ca. OVCAR-3 | 1.1 |
| Thymus | 0.6 | Ovarian ca. OVCAR-4 | 0.1 |
| Spleen | 2.5 | Ovarian ca. OVCAR-5 | 0.6 |
| Lymph node | 4.4 | Ovarian ca. OVCAR-8 | 1.1 |
| Colorectal | 3.8 | Ovarian ca. IGROV-1 | 0.8 |
| Stomach | 1.4 | Ovarian ca.* (ascites) SK-OV-3 | 2.9 |
| Small intestine | 3.2 | Uterus | 1.5 |
| Colon ca. SW480 | 1.1 | Placenta | 1.7 |
| Colon ca.* (SW480 met)SW620 | 0.7 | Prostate | 1.4 |
| Colon ca. HT29 | 1.7 | Prostate ca.* (bone met)PC-3 | 1.5 |
| Colon ca. HCT-116 | 1.6 | Testis | 0.7 |
| Colon ca. CaCo-2 | 0.8 | Melanoma Hs688(A).T | 3.4 |
| 83219 CC Well to Mod Diff | 5.3 | Melanoma* (met) Hs688(B).T | 1.6 |
| (ODO3866) | | | |
| Colon ca. HCC-2998 | 3.8 | Melanoma UACC-62 | 0.5 |
| Gastric ca.* (liver met) NCI-N87 | 4.7 | Melanoma M14 | 1.2 |
| Bladder | 8.4 | Melanoma LOX IMVI | 12.7 |
| Trachea | 3.1 | Melanoma* (met) SK-MEL-5 | 2.8 |
| Kidney | 1.2 | Adipose | 3.7 |

TABLE 53
Panel 2D

| | Relative | | Relative |
|---|---|---|---|
| | Expression (%) | | Expression (%) |
| | 2dtm4243t_{—} | | 2dtm4243t_{—} |
| Tissue Name | ag2431 | Tissue Name | ag2431 |
| Normal Colon GENPAK 061003 | 33.0 | Kidney NAT Clontech 8120608 | 1.1 |
| 83219 CC Well to Mod Diff | 21.8 | Kidney Cancer Clontech 8120613 | 1.4 |
| (ODO3866) | | | |
| 83220 CC NAT (ODO3866) | 19.8 | Kidney NAT Clontech 8120614 | 1.3 |
| 83221 CC Gr.2 rectosigmoid | 26.6 | Kidney Cancer Clontech 9010320 | 5.0 |
| (ODO3868) | | | |
| 83222 CC NAT (ODO3868) | 2.1 | Kidney NAT Clontech 9010321 | 3.0 |
| 83235 CC Mod Diff (ODO3920) | 26.1 | Normal Uterus GENPAK 061018 | 1.6 |
| 83236 CC NAT (ODO3920) | 13.9 | Uterus Cancer GENPAK 064011 | 7.1 |
| 83237 CC Gr.2 ascend colon | 49.7 | Normal Thyroid Clontech A+ | 1.4 |
| (ODO3921) | | 6570-1 | |
| 83238 CC NAT (ODO3921) | 8.4 | Thyroid Cancer GENPAK 064010 | 5.2 |
| 83241 CC from Partial | 13.9 | Thyroid Cancer INVITROGEN | 2.7 |
| Hepatectomy (ODO4309) | | A302152 | |
| 83242 Liver NAT (ODO4309) | 3.9 | Thyroid NAT INVITROGEN | 2.8 |
| | | A302153 | |
| 87472 Colon mets to lung | 5.6 | Normal Breast GENPAK 061019 | 3.2 |
| (OD04451-01) | | | |
| 87473 Lung NAT (OD04451-02) | 6.7 | 84877 Breast Cancer (OD04566) | 1.9 |
| Normal Prostate Clontech A+ | 1.4 | 85975 Breast Cancer (OD04590- | 3.4 |
| 6546-1 | | 01) | |
| 84140 Prostate Cancer (OD04410) | 7.4 | 85976 Breast Cancer Mets | 4.7 |
| | | (OD04590-03) | |
| 84141 Prostate NAT (OD04410) | 6.4 | 87070 Breast Cancer Metastasis | 8.0 |
| | | (OD04655-05) | |
| 87073 Prostate Cancer (OD04720- | 3.2 | GENPAK Breast Cancer 064006 | 7.9 |
| 01) | | | |
| 87074 Prostate NAT (OD04720- | 6.2 | Breast Cancer Res. Gen. 1024 | 4.9 |
| 02) | | | |
| Normal Lung GENPAK 061010 | 17.7 | Breast Cancer Clontech 9100266 | 9.4 |
| 83239 Lung Met to Muscle | 33.4 | Breast NAT Clontech 9100265 | 5.8 |
| (ODO4286) | | | |
| 83240 Muscle NAT (ODO4286) | 3.0 | Breast Cancer INVITROGEN | 19.2 |
| | | A209073 | |
| 84136 Lung Malignant Cancer | 7.3 | Breast NAT INVITROGEN | 5.0 |
| (OD03126) | | A2090734 | |
| 84137 Lung NAT (OD03126) | 6.6 | Normal Liver GENPAK 061009 | 0.6 |
| 84871 Lung Cancer (OD04404) | 5.0 | Liver Cancer GENPAK 064003 | 1.8 |
| 84872 Lung NAT (OD04404) | 4.9 | Liver Cancer Research Genetics | 1.2 |
| | | RNA 1025 | |
| 84875 Lung Cancer (OD04565) | 4.2 | Liver Cancer Research Genetics | 4.7 |
| | | RNA 1026 | |
| 84876 Lung NAT (OD04565) | 4.2 | Paired Liver Cancer Tissue | 1.1 |
| | | Research Genetics RNA 6004-T | |
| 85950 Lung Cancer (OD04237-01) | 19.5 | Paired Liver Tissue Research | 2.1 |
| | | Genetics RNA 6004-N | |
| 85970 Lung NAT (OD04237-02) | 8.7 | Paired Liver Cancer Tissue | 4.5 |
| | | Research Genetics RNA 6005-T | |
| 83255 Ocular Mel Met to Liver | 2.7 | Paired Liver Tissue Research | 1.0 |
| (ODO4310) | | Genetics RNA 6005-N | |
| 83256 Liver NAT (ODO4310) | 2.1 | Normal Bladder GENPAK 061001 | 19.6 |
| 84139 Melanoma Mets to Lung | 8.0 | Bladder Cancer Research Genetics | 8.7 |
| (OD04321) | | RNA 1023 | |
| 84138 Lung NAT (OD04321) | 6.9 | Bladder Cancer INVITROGEN | 16.5 |
| | | A302173 | |
| Normal Kidney GENPAK 061008 | 2.6 | 87071 Bladder Cancer (OD04718- | 20.3 |
| | | 01) | |
| 83786 Kidney Ca, Nuclear grade 2 | 7.9 | 87072 Bladder Normal Adjacent | 10.1 |
| (OD04338) | | (OD04718-03 | |
| 83787 Kidney NAT (OD04338) | 2.5 | Normal Ovary Res. Gen | 9.4 |
| 83788 Kidney Ca Nuclear grade | 6.6 | Ovarian Cancer GENPAK 064008 | 15.1 |
| 1/2 (OD04339) | | | |
| 83789 Kidney NAT (OD04339) | 1.4 | 87492 Ovary Cancer (OD04768- | 7.1 |
| | | 07) | |
| 83790 Kidney Ca, Clear cell type | 6.9 | 87493 Ovary NAT (OD04768-08) | 3.5 |
| (OD04340) | | | |
| 83791 Kidney NAT (OD04340) | 3.9 | Normal Stomach GENPAK | 4.7 |
| | | 061017 | |
| 83792 Kidney Ca, Nuclear grade 3 | 18.2 | Gastric Cancer Clontech 9060358 | 2.1 |
| (OD04348) | | | |
| 83793 Kidney NAT (OD04348) | 3.6 | NAT Stomach Clontech 9060359 | 17.7 |
| 87474 Kidney Cancer (OD04622- | 10.8 | Gastric Cancer Clontech 9060395 | 14.7 |
| 01) | | | |
| 87475 Kidney NAT (OD04622-03) | 1.2 | NAT Stomach Clontech 9060394 | 22.7 |
| 85973 Kidney Cancer (OD04450- | 6.0 | Gastric Cancer Clontech 9060397 | 100.0 |
| 01) | | | |
| 85974 Kidney NAT (OD04450-03) | 1.9 | NAT Stomach Clontech 9060396 | 14.8 |
| Kidney Cancer Clontech 8120607 | 3.2 | Gastric Cancer GENPAK 064005 | 32.3 |

TABLE 54
Panel 4D

| | Relative | | Relative |
|---|---|---|---|
| | Expression (%) | | Expression (%) |
| | 4dtm4244t_{—} | | 4dtm4244t_{—} |
| Tissue Name | ag2431 | Tissue Name | ag2431 |
| 93768_Secondary Th1_anti- | 97.9 | 93100_HUVEC (Endothelial)_IL- | 4.8 |
| CD28/anti-CD3 | | 1b | |
| 93769_Secondary Th2_anti- | 69.7 | 93779_HUVEC (Endothelial)_IFN | 15.2 |
| CD28/anti-CD3 | | gamma | |
| 93770_Secondary Tr1_anti- | 100.0 | 93102_HUVEC | 11.8 |
| CD28/anti-CD3 | | (Endothelial)_TNF alpha + IFN | |
| | | gamma | |
| 93573_Secondary Th1_resting day | 65.5 | 93101_HUVEC | 20.4 |
| 4-6 in IL-2 | | (Endothelial)_TNF alpha + IL4 | |
| 93572_Secondary Th2_resting day | 50.0 | 93781_HUVEC (Endothelial)_IL- | 14.7 |
| 4-6 in IL-2 | | 11 | |
| 93571_Secondary Tr1_resting day | 46.7 | 93583_Lung Microvascular | 32.1 |
| 4-6 in IL-2 | | Endothelial Cells_none | |
| 93568_primary Th1_anti- | 92.0 | 93584_Lung Microvascular | 38.2 |
| CD28/anti-CD3 | | Endothelial Cells_TNFa (4 ng/ml) | |
| | | and IL1b (1 ng/ml) | |
| 93569_primary Th2_anti- | 79.0 | 92662_Microvascular Dermal | 41.2 |
| CD28/anti-CD3 | | endothelium_none | |
| 93570_primary Tr1_anti- | 57.0 | 92663_Microsvasular Dermal | 34.9 |
| CD28/anti-CD3 | | endothelium_TNFa (4 ng/ml) and | |
| | | IL1b (1 ng/ml) | |
| 93565_primary Th1_resting dy 4-6 | 80.1 | 93773_Bronchial | 51.8 |
| in IL-2 | | epithelium_TNFa (4 ng/ml) and | |
| | | IL1b (1 ng/ml)** | |
| 93566_primary Th2_resting dy 4-6 | 61.6 | 93347_Small Airway | 18.8 |
| in IL-2 | | Epithelium_none | |
| 93567_primary Tr1_resting dy 4-6 | 31.6 | 93348_Small Airway | 43.2 |
| in IL-2 | | Epithelium_TNFa (4 ng/ml) and | |
| | | IL1b (1 ng/ml) | |
| 93351_CD45RA CD4 | 46.0 | 92668_Coronery Artery | 30.8 |
| lymphocyte_anti-CD28/anti-CD3 | | SMC_resting | |
| 93352_CD45RO CD4 | 40.1 | 92669_Coronery Artery | 20.7 |
| lymphocyte_anti-CD28/anti-CD3 | | SMC_TNFa (4 ng/ml) and IL1b (1 ng/ml) | |
| 93251_CD8 Lymphocytes_anti- | 29.5 | 93107_astrocytes_resting | 11.0 |
| CD28/anti-CD3 | | | |
| 93353_chronic CD8 Lymphocytes | 33.0 | 93108_astrocytes_TNFa (4 ng/ml) | 11.2 |
| 2ry_resting dy 4-6 in IL-2 | | and IL1b (1 ng/ml) | |
| 93574_chronic CD8 Lymphocytes | 38.4 | 92666_KU-812 (Basophil)_resting | 9.7 |
| 2ry_activated CD3/CD28 | | | |
| 93354_CD4_none | 20.0 | 92667_KU-812 | 18.9 |
| | | (Basophil)_PMA/ionoycin | |
| 93252_Secondary | 49.3 | 93579_CCD1106 | 46.0 |
| Th1/Th2/Tr1_anti-CD95 CH11 | | (Keratinocytes)_none | |
| 93103_LAK cells_resting | 85.9 | 93580_CCD1106 | 31.9 |
| | | (Keratinocytes)_TNFa and IFNg** | |
| 93788_LAK cells_IL-2 | 32.3 | 93791_Liver Cirrhosis | 4.9 |
| 93787_LAK cells_IL-2 + IL-12 | 24.1 | 93792_Lupus Kidney | 2.6 |
| 93789_LAK cells_IL-2 + IFN | 20.9 | 93577_NCI-H292 | 17.8 |
| gamma | | | |
| 93790_LAK cells_IL-2 + IL-18 | 14.0 | 93358_NCI-H292_IL-4 | 15.1 |
| 93104_LAK | 36.3 | 93360_NCI-H292_IL-9 | 17.3 |
| cells_PMA/ionomycin and IL-18 | | | |
| 93578_NK Cells IL-2_resting | 14.4 | 93359_NCI-H292_IL-13 | 5.0 |
| 93109_Mixed Lymphocyte | 29.7 | 93357_NCI-H292_IFN gamma | 13.5 |
| Reaction_Two Way MLR | | | |
| 93110_Mixed Lymphocyte | 27.2 | 93777_HPAEC_- | 14.3 |
| Reaction_Two Way MLR | | | |
| 93111_Mixed Lymphocyte | 34.9 | 93778_HPAEC_IL-1beta/TNA | 30.1 |
| Reaction_Two Way MLR | | alpha | |
| 93112_Mononuclear Cells | 16.6 | 93254_Normal Human Lung | 39.8 |
| (PBMCs)_resting | | Fibroblast_none | |
| 93113_Mononuclear Cells | 36.6 | 93253_Normal Human Lung | 38.7 |
| (PBMCs)_PWM | | Fibroblast_TNFa (4 ng/ml) and IL- | |
| | | 1b (1 ng/ml) | |
| 93114_Mononuclear Cells | 35.4 | 93257_Normal Human Lung | 63.7 |
| (PBMCs)_PHA-L | | Fibroblast_IL-4 | |
| 93249_Ramos (B cell)_none | 11.8 | 93256_Normal Human Lung | 55.5 |
| | | Fibroblast_IL-9 | |
| 93250_Ramos (B cell)_ionomycin | 13.7 | 93255_Normal Human Lung | 59.5 |
| | | Fibroblast_IL-13 | |
| 93349_B lymphocytes_PWM | 25.5 | 93258_Normal Human Lung | 78.5 |
| | | Fibroblast_IFN gamma | |
| 93350_B lymphoytes_CD40L and | 10.8 | 93106_Dermal Fibroblasts | 57.8 |
| IL-4 | | CCD1070_resting | |
| 92665_EOL-1 | 5.1 | 93361_Dermal Fibroblasts | 64.2 |
| (Eosinophil)_dbcAMP | | CCD1070_TNF alpha 4 ng/ml | |
| differentiated | | | |
| 93248_EOL-1 | 15.6 | 93105_Dermal Fibroblasts | 27.2 |
| (Eosinophil)_dbcAMP/PMAionomycin | | CCD1070_IL-1beta 1 ng/ml | |
| 93356_Dendritic Cells_none | 33.4 | 93772_dermal fibroblast_IFN | 33.2 |
| | | gamma | |
| 93355_Dendritic Cells_LPS 100 ng/ml | 31.0 | 93771_dermal fibroblast_IL-4 | 38.2 |
| 93775_Dendritic Cells_anti-CD40 | 25.7 | 93260_IBD Colitis 2 | 4.0 |
| 93774_Monocytes_resting | 29.9 | 93261_IBD Crohns | 7.9 |
| 93776_Monocytes_LPS 50 ng/ml | 14.4 | 735010_Colon_normal | 35.1 |
| 93581_Macrophages_resting | 28.3 | 735019_Lung_none | 38.4 |
| 93582_Macrophages_LPS 100 ng/ml | 35.1 | 64028-1_Thymus_none | 18.7 |
| 93098_HUVEC | 28.1 | 64030-1_Kidney_none | 52.1 |
| (Endothelial)_none | | | |
| 93099_HUVEC | 37.1 | | |
| (Endothelial)_starved | | | |

TABLE 55
Panel CNS_neurodegeneration_v1.0

| | Relative | | Relative |
|---|---|---|---|
| | Expression (%) | | Expression (%) |
| | tm6902t_{—} | | tm6902t_{—} |
| Tissue Name | ag2431_b1s2 | Tissue Name | ag2431_b1s2 |
| AD 1 Hippo | 9.1 | Control (Path) 3 Temporal Ctx | 2.0 |
| AD 2 Hippo | 0.0 | Control (Path) 4 Temporal Ctx | 0.0 |
| AD 3 Hippo | 0.0 | AD 1 Occipital Ctx | 2.0 |
| AD 4 Hippo | 0.0 | AD 2 Occipital Ctx (Missing) | 0.0 |
| AD 5 hippo | 61.4 | AD 3 Occipital Ctx | 3.6 |
| AD 6 Hippo | 6.2 | AD 4 Occipital Ctx | 0.0 |
| Control 2 Hippo | 0.0 | AD 5 Occipital Ctx | 4.1 |
| Control 4 Hippo | 0.0 | AD 6 Occipital Ctx | 34.0 |
| Control (Path) 3 Hippo | 0.0 | Control 1 Occipital Ctx | 0.0 |
| AD 1 Temporal Ctx | 2.9 | Control 2 Occipital Ctx | 4.5 |
| AD 2 Temporal Ctx | 0.0 | Control 3 Occipital Ctx | 3.4 |
| AD 3 Temporal Ctx | 0.0 | Control 4 Occipital Ctx | 4.1 |
| AD 4 Temporal Ctx | 0.0 | Control (Path) 1 Occipital Ctx | 5.7 |
| AD 5 Inf Temporal Ctx | 52.5 | Control (Path) 2 Occipital Ctx | 2.1 |
| AD 5 SupTemporal Ctx | 100.0 | Control (Path) 3 Occipital Ctx | 0.0 |
| AD 6 Inf Temporal Ctx | 6.6 | Control (Path) 4 Occipital Ctx | 9.5 |
| AD 6 Sup Temporal Ctx | 16.6 | Control 1 Parietal Ctx | 0.0 |
| Control 1 Temporal Ctx | 0.0 | Control 2 Parietal Ctx | 61.0 |
| Control 2 Temporal Ctx | 1.7 | Control 3 Parietal Ctx | 1.3 |
| Control 3 Temporal Ctx | 1.3 | Control (Path) 1 Parietal Ctx | 13.6 |
| Control 4 Temporal Ctx | 0.0 | Control (Path) 2 Parietal Ctx | 0.0 |
| Control (Path) 1 Temporal Ctx | 6.6 | Control (Path) 3 Parietal Ctx | 3.1 |
| Control (Path) 2 Temporal Ctx | 7.4 | Control (Path) 4 Parietal Ctx | 1.4 |

Panel 1.3D Summary Ag2431 The NON11 gene, a homolog of thymosin beta 10, is most highly expressed in the hippocampus (CT=24.2) and is expressed widely in the CNS. This gene appears to be important in the process of gliosis, which is a hallmark of all of the neurodegenerative diseases. Furthermore, the formation of a glial scar is one of the principle barriers to neuroregeneration in response to spinal cord injury and head trauma. Therefore, the selective down-regulation of this gene and/or its protein product may be beneficial in the treatment of spine or head injury, or in any of the neurodegenerative diseases (Alzheimer's, Parkinson's, Huntington's, spinocerebellar ataxia, etc).

The NOV11 gene also has moderate to low expression in many metabolic tissues including adipose, adrenal, adult and fetal heart, adult and fetal liver, adult and fetal skeletal muscle, pancreas, pituitary and thyroid. The gene appears to be expressed at higher levels in fetal skeletal muscle (CT=28) than in adult skeletal muscle (CT=31.7) and could potentially be used to distinguish between the adult and fetal phenotypes of this tissue. In addition, the greater expression in fetal skeletal muscle suggests that the NOV11 gene may play a role in muscular growth or development in the fetus and therefore could act in a regenerative capacity in an adult. Thus, therapeutic modulation of the NOV1I gene could be useful in the treatment of muscle related diseases and treatment with the protein product could restore muscle mass or function to weak or dystrophic muscle.

The NOV11 gene is expressed at significant levels in cell lines derived from breast cancer, liver cancer and melanoma when compared to expression in the corresponding normal tissues. Thus, the expression of this gene could be useful as a marker or as a therapeutic for breast and liver cancer, as well as melanomas. In addition, therapeutic modulation of the activity of the protein encoded by the NOV11 gene, through the use of peptides, antibodies, chimeric molecules or small molecule drugs, may be useful in the therapy of these cancers.

Panel 2D Summary Ag2431 Highest expression of the NOV11 gene is seen in a gastric cancer sample (CT=23.5). The expression of this gene in panel 2D shows an association with samples derived from ovarian, bladder, liver, breast, kidney and colon cancers when compared to the matched normal tissue. A lung cancer that has metastasized to muscle also shows increased expression of this gene when compared to the adjacent muscle tissue. Thus, expression of the NOV11 gene could be of use as a marker for these cancers. Furthermore, therapeutic modulation of the activity of the product of this gene, through the use of peptides, antibodies, chimeric molecules or small molecule drugs, may beneficial in the treatmen of these cancers.

Panel 4L) Summary Ag2431 The NOV11 gene is ubiquitously expressed throughout this panel in both normal cell types and cell lines, regardless of their activation status. This gene encodes a protein that has homology with Thymosin beta-10. Some reports indicate that thymosin beta 10 (as thymosin beta 4- which is functionally very similar) is an effective regulator of a large subset of actin filaments in living cells. Reduced expression of thymosin beta-10 may contribute to the senescent phenotype by reducing EC plasticity and thus impairing their response to migratory stimuli. Therefore, therapeutics designed with the protein encoded for by the NOV11 gene may play a role in maintaining or restoring normal function of lymphoid, lung, dermal fibroblasts, endothelial cells and could be beneficial in preventing aging of the cells.

Panel CNS_neurodegenerataion_v1.0 Summary Ag2431 Expression of the NOV11 gene is restricted to a few samples in this panel, with highest expression in the cerebral cortex of an Alzheimer's patient (CT=33.5). While no association between the expression of this gene and the presence of Alzheimer's disease is detected in this panel, these results confirm the expression of this gene in the brains of a further set of individuals. Please see Panel 1.3D for a discussion of potential utility of this gene in the central nervous system (Carpintero et al., Expression of the thymosin beta 10 gene in normal and kainic acid-treated rat forebrain. Brain Res Mol Brain Res. 70:141-6, 1999).

### Other Embodiments

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims.

What is claimed is:

1. An isolated polypeptide comprising an amino acid sequence selected from the group consisting of:
(a) a mature form of an amino acid sequence selected from the group consisting of SEQ ID NOS:2, 4, 6, 8, 10, 12, 14, 16, 18, 20, 22, 24, 26, 28, 30, 32 and/or 34;
(b) a variant of a mature form of an amino acid sequence selected from the group consisting of SEQ ID NOS:2, 4, 6, 8, 10, 12, 14, 16, 18, 20, 22, 24, 26, 28, 30, 32 and/or 34, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS:2, 4, 6, 8, 10, 12, 14, 16, 18, 20, 22, 24, 26, 28, 30, 32 and/or 34; and
(d) a variant of an amino acid sequence selected from the group consisting of SEQ ID NOS:2, 4, 6, 8, 10, 12, 14, 16, 18, 20, 22, 24, 26, 28, 30, 32 and/or 34 wherein one or more amino acid residues in said variant differs from the amino acid sequence of said mature form, provided that said variant differs in no more than 15% of amino acid residues from said amino acid sequence.

2 The polypeptide of claim 1, wherein said polypeptide comprises the amino acid sequence of a naturally-occurring allelic variant of an amino acid sequence selected from the group consisting of SEQ ID NOS:2, 4, 6, 8, 10, 12, 14, 16, 18, 20, 22, 24, 26, 28, 30, 32 and/or 34.

3. The polypeptide of claim 2, wherein said allelic variant comprises an amino acid sequence that is the translation of a nucleic acid sequence differing by a single nucleotide from a nucleic acid sequence selected from the group consisting of SEQ ID NOS:1, 3, 5, 7, 9, 11, 13, 15, 17, 19, 21, 23, 25, 27, 29, 31 and/or 33.

4. The polypeptide of claim 1, wherein the amino acid sequence of said variant comprises a conservative amino acid substitution.

5. An isolated nucleic acid molecule comprising a nucleic acid sequence encoding a polypeptide comprising an amino acid sequence selected from the group consisting of: (a) a mature form of an amino acid sequence selected from the group consisting of SEQ ID NOS:2, 4, 6, 8, 10, 12, 14, 16, 18, 20, 22, 24, 26, 28, 30, 32 and/or 34;
(b) a variant of a mature form of an amino acid sequence selected from the group consisting of SEQ ID NOS:2, 4, 6, 8, 10, 12, 14, 16, 18, 20, 22, 24, 26, 28, 30, 32 and/or 34, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS:2, 4, 6, 8, 10, 12, 14, 16, 18, 20, 22, 24, 26, 28, 30, 32 and/or 34;
(d) a variant of an amino acid sequence selected from the group consisting of SEQ ID NOS:2, 4, 6, 8, 10, 12, 14, 16, 18, 20, 22, 24, 26, 28, 30, 32 and/or 34, wherein one or more amino acid residues in said variant differs from the amino acid sequence of said mature form, provided that said variant differs in no more than 15% of amino acid residues from said amino acid sequence;
(e) a nucleic acid fragment encoding at least a portion of a polypeptide comprising an amino acid sequence chosen from the group consisting of SEQ ID NOS:2, 4, 6, 8, 10, 12, 14, 16, 18, 20, 22, 24, 26, 28, 30, 32
and/or 34, or a variant of said polypeptide, wherein one or more amino acid residues in said variant differs from the amino acid sequence of said mature form, provided that said variant differs in no more than 15% of amino acid residues from said amino acid sequence; and
(f) a nucleic acid molecule comprising the complement of (a), (b), (c), (d) or (e).

6. The nucleic acid molecule of claim 5, wherein the nucleic acid molecule comprises the nucleotide sequence of a naturally-occurring allelic nucleic acid variant.

7. The nucleic acid molecule of claim 5, wherein the nucleic acid molecule encodes a polypeptide comprising the amino acid sequence of a naturally-occurring polypeptide variant.

8. The nucleic acid molecule of claim 5, wherein the nucleic acid molecule differs by a single nucleotide from a nucleic acid sequence selected from the group consisting of SEQ ID NOS:1, 3, 5, 7, 9, 11, 13, 15, 17, 19, 21, 23, 25, 27, 29, 31 and/or 33.

9. The nucleic acid molecule of claim 5, wherein said nucleic acid molecule comprises a nucleotide sequence selected from the group consisting of (a) a nucleotide sequence selected from the group consisting of SEQ ID NOS:1, 3, 5, 7, 9, 11, 13, 15, 17, 19, 21, 23, 25, 27, 29, 31 and/or 33;
(b) a nucleotide sequence differing by one or more nucleotides from a nucleotide sequence selected from the group consisting of SEQ ID NOS:1, 3, 5, 7, 9, 11, 13, 15, 17, 19, 21, 23, 25, 27, 29, 31 and/or 33, provided that no more than 20% of the nucleotides differ from said nucleotide sequence;
(c) a nucleic acid fragment of (a); and
(d) a nucleic acid fragment of (b).

10. The nucleic acid molecule of claim 5, wherein said nucleic acid molecule hybridizes under stringent conditions to a nucleotide sequence chosen from the group consisting of SEQ ID NOS:1, 3, 5, 7, 9, 11, 13, 15, 17, 19, 21, 23, 25, 27, 29, 31 and/or 33, or a complement of said nucleotide sequence.

11. The nucleic acid molecule of claim 5, wherein the nucleic acid molecule comprises a nucleotide sequence selected from the group consisting of
(a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence;
(b) an isolated second polynucleotide that is a complement of the first polynucleotide; and
(c) a nucleic acid fragment of (a) or (b).

12. A vector comprising the nucleic acid molecule of claim 11.

13. The vector of claim 12, further comprising a promoter operably-linked to said nucleic acid molecule.

14. A cell comprising the vector of claim 12.

15. An antibody that immunospecifically-binds to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing the sample;
(b) contacting the sample with an antibody that binds immunospecifically to the polypeptide; and
(c) determining the presence or amount of antibody bound to said polypeptide, thereby determining the presence or amount of polypeptide in said sample.

19. A method for determining the presence or amount of the nucleic acid molecule of claim 5 in a sample, the method comprising:
(a) providing the sample;
(b) contacting the sample with a probe that binds to said nucleic acid molecule; and
(c) determining the presence or amount of the probe bound to said nucleic acid molecule, thereby determining the presence or amount of the nucleic acid molecule in said sample.

20. A method of identifying an agent that binds to a polypeptide of claim 1, the method comprising:
(a) contacting said polypeptide with said agent; and
(b) determining whether said agent binds to said polypeptide.

21. A method for identifying an agent that modulates the expression or activity of the polypeptide of claim 1, the method comprising:
(a) providing a cell expressing said polypeptide;
(b) contacting the cell with said agent; and
(c) determining whether the agent modulates expression or activity of said polypeptide,
whereby an alteration in expression or activity of said peptide indicates said agent modulates expression or activity of said polypeptide.

22. A method for modulating the activity of the polypeptide of claim 1, the method comprising contacting a cell sample expressing the polypeptide of said claim with a compound that binds to said polypeptide in an amount sufficient to modulate the activity of the polypeptide.

23. A method of treating or preventing a NOVX-associated disorder, said method comprising administering to a subject in which such treatment or prevention is desired the polypeptide of claim 1 in an amount sufficient to treat or prevent said NOVX-associated disorder in said subject.

24. The method of claim 23, wherein said subject is a human.

25. A method of treating or preventing a NOVX-associated disorder, said method comprising administering to a subject in which such treatment or prevention is desired the nucleic acid of claim 5 in an amount sufficient to treat or prevent said NOVX-associated disorder in said subject.

26. The method of claim 25, wherein said subject is a human.

27. A method of treating or preventing a NOVX-associated disorder, said method comprising administering to a subject in which such treatment or prevention is desired the antibody of claim 15 in an amount sufficient to treat or prevent said NOVX-associated disorder in said subject.

28. The method of claim 27, wherein the subject is a human.

29. A pharmaceutical composition comprising the polypeptide of claim 1 and a pharmaceutically-acceptable carrier.

30. A pharmaceutical composition comprising the nucleic acid molecule of claim 5 and a pharmaceutically-acceptable carrier.

31. A pharmaceutical composition comprising the antibody of claim 15 and a pharmaceutically-acceptable carrier.

32. A kit comprising in one or more containers, the pharmaceutical composition of claim 29.

33. A kit comprising in one or more containers, the pharmaceutical composition of claim 30.

34. A kit comprising in one or more containers, the pharmaceutical composition of claim 31.

35. The use of a therapeutic in the manufacture of a medicament for treating a syndrome associated with a human disease, the disease selected from a NOVX-associated disorder, wherein said therapeutic is selected from the group consisting of a NOVX polypeptide, a NOVX nucleic acid, and a NOVX antibody.

36. A method for screening for a modulator of activity or of latency or predisposition to a NOVX-associated disorder, said method comprising:
(a) administering a test compound to a test animal at increased risk for a NOVX-associated disorder, wherein said test animal recombinantly expresses the polypeptide of claim 1;
(b) measuring the activity of said polypeptide in said test animal after administering the compound of step (a);
(c) comparing the activity of said protein in said test animal with the activity of said polypeptide in a control animal not administered said polypeptide, wherein a change in the activity of said polypeptide in said test animal relative to said control animal indicates the test compound is a modulator of latency of or predisposition to a NOVX-associated disorder.

37. The method of claim 36, wherein said test animal is a recombinant test animal that expresses a test protein transgene or expresses said transgene under the control of a promoter at an increased level relative to a wild-type test animal, and wherein said promoter is not the native gene promoter of said transgene.

38. A method for determining the presence of or predisposition to a disease associated with altered levels of the polypeptide of claim 1 in a first mammalian subject, the method comprising:
(a) measuring the level of expression of the polypeptide in a sample from the first mammalian subject; and
(b) comparing the amount of said polypeptide in the sample of step (a) to the amount of the polypeptide present in a control sample from a second mammalian subject known not to have, or not to be predisposed to, said disease,
wherein an alteration in the expression level of the polypeptide in the first subject as compared to the control sample indicates the presence of or predisposition to said disease.

39. A method for determining the presence of or predisposition to a disease associated with altered levels of the nucleic acid molecule of claim 5 in a first mammalian subject, the method comprising:
(a) measuring the amount of the nucleic acid in a sample from the first mammalian subject; and
(b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

40. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising an amino acid sequence of at least one of SEQ ID NOS:2, 4, 6, 8, 10, 12, 14, 16, 18, 20, 22, 24, 26, 28, 30, 32 and/or 34, or a biologically active fragment thereof.

41. A method of treating a pathological state in a mammal, the method comprising administering to the mammal the antibody of claim 15 in an amount sufficient to alleviate the pathological state.
